(19) United States
(12) Patent Application Publication
(10) Pub. No.: US 2021/0122763 A1
(43) Pub. Date: Apr. 29, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Ana Isabel De Lucas Olivares, Bargas (ES); Francisca Delgado-Jiménez, Madrid (ES); Susana Conde-Ceide, Toledo (ES); Juan Antonio Vega Ramiro, Olias del Rey (ES)

(21) Appl. No.: 17/253,464

(22) PCT Filed: Jun. 20, 2019
(86) PCT No.: PCT/EP2019/066388

§ 371 Date: Dec. 17, 2020

(30) Foreign Application Priority Data
Jun. 20, 2018 (EP) 18382449,9

(51) Int. Cl. C07D 498/04; C07D 491/056; C07D 491/048; C07D 513/04; C07D 405/14; C07D 491/052

(52) U.S. Cl. CPC C07D 498/04; C07D 491/056; C07D 491/052; C07D 513/04; C07D 405/14; C07D 491/048

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

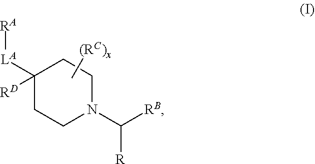

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in then-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain.

Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GcNAcylation of tau regulates the extent of phosphorylation as serine or threonine residues carrying 0-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (AD) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2008/012623 (Pfizer Prod. Inc., published 31 Jan. 2008) discloses 2-[(4-phenyl-1-piperidyl)methyl]-1H-benzimidazole and 2-[(3-phenylpyrrolidin-1-yl)methyl]-1H-benzimidazole derivatives and as an exception, 2-(3-benzylpyrrolidin-1-yl)methyl]-1H-benzimidazole as mGuR2 potentiators.

WO2007/115077 (AstraZeneca A. B. and NPS Pharma Inc., published 11 Oct. 2007) discloses mainly 1H-benzimidazol-2-ylmethyl substituted 4-piperidines and 3-pyrrolidines, bearing at the 4- or 3-position respectively a phenylalkyl substituent, such as for example, 2-[3-(4-fluorobenzyl)-piperidin-1-ylmethyl]-1-methyl-1H-benzoimidazole, as mGuR potentiators.

WO03/092678 (Schering A G, published 13 Nov. 2007) describes substituted imidazole derivatives as NOS inhibitors, and describes (3S)-3-(4-aminophenoxy)-1-[(1,3-benzodioxol-5-yl)methyl]piperidine as an intermediate of synthesis.

WO93/21181 (Merck Sharp & Dohme, published 28 Oct. 1993) discloses Tachykinin antagonists. Particular example 6, 2-[{(2R*,3R*)-3-((3,5-bis(trifluoromethyl)phenyl)methyloxy)-2-phenylpiperidino}methyl]benzimidazole, requires a phenyl substituent at the piperidine.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors.

WO2014/159234 (Merck Patent GMBH, published 2 Oct. 2014) discloses mainly 4-phenyl or benzyl-piperidine and piperazine compounds substituted at the 1-position with an acetamido-thiazolylmethyl or acetamidoxazolylmethyl substituent and the compound N-[5-[(3-phenyl-1-piperidyl)methyl]thiazol-2-yl]acetamide;

WO2016/0300443 (Asceneuron S. A., published 3 Mar. 2016), WO2017/144633 and

WO2017/0114639 (Asceneuron S. A., published 31 Aug. 2017) disclose 1,4-disubstituted piperidines or piperazines as OGA inhibitors;

WO2017/144637 (Asceneuron S. A, published 31 Aug. 2017.) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors;

WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

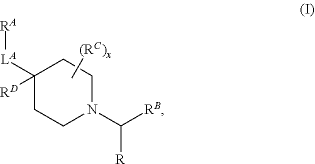

and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; or is phenyl; each of which may be optionally substituted with 1, 2 or 3 substituents, in particular 2 substituents, each independently selected from the group consisting of halo; cyano; OH; C₁₋₄alkyl optionally substituted with 1, 2, or 3 independently selected halo substituents; C₃₋₆cycloalkyl; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;

L^{A} is selected from the group consisting of a covalent bond, —CH₂—, —O—, —OCH₂—, —CH₂O—, —NH—, —N(CH₃)—, —NHCH₂— and —CH₂NH—;

R is H or CH₃; and

R^{B} is an aromatic heterobicyclic radical selected from the group consisting of (b-1) to (b-6)

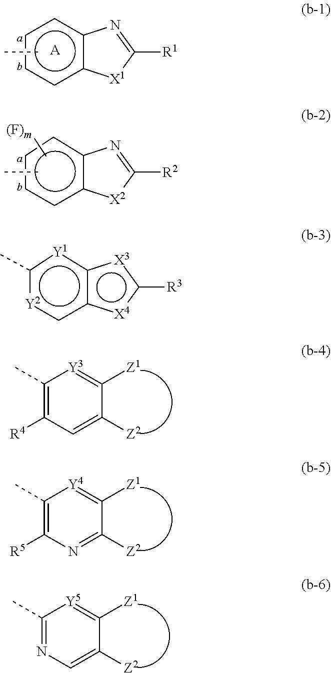

wherein

a and b represent the position of attachment to CHR;

ring A represents a 6-membered aromatic ring optionally having one Nitrogen atom; X¹ and X² each represent S or O;

m represents 1 or 2;

Y¹ and Y² are each independently selected from N and CF; with the proviso that when Y¹ is N, Y² is CF, and when Y¹ is CF, Y² is N;

X³ and X⁴ are each independently selected from N, S and O; with the proviso that when X³ is N then X⁴ is S or O, and when X⁴ is N then X³ is S or O;

Y³, Y⁴ and Y⁵ each represent CH, CF or N;

—Z¹-Z²— forms a bivalent radical selected from the group consisting of

—O(CH₂)ₙO— (c-1);

—O(CH₂)ₚ— (c-2);

—(CH₂)ₚO— (c-3);

—O(CH₂)_{q}NR⁶— (c-4);

—NR⁶(CH₂)_{q}O— (c-5);

wherein

n represents 1 or 2;

p represents 2 or 3;

q represents 2 or 3; in particular 2;

R¹, R², and R³ are each selected from C₁₋₄alkyl;

R⁴ and R⁵ are each selected from the group consisting of hydrogen, fluoro and methyl;

R⁶ represents hydrogen or C₁₋₄alkyl; in particular hydrogen;

R^{C} is selected from the group consisting of fluoro, methyl, hydroxy, methoxy, trifluoromethyl, and difluoromethyl;

R^{D} is selected from the group consisting of hydrogen, fluoro, methyl, hydroxy, methoxy, trifluoromethyl, difluoromethyl, and fluoromethyl; and

x represents 0, 1 or 2;

with the provisos that
a) R^{C} is not hydroxy or methoxy when present at the carbon atom adjacent to the nitrogen atom of the piperidinediyl ring;
b) R^{C} and R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;

and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as referred to herein, and the tautomers and the stereoisomeric forms thereof, wherein and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; each of which may be optionally substituted with 1, 2 or 3 substituents, in particular 2 substituents, each independently selected from the group consisting of halo; cyano; OH; C₁₋₄alkyl optionally substituted with 1, 2, or 3 independently selected halo substituents; C₃₋₆cycloalkyl; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents; or is phenyl optionally substituted with 1, 2 or 3 substituents, each independently selected from the group consisting of halo and C₁₋₄alkyl;

and the pharmaceutically acceptable salts and the solvates thereof.

In a particular embodiment, the invention is directed to compounds of Formula (I) as referred to herein, and the tautomers and the stereoisomeric forms thereof, wherein and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents, in particular 2 substituents, each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents; L^{A} is selected from the group consisting of a covalent bond, —CH₂—, —O—, —OCH₂—, —CH₂O—, —NH—, —N(CH₃)—, —NHCH₂— and —CH₂NH—; R is H or CH₃; and

R^{B} is an aromatic heterobicyclic radical selected from the group consisting of (b-1) to (b-6)

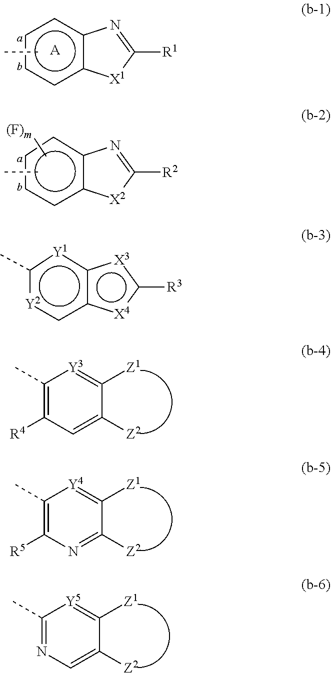

wherein

a and b represent the position of attachment to CHR;

ring A represents a 6-membered aromatic ring optionally having one Nitrogen atom;

X¹ and X² each represent S or O;

m represents 1 or 2;

Y¹ and Y² are each independently selected from N and CF; with the proviso that

when Y¹ is N, Y² is CF, and when Y¹ is CF, Y² is N;

X³ and X⁴ are each independently selected from N, S and O; with the proviso that when X³ is N then X⁴ is S or O, and when X⁴ is N then X³ is S or O;

Y³, Y⁴ and Y⁵ each represent CH, CF or N;

—Z¹-Z²— forms a bivalent radical selected from the group consisting of

—O(CH₂)ₙO— (c-1);

—O(CH₂)ₚ— (c-2);

—(CH₂)ₚO— (c-3);

wherein

n represents 1 or 2;

p represents 2 or 3;

R¹, R², and R³ are each selected from C₁₋₄alkyl;

R⁴ and R⁵ are each selected from the group consisting of hydrogen, fluoro and methyl;

R^{C} is selected from the group consisting of fluoro, methyl, hydroxy, methoxy, trifluoromethyl, and difluoromethyl;

R^{D} is selected from the group consisting of hydrogen, fluoro, methyl, hydroxy, methoxy, trifluoromethyl, and difluoromethyl; and

x represents 0, 1 or 2;

with the provisos that
a) R^{C} is not hydroxy or methoxy when present at the carbon atom adjacent to the nitrogen atom of the piperidinediyl ring;
b) R^{C} and R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;

and the pharmaceutically acceptable salts and the solvates thereof.

In a particular embodiment, the invention is directed to compounds of Formula (I) as referred to herein, and the tautomers and the stereoisomeric forms thereof, wherein R^{A} is a heteroaryl radical selected from the group consisting of pyridin-4-yl, pyrimidin-4-yl, and pyrazin-2-yl, each of which may be optionally substituted with 1, 2 or 3 substituents, in particular 2 substituents, each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents; and the pharmaceutically acceptable salts and the solvates thereof.

In a particular embodiment, the invention is directed to compounds of Formula (I), as referred to herein, and the tautomers and the stereoisomeric forms thereof, wherein R^{A} is a heteroaryl radical selected from the group consisting of pyridin-4-yl and pyrimidin-4-yl, each of which may be optionally substituted with 1, 2 or 3 substituents, in particular 2 substituents, each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In a further embodiment, the invention is directed to compounds of Formula (I), as referred to herein, and the tautomers and the stereoisomeric forms thereof, wherein R^{A} is a heteroaryl radical selected from the group consisting of pyridin-4-yl and pyrimidin-4-yl, each of which may be optionally substituted with 1 or 2 substituents, in particular 2 substituents, each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In a further embodiment, the invention is directed to compounds of Formula (I), as referred to herein, and the tautomers and the stereoisomeric forms thereof, wherein R^{A} is a heteroaryl radical selected from the group consisting of pyridin-4-yl and pyrimidin-4-yl, each of which may be optionally substituted with 1 or 2 substituents, in particular 2 substituents, each independently selected from the group consisting of C₁₋₄alkyl optionally substituted with 1, 2, or 3 independently selected halo substituents;

and the pharmaceutically acceptable salts and the solvates thereof.

In an additional embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, —NH—, —N(CH₃)—, —NHCH₂— and —CH₂NH—;

and the pharmaceutically acceptable salts and the solvates thereof.

In a further embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, and —NH—;

and the pharmaceutically acceptable salts and the solvates thereof.

In a further embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, and —NHCH₂—;

and the pharmaceutically acceptable salts and the solvates thereof.

In another embodiment, the invention is directed to compounds of Formula (I), as referred to herein, and the tautomers and the stereoisomeric forms thereof, wherein L^{A} is selected from the group consisting of —CH₂—, —O—, —OCH₂—, and —CH₂O—; and the pharmaceutically acceptable salts and the solvates thereof.

In a further embodiment, the invention is directed to compounds of Formula (I), as referred to herein, and the tautomers and the stereoisomeric forms thereof, wherein L^{A} is —O—; and the pharmaceutically acceptable salts and the solvates thereof.

In an additional embodiment, the invention is directed to compounds of Formula (I), as referred to herein, and the tautomers and the stereoisomeric forms thereof, wherein R^{B} is an aromatic heterobicyclic radical selected from the group consisting of (b-1), (b-2), (b-3), (b-4) and (b-5);

and the pharmaceutically acceptable salts and the solvates thereof.

In an additional embodiment, the invention is directed to compounds of Formula (I), as referred to herein, and the tautomers and the stereoisomeric forms thereof, wherein R^{B} is an aromatic heterobicyclic radical selected from the group consisting of (b-1), (b-2), (b-4) and (b-5); wherein

—Z¹-Z²— forms a bivalent radical selected from the group consisting of (c-1) and (c-2), wherein n and p each represent 2;

and the pharmaceutically acceptable salts and the solvates thereof.

In an additional embodiment, the invention is directed to compounds of Formula (I), as referred to herein, and the tautomers and the stereoisomeric forms thereof, wherein R^{B} is an aromatic heterobicyclic radical selected from the group consisting of (b-3) and (b-4); wherein

—Z¹-Z²— forms a bivalent radical selected from the group consisting of (c-1) and (c-2), wherein n and p each represent 2; and wherein Y¹ is N, Y² is CF, and R³ is C₁₋₄alkyl;

and the pharmaceutically acceptable salts and the solvates thereof.

In an additional embodiment, the invention is directed to compounds of Formula (I), as referred to herein, and the tautomers and the stereoisomeric forms thereof, wherein

R^{B} is an aromatic heterobicyclic radical selected from the group consisting of (b-1), (b-2), (b-4) and (b-5); wherein

X¹ and X² represent S;

Y³ represents CH or N;

—Z¹-Z²— forms a bivalent radical selected from the group consisting of (c-1) and (c-2), wherein n and p each represent 2;

R¹ and R² are each selected from C₁₋₄alkyl; and

R⁴ and R⁵ each represent hydrogen or fluoro;

and the pharmaceutically acceptable salts and the solvates thereof.

In yet another embodiment, the invention is directed to compounds of Formula (I), as referred to herein, and the tautomers and the stereoisomeric forms thereof, wherein

R^{B} is an aromatic heterobicyclic radical selected from the group consisting of

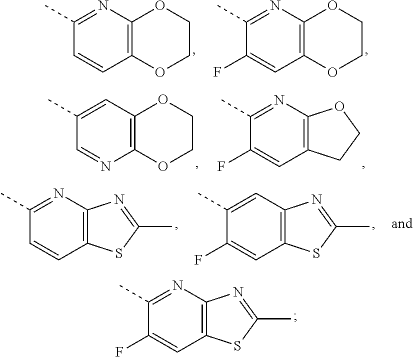

and the pharmaceutically acceptable salts and the solvates thereof.

In yet another embodiment, the invention is directed to compounds of Formula (I), as referred to herein, and the tautomers and the stereoisomeric forms thereof, wherein

R^{B} is an aromatic heterobicyclic radical selected from the group consisting of

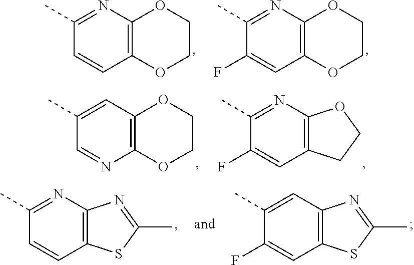

and the pharmaceutically acceptable salts and the solvates thereof.

In yet another embodiment, the invention is directed to compounds of Formula (I), as referred to herein, and the tautomers and the stereoisomeric forms thereof, wherein

R^{B} is an aromatic heterobicyclic radical selected from the group consisting of

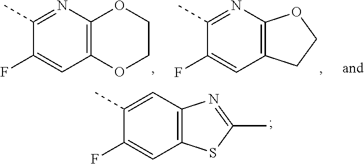

and the pharmaceutically acceptable salts and the solvates thereof.

In an additional embodiment, the invention is directed to compounds of Formula (I), as referred to herein, and the tautomers and the stereoisomeric forms thereof, wherein x is 0 or 1; and R^{C} when present, is fluoro or methyl, in particular methyl; and the pharmaceutically acceptable salts and the solvates thereof.

In an additional embodiment, the invention is directed to compounds of Formula (I), as referred to herein, and the tautomers and the stereoisomeric forms thereof, wherein x is 0; and the pharmaceutically acceptable salts and the solvates thereof.

In an additional embodiment, the invention is directed to compounds of Formula (I), as referred to herein, and the tautomers and the stereoisomeric forms thereof, wherein

R^{D} is hydrogen; and the pharmaceutically acceptable salts and the solvates thereof.

In a particular embodiment, the invention is directed to compounds of Formula (I) as referred to herein, and the tautomers and the stereoisomeric forms thereof, wherein and the tautomers and the stereoisomeric forms thereof, wherein

R^{A} is pyridin-4-yl or pyrimidin-4-yl, each of which may be optionally substituted with 1, 2 or 3 substituents, in particular 1 or 2 substituents, each independently selected from the group consisting of C₁₋₄alkyl optionally substituted with 1, 2, or 3 independently selected halo substituents;

L^{A} is selected from the group consisting of a —CH₂—, —O—, —OCH₂—,

—CH₂O—, and —NH—;

R is CH₃; and

R^{B} is an aromatic heterobicyclic radical selected from the group consisting of (b-1) to (b-6)

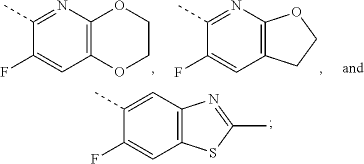

R^{D} is hydrogen; and

x represents 0;

and the pharmaceutically acceptable salts and the solvates thereof.

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₃₋₆cycloalkyl” shall denote cyclopropyl, cyclobutyl, cyclopentyl, and cyclohexyl; “C₁₋₄alkyloxy” shall denote an ether radical wherein C₁₋₄alkyl is as defined before. When reference is made to L^{A}, the definition is to be read from left to right, with the left part of the linker bound to R^{A} and the right part of the linker bound to the pyrrolidinediyl or piperidinediyl ring. Thus, when L^{A} is, for example, —O—CH₂—, then R^{A}-L^{A}- is R^{A}—O—CH₂—. When R^{C} is present more than once, where possible, it may be bound at the same carbon atom of the pyrrolidinediyl or piperidinediyl ring, and each instance may be different.

In general, whenever the term “substituted” is used in the present invention, it is meant, unless otherwise indicated or is clear from the context, to indicate that one or more hydrogens, in particular 1 to 3 hydrogens, preferably 1 or 2 hydrogens, more preferably 1 hydrogen, on the atom or radical indicated in the expression using “substituted” are replaced with a selection of substituents from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration.

Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds of Formula (I) can be prepared by reacting an intermediate compound of Formula (II) with a compound of Formula (III) followed by reaction of the formed imine derivative with an intermediate compound of Formula (IV) according to reaction scheme (1). The reaction is performed in a suitable reaction-inert solvent, such as, for example, anhydrous dichloromethane, a Lewis acid, such as, for example titanium tetraisopropoxide, under thermal conditions, such as, 0° C. to room temperature, for example, 0° C. or room temperature, for a sufficient period of time to drive the reaction to completion, for example for 1 hour to 24 hours. In reaction scheme (1) all variables are defined as in Formula (I), and wherein halo is chloro, bromo or iodo.

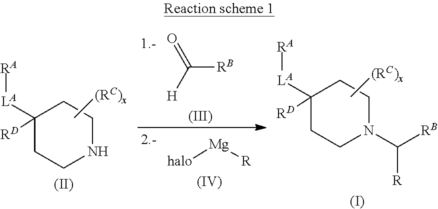

### Experimental Procedure 2

Additionally, final compounds of Formula (I) can be prepared by reacting an intermediate compound of Formula (II) with a compound of Formula (V) according to reaction scheme (2). The reaction is performed in a suitable reaction-inert solvent, such as, for example, acetonitrile, a suitable base, such as, for example, potassium carbonate, under thermal conditions, such as, room temperature to 70° C., for example room temperature or 70° C., for a sufficient period of time to drive the reaction to completion, for example for 1 hour to 24 hours. In reaction scheme (2) all variables are defined as in Formula (I), and wherein halo is chloro, bromo or iodo.

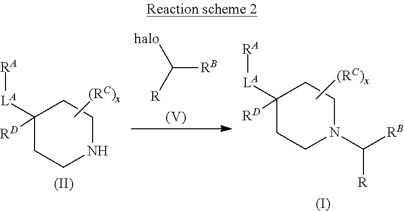

### Experimental Procedure 3

Intermediate compounds of Formula (II) can be prepared by cleaving a protecting group in an intermediate compound of Formula (VI) according to reaction scheme (3). In reaction scheme (3) all variables are defined as in Formula (I), and PG is a suitable protecting group of the nitrogen function such as, for example, tert-butoxycarbonyl (Boc). Suitable methods for removing such protecting groups are widely known to the person skilled in the art and comprise but are not limited to, treatment with a protic acid, such as, for example, trifluoroacetic acid, in a reaction inert solvent, such as, for example, 1,4-dioxane or with an acidic resin, such as for example, Amberlist® 15 hydrogen form in a reaction inert solvent such as methanol. In reaction scheme (3) all variables are defined as in Formula (I).

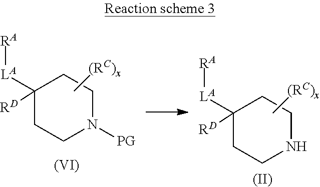

### Experimental Procedure 4

Intermediate compounds of Formula (VI) wherein L^{A} is —O— or —O—CH₂— can be prepared by reaction of an intermediate compound of Formula (VII) with a halo compound of Formula (VIII) according to reaction scheme (4). The reaction is performed in a suitable reaction-inert solvent, such as, for example, dimethylsulfoxide or dimethylformamide, and a suitable base, such as, for example, potassium or sodium tert-butoxide, sodium hydride or potassium carbonate, under thermal conditions, such as, room temperature to 70° C., for example at room temperature or 70° C., for a sufficient period of time to drive the reaction to completion, for example for 1 hour or 48 hours. In reaction scheme (4) all variables are defined as in Formula (I), PG is a suitable protecting group of the nitrogen function such as, for example, tert-butoxycarbonyl (Boc) and halo is chloro, bromo or iodo.

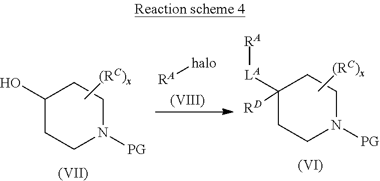

### Experimental Procedure 5

Intermediate compounds of Formula (VI) wherein L^{A} is —O— or —O—CH₂— can be prepared by reaction of an intermediate compound of Formula (VII) with a hidroxy compound of Formula (IX) under Mitsunobu reaction condition according to reaction scheme (5). The reaction is performed in a suitable reaction-inert solvent, such as, for example, THF, in the presence of a phosphine reagent, such as triphenylphospine, and a coupling reagent such as DIAD or DBAD, under thermal conditions, such as, room temperature to 120° C., for example at room temperature or 120° C., for a sufficient period of time to drive the reaction to completion, for example for 1 hour or 48 hours. In reaction scheme (5) all variables are defined as in Formula (I), PG is a suitable protecting group of the nitrogen function such as, for example, tert-butoxycarbonyl (Boc).

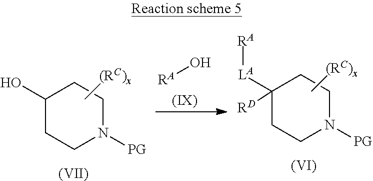

### Experimental Procedure 6

Intermediate compounds of Formula (VI) wherein L^{A} is or —CH₂—O— can be prepared by reaction of an intermediate compound of Formula (X) with a hidroxy compound of Formula (IX) under Mitsunobu reaction condition according to reaction scheme (5). The reaction is performed in a suitable reaction-inert solvent, such as, for example, THF, in the presence of a phosphine reagent, such as triphenylphospine, and a coupling reagent such as DIAD or DBAD, under thermal conditions, such as, room temperature to 120° C., for example at room temperature or 120° C., for a sufficient period of time to drive the reaction to completion, for example for 4 hour or 48 hours. In reaction scheme (6) all variables are defined as in Formula (I), PG is a suitable protecting group of the nitrogen function such as, for example, tert-butoxycarbonyl (Boc).

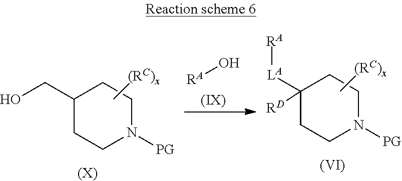

### Experimental Procedure 7

Intermediate compounds of Formula (VI) wherein L^{A} is —CH₂—O— can be prepared by reaction of an intermediate compound of Formula (X) with a halo compound of Formula (VIII) according to reaction scheme (4). The reaction is performed in a suitable reaction-inert solvent, such as, for example, dimethylsulfoxide or dimethylformamide, in the presence of a suitable base, such as, for example, potassium or sodium tert-butoxide, sodium hydride or potassium carbonate, under thermal conditions, such as, room temperature to 70° C., for example at room temperature or 70° C., for a sufficient period of time to drive the reaction to completion, for example for 1 hour or 48 hours. In reaction scheme (7) all variables are defined as in Formula (I), PG is a suitable protecting group of the nitrogen function such as, for example, tert-butoxycarbonyl (Boc) and halo is chloro, bromo or iodo.

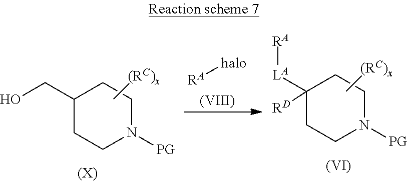

### Experimental Procedure 8

Intermediate compounds of Formula (VI) wherein L^{A} is —NH— can be prepared by reaction of an intermediate compound of Formula (XI) with a halo compound of Formula (VIII) according to reaction scheme (8). The reaction is performed in a suitable reaction-inert solvent, such as, for example, toluene, in the presence of a suitable base, such as, for example, potassium or sodium tert-butoxide, a suitable catalyst, such as for example, Pd₂dba₃, and a suitable phosphine, such as for example, XPhos, under thermal conditions, such as for example 120° C., for a sufficient period of time to drive the reaction to completion, for example for or 14 hours. In reaction scheme (8) all variables are defined as in Formula (I), PG is a suitable protecting group of the nitrogen function such as, for example, tert-butoxycarbonyl (Boc) and halo is chloro, bromo or iodo.

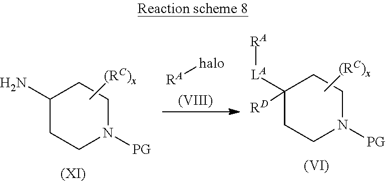

Intermediates of Formula (III), (IV), (V), (VII), (VIII), (IX), (X), (XI) are commercially available or can be prepared by known procedures to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and tauopathy diseases: In recent years the United States (US) National Institute for Aging and the International Working Group have proposed guidelines to better define the preclinical (asymptomatic) stages of AD (Dubois B, et al. Lancet Neurol. 2014; 13:614-629;

Sperling, R A, et al. Alzheimers Dement. 2011; 7:280-292). Hypothetical models postulate that A accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of A or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases A production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “min” means minutes, “h” means hours, “ACN” “CH₃CN” or “MeCN” mean acetonitrile, “aq.” means aqueous, “t-BuOH” means tert-butanol, “DMF” means dimethylformamide, “DMSO” means dimethylsulfoxide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “iPrOH” means isopropyl alcohol, “iPrNH₂” means isopropyl amine, “t-PrOH” means tert-butyl alcohol, “RP” means reversed phase, “Rₜ” means retention time (in minutes), “[M+H]⁺” means the protonated mass of the free base of the compound, “wt” means weight, “THF” means tetrahydrofuran, “EtOAc” means ethyl acetate, “DCM” means dichloromethane, “MeOH” means methanol, “sol.” means solution, “EtOH” means ethanol, “TFA” means trifluoroacetic acid, “TBAF” means tetrabutylammonium fluoride, “DMAP” means 4-(dimethylamino)pyridine, “NaH” means sodium hydride, “DIAD” means diisopropyl azodicarboxylate, “DBAD” means di-tert-butyl azodicarboxylate, “NaOtBu” means sodium tert-butoxide, “tBuOK” means potassium tert-butoxide, “Pd(OAc)₂” means palladium(II) acetate, “Pd₂dba₃” means tris(dibenzylideneacetone)dipalladium(0), “PdCl₂(PPh₃)₂” means bis(triphenylphosphine)palladium(II)dichloride, “PdCl₂(dppf)” means[1,1′-bis(diphenylphosphino)ferrocene]dichloropalladium(II), “m-CPBA” means 3-chloroperbenzoic acid, “XPhos” means 2-dicyclohexylphosphino-2′,4′,6′-triisopropylbiphenyl, “DMA” means N,N-dimethylacetamide, “NMP” means methylpyrrolidinone, “Dppf” means 1,1′-ferrocenediyl-bis(diphenylphosphine), “Me-THF” means 2-methyltetrahydrofuran, “n-BuLi” means n-butyl lithiu, “LiHMDS” means lithium bis(trimethylsilyl)amide, “Et₃N” means triethylamine, “AIBN” means 2,2′-azobis(2-methylpropionitrile), “DAST” means (diethylamino)sulfur trifluoride, “Ti(Oi-Pr)₄” means titanium(IV) isopropoxide. Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “*R” or “*S” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Thin layer chromatography (TLC) was carried out on silica gel 60 F254 plates (Merck) using reagent grade solvents.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or 971-FP systems from Agilent, or Isolera lSV systems from Biotage.

### Preparation of the Intermediates

### Preparation of Intermediate 1

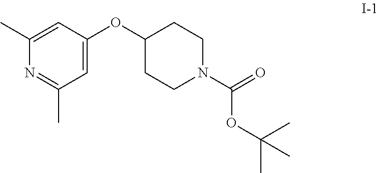 [SMILES: Cc1cc(OC2CCN(C(=O)OC(C)(C)C)CC2)cc(C)n1]

Method 1: potassium tert-butoxide (CAS: 865-47-4, 1.62 g, 14.41 mmol) was added portionwise to a stirred solution of tert-butyl 4-hydroxypiperidine-1-carboxylate (CAS: 109384-19-2; 1.45 g, 7.20 mmol) and 4-chloro-2,6-dimethyl-pyridine (CAS: 3512-75-2; 1.02 g, 7.20 mmol) in DMSO (14.5 mL) at rt. The mixture was stirred at 60° C. for 5 h. The residue was diluted with water and extracted with EtOAc. The organic layer was separated, dried (Na₂SO₄), filtered and evaporated in vacuo to yield intermediate 1 (2.31 g, 74%, 71% purity) as a brown syrup, used in the next step without further purification.

Method 2: A solution of tert-butyl 4-hydroxypiperidine-1-carboxylate (CAS: 109384-19-2; 11.82 g, 58.72 mmol) in DMF (20 mL) was added to a stirred suspension of sodium hydride (CAS: 7646-69-7; 60% dispersion in mineral oil, 2.58 g, 64.59 mmol) in DMF (90 mL) at 0° C. under N2. The mixture was stirred for 2 h and then a solution of 4-chloro-2,6-dimethyl-pyridine (CAS: 3512-75-2; 9.15 g, 64.59 mmol) in DMF (20 mL) was added dropwise at 0° C. The mixture was allowed to warm to rt and stirred for 3 days and then at 60° C. for 6 h. After cooling to rt, water was added and the mixture was extracted with EtOAc. The organic layer was separated, dried (Na₂SO₄), filtered and concentrated in vacuo. The residue was purified by flash chromatography (silica; EtOAc in heptane 30/70 to 100/0). The desired fractions were collected and concentrated in vacuo to yield intermediate 1 as colourless oil (2.24 g, 12%) and impure fractions, that were further purified by flash chromatography (silica; 7N solution of NH₃ in MeOH in DCM, 0/100 to 10/90) and then by RP HPLC (stationary phase: C18 XBridge 50×100 mm, 5 μm, mobile phase: gradient from 80% NH₄HCO₃ 0.25% solution in water, 20% CH₃CN to 0% NH₄HCO₃ 0.25% solution in water, 100% CH₃CN). The desired fractions were collected and evaporated in vacuo to yield additional intermediate 1 as colourless oil (3.82 g, 21%).

### Preparation of Intermediate 2

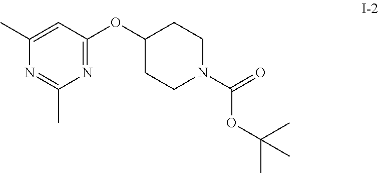 [SMILES: Cc1cc(OC2CCN(C(=O)OC(C)(C)C)CC2)nc(C)n1]

Intermediate 2 was prepared following analogous procedures to Method 1 and Method 2 described for the synthesis of intermediate 1 using tert-butyl 4-hydroxypiperidine-1-carboxylate (CAS: 109384-19-2) and 4-chloro-2,6-dimethyl-pyrimidine (CAS: 4472-45-1) as starting materials.

### Preparation of Intermediate 3

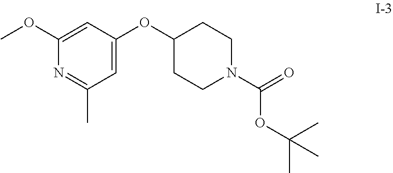 [SMILES: COc1cc(OC2CCN(C(=O)OC(C)(C)C)CC2)cc(C)n1]

Intermediate 3 was prepared following an analogous procedure to the one described as Method 2 for the synthesis of intermediate 1 using tert-butyl 4-hydroxypiperidine-1-carboxylate (CAS: 109384-19-2) and 4-bromo-2-methoxy-6-methylpyridine (CAS: 1083169-00-9) as starting materials.

### Preparation of Intermediate 4

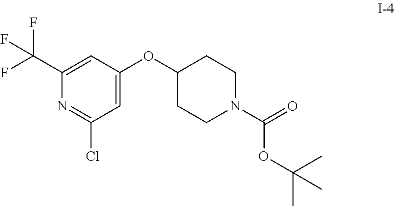 [SMILES: CC(C)(C)OC(=O)N1CCC(Oc2cc(Cl)nc(C(F)(F)F)c2)CC1]

Intermediate 4 was prepared following an analogous procedure to the one described as Method 2 for the synthesis of intermediate 1 using tert-butyl 4-hydroxypiperidine-1-carboxylate (CAS: 109384-19-2) and 2-chloro-4-iodo-6-(trifluoromethyl)pyridine (CAS: 205444-22-0) as starting materials.

### Preparation of Intermediate 5

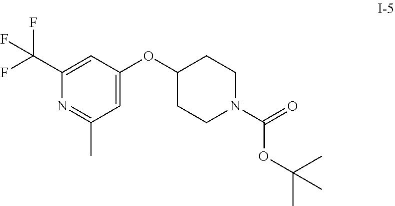 [SMILES: Cc1cc(OC2CCN(C(=O)OC(C)(C)C)CC2)cc(C(F)(F)F)n1]

Pd(OAc)₂ (CAS: 3375-31-3; 46.74 mg, 0.21 mmol) and tricyclohexylphosphonium tetrafluroborate (CAS: 58656-04-5; 153.33 mg, 0.42 mmol) were added to a stirred mixture of intermediate 4 (1.06 g, 2.78 mmol), trimethylboroxine (CAS: 823-96-1; 1.05 mL, 7.49 mmol) and K₂CO₃ (0.77 g, 5.55 mmol) in deoxygenated 1,4-dioxane (8.5 mL). The mixture was stirred at 100° C. for 4 h under N2. After cooling to rt, the mixture was diluted with water and extracted with DCM. The organic layer was separated, dried (MgSO₄), filtered and the solvents evaporated in vacuo. The crude product was purified by flash column chromatography (silica; EtOAc in heptane 0/100 to 30/70). The desired fractions were collected and concentrated in vacuo to yield intermediate 5 as brown oil (0.95 g, 95%).

### Preparation of Intermediate 6

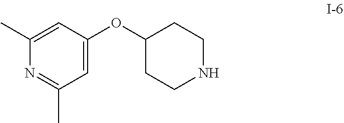

Method 1: Amberlyst® 15 hydrogen form, strongly acidic, cation exchanger resin (CAS: 39389-20-3, 7.78 g, loading 4.7 meq/g) was added to a stirred solution of intermediate 1 (2.24 g, 7.31 mmol) in MeOH (59.3 mL) at rt. The mixture was shaked in a solid phase reactor at rt for 16 h. The resin was filtered and washed with MeOH (this fraction was discarded) and then with a 7N solution of NH₃ in MeOH. The filtrate was concentrated in vacuo to yield intermediate 6 as brown oil, that crystallized upon standing (1.46 g, 97%).

Method 2: Trifluoroacetic acid (CAS: 76-05-1, 5 mL, 65.34 mmol) was added dropwise to a stirred solution of intermediate 1 (2.2 g, 5.46 mmol) in 1,4-dioxane (9.6 mL) at rt. The mixture was stirred at rt for 12 h and then evaporated in vacuo. The residue was dissolved in MeOH and Amberlyst® 15 hydrogen form, strongly acidic, cation exchanger resin (CAS: 39389-20-3, 6.4 g, loading 4.7 meq/g) was added. The mixture was shaked in a solid phase reactor at rt for 3 h. The resin was filtered and washed with MeOH (this fraction was discarded) and then with a 7N solution of NH₃ in MeOH. The filtrate was concentrated in vacuo to yield intermediate 6 as orange oil (0.98 g, 87%).

### Preparation of Intermediate 7

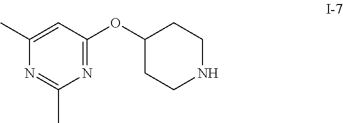

Intermediate 7 was prepared following analogous procedures to Method 1 and Method 2 described for the synthesis of intermediate 6 using intermediate 2 as starting material.

### Preparation of Intermediate 8

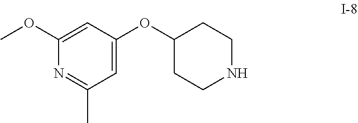 [SMILES: COc1cc(OC2CCNCC2)cc(C)n1]

Intermediate 8 was prepared following an analogous procedure to the one described as Method 1 for the synthesis of intermediate 6 using intermediate 3 as starting material.

### Preparation of Intermediate 9

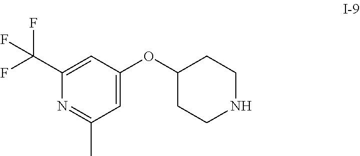 [SMILES: Cc1cc(OC2CCNCC2)cc(C(F)(F)F)n1]

Intermediate 9 was prepared following an analogous procedure to the one described as Method 1 for the synthesis of intermediate 6 using intermediate 5 as starting material.

### Preparation of Intermediate 40

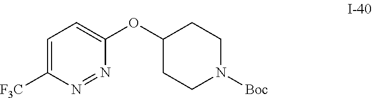 [SMILES: CC(C)(C)OC(=O)N1CCC(Oc2ccc(C(F)(F)F)nn2)CC1]

1-Boc-4-hydroxypiperidine (CAS: 109384-19-2; 200 mg, 1.00 mmol) in anhydrous DMF (2 mL) was added dropwise to a stirred solution of NaH (60% dispersion in mineral oil, 47.8 mg, 1.20 mmol) in anhydrous DMF (2 mL) at 0° C. The mixture was stirred at 0° C. for 30 min and 3-chloro-6-(trifluoromethyl)pyridazine (CAS: 258506-68-2; 200 mg, 1.09 mmol) dissolved in anhydrous DMF (2 mL) was added portionwise at 0° C. The reaction mixture was stirred at 80° C. for 18 h and concentrated in vacuo. The residue was diluted with water and extracted with a mixture of DCM and EtOAc. The combined organic layers were dried (Na₂SO₄), filtered and evaporated in vacuo. The crude product was purified by flash column chromatography (silica, heptane/EtOAc, gradient from 100:0 to 70:30). The desired fractions were collected and concentrated in vacuo to afford intermediate 40 (202 mg, 59%) as a white solid.

### Preparation of Intermediate 41

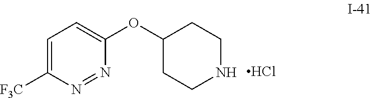 [SMILES: FC(F)(F)c1ccc(OC2CCNCC2)nn1 ; Cl]

HCl (4M in 1,4-dioxane, 1.61 mL, 6.45 mmol) was added to a stirred solution of intermediate 40 (202 mg, 0.58 mmol) in 1,4-dioxane (3.9 mL). The reaction mixture was stirred at room temperature for 20 h. The solvent was evaporated in vacuo to afford intermediate 41 (157 mg, 95%) as a white solid and which was used in next step without further purification.

### Preparation of Intermediate 42

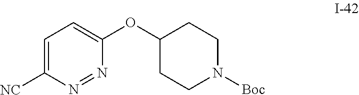 [SMILES: CC(C)(C)OC(=O)N1CCC(Oc2ccc(C#N)nn2)CC1]

Intermediate 42 was prepared following an analogous procedure to the one described for the synthesis of intermediate 40 using 1-Boc-4-hydroxypiperidine (CAS: 109384-19-2) and 6-chloropyridazine-3-carbonitrile (CAS: 35857-89-7) as starting materials. The crude product was purified by flash column chromatography (silica, heptane/EtOAc, gradient from 100:0 to 40:60). The desired fractions were collected and concentrated in vacuo to afford intermediate 42 (843 mg, 85%) as a white solid.

### Preparation of Intermediate 43

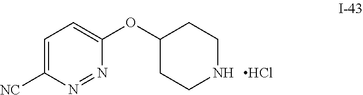 [SMILES: N#Cc1ccc(OC2CCNCC2)nn1 ; Cl]

Intermediate 43 was prepared following an analogous procedure to the one described for the synthesis of intermediate 41 using intermediate 42 as starting material. The crude product was used in the next step without any purification.

### Preparation of Intermediate 44

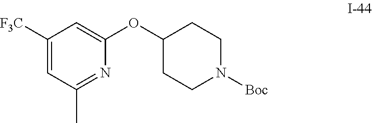 [SMILES: Cc1cc(C(F)(F)F)cc(OC2CCN(C(=O)OC(C)(C)C)CC2)n1]

To a solution of 1-Boc-4-hydroxypiperidine (CAS: 109384-19-2; 500 mg, 2.48 mmol) in anhydrous DMF (9 mL) at room temperature was added NaH (60% dispersion in mineral oi, 119 mg, 2.98 mmol) portion wise. The mixture was stirred for 60 min and 2-chloro-6-methyl-4-(trifluoromethyl)pyridine (CAS: 22123-14-4; 534 mg, 2.73 mmol) was added dropwise. The reaction mixture was stirred at 80° C. for 18 h. The mixture was cooled down and the volatiles were evaporated in vacuo. The residue was taken up in EtOAc and washed with NaHCO₃ (sat., aq.). The organic phase was evaporated in vacuo to give intermediate 44 (1.03 g, 77%, 67% purity) as a brown oil.

### Preparation of Intermediate 45

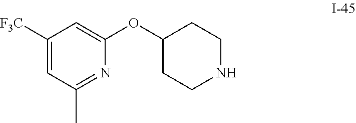 [SMILES: Cc1cc(C(F)(F)F)cc(OC2CCNCC2)n1]

A solution of intermediate 44 (1.49 g, 2.78 mmol, 67% purity) in MeOH (22.6 mL) was added to a solid phase reactor containing Amberlyst®15 hydrogen form (CAS: 39389-20-3; 2.96 g, 13.9 mmol). The mixture was shaken at room temperature for 16 h. The solvent was removed and the resin was washed with MeOH (3 times), filtered and the solvent was discarded. The product was eluted with NH₃ (7N in MeOH) (3 times) to afford intermediate 45 (684 mg, 68%, 72% purity) as a brown oil.

### Preparation of Intermediate 46

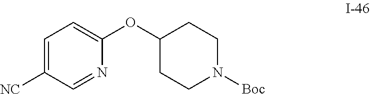 [SMILES: CC(C)(C)OC(=O)N1CCC(Oc2ccc(C#N)cn2)CC1]

Intermediate 46 was prepared following an analogous procedure to the one described for the synthesis of intermediate 44 using 1-Boc-4-hydroxypiperidine (CAS: 109384-19-2) and 6-chloro-3-pyridinecarbonitrile (CAS: 33252-28-7) as starting materials.

### Preparation of Intermediate 47

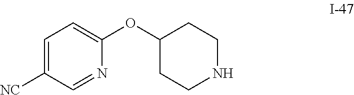 [SMILES: N#Cc1ccc(OC2CCNCC2)nc1]

Intermediate 47 was prepared following an analogous procedure to the one described for the synthesis of intermediate 45 using intermediate 46 as starting material.

### Preparation of Intermediate 48

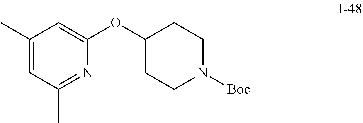 [SMILES: Cc1cc(C)nc(OC2CCN(C(=O)OC(C)(C)C)CC2)c1]

A solution of 1-Boc-4-hydroxypiperidine (CAS: 109384-19-2; 1.00 g, 4.97 mmol) in anhydrous DMF (4.16 mL) was added dropwise to a stirred solution of NaH (60% dispersion in mineral oil, 238 mg, 5.96 mmol) in anhydrous DMF (4.16 mL) at 0° C. The mixture was stirred at 0° C. for 30 min and a solution of 2-chloro-4,6-dimethylpyridine (CAS: 30838-93-8; 0.79 g, 5.47 mmol) in anhydrous DMF (4.16 mL) was added portionwise at 0° C. The reaction mixture was stirred at 60° C. for 16 h and concentrated in vacuo. The residue was diluted with water and extracted with EtOAc. The organic layer was dried (Na₂SO₄), filtered and evaporated in vacuo. The crude mixture was purified by flash column chromatography (silica, heptane/EtOAc, gradient from 100:0 to 70:30) to afford intermediate 48 (1.12 g, 74%) as a white solid.

### Preparation of Intermediate 49

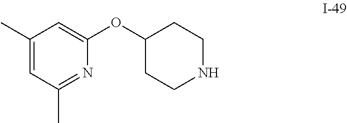

A solution of intermediate 48 (1.12 g, 3.67 mmol) in MeOH (28.1 mL) was added to a closed reactor containing Amberlyst®15 hydrogen form (CAS: 39389-20-3 3.89 g, 18.3 mmol). The mixture was shaken in a solid phase reactor at room temperature for 16 h. The resin was washed with MeOH (the fraction was discarded). NH₃ (7N in MeOH) (25 mL) was added. The mixture was shaken in the solid phase reactor for 2 h. The resin was filtered off and washed with NH₃ (7N in MeOH) (2×25 mL, 30 min shaken). The filtrates were concentrated in vacuo to afford intermediate 49 (763 mg, 87%, 86% purity) as a dark brown oil.

### Preparation of Intermediate 50

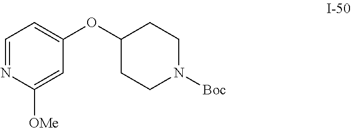 [SMILES: COc1cc(OC2CCN(C(=O)OC(C)(C)C)CC2)ccn1]

Intermediate 50 was prepared following an analogous procedure to the one described for the synthesis of intermediate 48 using 1-Boc-4-hydroxypiperidine (CAS: 109384-19-2) and 4-chloro-2-methoxypyridine (CAS: 72141-44-7) as starting materials. The residue was purified by flash column chromatography (silica, heptane/EtOAc, gradient from 100:0 to 70:30) to afford intermediate 50 (900 mg, 59%) as a white solid.

### Preparation of Intermediate 51

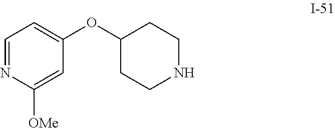 [SMILES: COc1cc(OC2CCNCC2)ccn1]

Intermediate 51 was prepared following an analogous procedure to the one described for the synthesis of intermediate 49 using intermediate 50 as starting material.

### Preparation of Intermediate 52

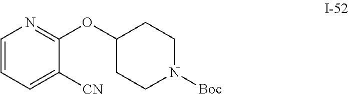 [SMILES: CC(C)(C)OC(=O)N1CCC(Oc2ncccc2C#N)CC1]

Intermediate 52 was prepared following an analogous procedure to the one described for the synthesis of intermediate 48 using 1-Boc-4-hydroxypiperidine (CAS: 109384-19-2) and 2-chloronicotinonitrile (CAS: 6602-54-6) as starting materials.

The residue was purified by flash column chromatography (silica, heptane/EtOAc, gradient from 100:0 to 70:30) to afford intermediate 52 (1.1 g, 73%) as a yellow oil.

### Preparation of Intermediate 53

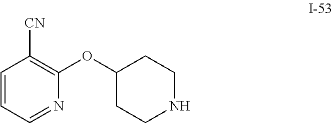 [SMILES: N#Cc1cccnc1OC1CCNCC1]

Intermediate 53 was prepared following an analogous procedure to the one described for the synthesis of intermediate 49 using intermediate 52 as starting material.

### Preparation of Intermediate 54

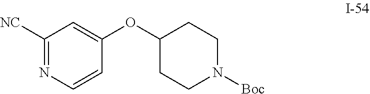 [SMILES: CC(C)(C)OC(=O)N1CCC(Oc2ccnc(C#N)c2)CC1]

Intermediate 54 was prepared following an analogous procedure to the one described for the synthesis of intermediate 48 using 1-Boc-4-hydroxypiperidine (CAS: 109384-19-2) and 4-chloro-pyridine-2-carbonitrile (CAS: 19235-89-3) as starting materials.

The residue was purified by flash column chromatography (silica, heptane/EtOAc, gradient from 100:0 to 70:30) to afford intermediate 54 (650 mg, 43%) as a yellow oil.

### Preparation of Intermediate 55

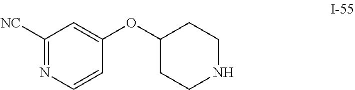 [SMILES: N#Cc1cc(OC2CCNCC2)ccn1]

Intermediate 55 was prepared following an analogous procedure to the one described for the synthesis of intermediate 46 using intermediate 54 as starting material.

### Preparation of Intermediate 56

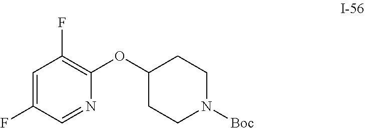 [SMILES: CC(C)(C)OC(=O)N1CCC(Oc2ncc(F)cc2F)CC1]

Intermediate 56 was prepared following an analogous procedure to the one described for the synthesis of intermediate 48 using 1-Boc-4-hydroxypiperidine (CAS: 109384-19-2) and 2,3,5-trifluoropyridine (CAS: 76469-41-5) as starting materials.

The residue was purified by flash column chromatography (silica, heptane/EtOAc, gradient from 100:0 to 70:30) to afford intermediate 56 (580 mg, 37%) as a colourless oil.

### Preparation of Intermediate 57

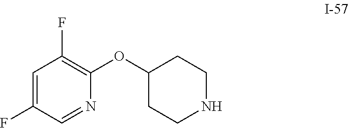 [SMILES: Fc1cnc(OC2CCNCC2)c(F)c1]

Intermediate 57 was prepared following an analogous procedure to the one described for the synthesis of intermediate 49 using intermediate 56 as starting material.

### Preparation of Intermediate 58

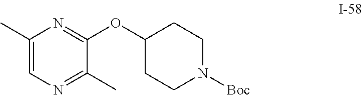 [SMILES: Cc1cnc(C)c(OC2CCN(C(=O)OC(C)(C)C)CC2)n1]

To a solution of 1-Boc-4-hydroxypiperidine (CAS: 109384-19-2; 250 mg, 1.24 mmol) in anhydrous DMF (4.2 mL) under N₂ atmosphere were added NaH (60% dispersion in mineral oil, 59.6 mg, 1.49 mmol) and 15-crown-5 (248 μL, 1.49 mmol). 3-Chloro-2,5-dimethylpyrazine (CAS: 95-89-6; 165 μL, 1.37 mmol) was added and the reaction mixture was stirred at 80° C. The mixture was diluted with water at 0° C. and extracted with DCM. The organic layer was dried, filtered and the solvents were concentrated in vacuo. The crude mixture was purified by flash column chromatography (silica, heptane/EtOAc, gradient from 100:0 to 40:60) to afford intermediate 58 (256 mg, 67%) a colourless oil.

### Preparation of Intermediate 59

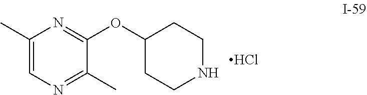

HCl (4M in 1,4-dioxane, 2.50 mL, 10.0 mmol) was added to a stirred solution of intermediate 58 (256 mg, 0.83 mmol) in 1,4-dioxane (7.1 mL). The reaction mixture was stirred at room temperature for 20 h. Then solvent was concentrated in vacuo to give intermediate 59 (195 mg, 96%) which was used as such in the next step.

### Preparation of Intermediate 60

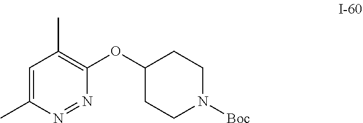 [SMILES: Cc1cc(C)c(OC2CCN(C(=O)OC(C)(C)C)CC2)nn1]

Intermediate 60 was prepared following an analogous procedure to the one described for the synthesis of intermediate 58 using 1-Boc-4-hydroxypiperidine (CAS: 109384-19-2) and 3-chloro-4,6-dimethylpyridazine (CAS: 17258-26-3) as starting materials.

The crude mixture was purified by flash column chromatography (silica, heptane/EtOAc, gradient from 100:0 to 40:60) to afford intermediate 60 (302 mg, 79%) as a yellow oil.

### Preparation of Intermediate 61

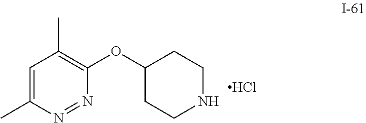

Intermediate 61 was prepared following an analogous procedure to the one described for the synthesis of intermediate 59 using intermediate 60 as starting material. The hydrochloride salt was used in the next step without any purification.

### Preparation of Intermediate 62

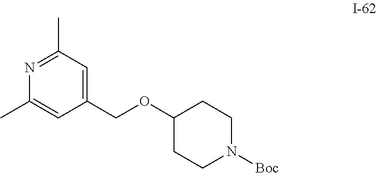 [SMILES: Cc1cc(COC2CCN(C(=O)OC(C)(C)C)CC2)cc(C)n1]

Intermediate 62 was prepared following an analogous procedure to the one described for the synthesis of intermediate 58 using 1-Boc-4-hydroxypiperidine (CAS: 109384-19-2) and 2,6-dimethyl-pyridin-4-ylmethyl chloride (CAS: 120739-87-9) as starting materials. The crude product was purified by RP HPLC (stationary phase: C18 XBridge 30×100 mm 5 μm), mobile phase: (NH₄HCO₃ 0.25% solution in water)/CH₃CN, gradient from 67:33 to 50:50) to afford intermediate 62 (81.5 mg, 16%).

### Preparation of Intermediate 63

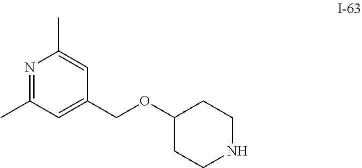 [SMILES: Cc1cc(COC2CCNCC2)cc(C)n1]

HCl (4M in 1,4-dioxane, 0.64 mL, 2.54 mmol) was added to a solution of intermediate 62 (81.5 mg, 0.25 mmol) in 1,4-dioxane (1.99 mL) in a sealed tube. The reaction mixture was stirred at room temperature for 4 h and concentrated in vacuo. The crude mixture was purified by ion exchange chromatography using an Isolute SCX-2 cartridge. The product was eluted with MeOH, then with NH₃ (7N in MeOH). The desired fractions were collected and evaporated in vacuo. The residue was purified by flash column chromatography (silica, MeOH in DCM, gradient from 0/100 to 10/90). The desired fractions were collected and evaporated in vacuo to give intermediate 63 (57.6 mg) as a yellow oil.

The product was converted into the corresponding 2HCl salt by stirring intermediate 63 in 1,4-dioxane in the presence of HCl at Rt for 1 h. The resulting precipitate was filtered, and the filtered cake was dried under vacuum giving intermediate 2HCl as a yellow solid.

### Preparation of Intermediate 64

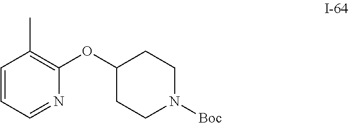 [SMILES: Cc1cccnc1OC1CCN(C(=O)OC(C)(C)C)CC1]

To a mixture of NaH (60% dispersion in mineral oil, 1.75 g, 45.7 mmol) in DMF (30 mL) was added 1-Boc-4-hydroxypiperidine (CAS: 109384-19-2; 5.41 g, 26.9 mmol) portionwise. The mixture was stirred at room temperature for 10 min, and 2-bromo-3-methylpyridine (CAS: 3430-17-9; 1.5 mL, 13.4 mmol) was added. The reaction mixture was heated in the microwave at 150° C. for 10 min. the mixture was diluted with water and extracted with EtOAc. The combined organic extracts were washed with brine, dried (Na₂SO₄), filtered and concentrated in vacuo. The crude mixture was purified by flash column chromatography (silica, DCM/MeOH—NH₃, 95:5) to afford intermediate 64 (2.18 g, 55%).

### Preparation of Intermediate 65

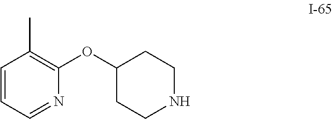

Intermediate 64 (2.18 g, 7.46 mmol) was dissolved in DCM (75 mL) and TFA (10 mL) was added. The reaction mixture was stirred at room temperature for 2 h, and the solvent was removed in vacuo. The crude mixture was dissolved in DCM, washed with NaHCO₃ (sat. aq.), brine, dried (Na₂SO₄), filtered and concentrated in vacuo to afford a first fraction of intermediate 65 (517 mg, 36%). The aqueous phase was extracted with a mixture of EtOAc and THF to afford a second fraction of intermediate 65 (525 mg, 37%).

### Preparation of Intermediate 66

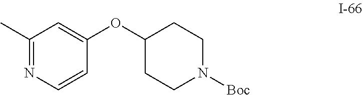 [SMILES: Cc1cc(OC2CCN(C(=O)OC(C)(C)C)CC2)ccn1]

A solution of 1-Boc-4-hydroxypiperidine (CAS: 109384-19-2; 1.00 g, 4.97 mmol) in anhydrous DMF (7 mL) was added dropwise to a stirred solution of NaH (60% dispersion in mineral oil, 238 mg, 5.96 mmol) in anhydrous DMF (7 mL) at 0° C. The mixture was stirred at 0° C. for 30 min and 4-chloro-2-methylpyridine (CAS: 3678-63-5; 697 mg, 5.47 mmol) dissolved in anhydrous DMF (3 mL) was added dropwise at 0° C. The reaction mixture was stirred at 60° C. for 16 h, then at 140° C. for 45 min under microwave irradiation The mixture was concentrated in vacuo and the residue was diluted with water. The aqueous phase was extracted with EtOAc. The combined organic layers were dried (Na₂SO₄), filtered and evaporated in vacuo. The residue was purified by flash column chromatography (silica, DCM/MeOH, gradient from 100:0 to 70:70) to afford intermediate 66 (261 mg, 18%) as a colorless oil.

### Preparation of Intermediate 67

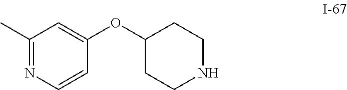

HCl (4M in 1,4-dioxane, 5.34 mL, 21.4 mmol) was added to intermediate 66 (261 mg, 0.89 mmol) at room temperature. The reaction mixture was stirred for 18 h and the volatiles were evaporated in vacuo. The residue was dissolved in MeOH and passed through an Isolute SCX-2 cartridge. The product was eluted with NH₃ (7N in MeOH) to afford intermediate 67 (170 mg, 99%) as a colorless oil.

### Preparation of Intermediate-68

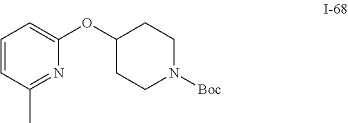 [SMILES: Cc1cccc(OC2CCN(C(=O)OC(C)(C)C)CC2)n1]

To a solution of 1-Boc-4-hydroxypiperidine (CAS: 109384-19-2; 1.00 g, 4.97 mmol) in anhydrous DMF (3.86 mL) at room temperature was added NaH (60% dispersion in mineral oil, 238 mg, 5.96 mmol) portion wise. The mixture was stirred for 1.5 h. 2-Chloro-6-methylpyridine (CAS: 18368-63-3; 697 mg, 5.47 mmol) was added and the mixture was heated at 140° C. for 45 min under microwave irradiation. The mixture was concentrated in vacuo. The residue was dissolved in MeOH and passed through an Isolute SCX-2 cartridge. The product was eluted with NH₃ (7N in MeOH) to afford intermediate 68 (449 mg, 31%) as a pale brown oil.

### Preparation of Intermediate 69

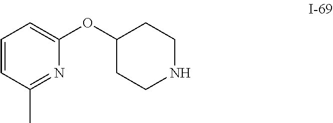

Intermediate 69 was prepared following an analogous procedure to the one described for the synthesis of intermediate 67 using intermediate 68 as starting material.

### Preparation of Intermediate 70

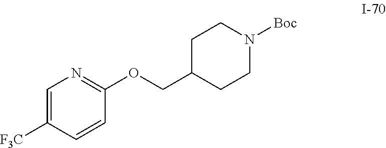 [SMILES: CC(C)(C)OC(=O)N1CCC(COc2ccc(C(F)(F)F)cn2)CC1]

N-Boc-4-piperidinemethanol (CAS: 123855-51-6; 46.0 g, 214 mmol), triphenylphosphine (92.0 g, 351 mmol) and DIAD (CAS: 1972-28-7; 61.0 g, 350 mmol) were dissolved in THF (1.0 L). The mixture was cooled to 0° C. and 2-hydroxy-5-(trifluoromethyl)pyridine (CAS: 33252-63-0; 35.0 g, 215 mmol) was added. The reaction mixture was stirred at room temperature for 4 h and evaporated in vacuo. The crude product was purified by flash column chromatography (silica, petroleum ether/EtOAc, gradient from 50:1 to 5:1) to afford intermediate 70 (42 g, 55%).

### Preparation of Intermediate 71

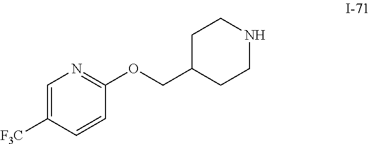 [SMILES: FC(F)(F)c1ccc(OCC2CCNCC2)nc1]

Intermediate 70 (42.0 g, 117 mmol) was added into HCl (4M in MeOH, 300 mL, 1.20 mol). The reaction mixture was stirred at room temperature for 2 h and concentrated in vacuo to afford intermediate 71 (26.55 g).

### Preparation of Intermediate 72

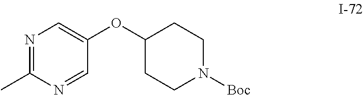 [SMILES: Cc1ncc(OC2CCN(C(=O)OC(C)(C)C)CC2)cn1]

Triphenylphosphine (619 mg, 2.36 mmol) was added to a mixture of 2-methylpyrimidin-5-ol (CAS: 35231-56-2; 200 mg, 1.82 mmol), 1-Boc-4-hydroxypiperidine (CAS: 109384-19-2; 366 mg, 1.82 mmol) and DBAD (CAS: 870-50-8; 544 mg, 2.36 mmol) in THF (4 mL). The reaction mixture was stirred at room temperature for 18 h and concentrated to dryness. The crude mixture was purified by flash column chromatography (silica, heptane/EtOAc, gradient from 100:0 to 80:20) to afford intermediate 72 (893 mg, 80%, 48% purity) as a white solid.

### Preparation of Intermediate 73

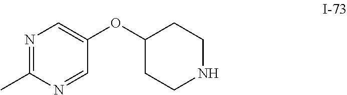

HCl (4M in 1,4-dioxane, 4.38 mL, 17.5 mmol) was added to a stirred solution of intermediate 72 (893 mg, 1.46 mmol, 48% purity) in 1,4-dioxane (12.5 mL). The reaction mixture was stirred at room temperature for 3 h and the solvent was concentrated in vacuo. A solution of the residue in MeOH (4.5 mL) was added to a closed reactor containing Amberlyst®15 hydrogen form (CAS: 39389-20-3; 1.55 g, 7.31 mmol). The mixture was shaken in a solid phase reactor at room temperature for 16h. The resin was washed with MeOH. NH₃ (7N in MeOH) was added and the mixture was shaken in the solid phase reactor for 2 h. The resin was filtered off and washed with NH₃ (7N in MeOH). The filtrates were combined and concentrated in vacuo to afford intermediate 73 (246 mg, 87%) as a yellow oil.

### Preparation of Intermediate 74

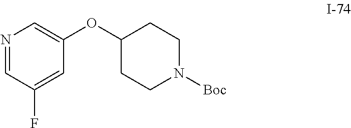 [SMILES: CC(C)(C)OC(=O)N1CCC(Oc2cncc(F)c2)CC1]

DBAD (CAS: 870-50-8; 1.72 g, 7.45 mmol) was added to a stirred mixture of 1-Boc-4-hydroxypiperidine (CAS: 109384-19-2; 1.00 g, 4.97 mmol), 5-fluoropyridin-3-ol (CAS: 209328-55-2; 618 mg, 5.47 mmol) and triphenylphosphine (1.96 g, 7.45 mmol) in THF (12.1 mL) at 0° C. in a sealed tube and under N₂ atmosphere. The reaction mixture was stirred at room temperature for 2 h. The mixture was diluted with EtOAc and washed with NaOH (5N). The organic layer was dried (Na₂SO₄), filtered and concentrated in vacuo. The crude mixture was purified twice by flash column chromatography (silica, heptane/EtOAc, gradient from 100:0 to 70:30) to afford intermediate 74 (890 mg, 60%).

### Preparation of Intermediate 75

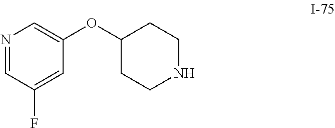 [SMILES: Fc1cncc(OC2CCNCC2)c1]

A solution of intermediate 74 (0.89 g, 3.00 mmol) in MeOH (23 mL) was added to a closed reactor containing Amberlyst®15 hydrogen form (CAS: 39389-20-3; 3.2 g, 15.0 mmol). The mixture was shaken in a solid phase reactor at room temperature for 16 h. The resin was washed with MeOH (the fraction was discarded), then NH₃ (7N solution in MeOH) (23 ml) was added. The mixture was shaken in the solid phase reactor for 2 h. The resin was filtered and washed with NH₃ (7N solution in MeOH) (3×23 mL; 30 min shaken). The filtrates were concentrated in vacuo to yield intermediate 75 (550 mg, 93%).

### Preparation of Intermediates 76 and 77

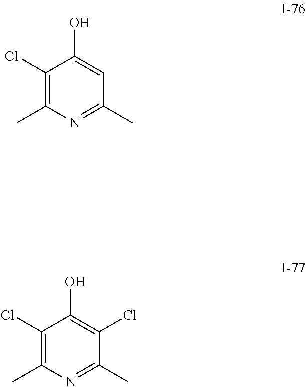

A suspension of 2,6-dimethylpyridin-4-ol (CAS: 13603-44-6; 1.00 g, 8.12 mmol) and N-chlorosuccinimide (1.46 g, 10.9 mmol) in a mixture of MeOH (10 ml) and DCM (25 ml) was stirred under inert atmosphere at room temperature overnight. The precipitate was filtered and the filtrate was concentrated to dryness. The residue was triturated with CH₃CN. The precipitate was filtered, washed with CH₃CN, and dried under vacuum to yield a mixture of intermediates 76 and 77 (940 mg, 73%) as a white solid.

### Preparation of Intermediate 78

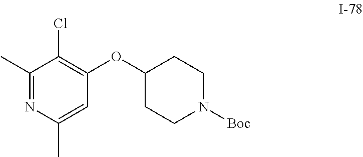 [SMILES: Cc1cc(OC2CCN(C(=O)OC(C)(C)C)CC2)c(Cl)c(C)n1]

DBAD (CAS: 870-50-8; 1.52 g, 6.60 mmol) was added to a mixture of intermediates 76 and 77 (800 mg, 5.08 mmol), 1-Boc-4-hydroxypiperidine (CAS: 109384-19-2; 1.33 g, 6.60 mmol) and triphenylphosphine (1.73 g, 6.60 mmol) in toluene (16 mL). The reaction mixture was stirred at room temperature for 1 h and at 85° C. for 1 h. The reaction mixture was concentrated to dryness and the residue was purified by flash column chromatography (silica, heptane/EtOAc, gradient from 100:0 to 70:30). The desired fractions were collected and concentrated in vacuo to afford intermediate 78 (3.7 g, 77%, 36% purity).

### Preparation of Intermediate 79

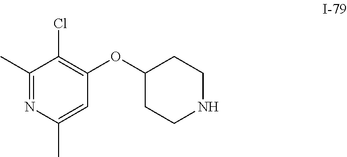 [SMILES: Cc1cc(OC2CCNCC2)c(Cl)c(C)n1]

Intermediate 79 was prepared following an analogous procedure to the one described for the synthesis of intermediate 67 using intermediate 78 as starting material.

The residue was purified by ion exchange chromatography using an Isolute SCX-2 cartridge. The product was eluted with MeOH, then with NH₃ (7N in MeOH). The desired fractions were collected and evaporated in vacuo to afford intermediate 79 (0.90 g, 96%) as a colorless oil which solidified upon standing.

### Preparation of Intermediate 80

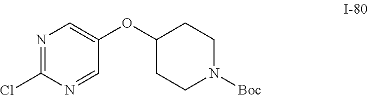 [SMILES: CC(C)(C)OC(=O)N1CCC(Oc2cnc(Cl)nc2)CC1]

Intermediate 80 was prepared following an analogous procedure to the one described for the synthesis of intermediate 78 using in 1-Boc-4-hydroxypiperidine (CAS: 109384-19-2) and 2-chloropyrimidine-5-ol (CAS: 4983-28-2) as starting materials.

The crude mixture was purified by flash column chromatography (silica, heptane/EtOAc, gradient from 100:0 to 70:30) to afford intermediate 80 (2.3 g, 96%) as a white solid.

### Preparation of Intermediate 81

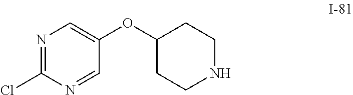 [SMILES: Clc1ncc(OC2CCNCC2)cn1]

HCl (4M in 1,4-dioxane, 12.1 mL, 48.4 mmol) was added to intermediate 80 (1.90 g, 6.06 mmol) and the reaction mixture was stirred at room temperature for 3 h. The reaction mixture was concentrated to dryness. The residue was suspended in DCM and basified with NH₄OH. The organic layer was separated and the aqueous layer was further extracted with DCM. The combined organic layers were dried (MgSO₄), filtered and the solvent was evaporated in vacuo to give intermediate 81 (1.28 g, 99%) as a white solid.

### Preparation of Intermediate 82

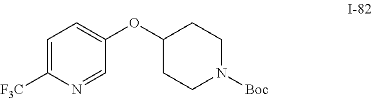 [SMILES: CC(C)(C)OC(=O)N1CCC(Oc2ccc(C(F)(F)F)nc2)CC1]

DBAD (CAS: 870-50-8; 642 mg, 2.79 mmol) was added to a solution of 6-(trifluoromethyl)pyridine-3-ol (CAS: 216766-12-0; 350 mg, 2.15 mmol), 1-Boc-4-hydroxypiperidine (CAS: 109384-19-2; 561 mg, 2.79 mmol) and triphenylphosphine (732 mg, 2.79 mmol) in THF (3.5 mL). The reaction mixture was stirred at room temperature for 18 h and concentrated to dryness. The residue was purified by flash column chromatography (silica, heptane/EtOAc, gradient from 100:0 to 85:15) to afford intermediate 82 (580 mg, 78%) as a white solid.

### Preparation of Intermediate 83

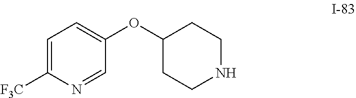 [SMILES: FC(F)(F)c1ccc(OC2CCNCC2)cn1]

Intermediate 83 was prepared following an analogous procedure to the one described for the synthesis of intermediate 81 using intermediate 82 as starting material.

### Preparation of Intermediate 84

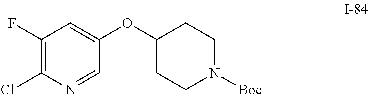 [SMILES: CC(C)(C)OC(=O)N1CCC(Oc2cnc(Cl)c(F)c2)CC1]

Intermediate 84 was prepared following an analogous procedure to the one described for the synthesis of intermediate 82 using 1-Boc-4-hydroxypiperidine (CAS: 109384-19-2) and 6-chloro-5-fluoropyridin-3-ol (CAS: 870062-76-3) as starting materials.

The residue was purified by flash column chromatography (silica, heptane/EtOAc, gradient from 100:0 to 85:15) to afford intermediate 84 (880 mg, 78%) as a white solid.

### Preparation of Intermediate 85

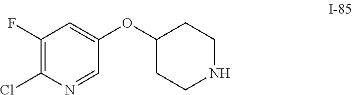 [SMILES: Fc1cc(OC2CCNCC2)cnc1Cl]

Intermediate 85 was prepared following an analogous procedure to the one described for the synthesis of intermediate 81 using intermediate 84 as starting material.

### Preparation of Intermediate 86

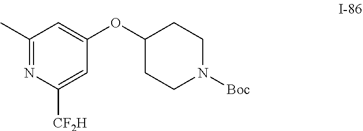 [SMILES: Cc1cc(OC2CCN(C(=O)OC(C)(C)C)CC2)cc(C(F)F)n1]

To a mixture of NaH (60% dispersion in mineral oil, 162 mg, 4.05 mmol) in DMF (6 mL) at 0° C. was added 1-Boc-4-hydroxypiperidine (CAS: 109384-19-2; 326 mg, 1.62 mmol) and 15-crown-5 (270 μL, 1.62 mmol). The mixture was stirred for 30 min and 4-bromo-2-(difluoromethyl)-6-methylpyridine (CAS: 1226800-12-9; 300 mg, 1.35 mmol) was added slowly. The reaction mixture was stirred at 70° C. for 18 h, cooled to 0° C. and quenched with water. The product was extracted with EtOAc. The organic layer was dried (MgSO₄), filtered and the solvent was evaporated in vacuo. The crude product was purified by flash column chromatography (silica, EtOAc in DCM, gradient from 0:100 to 50:50) to afford intermediate 86 (435 mg, 94%) as a colourless oil.

### Preparation of Intermediate 87

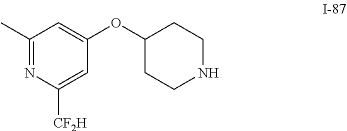 [SMILES: Cc1cc(OC2CCNCC2)cc(C(F)F)n1]

HCl (4M in 1,4-dioxane, 8.6 mL, 35.0 mmol) was added to intermediate 86 (435 mg, 1.27 mmol) and the reaction mixture was stirred at room temperature for 3 h. The reaction mixture was concentrated to dryness. The residue was purified by ion exchange chromatography using an Isolute SCX-2 cartridge. The product was eluted with MaOH, then with NH₃ (7M in MeOH). The desired fraction were collected and concentrated in vacuo to afford intermediate 87 (300 mg, 97%) as a colorless oil.

### Preparation of Intermediate 88

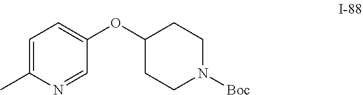 [SMILES: Cc1ccc(OC2CCN(C(=O)OC(C)(C)C)CC2)cn1]

To a stirred mixture of 1-Boc-4-hydroxypiperidine (CAS: 109384-19-2; 3.00 g, 3.70 mmol), 5-hydroxy-2-methylpyridine (CAS: 1121-78-4; 0.41 g, 3.70 mmol), triphenylphosphine polymer bound (1.88 mmol/g, 3.63 g, 6.80 mmol) and THF (48 mL) cooled with an ice-water bath was added dropwise DIAD (CAS: 2446-83-5; 1.38 mL, 7.00 mmol). The reaction mixture was stirred in a microwave at 120° C. for 20 min. The mixture was filtered over Celite® and the filtrate was evaporated till dryness in vacuo. The residue was purified by flash column chromatography (silica, DCM/MeOH, gradient from 100:0 to 96:4) to afford intermediate 88 (3.53 g, 81%).

### Preparation of Intermediate 89

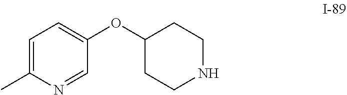

A mixture of intermediate 88 (3.67 g, 12.6 mmol) and TFA (21 mL) in CHCl₃ (95 mL) was stirred at room temperature for 3 h. The mixture was concentrated in vacuo. The residue was treated with water and DCM. The aqueous layer was separated and basified with NaOH (50%, aq.). The aqueous phase was extracted with DCM, dried (Na₂SO₄), filtered and evaporated in vacuo to dryness to afford intermediate (2.01 g, 83%).

### Preparation of Intermediate 90

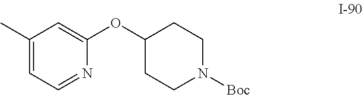 [SMILES: Cc1ccnc(OC2CCN(C(=O)OC(C)(C)C)CC2)c1]

To a stirred mixture of NaH (60% dispersion in mineral oil, 1.96 g, 49.1 mmol) in DME (57 mL) was added 1-Boc-4-hydroxypiperidine (CAS: 109384-19-2; 5.81 g, 28.9 mmol) portionwise. The mixture was stirred at room temperature for 1 h, and 2-bromo-4-methylpyridine (CAS: 4926-28-7; 1.60 mL, 14.4 mmol) was added. The reaction mixture was stirred under reflux for 4 days. The mixture was cooled down and carefully treated with water. The aqueous phase was extracted with EtOAc. The combined organic layers were dried (Na₂SO₄), filtered and evaporated to dryness in vacuo. The crude product was purified by flash column chromatography (silica, DCM/MeOH, gradient from 100:0 to 96:4) to afford intermediate 90 (2.74 g, 65%).

### Preparation of Intermediate 91

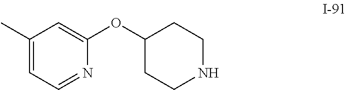

Intermediate 91 was prepared following an analogous procedure to the one described for the synthesis of intermediate 89 using intermediate 90 as starting material.

### Preparation of Intermediate 92

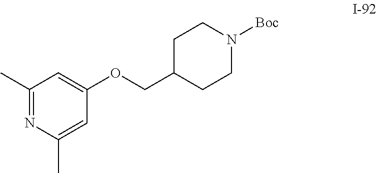 [SMILES: Cc1cc(OCC2CCN(C(=O)OC(C)(C)C)CC2)cc(C)n1]

To a mixture of N-Boc-4-piperidinemethanol (CAS: 123855-51-6; 6.94 g, 32.3 mmol) in DMF (40 ml) was added NaH (60% dispersion in mineral oil, 1.42 g, 34.5 mmol) portionwise under N₂ atmosphere. The mixture was stirred at 80° C. for 30 min, and a solution of 4-bromo-2,6-dimethylpyridine (CAS: 5093-70-9; 3.00 g, 16.1 mmol) in DMF (10 mL) was added dropwise. The reaction mixture was stirred at 80° C. overnight. Water (50 mL) was added and the mixture was extracted with DCM (5×200 mL). The combined organic extracts were washed with brine (5×50 mL), dried (Na₂SO₄), filtered and concentrated in vacuo. The crude product was purified by flash column chromatography (silica, petroleum ether/EtOAC, gradient from 100:0 to 2:1). The pure fractions were collected and the solvent was evaporated in vacuo to afford intermediate 92 (1.2 g, 23%) as a yellow oil.

### Preparation of Intermediate 93

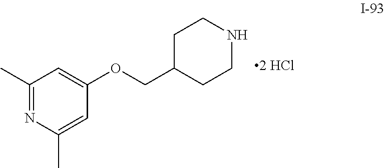 [SMILES: Cc1cc(OCC2CCNCC2)cc(C)n1]

A mixture of intermediate 92 (1.20 g, 3.75 mmol) in HCl (4M in 1,4-dioxane, 20 mL, 80 mmol) was stirred at 25° C. for 1 h and concentrated in vacuo to afford a yellow solid which was triturated with tert-butyl methyl ether (2×20 mL) to give intermediate 93 (1.0 g, 91%).

### Preparation of Intermediate 94

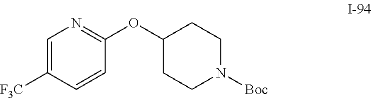 [SMILES: CC(C)(C)OC(=O)N1CCC(Oc2ccc(C(F)(F)F)cn2)CC1]

NaOtBu (4.78 g, 49.7 mmol) was added to a stirred solution of 1-Boc-4-hydroxypiperidine (CAS: 109384-19-2; 5.00 g, 24.8 mmol) and 2-chloro-5-(trifluoromethyl)pyridine (4.51 g, 24.8 mmol) in DMSO (28 mL). The reaction mixture was stirred at room temperature for 24 h and diluted with water. The aqueous phase was extracted with EtOAc. The combined organic layers were dried (Na₂SO₄), filtered and the solvents were evaporated in vacuo to afford intermediate 94 (8.28 g, 96%) as a solid which was used in the next step without further purification.

### Preparation of Intermediate 95

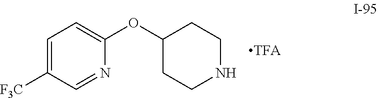 [SMILES: FC(F)(F)c1ccc(OC2CCNCC2)nc1]

TFA (18.4 mL, 239 mmol) was added to a stirred solution of intermediate 94 (8.28 g, 23.9 mmol) in DCM (83 mL) at 0° C. The mixture was stirred at room temperature for 2 h and concentrated in vacuo. The residue was diluted with water and basified with 10% NaOH. The aqueous phase was extracted with EtOAc. The organic layer was dried (Na₂SO₄), filtered and the solvent was evaporated in vacuo. The crude product was purified by flash column chromatography (silica, DCM/MeOH, gradient from 100:0 to 80:20). The desired fractions were collected and concentrated in vacuo to afford intermediate 95 (3.27 g, 38%).

### Preparation of Intermediate 96

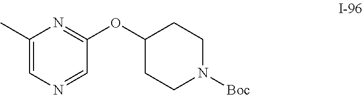 [SMILES: Cc1cncc(OC2CCN(C(=O)OC(C)(C)C)CC2)n1]

Intermediate 96 was prepared following an analogous procedure to the one described for the synthesis of intermediate 94 using 1-Boc-4-hydroxypiperidine (CAS: 109384-19-2) and 2-chloro-6-methylpyrazine (CAS: 38557-71-0) as starting materials.

The crude product was purified by flash column chromatography (silica, heptane/EtOAc, gradient from 100:0 to 50:50) to afford intermediate 96 (2.25 g, 82%) as a yellow oil.

### Preparation of Intermediate 97

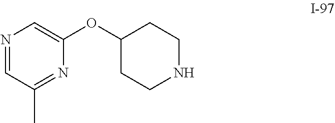

A solution of intermediate 96 (2.25 g, 7.67 mmol) in MeOH (62.2 mL) was added to a solid phase reactor containing Amberlyst®15 hydrogen form (CAS: 39389-20-3; 8.16 g, 38.3 mmol). The mixture was shaken at room temperature for 16 h. The solvent was removed and the resin was washed with MeOH (×3), filtered and the solvent was discarded. The product was eluted with NH₃ (7N in MeOH) (×3). The filtrates were combined and concentrated in vacuo to afford intermediate 97 (1.40 g, 95%) as a yellow oil.

### Preparation of Intermediate 98

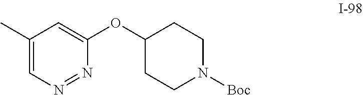 [SMILES: Cc1cnnc(OC2CCN(C(=O)OC(C)(C)C)CC2)c1]

Intermediate 98 was prepared following an analogous procedure to the one described for the synthesis of intermediate 94 using 1-Boc-4-hydroxypiperidine (CAS: 109384-19-2) and 3-chloro-5-methylpyridazine (CAS: 89283-31-8) as starting materials.

The crude product was purified by flash column chromatography (silica, heptane/EtOAc, gradient from 100:0 to 50:50) to afford intermediate 98 (0.77 g, 51%) as a yellow oil.

### Preparation of Intermediate 99

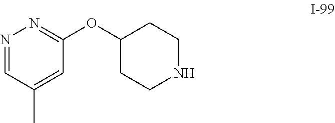

Intermediate 99 was prepared following an analogous procedure to the one described for the synthesis of intermediate 97 using intermediate 98 as starting material.

### Preparation of Intermediate 100

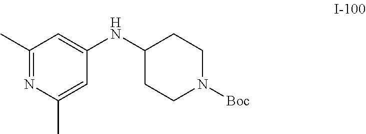 [SMILES: Cc1cc(NC2CCN(C(=O)OC(C)(C)C)CC2)cc(C)n1]

A solution of 4-amino-1-Boc-piperidine (CAS: 5399-92-8; 2.00 g, 9.98 mmol), 4-bromo-2,6-dimethylpyridine (CAS: 5093-70-9; 1.86 g, 9.88 mmol), Pd₂dba₃ (183 mg, 0.2 mmol) and XPhos (143 mg, 0.3 mmol) in toluene (8 mL) was degassed. tBuOK (2.24 g, 20 mmol) was added. The reaction vessel was sealed and heated at 120° C. for 14 h. The reaction mixture was cooled to room temperature and filtered through Celite®. The mixture was washed with EtOAc. The filtrate was evaporated in vacuo and the crude mixture was purified by flash column chromatography (silica, NH₃ (7N in MeOH)/DCM, gradient from 0:100 to 90:10). The desired fractions were collected and evaporated in vacuo. A second purification was performed by flash column chromatography (silica, NH₃ (7N in MeOH)/DCM, gradient from 0:100 to 98:2). The desired fractions were collected and concentrated in vacuo to afford intermediate 100 (352 mg, 12%) as a yellow oil.

### Preparation of Intermediate 101

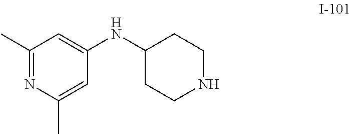

Intermediate 101 was prepared following an analogous procedure to the one described for the synthesis of intermediate 97 using intermediate 100 as starting material.

### Preparation of Intermediate 102

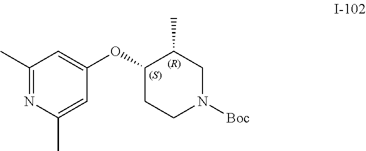

tBuOK (261 mg, 2.32 mmol) was added to a stirred solution of (3S,4R)-4-hydroxy-3-methylpiperidine-1-carboxylate (CAS: 955028-93-0; 250 mg, 1.16 mmol) in DMSO (3.1 mL) at room temperature, followed by the addition of 4-chloro-2,6-dimethylpyridine (CAS: 3512-75-2; 164 mg, 1.16 mmol) in a microwave vial under N₂ atmosphere. The reaction mixture was stirred at 60° C. for 18 h. The mixture was cooled down to room temperature, treated with water and extracted with EtOAc. The combined organic layers were dried (Na₂SO₄), filtered and the solvent was evaporated in vacuo to afford intermediate 102 which was used as such in the next step.

### Preparation of Intermediate 103

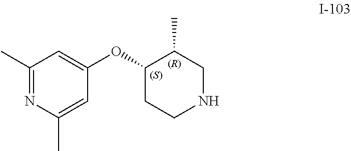

The resin Amberlyst®15 hydrogen form (CAS: 39389-20-3; 4.11 mmol/g) was added to intermediate 102 in MeOH (62 mL). The reaction was shaken for 24 h. The solvent was removed and discarded. The resin was washed few times with MeOH. Then NH₃ (7N in MeOH) was added to the resin and the mixture was shaken for 4 h. The solvent was removed and the resin was washed few times with NH₃ (7N in MeOH). The organic solvent was evaporated in vacuo to afford intermediate 103 (240 mg).

### Preparation of Intermediate 104

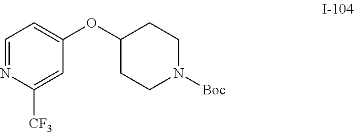 [SMILES: CC(C)(C)OC(=O)N1CCC(Oc2ccnc(C(F)(F)F)c2)CC1]

NaH (60% dispersion in mineral oil, 46 mg, 0.1.19 mmol) was added portionwise to a stirred solution of 1-Boc-4-hydroxypiperidine (CAS: 109384-19-2; 200 mg, 0.99 mmol) in DMF (3 mL) in a sealed tube and under N₂ at 0° C. The reaction mixture was stirred at 0° C. for 30 min and a solution of 4-chloro-2-(trifluoromethyl)pyridine (CAS: 131748-14-6; 271 mg, 1.49 mmol) in DMF (2 mL) was added dropwise at 0° C. The reaction mixture was stirred at 60° C. for 48 h. The mixture was concentrated in vacuo. The residue was diluted with water and extracted with EtOAc. The organic layer was dried (Na₂SO₄), filtered and the solvents were evaporated in vacuo. The crude product was purified by flash column chromatography (silica, EtOAc in DCM, gradient from 0:100 to 50:50). The desired fractions were collected and concentrated in vacuo to afford intermediate 104 (212 mg, 62%) as colorless oil which solidified to a white solid upon standing.

### Preparation of Intermediate 105

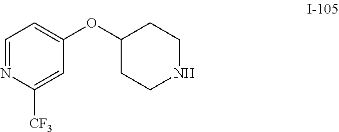 [SMILES: FC(F)(F)c1cc(OC2CCNCC2)ccn1]

A solution of intermediate 104 (212 mg, 0.61 mmol) in MeOH (5 mL) was added to a solid phase reactor containing Amberlyst®15 hydrogen form (CAS: 39389-20-3; 0.65 g, 3.06 mmol). The mixture was shaken at room temperature for 16 h. The solvent was removed and the resin was washed with MeOH (3 times), filtered and the solvent discarded. The product was eluted with NH₃ (7N in MeOH) (3 times). The solvent was evaporated in vacuo to afford intermediate 105 (144 mg, 95%) as a brown oil.

### Preparation of Intermediate 106

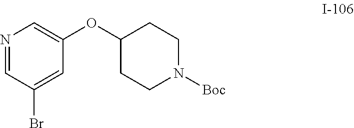 [SMILES: CC(C)(C)OC(=O)N1CCC(Oc2cncc(Br)c2)CC1]

NaH (60% dispersion in mineral oil, 0.24 g, 5.96 mmol) was added to a stirred solution of 1-Boc-4-hydroxypiperidine (CAS: 109384-19-2; 1.00 g, 4.97 mmol) in anhydrous DMF (6.25 mL) at 0° C. The mixture was stirred at 0° C. for 30 min, and 3-bromo-5-fluoropyridine (CAS: 407-20-5; 0.98 g, 5.47 mmol) in anhydrous DMF (6.25 mL) was added. The reaction mixture was stirred at room temperature for 16 h and concentrated in vacuo. The residue was diluted with water and extracted with EtOAc. The organic layer was dried (Na₂SO₄), filtered and evaporated in vacuo. The crude product was purified by flash column chromatography (silica, heptane/EtOAc, gradient from 100:0 to 70:30) to afford intermediate 106 (1.08 g, 61%) as a white sticky solid.

### Preparation of Intermediate 107

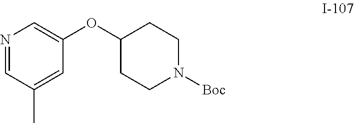 [SMILES: Cc1cncc(OC2CCN(C(=O)OC(C)(C)C)CC2)c1]

Pd(OAc)₂ (23.6 mg, 0.11 mmol) and tricyclohexylphosphine tetrafluoroborate (77.3 mg, 0.21 mmol) were added to a stirred mixture of intermediate 106 (500 mg, 1.40 mmol), trimehtylboroxine (0.53 mL, 3.78 mmol) and K₂CO₃ (387 mg, 2.80 mmol) in degassed 1,4-dioxane (4.3 mL) in a sealed tube. The mixture was purged with N₂ for 5 min and stirred at 100° C. for 16 h under N₂ atmosphere. The mixture was cooled down, washed with H2O and extracted with DCM. The organic layer was dried (MgSO₄), filtered and the solvents were evaporated in vacuo. The residue was dissolved in MeOH and passed through an Isolute SCX-2 cartridge. The product was eluted with NH₃ (7N in MeOH) to afford intermediate 107 (420 mg, 74%, 72% purity) as a colorless oil.

### Preparation of Intermediate 108

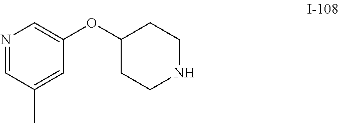

HCl (4M in 1,4-dioxane, 6.2 mL, 24.7 mmol) was added to intermediate 107 (420 mg, 1.03 mmol, 72% purity). The reaction mixture was stirred at room temperature for 18 h. The volatiles were evaporated in vacuo. The residue was dissolved in MeOH and passed through an Isolute SCX-2 cartridge. The product was eluted with NH₃ (7N in MeOH) to afford intermediate 108 (298 mg, 72%, 48% purity) as a colorless oil.

### Preparation of Intermediate 109

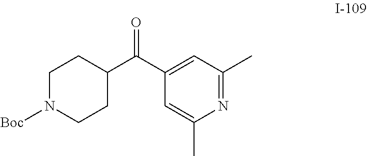 [SMILES: Cc1cc(C(=O)C2CCN(C(=O)OC(C)(C)C)CC2)cc(C)n1]

n-BuLi (2.5 M in hexane, 3.67 mL, 9.16 mmol) was added to a mixture of 4-bromo-2,6-dimethylpyridine (CAS: 5093-70-9; 1.55 g, 8.33 mmol) in THF (25 ml) at −78° C. under N₂ atmosphere. The mixture was stirred at −78° C. for 30 min and then a solution of tert-butyl 4-(methoxy(methyl)carbamoyl)piperidine-1-carboxylate (CAS: 139290-70-3; 2.50 g, 9.16 mmol) in THF (5 ml) was added at −78° C. The reaction mixture was stirred at −78° C. for 1 h. NH₄Cl (sat., aq.) was added and the mixture was extracted with EtOAc (2×10 mL). The organic layer was dried (Na₂SO₄), filtered and concentrated in vacuo. The crude product was purified by flash column chromatography (silica, heptane/EtOAc, gradient from 100:0 to 80:20). The desired fractions were collected and concentrated in vacuo to afford intermediate 109 (1.44 g, 54%) as a yellow oil that solidified upon standing.

### Preparation of Intermediate 110

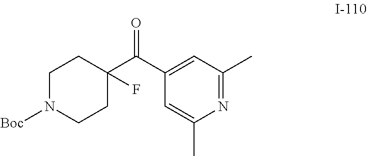 [SMILES: Cc1cc(C(=O)C2(F)CCN(C(=O)OC(C)(C)C)CC2)cc(C)n1]

LiHMDS (1M solution, 4.98 mL, 7.98 mmol) was added to a mixture of intermediate 109 (1.44 g, 4.52 mmol) in THF (111 ml), at −78° C. The mixture was stirred at −10° C. for 1 h and the mixture was cooled down to −78° C. A solution of N-benzenfluorosulfonamide (CAS: 133745-75-2; 1.57 g, 4.98 mmol) in THF (12.3 mL) was added. The reaction mixture was stirred at −78° C. for 1 h, and at −50° C. for 2 h. NH₄Cl (sat., aq.) was added and the mixture was extracted with EtOAc. The organic layer was dried (Na₂SO₄), filtered and evaporated in vacuo. The crude mixture was purified by flash column chromatography (silica, DCM/MeOH, gradient from 100:0 to 93:7, then heptane/EtOAc, gradient from 100:0 to 0:100). The desired fractions were collected and concentrated in vacuo to afford intermediate 110 (963 mg, 41%, 65% purity) as a yellow oil that solidified upon standing.

### Preparation of Intermediate 111

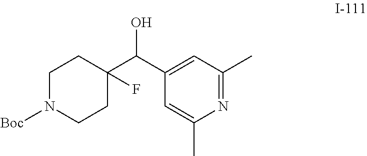 [SMILES: Cc1cc(C(O)C2(F)CCN(C(=O)OC(C)(C)C)CC2)cc(C)n1]

NaBH₄ (0.13 g, 3.44 mmol) was added to a mixture of intermediate 110 (963 mg, 2.86 mmol, 65% purity) in MeOH (19.3 mL) at 0° C. The reaction mixture was stirred at room temperature for 2 h and quenched with NaOH (1 M) (2 mL). The aqueous phase was extracted with EtOAc (2×30 mL). The combined organic layers were dried (Na₂SO₄), filtered and concentrated in vacuo to afford intermediate 111 (1.07 g, 81%, 73% purity).

### Preparation of Intermediate 112

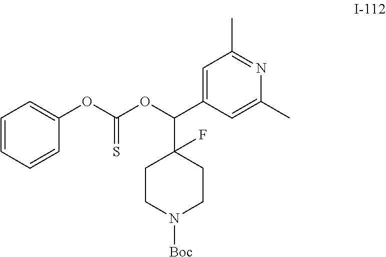 [SMILES: Cc1cc(C(OC(=S)Oc2ccccc2)C2(F)CCN(C(=O)OC(C)(C)C)CC2)cc(C)n1]

O-Phenyl chlorothionoformate (CAS: 1005-56-7; 1.43 g, 8.27 mmol) was added to a mixture of intermediate 111 (1.40 g, 4.14 mmol, 73% purity) and DMAP (75.8 mg, 0.62 mmol) in DCM (33.6 mL). Et₃N (1.44 mL, 10.3 mmol) was added and the reaction mixture was stirred at room temperature for 72 h. NH₄Cl (sat., aq.) was added and the mixture was extracted with EtOAc. The organic layer was washed with brine, dried (Na₂SO₄), filtered and concentrated in vacuo. The crude mixture was purified by flash column chromatography (silica, EtOAc in DCM, gradient from 0:100 to 100:0, then MeOH in DCM, gradient from 0:100 to 15:85). The desired fractions were collected concentrated in vacuo to give intermediate 112 (623 mg, 32%) as a light yellow foam.

### Preparation of Intermediate 113

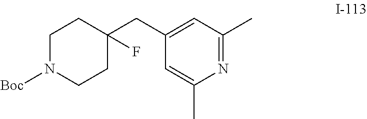 [SMILES: Cc1cc(CC2(F)CCN(C(=O)OC(C)(C)C)CC2)cc(C)n1]

Tributyltin hydride (CAS: 688-73-3; 1.07 mL, 3.98 mmol) was added to a mixture of intermediate 112 (630 mg, 1.33 mmol) and AIBN (CAS: 78-67-1; 21.8 mg, 0.13 mmol) in toluene (19 mL). The reaction mixture was stirred at 110° C. for 2 h. The mixture was cooled down and the solvent was evaporated in vacuo. The crude mixture was purified by flash column chromatography (silica, heptane/DCM, gradient from 100:0 to 0:100, then DCM/MeOH, gradient from 100:0 to 85:15). The desired fractions were collected and concentrated in vacuo to afford intermediate 113 (457 mg, 88%, 82% purity) as a light yellow oil.

### Preparation of Intermediate 114

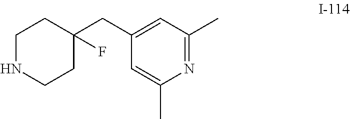 [SMILES: Cc1cc(CC2(F)CCNCC2)cc(C)n1]

TFA (0.92 mL, 11.9 mmol) was added to a mixture of intermediate 113 (457 mg, 1.42 mmol, 82% purity) in DCM (2.3 mL). The reaction mixture was stirred at room temperature for 3 h and the solvent was evaporated in vacuo.

A fraction of the residue (150 mg) was neutralized with NaHCO₃ (sat., aq.) and extracted with DCM (2×10 mL) and with MeOH and DCM (2:8). The organic layer was dried (Na₂SO₄), filtered and concentrated in vacuo to afford intermediate 114 (100 mg) as an orange oil which was used in the next step without further purification.

### Preparation of Intermediate 115

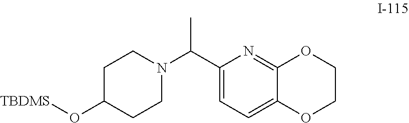

Ti(O-iPr)₄ (13.4 mL, 45.4 mmol) was added to a stirred solution of 4-(tert-butyldimethylsiloxy)piperidine (CAS: 97231-91-9; 6.52 g, 30.3 mmol) and 2,3-dihydro-[1,4]dioxino[2,3-b]pyridine-6-carbaldehyde (CAS: 615568-24-6; 5.00 g, 30.3 mmol) in anhydrous DCM (170 mL) at room temperature and under N₂ atmosphere. The reaction mixture was stirred for 20 h, cooled to 0° C. and methylmagnesium bromide (3.2M in Me-THF, 28.4 mL, 90.8 mmol) was added dropwise. The reaction mixture was stirred at this temperature for 15 min and at room temperature for 1 h. NH₄Cl (40 mL) was added and the mixture was cooled with an ice bath. A yellow solid formed and the mixture was diluted with water (500 mL). The mixture was extracted with DCM (2×200 mL). The combined organic layers were washed with brine (4×100 mL), dried (Na₂SO₄), filtered and concentrated in vacuo. The crude product was purified by flash column chromatography (silica, NH₃ (7N in MeOH)/DCM, gradient from 0:100 to 2:98). The desired fractions were collected and the solvents were evaporated in vacuo to afford 2 fractions of intermediate 115 (fraction A: 1.42 g, 12%, 98% purity; and fraction B: 6.83 g, 55%, 92% purity) as orange oils.

### Preparation of Intermediates 116, 117 AND 118

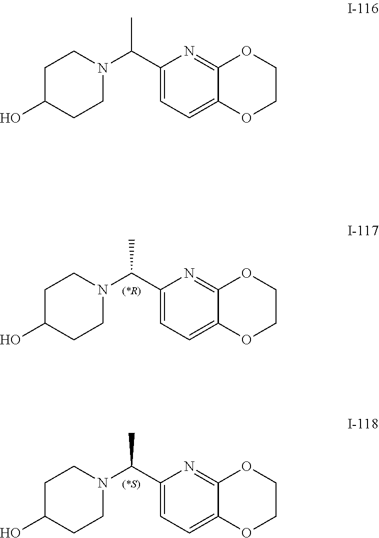 [SMILES: CC(c1ccc2c(n1)OCCO2)N1CCC(O)CC1 ; C[C@H](c1ccc2c(n1)OCCO2)N1CCC(O)CC1 ; C[C@@H](c1ccc2c(n1)OCCO2)N1CCC(O)CC1]

TBAF (1M solution, 28.1 mL, 28.1 mmol) was added to a stirred solution of intermediate 115 (8.25 g, 20.1 mmol, 92% purity) in anhydrous THF (207 mL) at 0° C. under N₂ atmosphere. The reaction mixture was stirred at room temperature for 20 h. The mixture was diluted with water and extracted with EtOAc. The combined organic layers were washed with brine, dried (Na₂SO₄), filtered and the solvent was evaporated in vacuo. The crude product was purified by flash column chromatography (silica, NH₃ (7N in MeOH)/DCM, gradient from 0:100 to 5:95) to afford intermediate 116 (3.0 g, 57%) as an orange solid. A purification of intermediate 116 (1.27 g) was performed via chiral SFC (stationary phase: CHIRACEL OJ-H 5 μm 250*30 mm, mobile phase: 90% CO₂, 10% MeOH (0.9% i-PrNH₂)) delivered intermediate 117 (593 mg) and intermediate 118 (593 mg).

### Preparation of Intermediate 119

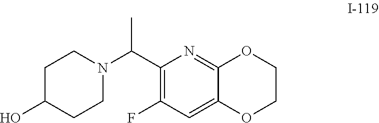 [SMILES: CC(c1nc2c(cc1F)OCCO2)N1CCC(O)CC1]

A mixture of 4-hydroxypiperidine (CAS: 5382-16-20; 4.65 g, 45.9 mmol) and K₂CO₃ (9.53 g, 68.9 mmol) in CH₃CN (100 mL) was stirred at 25° C. under N₂ atmosphere for 10 min. Intermediate 20 (5.00 g, 22.9 mmol) was added dropwise and the reaction mixture was stirred at 80° C. under N₂ atmosphere overnight. The mixture was evaporated in vacuo. The crude product was combined with another fraction (11.5 mmol) and purified by flash column chromatography (silica, petroleum ether/EtOAc, gradient from 100:0 to 3:1) to afford intermediate 119 (8.05 g, 80%) as a white solid.

### Preparation of Intermediate 120

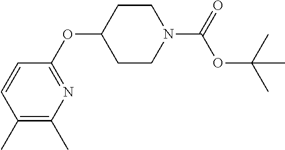 [SMILES: Cc1ccc(OC2CCN(C(=O)OC(C)(C)C)CC2)nc1C]

To a mixture of 1-Boc-4-hydroxypiperidine (CAS: 109384-19-2,200 mg, 0.99 mmol) in DMF (3.847 mL), were added NaH (60% dispersion in mineral oil, 79.5 mg, 1.99 mmol) and 15-crown-5 (198.4 μL, 1.19 mmol). Then 6-chloro-2,3-dimethylpyridine (154.78 mg, 1.09 mmol) was added and the mixture was stirred at 80° C. for 16 h. Then additional NaH (60% dispersion in mineral oil, 39.75 mg, 0.99 mmol) was added and the mixture was stirred at 80° C. for 20 h. Then water was added at 0° C. and the mixture was extracted with DCM. The organic layer was separated, dried, filtered and the solvents concentrated in vacuo. The crude was purified by flash column chromatography (silica, EtOAc in heptane 0/100 to 70/30). The desired fractions were collected and the solvents concentrated in vacuo to give intermediate 120 (134.1 mg, 44%) as a colourless oil.

### Preparation of Intermediate 121

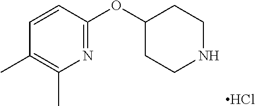

Intermediate 121 was prepared following an analogous procedure to the one described for the synthesis of intermediate 59.

### Preparation of Intermediate 148

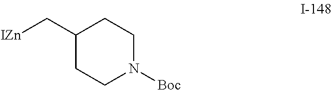

A solution of chlorotrimethylsilane (1.25 mL, 9.85 mmol) and 1-bromo-2-chloroethane (0.2 mL, 2.41 mmol) in THF (10 mL) was prepared under N₂ atmosphere in a dried flask and was passed through a column containing Zn (10 g) using a syringe pump at 40° C. and at a flow rate of 1 mL/min. A solution of 1-Boc-4-iodomethylpiperidine (CAS: 145508-94-7; 1.00 g, 3.08 mmol) in THF (10 mL) was passed through the column containing activated Zn using a syringe pump at 40° C. and at a flow rate of 0.5 m/min. The outcoming solution was collected in a closed flask under N₂ atmosphere. Titration with 12 revealed that a 0.2M solution was obtained which was used as such in the next step.

### Preparation of Intermediate 149

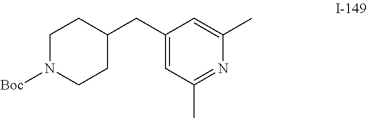 [SMILES: Cc1cc(CC2CCN(C(=O)OC(C)(C)C)CC2)cc(C)n1]

PdCl₂(dppf).DCM (94.5 mg, 0.12 mmol) and CuI (21.9 mg, 0.12 mmol) were added to a stirred solution of 4-bromo-2,6-dimethylpyridine (CAS: 5093-70-9; 215 mg, 1.15 mmol) in DMA (5 mL) at room temperature under N₂ atmosphere. The reaction mixture was bubbled with N₂ for 10 min. Then, intermediate 148 (0.2M solution, 586 mg, 1.5 mmol) was added to the stirred suspension under N₂ atmosphere at room temperature. The reaction mixture was bubbled with N₂ for 10 min and stirred at 80° C. for 16 h. The mixture was diluted with water and extracted with EtOAc. The organic layer was dried (MgSO₄), filtered and the solvents were evaporated in vacuo. The crude product was purified by flash column chromatography (silica, heptane/EtOAc, gradient from 100:0 to 80:20). The desired fractions were collected and concentrated in vacuo to afford intermediate 149 (220 mg, 63%).

### Preparation of Intermediate 150

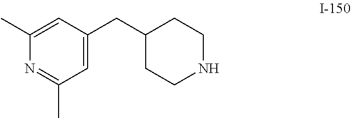

TFA (1.07 mL, 14.4 mmol) was added to a stirred solution of intermediate 149 (220 mg, 0.72 mmol) in DCM (3.69 mL) at 0° C. The reaction mixture was stirred at room temperature for 1.5 h. The solvent was removed in vacuo. The residue was dissolved in MeOH and Amberlyst®A26 hydroxide form (CAS: 39389-850; 226 mg, 0.72 mmol) was added. The mixture was stirred at room temperature for 45 min. The reaction was filtered and washed with MeOH (several times). The filtrates were evaporated in vacuo to afford intermediate 150 (148 mg, 99%) as a red foamy solid.

### Preparation of Intermediate 151

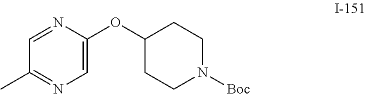 [SMILES: Cc1cnc(OC2CCN(C(=O)OC(C)(C)C)CC2)cn1]

NaH (60% in mineral oil, 103 mg, 2.57 mmol) was added to a stirred solution of 1-Boc-4-hydroxypiperidine (CAS: 109384-19-2; 470 mg, 2.33 mmol) in DMF (10 mL) at 0° C. under N₂ atmosphere. The mixture was stirred at room temperature for 1 h. 2-Chloro-5-methylpyrazine (CAS: 59303-10-5; 300 mg, 2.33 mmol) was added to the mixture under N₂ atmosphere and the reaction mixture was stirred at 50° C. for 16 h. A solution of 1-Boc-4-hydroxypiperidine (CAS: 109384-19-2) in DMF which was stirred for 1 h at room temperature was added, and the reaction mixture was stirred at 80° C. for another 16 h. The mixture was diluted with water and extracted with DCM. The organic layer was dried (MgSO₄), filtered and the solvents were evaporated in vacuo. The crude product was purified by flash column chromatography (silica, heptane/EtOAc, gradient from 100:0 to 80:20). The desired fractions were collected and concentrated in vacuo to afford intermediate 151 (320 mg, 47%) as a white solid.

### Preparation of Intermediate 152

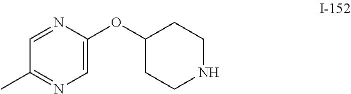

Intermediate 151 (320 mg, 1.09 mmol) was dissolved in HCl (4M in 1,4-dioxane, 4.0 mL, 16.0 mmol). The reaction mixture was stirred at room temperature for 16 h and concentrated in vacuo. The crude product was purified by flash column chromatography (silica, MeOH:NH₃ in DCM, gradient from 0:100 to 10:90). The desired fractions were collected and concentrated in vacuo to give intermediate 152 (189 mg, 87%) as a white solid.

### Preparation of Intermediate 153

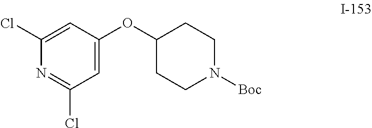 [SMILES: CC(C)(C)OC(=O)N1CCC(Oc2cc(Cl)nc(Cl)c2)CC1]

1-Boc-4-hydroxypiperidine (CAS: 109384-19-2; 3.27 g, 15.8 mmol) was added to a stirred solution of NaH (60% dispersion in mineral oil, 661 mg, 16.5 mmol) in anhydrous THF (20 mL) at 0° C. under N₂ atmosphere. The mixture was warmed to room temperature and stirred for 30 min. Then, 4-nitro-2,6-dichloropyridine (CAS: 25194-01-8; 2.90 g, 15.0 mmol) was added to the mixture at 0° C. and the reaction mixture was stirred at 50° C. for 2 h. The mixture was diluted with water and extracted with EtOAc. The organic layer was dried (MgSO₄), filtered and the solvents were evaporated in vacuo. The crude product was purified by flash column chromatography (silica, heptane/EtOAc, gradient from 100:0 to 80:20). The desired fractions were collected and concentrated in vacuo to give intermediate 153 (4.2 g, 80%) as a pale yellow solid.

### Preparation of Intermediate 154

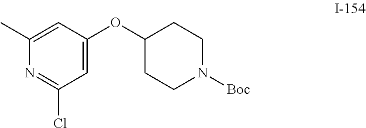 [SMILES: Cc1cc(OC2CCN(C(=O)OC(C)(C)C)CC2)cc(Cl)n1]

Methylmagnesium bromide (1.4M solution, 11.7 mL, 16.4 mmol) was added dropwise to a stirred mixture of intermediate 153 (4.20 g, 11.7 mmol) and iron(III)acetylacetonate (125 mg, 0.35 mmol) in anhydrous THF (58 mL) and anhydrous NMP (11.5 mL) at 0° C. The reaction mixture was stirred at 10° C. for 1 h, diluted with water and extracted with EtOAc. The organic layer was dried (MgSO₄), filtered and the solvents were evaporated in vacuo. The crude product was purified by flash column chromatography (silica, heptane/EtOAc, gradient from 100:0 to 80:20). The desired fractions were collected and concentrated in vacuo to give intermediate 154 (3.08 g, 79%) as colorless solid.

### Preparation of Intermediate 155

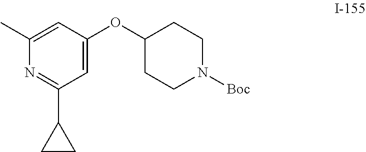 [SMILES: Cc1cc(OC2CCN(C(=O)OC(C)(C)C)CC2)cc(C2CC2)n1]

Intermediate 154 (150 mg, 0.46 mmol), cyclopropylboronic acid (80.5 mg, 0.92 mmol) and tricyclohexylphosphine (11.5 mg, 40.8 μmol) were added to a stirred solution of K₃PO₄ (305 mg, 1.44 mmol) in toluene (4.88 mL) and H₂O (0.57 mL) under N₂ atmosphere. Then Pd(OAc)₂ (4.53 mg, 20.2 μmol) was added. The reaction mixture was stirred at 105° C. for 16 h. The mixture was diluted with water and extracted with EtOAc. The organic layer was dried (MgSO₄), filtered and the solvents were evaporated in vacuo. The crude product was purified by flash column chromatography (silica, heptane/EtOAc, gradient from 100:0 to 50:50) The desired fractions were collected and concentrated in vacuo to afford intermediate 155 (150 mg, 97%) as a colorless sticky solid.

### Preparation of Intermediate 156

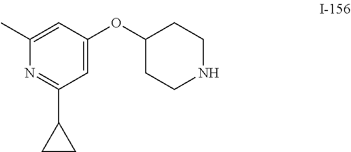 [SMILES: Cc1cc(OC2CCNCC2)cc(C2CC2)n1]

TFA (0.77 mL, 10.4 mmol) was added to a stirred solution of intermediate 155 (172.8 mg, 0.52 mmol) in DCM (2.66 mL) at 0° C. The reaction mixture was stirred at room temperature for 1.5 h and the solvent was removed in vacuo. The residue was dissolved in MeOH and Amberlyst® A26 hydroxide form (CAS: 39389-85-0; 650 mg, 2.08 mmol) was added. The mixture was stirred at room temperature for 45 min, filtered and washed with MeOH several times. The filtrate was evaporated in vacuo to afford intermediate 156 (134 mg, quant, 90% purity) as a beige foamy solid.

### Preparation of Intermediate 157

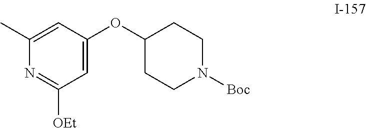 [SMILES: CCOc1cc(OC2CCN(C(=O)OC(C)(C)C)CC2)cc(C)n1]

A solution of sodium (52.8 mg, 2.30 mmol) in EtOH (2.5 mL) under N₂ atmosphere was added dropwise to a solution of intermediate 154 (500 mg, 1.53 mmol) in EtOH (1 mL) at 0° C. The reaction mixture was stirred for 16 h, diluted with NH₄Cl and extracted with EtOAc. The organic layer was dried (MgSO₄), filtered and the solvents were evaporated in vacuo. The crude mixture was purified by flash column chromatography (silica, heptane/EtAOc, gradient from 100:0 to 90:10). The desired fractions were collected and concentrated in vacuo to afford intermediate 157 (224 mg, 44%) as a yellow oil.

### Preparation of Intermediate 158

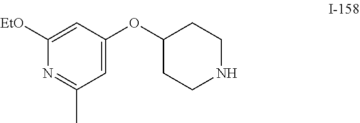 [SMILES: CCOc1cc(OC2CCNCC2)cc(C)n1]

Intermediate 157 (224 mg, 0.67 mmol) was dissolved in HCl (4M in 1,4-dioxane, 0.83 mL, 3.33 mmol). The reaction mixture was stirred at room temperature for 16 h and concentrated in vacuo. The residue was dissolved in MeOH (1 mL) and Amberlyst® A26 hydroxide form (CAS: 39339-85-0; 888 mg, 2.66 mmol) was added. The mixture was stirred at room temperature until pH 7. The resin was removed by filtration and the solvents were evaporated in vacuo. The crude product was purified by flash column chromatography (silica, MeOH:NH₃ in DCM, gradient from 0:100 to 100:0). The desired fractions were collected and concentrated in vacuo to give intermediate 158 (104 mg, 66%) as a colourless oil.

### Preparation of Intermediate 159

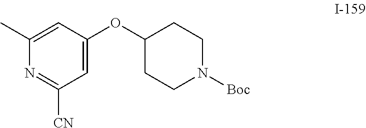 [SMILES: Cc1cc(OC2CCN(C(=O)OC(C)(C)C)CC2)cc(C#N)n1]

Dppf (71.2 mg, 0.13 mmol) and Pd₂dba₃ (59.2 mg, 62.7 μmol) were added to DMA (22 mL) while the solvent was degassed with N₂ at 45° C. The mixture was stirred under N₂ atmosphere at 45° C. for 5 min. Zn (16.7 mg, 0.25 mmol) and Zinc cyanide (84.2 mg, 0.70 mmol) were added under N₂ at 45° C. Intermediate 154 (410 mg, 1.26 mmol) was added under N₂ at 45° C. The reaction mixture was stirred in a sealed tube at 120° C. for 16 h The mixture was cooled down, diluted with NaHCO₃ (sat., aq.) and extracted with EtOAc. The organic layer was washed with water, dried (MgSO₄), filtered and the solvents were evaporated in vacuo. The crude product was purified by flash column chromatography (silica, heptane/EtOAc, gradient from 100:0 to 85:15). The desired fractions were collected and concentrated in vacuo to afford intermediate 159 (399 mg, 99%) as a pink solid.

### Preparation of Intermediate 160

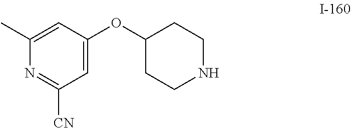 [SMILES: Cc1cc(OC2CCNCC2)cc(C#N)n1]

Intermediate 160 was prepared following an analogous procedure to the one described for the synthesis of intermediate 156 using intermediate 159 as starting material.

### Preparation of Intermediate 161

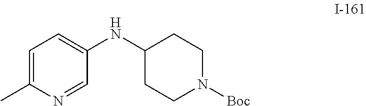 [SMILES: Cc1ccc(NC2CCN(C(=O)OC(C)(C)C)CC2)cn1]

Pd₂dba₃ (79.8 mg, 87.2 μmol) was added to a solution of Cs₂CO₃ (1.71 g, 5.23 mmol) and XPhos (101 mg, 0.17 mmol) in toluene (26 mL) while N₂ was bubbling and the mixture was stirred at 40° C. for 2 min. tert-Butyl 4-amino-1-piperidinecarboxylate (CAS: 87120-72-7; 349 mg, 1.74 mmol) was added while N₂ was bubbling. The mixture was stirred at 40° C. for 5 min and 5-bromo-2-methylpyridine (CAS: 3430-13-5; 300 mg, 1.74 mmol) was added. The reaction mixture was stirred at 105° C. for 18 h. Water was added and the mixture was extracted with EtOAc (3 times). The combined organic layers were dried (MgSO₄), filtered and evaporated in vacuo. The crude mixture was purified by flash column chromatography (silica, heptane/EtOAc, gradient from 100:0 to 50:50). The desired fractions were collected and concentrated in vacuo to give intermediate 161 (409 mg, 80%) as a white solid.

### Preparation of Intermediate 162

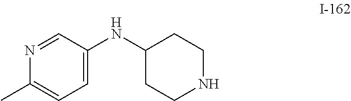

Intermediate 162 was prepared following an analogous procedure to the one described for the synthesis of intermediate 156 using intermediate 161 as starting material.

### Preparation of Intermediate 163

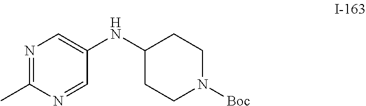 [SMILES: Cc1ncc(NC2CCN(C(=O)OC(C)(C)C)CC2)cn1]

Intermediate 163 was prepared following an analogous procedure to the one described for the synthesis of intermediate 161 using 5-bromo-2-methylpyrimidine (CAS: 7752-78-5) and tert-butyl 4-amino-1-piperidinecarboxylate (CAS: 87120-72-7) as starting materials.

The crude mixture was purified by flash column chromatography (silica, EtOAc in heptane, gradient from 0:100 to 50:50). The desired fractions were collected and concentrated in vacuo to afford intermediate 163 (621 mg, 73%) as a white solid.

### Preparation of Intermediate 164

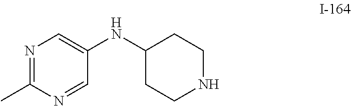

Intermediate 164 was prepared following an analogous procedure to the one described for the synthesis of intermediate 156 using intermediate 163 as starting material.

### Preparation of Intermediate 165

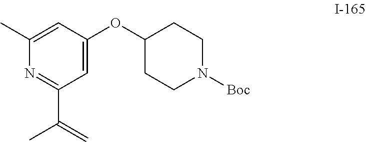 [SMILES: C=C(C)c1cc(OC2CCN(C(=O)OC(C)(C)C)CC2)cc(C)n1]

Pd(dppf)Cl₂.DCM (60.0 mg, 73.4 μmol) was added to a mixture of intermediate 154 (400 mg, 1.22 mmol), potassium trifluoro(prop-1-en-2-yl)borate (CAS: 395083-14-4; 272 mg, 1.84 mmol) and Cs₂CO₃ (1.40 g, 2.94 mmol) in H₂O (1.12 mL) and 1,4-dioxane (9 mL) at room temperature while N₂ was bubbling. The reaction mixture was stirred at 90° C. in a sealed tube for 48 h. Water was added and the mixture was extracted with EtOAc (3 times). The combined organic extracts were dried (MgSO₄), filtered and the solvent was removed in vacuo. The crude product was purified by flash column chromatography (silica, heptane/EtOAc, gradient from 100:0 to 85:15). The desired fractions were collected and concentrated in vacuo to give intermediate 165 (383 mg, 94%) as a colorless oil.

### Preparation of Intermediate 166

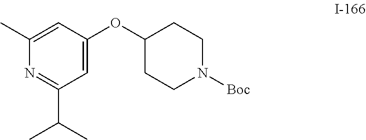 [SMILES: Cc1cc(OC2CCN(C(=O)OC(C)(C)C)CC2)cc(C(C)C)n1]

Pd/C (123 mg, 0.12 mmol, 10% purity) was added to a stirred solution of intermediate 165 (383 mg, 1.15 mmol) in MeOH (7.5 mL) at room temperature under N₂ atmosphere. The mixture was purged with H2 and stirred at room temperature for 4 h under H2 atmosphere. The mixture was filtered over Celite®. The filtrate was extracted with EtOAc and MeOH and the solvent was removed in vacuo to afford intermediate 166 (381 mg, 99%) as a black oil.

### Preparation of Intermediate 167

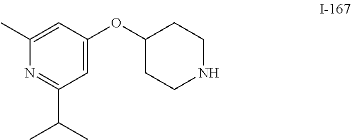 [SMILES: Cc1cc(OC2CCNCC2)cc(C(C)C)n1]

TFA (0.51 mL, 6.84 mmol) was added to a stirred solution of intermediate 166 (381 mg, 0.34 mmol, 30% purity) in DCM (1.75 mL) at 0° C. The reaction mixture was stirred at room temperature for 1.5 h and the solvent was evaporated in vacuo. Amberlyst®A26 hydroxide form (CAS: 39339-85-0; 1.03 g, 3.3 mmol) was added to a solution of the residue (355 mg) in MeOH (2 mL) and the mixture was stirred at room temperature until the pH of the mixture was basic (2 h). The mixture was filtered and washed with MeOH. The solvent was removed in vacuo to afford intermediate 167 which was used as such in next step.

### Preparation of Intermediate 168

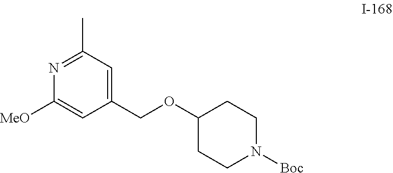 [SMILES: COc1cc(COC2CCN(C(=O)OC(C)(C)C)CC2)cc(C)n1]

NaH (60% in mineral oil, 87.7 mg, 2.19 mmol) was added to a stirred solution of 1-Boc-4-hydroxypiperidine (442 mg, 2.19 mmol) in anhydrous THF 1.58 mL) at 0° C. and the mixture was stirred for 10 min at 0° C. and 20 min at room temperature. 4-(Bromoethyl)-2-methoxy-6-methylpyridine (158 mg, 0.73 mmol) was added and the reaction mixture was stirred for 16 h at room temperature. The solvent was removed in vacuo. Water was added to the residue and the mixture was extracted with EtOAc. The organic layer was dried (MgSO₄), filtered and the solvent was removed in vacuo. The crude product was purified by flash column chromatography (silica, EtOAc in heptane, gradient from 0/100 to 50/50). The desired fractions were collected and concentrated in vacuo to give intermediate 168 (170 mg, 69%) as a colorless oil.

### Preparation of Intermediate 169

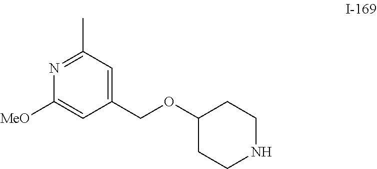 [SMILES: COc1cc(COC2CCNCC2)cc(C)n1]

Intermediate 169 was prepared following an analogous procedure to the one described for the synthesis of intermediate 167 using intermediate 168 as starting material.

### Preparation of Intermediate 170

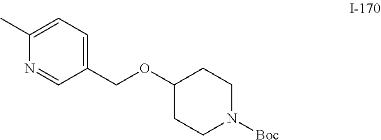 [SMILES: Cc1ccc(COC2CCN(C(=O)OC(C)(C)C)CC2)cn1]

NaH (60% dispersion in mineral oil, 233 mg, 1.94 mmol) was added to a stirred solution of 1-Boc-4-hydroxypiperidine (1.17 g, 5.83 mmol) in anhydrous THF (4 mL) at 0° C. The reaction mixture was stirred for 10 min at 0° C. and 20 min at room temperature. 5-(bromomethyl)-2-methylpyridine (CAS: 792187-67-8; 362 mg, 1.94 mmol) was added and the reaction mixture was stirred for 18 h at 60° C. The solvent was removed in vacuo. Water was added and the mixture was extracted with EtOAc. The organic layer was dried (MgSO₄), filtered and concentrated in vacuo. The crude product was purified by flash column chromatography (silica, EtOAc in heptane, gradient from 0/100 to 80/20). The desired fractions were collected and concentrated in vacuo to afford intermediate 170 (106 mg, 18%) as a colorless oil.

### Preparation of Intermediate 171

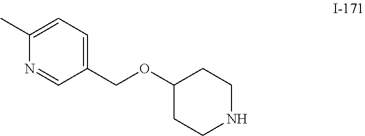

Intermediate 171 was prepared following an analogous procedure to the one described for the synthesis of intermediate 167 using intermediate 170 as starting material.

### Preparation of Intermediate 172

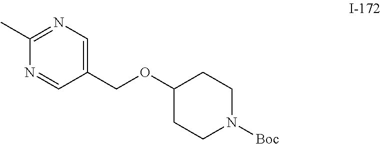 [SMILES: Cc1ncc(COC2CCN(C(=O)OC(C)(C)C)CC2)cn1]

Intermediate 172 was prepared following an analogous procedure to the one described for the synthesis of intermediate 170 using 1-Boc-4-hydroxypiperidine and 5-bromomethyl-2-methyl-pyrimidine as starting materials.

### Preparation of Intermediate 173

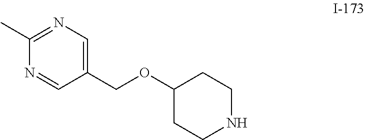

Intermediate 173 was prepared following an analogous procedure to the one described for the synthesis of intermediate 167 using intermediate 172 as starting material.

### Preparation of Intermediate 174

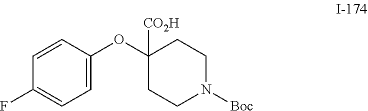 [SMILES: CC(C)(C)OC(=O)N1CCC(Oc2ccc(F)cc2)(C(=O)O)CC1]

NaH (60% in mineral oil, 1.27 g, 31.8 mmol) was added to a stirred solution of 4-fluorophenol (1.00 g, 8.92 mmol) in anhydrous THF (30 mL) at room temperature and the mixture was stirred for 3 h. 1-Boc-4-piperidone (CAS: 79099-07-3; 4.68 g, 23.5 mmol) was added. The mixture was cooled to 0° C. and anhydrous CHCl₃ (2.82 mL) was added dropwise. The reaction mixture was stirred at 0° C. for 1 h, then at 40° C. for 3 h. The mixture was cooled to room temperature and was stirred for 48 h. The solvent was removed in vacuo. The mixture was suspended in water (30 mL) and washed with Et₂O (30 mL). The aqueous layer was acidified with HCl 6N until pH 5, filtered and extracted with DCM. The combined organic extracts were dried (MgSO₄), filtered and the solvent was evaporated in vacuo. The crude product was purified by flash column chromatography (silica, MeOH in DCM, gradient from 0/100 to 5/95). The desired fractions were collected and concentrated in vacuo to afford intermediate 174 (2.88 g, 79%, 83% purity) as a white sticky solid.

### Preparation of Intermediate 175

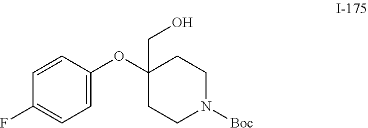 [SMILES: CC(C)(C)OC(=O)N1CCC(CO)(Oc2ccc(F)cc2)CC1]

LiAlH₄ (338 mg, 8.45 mmol) was added portion wise to a stirred solution of intermediate 174 (2.88 g, 7.04 mmol, 83% purity) in anhydrous THF (30 mL) at −20° C. under N₂ atmosphere. The reaction mixture was stirred at 65° C. for 1.5 h. NaOH (2N, aq.) and water were added. The mixture was filtered on Celite®. The organic layer was separated, dried (MgSO₄), filtered and the solvent was removed in vacuo. The crude product was purified by flash column chromatography (silica, EtOAc in heptane, gradient from 0/100 to 50/50). The desired fractions were collected and concentrated in vacuo to afford intermediate 175 (1.28 g, 56%) as a colourless oil.

### Preparation of Intermediate 176

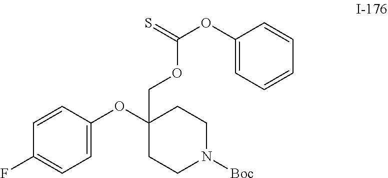 [SMILES: CC(C)(C)OC(=O)N1CCC(COC(=S)Oc2ccccc2)(Oc2ccc(F)cc2)CC1]

O-Phenylchlorothionoformiate (0.61 mL, 4.33 mmol) in DCM (29.1 mL) was added portionwise to a stirred solution of intermediate 175 (1.28 g, 3.93 mmol) in pyridine (0.48 mL) and DCM (29.1 mL) under N₂ atmosphere at 0° C. The mixture was stirred at room temperature for 1 h, quenched with the addition of MeOH (0.26 mL) and concentrated in vacuo. The residue was diluted in DCM and washed with HCl (2M, aq.) and water. The organic layer was dried (MgSO₄), filtered and the solvents were evaporated in vacuo. The crude product was purified by flash column chromatography (silica, heptane/EtOAc, gradient from 100:0 to 80:20) to afford intermediate 176 (1.5 g, 74%) as a yellow oil.

### Preparation of Intermediate 177

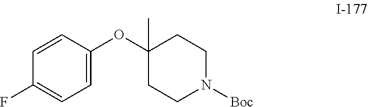 [SMILES: CC(C)(C)OC(=O)N1CCC(C)(Oc2ccc(F)cc2)CC1]

Tributyltin hydride (CAS: 688-73-3; 6.42 mL, 23.4 mmol) and AIBN (CAS: 78-67-1; 520 mg, 3.07 mmol) were added to a stirred solution of intermediate 176 (1.35 g, 2.93 mmol) in toluene (96.3 mL) at room temperature. The reaction mixture was stirred at 100° C. for 90 min and the solvent was evaporated in vacuo. The crude product was purified by flash column chromatography (silica, EtOAc in heptane, gradient from 0:100 to 15:85). The desired fractions were collected and concentrated in vacuo to afford intermediate 177 (205 mg, 11%, 50% purity) as a brown oil.

### Preparation of Intermediate 178

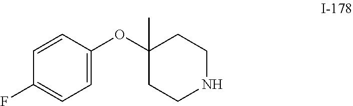

TFA (0.49 mL, 6.63 mmol) was added to a stirred solution of intermediate 177 (205 mg, 0.33 mmol, 50% purity) in DCM (1 mL) at 0° C. The reaction mixture was stirred at room temperature for 1.5 h. The solvent was evaporated in vacuo. Amberlyst®A26 (CAS: 39339-85-0; 2.05 gm 6.56 mmol) was added to a solution of the residue (143 mg) in MeOH (5 mL) and the mixture was stirred until the pH of the solution was basic. The mixture was filtered, washed with MeOH and concentrated in vacuo to give intermediate 178 (67.9 mg) as a yellow oil.

### Preparation of Intermediate 179

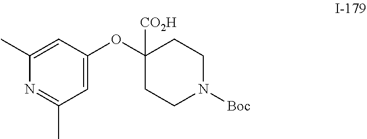 [SMILES: Cc1cc(OC2(C(=O)O)CCN(C(=O)OC(C)(C)C)CC2)cc(C)n1]

Intermediate 179 was prepared following an analogous procedure to the one described for the synthesis of intermediate 174 using 1-Boc-4-piperidone (CAS: 79099-07-3) and 2,6-dimethyl-4-hydroxypyridine (CAS: 13603-44-6) as starting materials.

### Preparation of Intermediate 180

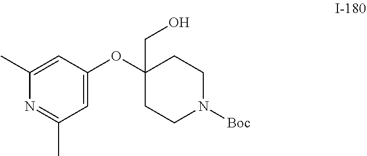 [SMILES: Cc1cc(OC2(CO)CCN(C(=O)OC(C)(C)C)CC2)cc(C)n1]

Intermediate 180 was prepare following an analogous procedure to the one described for the synthesis of intermediate 175 using intermediate 179 as starting material.

### Preparation of Intermediate 181

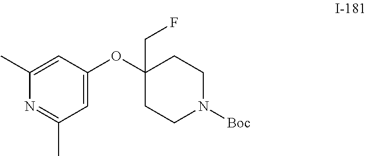 [SMILES: Cc1cc(OC2(CF)CCN(C(=O)OC(C)(C)C)CC2)cc(C)n1]

DAST (328 μL, 2.68 mmol) was added to a stirred solution of intermediate 180 (300 mg, 0.89 mmol) in anhydrous DCM (6.69 mL) at room temperature under N₂ atmosphere. The reaction mixture was stirred for 16 h, quenched with NaHCO₃ (sat., aq.) and extracted with DCM. The organic layer was dried (MgSO₄), filtered and the solvents were evaporated in vacuo. The crude product was purified by flash column chromatography (silica, EtOAc in heptane, gradient from 0/100 to 50/50) to afford intermediate 181 (167 mg 55%) as a colorless oil.

### Preparation of Intermediate 182

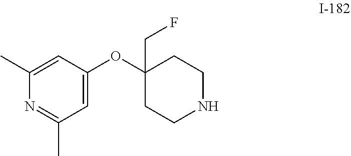 [SMILES: Cc1cc(OC2(CF)CCNCC2)cc(C)n1]

Intermediate 182 was prepared following an analogous procedure to the one described for the synthesis of intermediate 178 using intermediate 181 as starting material.

### Preparation of Intermediate 183

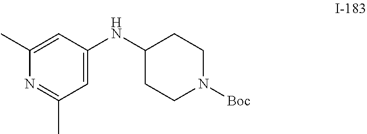 [SMILES: Cc1cc(NC2CCN(C(=O)OC(C)(C)C)CC2)cc(C)n1]

Pd₂dba₃ (73.8 mg, 80.6 μmol) was added to a mixture of Cs₂CO₃ (1.57 g, 4.84 mmol) and DavePhos (63.5 mg, 0.16 mmol) in toluene (15 mL) while N₂ was bubbling. The mixture was stirred for 2 min at 40° C. and 4-bromo-2,6-dimethylpyridine (CAS: 5093-70-9; 300 mg, 1.61 mmol) was added. The mixture was stirred at 40° C. for 5 min and 1-Boc-4-aminopiperidine (CAS: 87120-72-7; 323 mg, 1.61 mmol) was added. The reaction mixture was stirred for 24 h at 95° C. The solvent was removed in vacuo. Water was added to the residue and the mixture was extracted with EtOAc (3 times). The combined organic layers were dried (MgSO₄), filtered and evaporated in vacuo. The crude product was purified by flash column chromatography (silica, EtOAc in heptane, gradient from 0/100 to 100/100). The desired fractions were collected and concentrated in vacuo to afford intermediate 183 (370 mg, 75%) as a yellow solid.

### Preparation of Intermediate 184

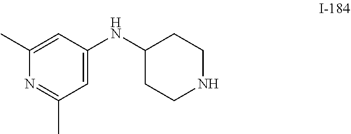

Intermediate 184 was prepared following an analogous procedure to the one described for the synthesis of intermediate 178 using intermediate 183 as starting material.

### Preparation of Intermediate 199

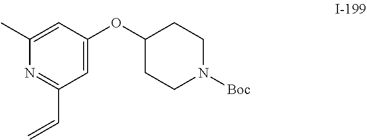 [SMILES: C=Cc1cc(OC2CCN(C(=O)OC(C)(C)C)CC2)cc(C)n1]

Pd(dppf)Cl₂.DCM (60 mg, 73.4 μmol) was added to a mixture of intermediate 154 (400 mg, 1.22 mmol), potassium trifluoro(vinyl)borate (180 mg, 1.35 mmol) and Cs₂CO₃ (1.4 g, 2.94 mmol) in 1,4-dioxane (9 mL) and water (1.12 mL). under N₂ atmosphere. The reaction mixture was stirred at 90° C. in a sealed tube for 16 h. The layers were separated and the aqueous phase was extracted with EtOAc. The combined organic fractions were washed with water and brine, dried (MgSO₄), filtered and the solvents were evaporated in vacuo. The crude mixture was purified by flash column chromatography (silica, EtOAc in heptane, gradient from 0/100 to 20/80). The desired fractions were collected and concentrated in vacuo to afford intermediate 199 (168 mg, 43%) as a yellow oil.

### Preparation of Intermediate 200

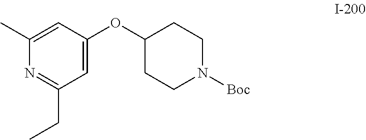 [SMILES: CCc1cc(OC2CCN(C(=O)OC(C)(C)C)CC2)cc(C)n1]

Pd/C (10%, 56.1 mg, 52.8 μmol) was added to a stirred solution of intermediate 199 (168 mg, 0.53 mmol) in MeOH (4 mL) at room temperature. The mixture was purge with H2 and the reaction mixture was stirred for 4 h under H2 atmosphere. The mixture was filtered on a pad of Celite® and the filtrate was extracted with EtOAc and MeOH. The solvent was removed in vacuo to give intermediate 200 (167 mg, 99%) as a black oil.

### Preparation of Intermediate 201

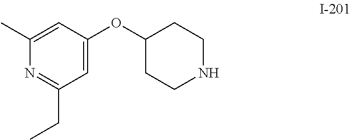 [SMILES: CCc1cc(OC2CCNCC2)cc(C)n1]

TFA (0.78 mL, 10.4 mmol) was added to a stirred solution of intermediate 200 (168 mg, 0.52 mmol) in DCM (2.7 mL) at 0° C. The reaction mixture was stirred at room temperature for 1.5 h and the solvent was evaporated in vacuo. Amberlyst®A26 hydroxide form (CAS: 39339-85-0) was added to the residue dissolved in MeOH and the mixture was stirred at room temperature until pH was basic (2 h). The mixture was filtered and washed with MeOH. The solvent was removed to afford intermediate 201 which was used in the next step without any purification.

### Preparation of Intermediate 202

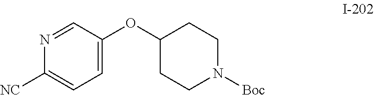 [SMILES: CC(C)(C)OC(=O)N1CCC(Oc2ccc(C#N)nc2)CC1]

NaH (60% dispersion in mineral oil, 109 mg, 2.73 mmol) was added to a stirred solution of 1-Boc-4-hydroxypiperidine (CAS: 109384-19-2; 500 mg, 2.48 mmol) in DMF (10 mL) at 0° C. under N₂ atmosphere. The reaction mixture was stirred at room temperature for 1 h. Then, 5-chloro-2-cyanopyridine (CAS: 80809-64-3; 344 mg, 2.48 mmol) was added. The reaction mixture was stirred at 50° C. for 16 h. The mixture was diluted with water and extracted with DCM. The organic layer was dried (MgSO₄), filtered and the solvents were evaporated in vacuo. The crude product was purified by flash column chromatography (silica, EtOAc in heptane, gradient from 0/100 to 20/80). The desired fractions were collected and concentrated in vacuo to afford intermediate 202 (722 mg, 96%) as a white solid.

### Preparation of Intermediate 203

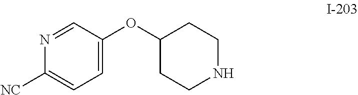 [SMILES: N#Cc1ccc(OC2CCNCC2)cn1]

TFA (1.82 mL, 23.8 mmol) was added to a stirred solution of intermediate 202 (722 mg, 2.38 mmol) in DCM (10.6 mL) at 0° C. The reaction mixture was stirred at room temperature for 24 h. The solvent was evaporated in vacuo. The crude product was purified by flash column chromatography (silica, MeOH in DCM, gradient from 0/100 to 10/90). The desired fractions were collected and concentrated in vacuo to afford intermediate 203 (385 mg, 80%) as a yellow oil.

### Preparation of Intermediate 204

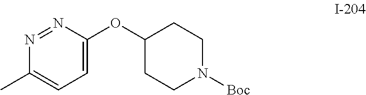 [SMILES: Cc1ccc(OC2CCN(C(=O)OC(C)(C)C)CC2)nn1]

NaOtBu (2.24 g, 23.3 mmol) was added to a solution of 1-tert-butoxycarbonyl-4-hydroxypiperidine (CAS: 109384-19-2; 1.56 g, 7.78 mmol) in DMSO (3 mL), The reaction mixture was stirred at room temperature for 1 h. Then, 3-chloro-6-methylpyridazine (CAS: 1121_79-5; 1.00 g, 7.78 mmol) was added and the reaction mixture was stirred at 50° C. for 16 h. The mixture was cooled to room temperature and water was added. The mixture was extracted with EtOAc (3 times). The combined organic layers were washed with NaHCO₃ and brine, dried (MgSO₄), filtered and the solvents were evaporated in vacuo. The crude product was purified by flash column chromatography (silica, heptane/EtOAc, gradient from 0/100 to 10/90). A second purification was performed by reverse phase chromatography ([25 mM NH₄HCO₃]/[MeCN:MeOH 1:1], gradient from 70/30 to 27/73). The desired fractions were collected and concentrated in vacuo to afford intermediate 204 (318 mg, 14%) as a white solid.

### Preparation of Intermediate 205

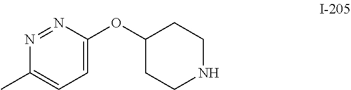

HCl (4M in 1,4-dioxane, 1.35 mL, 5.42 mmol) was added to intermediate 204 (318 mg, 1.08 mmol). The reaction mixture was stirred at room temperature for 16 h. The solvent was evaporated in vacuo. The crude product was purified by flash column chromatography (silica, MeOH:NH₃ in DCM, gradient from 0/100 to 10/90). The desired fractions were collected and concentrated in vacuo to afford intermediate 205 (206 mg, 98%) as a yellow oil.

### Preparation of Intermediate 10

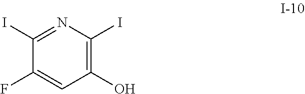

To a solution of 3-fluoro-5-hydroxypyridine (CAS: 209328-55-2, 2 g, 17.7 mmol) in Na₂CO₃ (30 mL, aq. sat. sol.) and water (10 mL), I₂ (CAS: 7553-56-2, 9.2 g, 36.25 mmol) was added and the mixture was stirred at rt for 16 h. The reaction mixture was quenched with an aq. sat. sol. of Na₂S₂O₃ and the solution pH was adjusted to pH=5 by addition of aqueous HCl. The mixture was extracted with EtOAc (3×70 mL) and the combined organic layers was separated, dried (MgSO₄), filtered and evaporated in vacuo to yield intermediate 10 as a yellow solid (6.02 g, 93%).

### Preparation of Intermediate 11

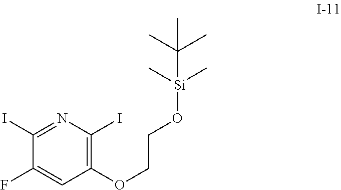 [SMILES: CC(C)(C)[Si](C)(C)OCCOc1cc(F)c(I)nc1I]

A mixture of intermediate 10 (6.1 g, 16.7 mmol), (2-bromoethoxy)dimethyl-tert-butylsilane (CAS: 86864-60-0, 4.4 g, 18.4 mmol), and potassium tert-butoxide (CAS: 865-47-4, 5.08 g, 36.78 mmol) in DMF (15 mL) was stirred at 90° C. for 5 h. The cooled mixture was diluted with water and extracted with EtOAc (2×20 mL). The combined organic layers were separated, dried (MgSO₄), filtered and the solvents evaporated in vacuo. The crude product was purified by flash column chromatography (silica; EtOAc in heptane 0/100 to 20/80). The desired fractions were collected and the solvents concentrated in vacuo to yield intermediate 11 as an oil (8.1 g, 93%).

### Preparation of Intermediate 12

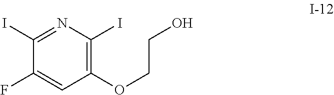 [SMILES: OCCOc1cc(F)c(I)nc1I]

Tetrabutylammonium fluoride (CAS: 429-41-4, 15.3 mL, 15.3 mmol, 1M solution in THF) was added to a solution of intermediate 11 (8 g, 15.3 mmol) in THF (120 mL). The mixture was stirred at rt for 3 h. The mixture was diluted with water and extracted with EtOAc. The organic phase was separated, dried (Na₂SO₄), filtered and evaporated in vacuo. The crude product was purified by flash column chromatography (silica, MeOH in DCM 0/100 to 5/95). The desired fractions were collected and the solvents concentrated in vacuo to yield intermediate 12 as an oil (5.8 g, 92%).

### Preparation of Intermediate 13

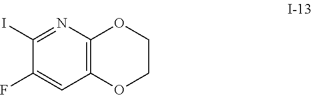 [SMILES: Fc1cc2c(nc1I)OCCO2]

Potassium tert-butoxide (CAS: 865-47-4, 206 mg, 1.83 mmol) was added to a solution of intermediate 12 (5 g, 12.2 mmol) in t-BuOH (6.91 mL) at rt. The mixture was stirred at 90° C. for 3 h. After cooling, the solvent was removed in vacuo and the residue was diluted with water and extracted with EtOAc (3×12 mL). The combined organic layers were washed with brine (2×10 mL), dried (Na₂SO₄), filtered and concentrated in vacuo. The crude product was purified by flash column chromatography (silica, MeOH in DCM 0/100 to 5/95). The desired fractions were collected and concentrated in vacuo to yield intermediate 13 as a white solid (1.6 g, 47%).

### Preparation of Intermediate 14

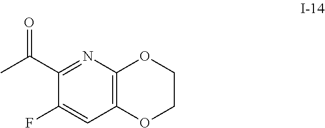

Bis(triphenylphosphine)palladium(II) dichloride (CAS: 13965-03-2, 400 mg, 0.57 mmol) and tributyl(1-ethoxyvinyl)tin (CAS: 97674-02-7; 2.5 mL, 7.4 mmol) were added to a stirred solution of intermediate 13 (1.6 g, 5.7 mmol) in toluene (15 mL). The mixture was heated at 92° C. for 16 h, then the mixture was cooled and treated with aqueous 2N HCl (5 mL) and the mixture was stirred for 2 h. The crude was neutralised with an aq. sat. sol. of NaHCO₃ and extracted with EtOAc. The combined organic layers were separated, dried (Na₂SO₄), filtered and evaporated in vacuo. The crude product was purified (silica, MeOH in DCM 0/100 to 5/95). The desired fractions were collected and concentrated in vacuo to yield intermediate 14 as an orange solid (0.85 g, 76%).

### Preparation of Intermediate 15

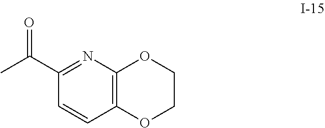

Intermediate 15 was prepared following an analogous procedure to the one described for the synthesis of intermediate 14 using 6-iodo-2,3-dihydro-[1,4]dioxino[2,3-b]pyridine (CAS: 1246088-42-5) as starting material.

### Preparation of Intermediate 16

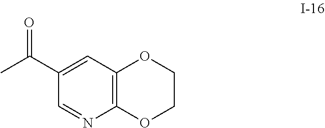

Intermediate 16 was prepared following an analogous procedure to the one described for the synthesis of intermediate 14 using 7-bromo-2,3-dihydro-[1,4]dioxino[2,3-b]pyridine (CAS: 95897-49-7) as starting material.

### Preparation of Intermediate 17

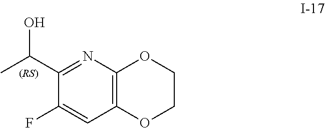

Sodium borohydride (CAS: 137141-62-9, 0.73 g, 19.33 mmol) was added to a stirred solution of intermediate 14 (1 g, 4.83 mmol) in MeOH (6.91 mL) at 0° C. The mixture was stirred at rt for 10 min and then diluted with water and extracted with DCM (3×80 mL). The combined organic layers were dried (Na₂SO₄), filtered and the solvents concentrated in vacuo to yield intermediate 17 (0.86 g, 89%) as colourless oil, used in the next step without further purification.

### Preparation of Intermediate 18

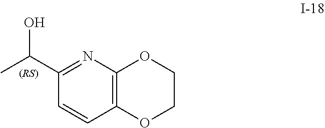

Intermediate 18 was prepared following an analogous procedure to the one described for the synthesis of intermediate 17 using intermediate 15 as starting material.

### Preparation of Intermediate 19

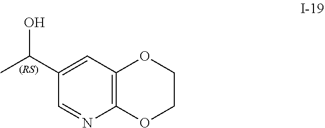

Intermediate 19 was prepared following an analogous procedure to the one described for the synthesis of intermediate 17 using intermediate 16 as starting material.

### Preparation of Intermediate 20

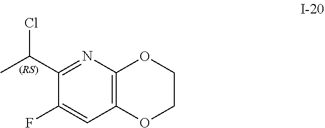

Thionyl chloride (CAS: 7719-09-7, 1.26 mL, 17.27 mmol) was added to a stirred solution of intermediate 17 (0.86 g, 4.32 mmol) in DCM (29 mL) at 0° C. The mixture was stirred at rt for 12 h and then diluted with water and extracted with DCM. The organic layer was dried (Na₂SO₄), filtered and the solvents concentrated in vacuo to yield intermediate 20 (0.89 g, 95%) as cream solid, used in the next step without further purification.

### Preparation of Intermediate 21

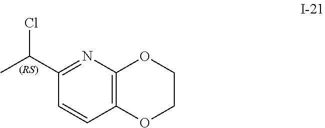

Intermediate 21 was prepared following an analogous procedure to the one described for the synthesis of intermediate 20 using intermediate 18 as starting material.

### Preparation of Intermediate 22

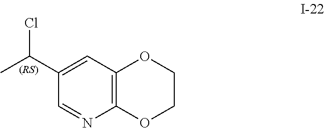

Intermediate 22 was prepared following an analogous procedure to the one described for the synthesis of intermediate 20 using intermediate 19 as starting material.

### Preparation of Intermediate 23

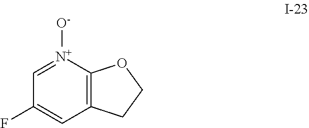 [SMILES: [O-][n+]1cc(F)cc2c1OCC2]

m-Chloroperbenzoic acid (CAS: 937-14-4; 806 mg, 4.7 mmol) was added to a mixture of 5-fluoro-2,3-dihydrofuro[2,3-b]pyridine (CAS: 1356542-41-0; 500 mg, 3.6 mmol) in DCM (15 mL) at rt. The mixture was stirred at 25° C. for 36 h. The solvent was removed in vacuo, and the crude product was purified by flash column chromatography (silica, EtOAc in heptane 0/100 to 30/70 then DCM in MeOH 0/100 to 6/94). The desired fractions were collected and the solvents evaporated in vacuo to yield intermediate 23 as a white solid (400 mg, 72%).

### Preparation of Intermediate 24

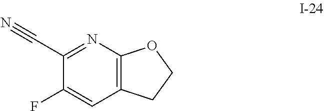 [SMILES: N#Cc1nc2c(cc1F)CCO2]

Trimethylsilyl cyanide (CAS: 7677-24-9; 1.29 mL, 10.3 mmol) and triethylamine (0.9 mL, 6.47 mmol) were added to a mixture of intermediate 23 (400 mg, 2.57 mmol) in acetonitrile (7 mL). The mixture was stirred at 90° C. for 24 h. The mixture was cooled, diluted with water and extracted with EtOAc (2×10 mL). The combined organic extracts were dried (MgSO₄), filtered and the solvent evaporated in vacuo. The residue was purified by flash column chromatography (silica, EtOAc in heptane 0/100 to 40/60). The desired fractions were collected and concentrated in vacuo to yield intermediate 24 as an oil (320 mg, 76%).

### Preparation of Intermediate 25

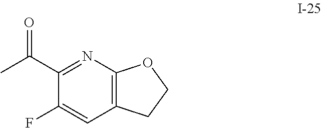

Methyl magnesium bromide (CAS: 75-16-1, 2.071 mL, 2.9 mmol, 1.4 M in THF/toluene) was added dropwise to a solution of intermediate 24 (340 mg, 2.071 mmol) in dry THF (20 mL) at 0° C. After completion of the addition, the reaction was stirred at rt for 16 h. The mixture was quenched with 1M aq HCl and stirred for 30 min, then the crude was basified with NH₄OH until pH 8. The solution was extracted with EtOAc (2×5 mL) The combined organic extracts were dried (Na₂SO₄), filtered and evaporated in vacuo. The crude product was purified by flash column chromatography (silica, EtOAc in heptane 0/100 to 20/80). The desired fractions were collected and concentrated in vacuo to yield intermediate 25 as colourless oil (150 mg, 40%).

### Preparation of Intermediate 26

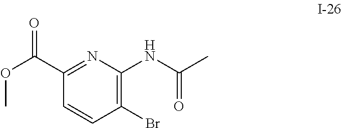

Acetic anhydride (CAS: 108-24-7; 13.2 g, 129.8 mmol) was added to a stirred mixture of methyl 6-amino-5-bromopyridine-2-carboxylate (CAS: 178876-82-9; 30 g, 129.8 mmol) in toluene (600 mL) under N₂. The mixture was stirred at 100° C. for 36 h and then the solvent was evaporated in vacuo. The residue was purified by flash column chromatography (silica; EtOAc in petroleum ether 0/100 to 50/50). The desired fractions were collected and concentrated in vacuo to yield intermediate 26 as a white solid (14.0 g, 40%).

### Preparation of Intermediate 27

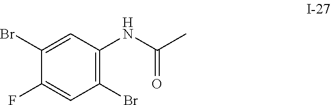

Intermediate 27 was prepared following an analogous procedure to the one described for the synthesis of intermediate 26 using 2,5-dibromo-4-fluoroaniline (CAS: 172377-05-8) as starting material.

### Preparation of Intermediate 28

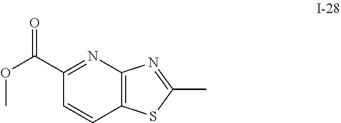

Phosphorus pentasulfide (CAS: 1314-80-3; 13.7 g, 61.5 mmol) was added to a suspension of intermediate 26 (14.0 g, 51.3 mmol) in THF (200 mL) under N₂. The mixture was stirred at rt for 16 h and then at 70° C. for 48 h. The solvent was evaporated in vacuo and the residue purified by flash column chromatography (silica; EtOAc in petroleum ether 0/100 to 50/50). The desired fractions were collected and concentrated in vacuo to yield intermediate 28 as a yellow solid (7.5 g, 69%).

### Preparation of Intermediate 29

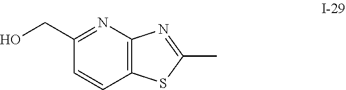

Sodium borohydride (CAS: 16940-66-2; 6.81 g, 180.0 mmol) was added to a stirred suspension of intermediate 28 (7.55 g, 36.0 mmol) in THF (60 mL). The mixture was stirred at 25° C. for 5 h and then a aq. sat. sol. NH₄C (100 mL) was added. The mixture was extracted with DCM and the organic layer was separated, dried (Na₂SO₄), filtered and the solvents evaporated in vacuo to yield intermediate 29 as a yellow solid (3.1 g, 51%).

### Preparation of Intermediate 30

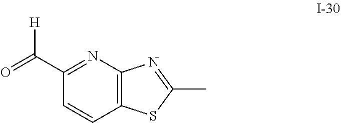

MnO₂ (CAS: 1313-13-9; 7.48 g, 86.0 mmol) was added to a stirred suspension of intermediate 29 (3.1 g, 17.2 mmol) in 1,4-dioxane (50 mL). The mixture was stirred at 80° C. for 16 h and then filtered through a Celite® pad. The filtrate was evaporated in vacuo and the residue purified by flash column chromatography (silica; EtOAc in petroleum ether 0/100 to 50/50). The desired fractions were collected and concentrated in vacuo to yield intermediate 30 as a yellow solid (2.0 g, 65%).

### Preparation of Intermediate 31

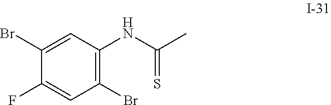

Phosphorus pentasulfide (CAS: 1314-80-3; 0.9 g, 4.06 mmol) was added to a suspension of intermediate 27 (0.97 g, 3.12 mmol) in THF (17 mL) under N₂. The mixture was stirred at rt for 16 h. Then Cs₂CO₃ (1.63 g, 4.99 mmol) was added and the mixture was stirred at 70° C. for 16 h. Then, the mixture was diluted with water and 2N aq. NaOH were added and extracted with EtOAc. The organic layer was separated, dried (MgSO₄), filtered and the solvents evaporated in vacuo. The crude product was purified by flash column chromatography (silica; EtOAc in heptane 0/100 to 80/20).

The desired fractions were collected and concentrated in vacuo to yield intermediate 31 as a yellow solid (0.62 g, 61%).

### Preparation of Intermediate 32

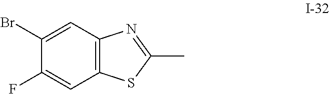

Intermediate 31 (620 mg, 1.9 mmol) was added to a stirred suspension of sodium hydride (CAS: 7646-69-7; 60% dispersion in mineral oil, 91 mg, 2.28 mmol) in toluene (8.51 mL). The mixture was stirred at rt for 2 h and then, DMF (1.7 mL) was added and the resulting reaction mixture was stirred at 110° C. for 16 h. The mixture was diluted with aq. sat. sol. NaCl and extracted with EtOAc. The organic layer was separated, dried (MgSO₄), filtered and the solvents evaporated in vacuo to yield intermediate 32 (0.43 g, 92%) as a white solid, used in the next step without further purification.

### Preparation of Intermediate 33

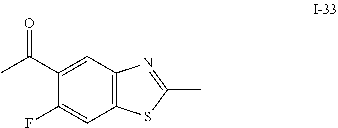

Intermediate 33 was prepared following an analogous procedure to the one described for the synthesis of intermediate 14 using intermediate 32 as starting material.

### Preparation of Intermediate 34

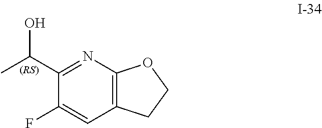

Intermediate 34 was prepared following an analogous procedure to the one described for the synthesis of intermediate 17 using intermediate 25 as starting material.

### Preparation of Intermediate 35

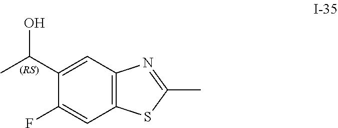

Intermediate 35 was prepared following an analogous procedure to the one described for the synthesis of intermediate 17 using intermediate 33 as starting material.

### Preparation of Intermediate 36

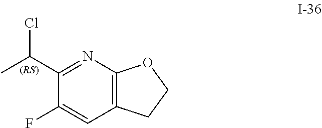

Intermediate 36 was prepared following an analogous procedure to the one described for the synthesis of intermediate 20 using intermediate 34 as starting material.

### Preparation of Intermediate 37

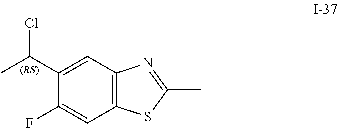

Intermediate 37 was prepared following an analogous procedure to the one described for the synthesis of intermediate 20 using intermediate 35 as starting material.

### Preparation of Intermediate 122

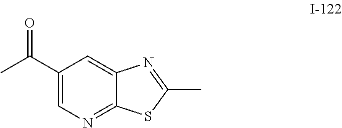

To a mixture of 6-bromo-2-methyl-[1,3]thiazolo[5,4-b]pyridine (CAS: 886372-92-5; 1.26 g, 5.50 mmol) in toluene (19.3 mL) were added PdCl₂(PPh₃)₂ (425 mg, 061 mmol) and tributyl(1-ethoxyvinyl)tin (CAS: 97674-02-7; 2.60 mL, 7.70 mmol). The reaction mixture was stirred at 92° C. for 16 h. HCl (2N, 1 mL) was added the mixture was stirred for 3 h at room temperature. The crude mixture was neutralized with NaHCO₃ (sat., aq.) and extracted with EtOAc. The combined organic extracts were dried (MgSO₄), filtered and concentrated in vacuo. The crude mixture was purified by flash column chromatography (silica, heptane/EtOAc, gradient from 100:0 to 0:100). The desired fractions were collected and concentrated in vacuo to afford intermediate 122 (872 mg, 82%) as a yellow solid.

### Preparation of Intermediate 123

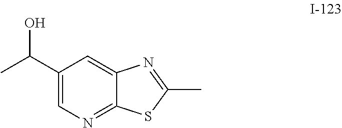

NaBH₄ (644 mg, 17.0 mmol) was added to a solution of intermediate 122 (818 mg, 4.26 mmol) in EtOH (20 mL) at 0° C. The reaction mixture was stirred at room temperature for 10 min and water was added. the aqueous phase was extracted with DCM (3×20 mL). The combined organic layers were dried (Na₂SO₄), filtered and concentrated in vacuo. The aqueous phase was further extracted with EtOAc and THF (8:2). The organic layer was dried (Na₂SO₄), filtered and concentrated in vacuo to afford intermediate 123 (838 mg, 99%) as a light yellow oil.

### Preparation of Intermediate 124

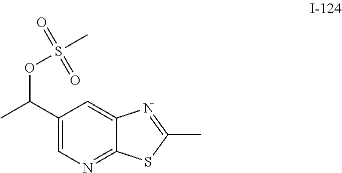 [SMILES: Cc1nc2cc(C(C)OS(C)(=O)=O)cnc2s1]

Methanesulfonyl chloride (27.1 L, 0.35 mmol) was added to a stirred solution of intermediate 123 (40.8 mg, 0.21 mmol) and Et₃N (58.5 μL, 0.42 mmol) in anhydrous DCM (2 mL) at 0° C. The reaction mixture was stirred at room temperature for 2 h. The mixture was diluted with water and extracted with DCM. The combined organic layers were dried (Na₂SO₄), filtered and the solvent was evaporated in vacuo to afford intermediate 124 which was used in the next step without further purification.

### Preparation of Intermediate 125

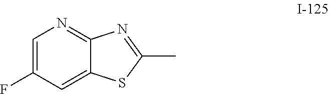

Phosphorus pentasulfide (8.74 g, 39.3 mmol) was added to a suspension of 2-acetamido-3-bromo-5-fluoropyridine (CAS: 1065074-95-4; 7.05 g, 30.3 mmol) in THF (165 mL).

The mixture was stirred at room temperature for 16h. Additional quantity of phosphorus pentasulfide (2.02 g, 9.1 mmol) was added and the mixture was stirred at for another 16 h. Cs₂CO₃ (15.8 g, 48.4 mmol) was added and the mixture was stirred at 70° C. for 16 h. Additional quantity of Cs₂CO₃ (15.8 g, 48.4 mmol) was added and the mixture was stirred at 70° C. for 3 days. The mixture was diluted with water and extracted with EtOAc. The crude product was purified by flash column chromatography (silica, heptane/EtOAc, gradient from 100:0 to 40:60). The desired fractions were concentrated in vacuo to yield intermediate 125 (3.82 g, 75%) as a yellow solid.

### Preparation of Intermediate 126

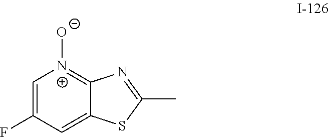

Methyltrioxorhenium(VII) (CAS: 70197-13-6; 311 mg, 1.25 mmol) was added to a stirred solution of intermediate 125 (1.40 g, 8.32 mmol) in anhydrous DCM (22.3 mL) and H₂O₂ (30% purity, 3.4 mL, 33.3 mmol) at room temperature under N₂ atmosphere. The reaction mixture was stirred at for 40 h, and manganese(IV) oxide (activated, 134 mg, 1.54 mmol) was added. After gas evolution stopped, magnesium sulfate was added. The mixture was filtered and washed with DCM, a mixture of DCM and EtOH (9:1) and MeOH. The filtrate was evaporated in vacuo. The crude mixture was combined with another fraction (5.95 mmol) and purified by flash column chromatography (silica, DCM/MeOH, gradient from 100:0 to 90:10) to afford intermediate 126 (850 mg, 34%) as a cream solid

### Preparation of Intermediate 127

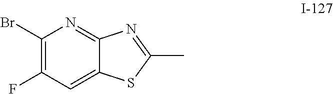

DCM (60.4 mL) was added to a mixture of tetrabutylammonium bromide (3.15 g, 9.77 mmol), molecular sieves and intermediate 126 (1.20 g, 6.52 mmol). The reaction mixture was stirred at room temperature for 10 min, and p-toluenesulfonic anhydride (3.19 g, 9.77 mmol) was added. The reaction mixture was stirred for 16 h. The mixture was filtered and the solvent was evaporated in vacuo. The crude mixture was purified by flash column chromatography (silica, DCM) to afford intermediate 127 (1.03 g, 64%) as a white solid.

### Preparation of Intermediate 128

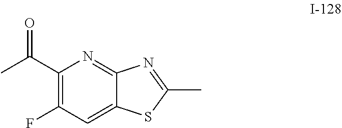

Tributyl(1-ethoxyvinyl)tin (CAS: 97674-02-7; 1.64 mL, 4.86 mmol) followed by PdCl₂(PPh₃)₂ (284 mg, 0.41 mmol) were added to a stirred solution of intermediate 127 (1.00 g, 4.05 mmol) in toluene (19.9 mL) in a sealed tube and under N₂ atmosphere. The reaction mixture was stirred at 80° C. for 48 h. Then HCl (1N, 2 mL) was added and the mixture was stirred at 70° C. for 7 h. NaHCO₃ (sat., aq.) was added and the mixture was extracted with EtOAc. The organic layer was dried (Na₂SO₄), filtered and concentrated in vacuo. The residue was purified by flash column chromatography (silica, DCM/EtOAc, gradient from 100:0 to 80:20). The desired fractions were collected and concentrated in vacuo to afford intermediate 128 (620 mg, 73%) as a light orange solid.

### Preparation of Intermediate 129

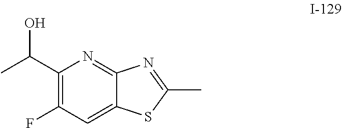

NaBH₄ (241 mg, 6.38 mmol) was added to a solution of intermediate 128 (670 mg, 3.19 mmol) in EtOH (16.4 mL) at 0° C. The reaction mixture was stirred at 0° C. for 90 min. Water was added and the mixture was extracted with DCM. The combined organic layers were dried (Na₂SO₄), filtered and concentrated in vacuo to give intermediate 129 (663 mg) which was used in the next reaction step without further purification.

### Preparation of Intermediate 130

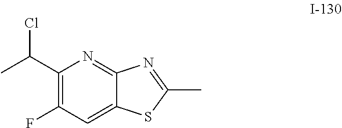

Carbon tetrachloride (3.02, mL, 31.3 mmol) was added to a mixture of intermediate 129 (663 mg, 3.13 mmol) and triphenylphosphine (1.64 g, 6.2 mmol) in CHCl₃ (2.65 mL) at 0° C. The reaction mixture was stirred at room temperature for 3 days. Additional amounts of triphenylphosphine (0.41 g, 1.61 mmol) and carbon tetrachloride (0.60 mL, 6.2 mmol) were added and the mixture was stirred for another 5 h. The solvents were evaporated in vacuo. The residue was purified by flash column chromatography (silica, heptane/EtOAc, gradient from 100:0 to 80:20) to afford intermediate 130 (488 mg, 68%) as a white solid.

### Preparation of Intermediate 131

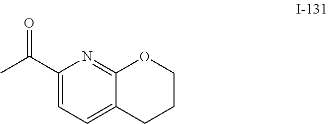

Methylmagnesium bromide (1.4M solution, 0.36 mL, 0.5 mmol) was added to a mixture of 2H, 3H, 4H-pyrano[2,3-b]pyridine-7-carbonitrile (CAS: 1824095-79-5; 80.0 mg, 0.5 mmol) in anhydrous THF (1.45 mL) at 0° C. The reaction mixture was stirred for 16 h at room temperature. Additional quantity of methylmagnesium bromide (1.4M solution, 0.36 mL, 0.5 mmol) was added and the mixture was stirred for another 16 h. The reaction was quenched with NH₄Cl (sat., aq.) and the mixture was extracted with EtOAc. The organic layer was dried (Na₂SO₄), filtered and evaporated to dryness. The crude mixture was purified by flash column chromatography (silica, heptane/EtOAc, gradient from 100:0 to 70:30) to afford intermediate 131 (46 mg, 52%) as a white solid.

### Preparation of Intermediate 132

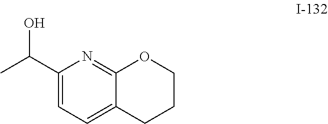

Sodium methoxide (25% purity, 1.44 μL, 6.3 μmol) was added to a stirred solution of intermediate 131 (46.0 mg, 0.26 mmol) in MeOH (0.70 mL) at 0° C. under N₂ atmosphere. NaBH₄ (9.82 mg, 0.26 mmol) was added portionwise and the reaction mixture was stirred at 0° C. for 10 min. Water was added and the mixture was extracted with DCM. The organic layer was dried (MgSO₄), filtered and concentrated in vacuo to afford intermediate 132 (26 mg, 56%) as a colorless oil

### Preparation of Intermediate 133

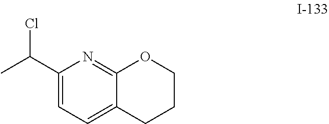

Thionyl chloride (42.5 μL, 0.58 mmol) was added to a solution of intermediate 132 (26 mg, 0.15 mmol) in DCM (067 mL) at 0° C. The reaction mixture was stirred at room temperature for 16 h. NaHCO₃ (sat., aq.) was added and the mixture was extracted with DCM. The organic layer was dried (Na₂SO₄), filtered and the solvent was evaporated in vacuo to afford intermediate 133 (26 mg, 90%) as an oil which was used in the next reaction step without further purification.

### Preparation of Intermediate 134

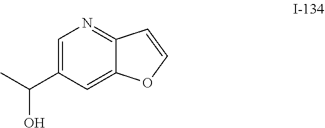

Sodium methoxide (25% purity, 13.4 μL, 58.7 μmol) was added to a stirred solution of 1{furo[3,2-b]pyridine-6-yl}ehtan-1-one (CAS: 1203499-00-6; 390 mg, 2.42 mmol) in MeOH (6.5 mL) at 0° C. under N₂ atmosphere. NaBH₄ (91.5 mg, 2.42 mmol) was added portionwise and the reaction mixture was stirred for 10 min. Water was added and the mixture was extracted with DCM. The organic layer was dried (MgSO₄), filtered and concentrated in vacuo. The residue was purified by flash column chromatography (silica, heptane/EtOAc, gradient from 100:0 to 0:100) to afford intermediate 134 (350 mg, 89%) as a brown oil.

### Preparation of Intermediate 135

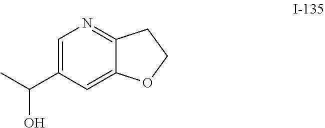

A solution of intermediate 134 (310 mg, 1.90 mmol) in EtOH (41.5 mL) was hydrogenated in a H-cube reactor (1 mL/min, 35 mm Pd/C 10% cartridge, full H2 mode, 70° C., 3 cycles). The solvent was evaporated in vacuo to afford intermediate 135 (290 mg, 92%) as a colorless oil.

### Preparation of Intermediate 136

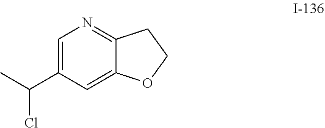

Thionyl chloride (177 μL, 2.43 mmol) was added to a solution of intermediate 135 (100 mg, 0.61 mmol) in DCM (2.78 mL) at 0° C. The reaction mixture was stirred at room temperature for 24 h and NaHCO₃ (sat., aq.) was added. The mixture was extracted with DCM. The organic layer was dried (Na₂SO₄), filtered and the solvent was evaporated in vacuo to afford intermediate 136 (88 mg, 79%) as an oil which was used in the next reaction step without further purification.

### Preparation of Intermediate 137

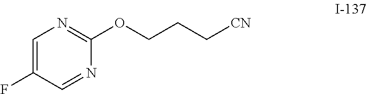 [SMILES: N#CCCCOc1ncc(F)cn1]

A solution 4-pentyn-1-ol (0.53 mL, 5.66 mmol) in THF (2.5 ml) was added dropwise to a suspension of NaH (60% dispersion in mineral oil, 235 mg, 5.89 mmol) in THF (15 mL) under N₂ atmosphere at 0° C. The mixture was stirred at 10° C. for 1 h. The temperature was cooled at 0° C. and a solution of 2-chloro-5-fluoropyrimidine (CAS: 62802-42-3; 500 mg, 3.77 mmol) in THF (2.5 mL) was added dropwise at 0° C. The reaction mixture was stirred at room temperature for 1 h. The reaction was quenched with water and the crude was extracted with EtOAc. The combined organic phases were dried (MgSO₄), filtered and concentrated in vacuo. The crude mixture was purified by flash column chromatography (silica, DCM) to afford intermediate 137 (460 mg, 68%) as a colorless oil.

### Preparation of Intermediate 138

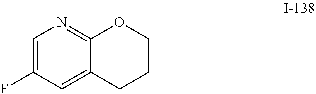 [SMILES: Fc1cnc2c(c1)CCCO2]

A mixture of intermediate 137 (3.23 g, 17.9 mmol) in nitrobenzene (24 mL) was heated at 225° C. for 6 days. The mixture was treated with a solution of HCl (2N). The mixture was stirred for 1 h and the aqueous layer was separated and treated with Na₂CO₃ to pH basic. The crude was extracted with EtOAc. The organic layer was dried (Na₂SO₄), filtered and the solvent was evaporated in vacuo. The residue was purified by flash column chromatography (silica, heptane/EtOAc, gradient from 100:0 to 70:30) to afford intermediate 138 (470 mg, 17%) as a yellow oil.

### Preparation of Intermediate 139

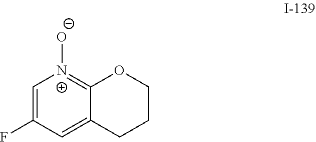 [SMILES: O[n+]1cc(F)cc2c1OCCC2]

m-CPBA (847 mg, 4.91 mmol) was added portionwise to a solution of intermediate 138 (470 mg, 3.07 mmol) in DCM (6.2 mL) at 0° C. The reaction mixture was stirred at room temperature for 24 h. The mixture was loaded to a column chromatography and purified via flash column chromatography (silica, NH₃ (7M in MeOH)/DCM, gradient from 0:100 to 4:96). The desired fractions were collected and the solvents were evaporated in vacuo to afford intermediate 139 (440 mg, 85%) as a white solid.

### Preparation of Intermediate 140

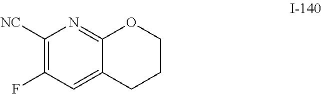 [SMILES: N#Cc1nc2c(cc1F)CCCO2]

Trimethylsilyl cyanide (1.24 mL, 9.91 mmol) was added to a mixture of intermediate 139 (406 mg, 2.40 mmol) and Et₃N (0.86 mL, 6.19 mmol) in CH₃CN (6.21 mL). The reaction mixture was stirred at 85° C. for 16 h, cooled down and treated with water. The mixture was extracted with EtOAc. The organic layer was dried (Na₂SO₄), filtered and evaporated in vacuo. The crude mixture was purified by flash column chromatography (silica, heptane/EtOAC, gradient from 100:0 to 40:60) to afford intermediate 140 (390 mg, 91%) as an off-white solid.

### Preparation of Intermediate 141

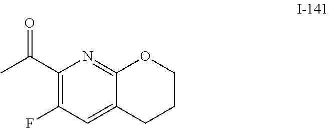

Methylmagnesium bromide (3.2M in Me-THF, 065 mL, 2.07 mmol) was added to a mixture of intermediate 140 (335 mg, 1.88 mmol) in anhydrous THF (5.46 mL) at 0° C. After completion of the addition, the reaction mixture was stirred for 16 h at room temperature. Additional quantity of methylmagnesium bromide (0.3 mL, 1.00 mmol) was added at 0° C. and the reaction mixture was stirred for 16 h. NH₄Cl (sat., aq.) was added and the mixture was extracted with EtOAc. The organic layer was dried (Na₂SO₄), filtered and evaporated to dryness. The crude mixture was purified by flash column chromatography (silica, heptane/EtOAc, gradient from 100:0 to 70:30). A second purification was performed by RP HPLC (stationary phase: C18 XBridge 30×100 mm 5 μm), mobile phase: NH₄HCO₃ (0.25% solution in water)/CH₃CN, gradient from 85:15 to 55:45) to afford intermediate 141 (46 mg, 13%) as a white solid.

### Preparation of Intermediate 142

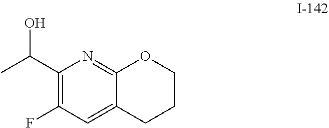

Sodium methoxide (25% purity, 1.65 μL, 7.21 μmol) was added to a stirred solution of intermediate 141 (58.0 mg, 0.30 mmol) in MeOH (0.80 mL) at 0° C. under N₂ atmosphere. NaBH₄ (11.2 mg, 0.30 mmol) was added portionwise. The reaction mixture was stirred at 0° C. for 10 min and at room temperature for 1 h. Water was added and the mixture was extracted with DCM. The organic layer was dried (MgSO₄), filtered and concentrated in vacuo to afford intermediate 142 (54 mg, 92%) as a colorless oil.

### Preparation of Intermediate 143

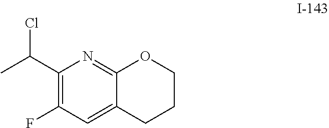

Thionyl chloride (80.3 μL, 1.10 mmol) was added to a solution of intermediate 142 (54.0 mg, 0.27 mmol) in DCM (1.26 mL) at 0° C. The reaction mixture was stirred at room temperature for 24 h. NaHCO₃ (sat., aq.) was added and the mixture was extracted with DCM. The organic layer was dried (Na₂SO₄), filtered and the solvent was evaporated in vacuo to afford intermediate 143 (46 mg, 78%) as an oil which was used in the next reaction step without further purification.

### Preparation of Intermediate 144

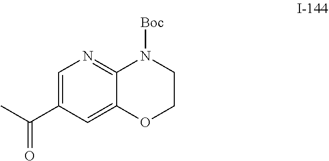 [SMILES: CC(=O)c1cnc2c(c1)OCCN2C(=O)OC(C)(C)C]

Tributyl(1-ethoxyvinyl)tin (CAS: 97674-02-7; 9.79 mL, 28.9 mmol) followed by PdCl₂(PPh₃)₂ (1.85 g, 2.63 mmol) were added to a stirred solution of tert-butyl-7-bromo-2,3-dihydro-4H-pyrido[3,2-b][1,4]oxazine-4-carboxylate (CAS: 335030-30-3; 8.30 g, 26.3 mmol) in 1,4-dioxane (166 mL) in a sealed tube and under N₂ atmosphere. The reaction mixture was stirred at 80° C. overnight. Then HCl (1M in H₂O, 13.2 mL, 13.2 mmol) was added and the mixture was stirred at room temperature for 30 min. The mixture was treated with NaHCO₃ (sat., aq.) and ice water and extracted with DCM. The organic layer was dried (MgSO₄), filtered and the solvents were evaporated in vacuo. The crude mixture was purified by flash column chromatography (silica, EtOAc in DCM, gradient from 0:100 to 20:80, then EtOAc in heptane, gradient from 0:100 to 50:50). The desired fractions were collected and concentrated in vacuo to afford intermediate 144 (5.6 g, 76%) as a white solid.

### Preparation of Intermediate 145

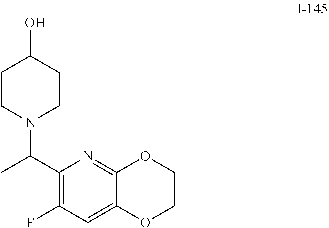 [SMILES: CC(c1nc2c(cc1F)OCCO2)N1CCC(O)CC1]

A mixture of 4-hydroxypiperidine (CAS: 5382-16-1; 4.65 g, 45.9 mmol) and K₂CO₃ (9.53 g, 68.9 mmol) in CH₃CN (100 mL) was stirred at room temperature under N₂ atmosphere for 10 min. Intermediate 20 (5.00 g, 23.0 mmol) was added dropwise and the reaction mixture was stirred at 80° C. overnight. The mixture was evaporated in vacuo.

The crude product was combined with another fraction (11.7 mmol) and purified by flash column chromatography (silica, petroleum ether/EtOAc, gradient from 100:0 to 3:1). The pure fractions were collected and the solvent was evaporated in vacuo to give intermediate 145 (8.04 g, 48%) as a white solid.

### Preparation of Intermediate 146

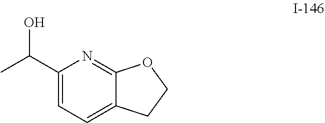

NaBH₄ (185 mg, 4.90 mmol) was added to a stirred solution of 6-acetyl-2,3-dihydrofuro[2,3-b]pyridine (200 mg, 1.23 mmol) in EtOH (7 mL) at 0° C. The reaction mixture was stirred at 0° C. for 15 min and then at room temperature for 30 min. The mixture was diluted with water and extracted with DCM (3×5 mL). The organic layer was separated, dried (Na₂SO₄), filtered and the solvents were evaporated in vacuo to afford intermediate 146 (160 mg, 79%) as yellow oil.

### Preparation of Intermediate 147

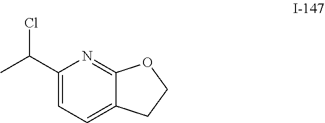

Thionyl chloride (0.28 mL, 3.89 mmol) was added to a solution of intermediate 146 (160 mg, 0.97 mmol) in DCM (5 mL) at 0° C. The reaction mixture was stirred at room temperature for 2 h. Water was added and the mixture was extracted with DCM. The combined organic layers were dried (MgSO₄), filtered and evaporated in vacuo to yield intermediate 147 (170 mg, 96%) as yellow oil.

### Preparation of Intermediates 185 AND 186

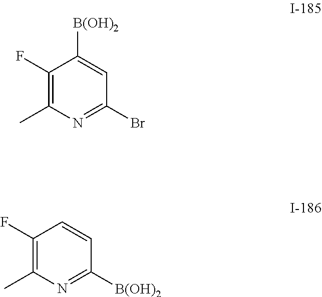

To a solution of 6-bromo-3-fluoro-2-methylpyridine (CAS: 374633-38-2; 500 mg, 2.63 mmol) in anhydrous THF (10 mL) was added was added n-BuLi (2.5M in hexane, 1.05 mL, 2.6 mmol) dropwise at −78° C. and under N₂ atmosphere. The reaction mixture was stirred at −78° C. for 1 h and a solution of triisopropyl borate (CAS: 5419-55-6; 1.34 mL, 5.79 mmol) in anhydrous THF (5 mL) was added. The reaction mixture was stirred at −78° C. for 1 h, quenched with water and concentrated in vacuo to afford a mixture of intermediates 185 and 186 (615 mg, quant.) which was used in the next step without any purification.

### Preparation of Intermediate 187

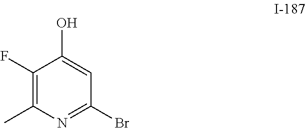

To a suspension of intermediates 185 and 186 in a mixture of THF (15 mL) and water (5 mL) was added H₂O₂ (30% purity, 1.61 mL, 15.8 mmol). The reaction mixture was stirred at room temperature for 18 h and concentrated in vacuo. The residue was partitioned between EtOAc and water. The organic layer was separated and the aqueous phase was extracted with EtOAc. The combined organic layers were dried (MgSO₄), filtered and the solvent was concentrated in vacuo. The residue was purified by flash column chromatography (silica, EtOAc in DCM, gradient from 0:100 to 20:80). The desired fractions were collected and concentrated in vacuo to afford intermediate 187 (132 mg, 24%) as a white solid.

### Preparation of Intermediate 188 AND FINAL COMPOUND 167

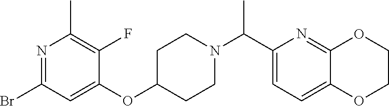 [SMILES: Cc1nc(Br)cc(OC2CCN(C(C)c3ccc4c(n3)OCCO4)CC2)c1F]

DBAD (CAS: 870-50-8; 218 mg, 0.95 mmol) was added to a mixture of intermediate 187 (150 mg, 0.73 mmol), intermediate 116 (202 mg, 0.77 mmol) and triphenylphosphine (248 mg, 0.95 mmol) in toluene (3.92 mL). The reaction mixture was stirred at 80° C. for 24 h and the solvent was removed in vacuo. The crude product was purified by flash chromatography (silica, MeOH in DCM, gradient from 0:100 to 5:95). The desired fractions were collected and concentrated in vacuo to afford intermediate 188 (105 mg, 32%) as a white solid.

### Preparation of Intermediate 189

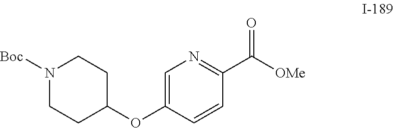 [SMILES: COC(=O)c1ccc(OC2CCN(C(=O)OC(C)(C)C)CC2)cn1]

Triphenylphosphine (1.17 g, 4.45 mmol) was added to a stirred mixture of methyl 5-hydroxypyridine-2-carboxylate (CAS: 30766-12-2; 500 mg, 3.27 mmol) and 1-Boc-4-hydroxypiperidine (CAS: 109384-19-2; 597 mg, 2.97 mmol) in anhydrous THF (30 mL) under N₂ atmosphere. The reaction mixture was stirred at room temperature for 15 min, and DIAD (CAS: 2446-83-5; 0.88 mL, 4.45 mmol) was added dropwise at 0° C. The reaction mixture was stirred at room temperature overnight. The mixture was diluted with water and extracted with EtOAc. The organic layer was dried (MgSO₄), filtered and the solvents were evaporated in vacuo. The crude product was purified by flash column chromatography (silica, heptane/EtOAc, gradient from 100:0 to 30:70). The desired fractions were collected and concentrated in vacuo to afford intermediate 189 (750 mg, 74%) as a colorless oil.

### Preparation of Intermediate 190

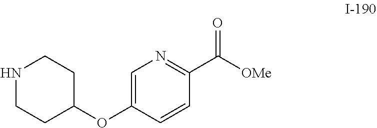 [SMILES: COC(=O)c1ccc(OC2CCNCC2)cn1]

TFA (5.68 mL, 73.6 mmol) was added to a stirred solution of intermediate 189 (0.75 g, 2.23 mmol) in DCM (18.6 mL). The reaction mixture was stirred at room temperature for 20 h. The solvent was removed in vacuo. The crude product was purified by flash column chromatography (silica, MeOH:NH₃ in DCM, gradient from 0:100 to 10:90). The desired fractions were collected and concentrated in vacuo to give intermediate 190 (536 mg, 99%) as a colorless oil.

### Preparation of Intermediate 191

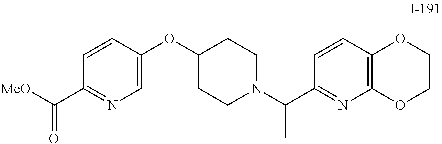 [SMILES: COC(=O)c1ccc(OC2CCN(C(C)c3ccc4c(n3)OCCO4)CC2)cn1]

Intermediate 21 (118 mg, 0.59 mmol) was added to a mixture of intermediate 191 (116 mg, 0.49 mmol) and K₂CO₃ (136 mg, 0.98 mmol) in CH₃CN (5 mL) at room temperature.

The reaction mixture was stirred at 79° C. for 24 h. The mixture was diluted with NaHCO₃ (sat., aq.) and extracted with EtOAc. The organic layer was dried (Na₂SO₄), filtered and the solvents were evaporated in vacuo. The crude product was purified by flash column chromatography (silica, MeOH in DCM, gradient from 0:100 to 4:96). The desired fractions were collected and concentrated in vacuo to yield intermediate 191 (70 mg, 35%) as a white sticky solid.

### Preparation of Intermediate 192

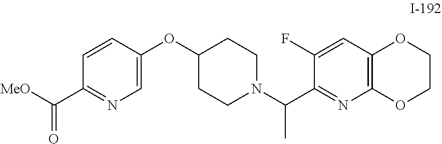 [SMILES: COC(=O)c1ccc(OC2CCN(C(C)c3nc4c(cc3F)OCCO4)CC2)cn1]

Intermediate 192 was prepared following an analogous procedure to the one described for the synthesis of intermediate 191 using intermediate 20 and intermediate 190 as starting materials.

The crude product was purified by flash column chromatography (silica, MeOH in DCM, gradient from 0:100 to 4:96) to afford intermediate 192 (102 mg, 50%) as a colorless oil.

### Preparation of Intermediate 193

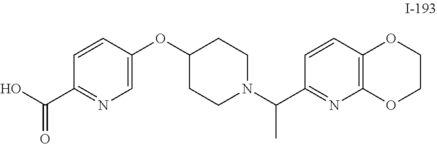 [SMILES: CC(c1ccc2c(n1)OCCO2)N1CCC(Oc2ccc(C(=O)O)nc2)CC1]

LiOH.H₂O (8.83 mg, 0.21 mmol) was added to a solution of intermediate 191 (70.0 mg, 0.18 mmol) in THF (1.43 mL) and H₂O (0.36 mL). The reaction mixture was stirred for 16 h at room temperature. The mixture was acidified with HCl (2M) to pH 2-3 and concentrated in vacuo to give intermediate 193 which was used as such in the next step.

### Preparation of Intermediate 194

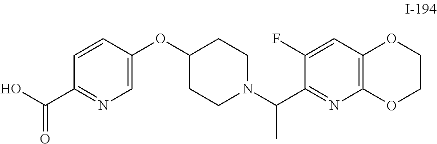 [SMILES: CC(c1nc2c(cc1F)OCCO2)N1CCC(Oc2ccc(C(=O)O)nc2)CC1]

Intermediate 194 was prepared following an analogous procedure to the one described for the synthesis of intermediate 193 using intermediate 192 as starting material. The crude product was used in the next step without any purification.

### Preparation of Intermediate 195

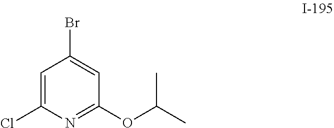

NaH (60% in mineral oil, 194 mg, 4.85 mmol) was added to a stirred solution of isopropyl alcohol (4 mL, 52.3 mmol) in THF (24 mL) at 0° C. under N₂ atmosphere. The mixture was stirred at room temperature for 1 h. 4-Bromo-2,6-dichloropyridine (CAS: 98027-80-6; 1.00 g, 4.41 mmol) was added and the reaction mixture was stirred at room temperature for 16 h. The mixture was diluted with water and extracted with EtOAc. The organic layer was dried (MgSO₄), filtered and the solvents were evaporated in vacuo. The crude product was purified by flash column chromatography (silica, heptane/EtOAc, gradient from 100:0 to 95:5). The desired fractions were collected and concentrated in vacuo to afford intermediate 195 (902 mg, 82%) as a colorless oil.

### Preparation of Intermediate 196

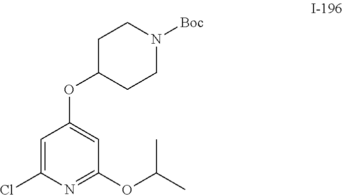 [SMILES: CC(C)Oc1cc(OC2CCN(C(=O)OC(C)(C)C)CC2)cc(Cl)n1]

NaOtBu (369 mg, 3.84 mmol) was added to a solution of 1-tert-butoxycarbonyl-4-hydroxypiperidine (CAS: 109384-19-2; 644 mg, 3.20 mmol) in DMSO (20 mL). The reaction mixture was stirred for 1 h at 0° C. Intermediate 195 (802 mg, 3.20 mmol) was added and the reaction mixture was stirred at 50° C. for 16 h. The mixture was cooled to room temperature and water was added. The mixture was extracted with EtOAc. The combined organic layers were washed with NaHCO₃ and brine, dried, filtered and concentrated in vacuo. The crude product was purified by flash column chromatography (silica, heptane/EtOAc, gradient from 100:0 to 90:10). The desired fractions were collected and concentrated in vacuo to afford intermediate 196 (876 mg, 74%) as a colorless oil.

### Preparation of Intermediate 197

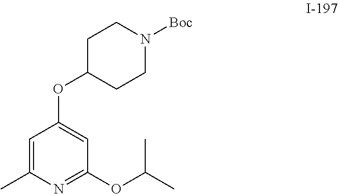 [SMILES: Cc1cc(OC2CCN(C(=O)OC(C)(C)C)CC2)cc(OC(C)C)n1]

Intermediate 196 (776 mg, 2.09 mmol) and methylboronic acid (320 mg, 5.23 mmol) were added to a stirred mixture of Na₂CO₃ (665 mg, 6.28 mmol) 1,4-dioxane (5.23 mL) and water (1.31 mL) under N₂ atmosphere. Pd(dppf)Cl₂.DCM (85.4 mg, 0.11 mmol) was added. The reaction mixture was stirred at 105° C. for 72 h. The mixture was diluted with NaHCO₃ (sat., aq.) and extracted with EtOAc. The organic layer was dried (MgSO₄), filtered and the solvents were evaporated in vacuo. The crude product was purified by flash column chromatography (silica, heptane/EtOAc, gradient from 95:5 to 80:20). The desired fractions were collected and concentrated in vacuo to afford intermediate 197 (599 mg, 81%) as a colorless oil.

### Preparation of Intermediate 198

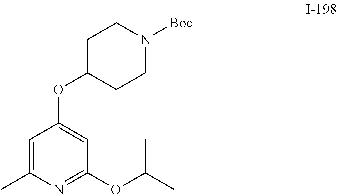 [SMILES: Cc1cc(OC2CCN(C(=O)OC(C)(C)C)CC2)cc(OC(C)C)n1]

HCl (4M in 1,4-dioxane, 2.14 mL, 8.56 mmol) was added dropwise to intermediate 197 (599 mg, 1.71 mmol) at 0° C. The reaction mixture was stirred at room temperature for 16 h and the solvent was evaporated in vacuo. The crude product was purified by flash column chromatography (silica, MeOH:NH₃ in DCM, gradient from 0/100 to 10/90). The desired fractions were collected and concentrated in vacuo to give intermediate 198 (395 mg, 92%) as a white solid.

### Preparation of Intermediate 199

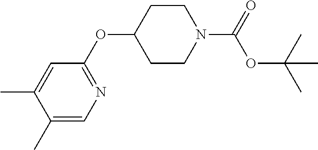 [SMILES: Cc1cnc(OC2CCN(C(=O)OC(C)(C)C)CC2)cc1C]

Intermediate 199 was prepared following an analogous procedure to the one described for the synthesis of intermediate XX using 1-Boc-4-hydroxypiperidine and 2-chloro-4,5-dimethylpyridine (CAS: 343268-69-9) as starting materials. The crude was purified by flash column chromatography (silica, EtOAc in heptane 0/100 to 70/30). The desired fractions were collected and the solvents concentrated in vacuo to yield intermediate 199 (106.8 mg, 35%) as a colourless oil.

### Preparation of Intermediate 200

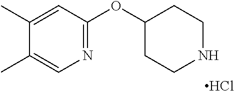

Intermediate 200 was prepared following an analogous procedure to the one described for the synthesis of intermediate 59.

### Preparation of Intermediate 201

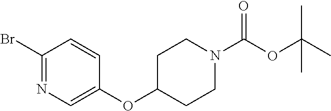 [SMILES: CC(C)(C)OC(=O)N1CCC(Oc2ccc(Br)nc2)CC1]

Intermediate 201 was prepared following an analogous procedure to the one described for the synthesis of intermediate 72 using 4-hydroxy-1-piperidinecarboxylic acid 1,1-dimethylethyl ester (CAS: 109384-19-2) and 6-bromopyridin-3-ol (CAS: 55717-40-3) as starting materials. The crude was purified by flash column chromatography (silica: EtOAc acetate in heptane, 0/100 to 30/70). The desired fractions were collected and concentrated in vacuo to yield intermediate 201 (130 mg, 63%) as a colourless oil.

### Preparation of Intermediate 202

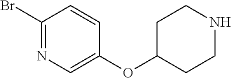 [SMILES: Brc1ccc(OC2CCNCC2)cn1]

HCl (4M in dioxane, 2.361 mL, 9.445 mmol) was added to intermediate 201 (125 mg, 0.35 mmol) and the reaction mixture was stirred at room temperature for 3 h. The reaction was concentrated to dryness. Then the residue was purified by ion exchange chromatography using an ISOLUTE SCX2 cartridge eluting first with methanol and then with 7M solution of ammonia in methanol. The desired fraction were collected and concentrated in vacuo to yield intermediate 202 (86 mg, 96%) as a colourless oil, which was used in the following step without further purification.

### Preparation of Intermediate 203

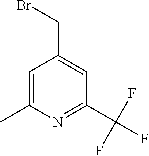

Phosphorous tribromide (365.20 μL 3.85 mmol) was added to a solution of 2-methyl-6-(trifluoromethyl)-4-pyridinemethanol (CAS: 1936597-62-4, 490 mg, 2.563 mmol) in DCM (10 mL) dropwise at 0° C. and the mixture was stirred for 2 hours at r.t. The mixture was diluted with DCM washed with NaHCO₃. The organic layer was dried over MgSO₄, filtered and the solvent removed. The crude product was purified by flash column chromatography (silica; EtOAc in heptane 0/100 to 30/70). The desired fractions were collected and concentrated in vacuo to yield intermediate 203 (477 mg, 73%) as a colourless oil.

### Preparation of Intermediate 204

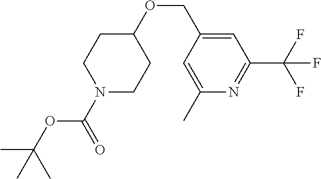 [SMILES: Cc1cc(COC2CCN(C(=O)OC(C)(C)C)CC2)cc(C(F)(F)F)n1]

Intermediate 204 was prepared following an analogous procedure to the one described for the synthesis of intermediate 170 using 1-Boc-4-hydroxypiperidine and intermediate 203 as starting material. The crude product was purified by flash column chromatography (silica; EtOAc in heptane from 0/100 to 100/0). The desired fractions were collected and concentrated to yield intermediate 204 (478 mg, 68%) as a colourless oil.

### Preparation of Intermediate 205

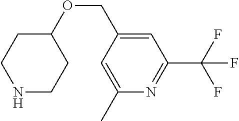 [SMILES: Cc1cc(COC2CCNCC2)cc(C(F)(F)F)n1]

Intermediate 205 was prepared following an analogous procedure to the one described for the synthesis of intermediate 150 using intermediate 204 as starting material. Intermediate 205 (106.4 mg, 61%) was isolated as a red foamy solid, which was used without further purification.

### Preparation of Intermediate 206

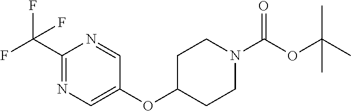 [SMILES: CC(C)(C)OC(=O)N1CCC(Oc2cnc(C(F)(F)F)nc2)CC1]

Intermediate 206 was prepared following an analogous procedure to the one described for the synthesis of intermediate 72 using 2-(trifluoromethyl)-5-pyrimidinol and 1-Boc-4-hydroxypiperidine as starting materials. The crude product was purified by flash column chromatography (silica: ethyl acetate in heptane, 0/100 to 30/70). The desired fractions were collected and concentrated in vacuo to yield intermediate 206 (980 mg, 65%) as a light yellow solid.

### Preparation of Intermediate 207

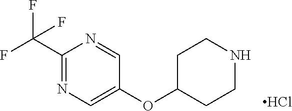 [SMILES: FC(F)(F)c1ncc(OC2CCNCC2)cn1 ; Cl]

Intermediate 207 was prepared following an analogous procedure to the one described for the synthesis of intermediate 41 using intermediate 206 as starting material. The crude product (540 mg, 96%) was isolated as a white solid and used without further purification.

### Preparation of Intermediate 208

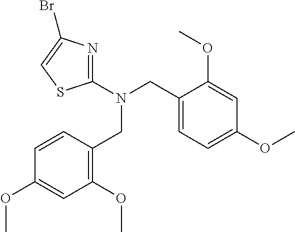 [SMILES: COc1ccc(CN(Cc2ccc(OC)cc2OC)c2nc(Br)cs2)c(OC)c1]

2,4-Dibromo-thiazole (CAS: 4175-77-3, 50 g, 205.83 mmol), N-[(2,4-dimethoxyphenyl)methyl]-2,4-dimethoxy-benzenemethanamine (CAS: 20781-23-1, 65.33 g, 205, 83 mmol) and Na₂CO₃ (65.51 g, 618 mmol) in CH₃CN (500 mL) was heated for 36 hours. The mixture was concentrated and dissolved in EtOAc (1000 mL). The mixture was washed with water (50 mL) and brine, dried over MgSO₄, and concentrated to give crude product, which was purified by column chromatography on silica gel (petroleum ether/EtOAc, from 100/0 to 70/30) to give intermediate 208 (70 g, 70%) as a yellow solid.

### Preparation of Intermediate 209

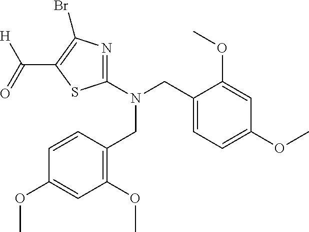 [SMILES: COc1ccc(CN(Cc2ccc(OC)cc2OC)c2nc(Br)c(C=O)s2)c(OC)c1]

To a solution of intermediate 208 (15 g, 31.29 mmol) in anhydrous THF (20 mL) was added dropwise LDA (34.42 mL, 34.42 mmol) at a rate so the temperature did not exceed −70° C. The resulting solution was stirred at −78° C. for 30 min. Then DMF (2.52 g, 34.42 mmol) was added dropwise as a solution in THF (20 mL) and the mixture was allowed to warm up to room temperature. The reaction was quenched with saturated NH₄Cl (30 mL). The mixture was extracted with EtOAc (2×50 mL). The combined organic layers were washed with brine, dried over MgSO₄, and concentrated. The crude was purified by flash chromatography on silica gel (petroleum ether/EtOAc, from 100/0 to 80/20) to yield intermediate 209 (8 g, 45%) as a light yellow solid.

### Preparation of Intermediate 210

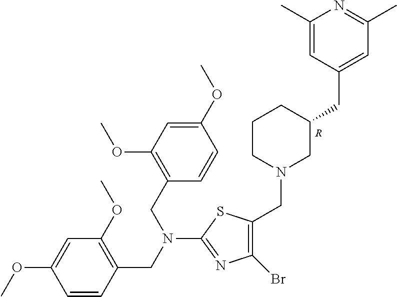

Intermediate 209 (2006.23 mg, 3.95 mmol) was added to intermediate (3R)-34 from WO2018/109202 (729 mg, 3.57 mmol) at RT. After 30 min, sodium triacetoxyborohydride (1512.43 mg, 7.14 mmol) was added to the mixture at RT and the RM was stirred for 48 h at RT. The crude was quenched with NH₃/H₂O and extracted with EtOAc. The organic layer was separated, dried (Na₂SO₄), filtered and the solvent was evaporated in vacuo. The residue was purified by automated flash chromatography (silica, 10% MeOH in DCM 0/100 to 5/95). Desired fractions were collected, concentrated under vacuo to yield intermediate 210 (1.1 g, 44%) as a sticky solid.

### Preparation of Intermediate 211

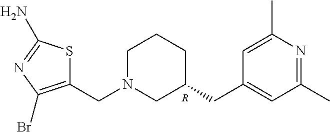

A mixture of intermediate 210 (1050 mg, 1.51 mmol) in TFA (26.25 mL) was stirred at RT under a nitrogen atmosphere for 1.5 h. The solvent was evaporated and the mixture was taken in water, basified with K₂CO₃ and extracted with DCM. The organic layer was dried over MgSO₄ and concentrated. The residue was purified on a column with silica gel, eluent DCM/MeOH (100/0 to 90/10). The pure fractions were evaporated, yielding intermediate 211 (521 mg, 87%) as a white solid.

### Preparation of Intermediate 212

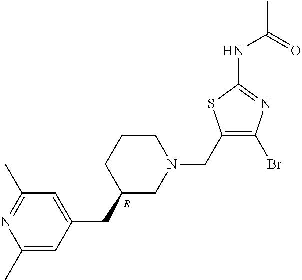

Acetic anhydride (7.75 mg, 0.076 mmol) was added dropwise to a solution of intermediate 211 (20 mg, 0.051 mmol) in 1,4-dioxane (15 mL) while stirring. After the addition was complete, the reaction was heated at 60° C. for 2 h, then at 110° C. for 4 h.

The RM was evaporated, taken up in water/0.5 g NaHCO₃/DCM. The organic layer was separated, dried over MgSO₄ and concentrated. The residue was purified on a column with silica gel, eluent: DCM/MeOH (100/0 to 95/5). The pure fractions were concentrated, yielding intermediate 212 (135 mg, 41%) as a pale yellow foam.

### PREPARATION OF [³H]-LIGAND FOR OCCUPANCY STUDY

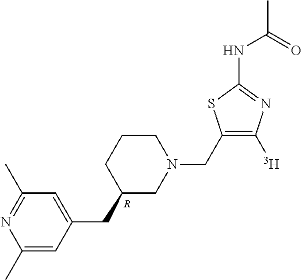

Compound 28 from WO2018/109202 was labelled with [³H] as follows: Intermediate 212 (4.10 mg, 9.38 μmol) and Palladium supported on Carbon (10%, 14.4 mg) were suspended in DMF (0.2 mL) and DIPEA (12 μL, 70.6 μmol) was added. The suspension was degassed three times and stirred under an atmosphere of Tritium gas (4.2 Ci, 525 mbar initial pressure) for 2 h 47 min at RT (end pressure was 311 mbar, no more consumption of gas was observed). The solvent was removed in vacuo, and labile tritium was exchanged by adding MeOH (0.3 mL), stirring the solution, and removing the solvent again under vacuo. This process was repeated twice. Finally, the well dried solid was extracted with EtOH (5 mL) and the suspension was filtered through a 0.2 μm nylon membrane (Macherey-Nagel Polyamide syringe filter CHROMAFIL®Xtra PA-20/25), obtaining a clear solution.

The radiochemical purity (RCP) of the crude material was determined to be 56% using the following HPLC system: Waters Atlantis T3, 5 μm, 4.6×250 mm; solvents A: water+0.05% TFA, B: acetonitrile+0.05% TFA; 0 min 0% B; 10 min 30% B; 10.2-14.5 min 95% B; 15 min 0% B; 254 nm; 1.0 mL/min; 30° C.

The crude was purified by HPLC: Waters Atlantis T3, 5 μm, 10×250 mm; solvents A: water+0.1% TFA; B: acetonitrile+0.1% TFA; 0 min 0% B, 15 min 45% B; 4.7 mL/min; 25° C. The target compound eluted at 9.5 min, and isolated from the HPLC solvent mixture by solid phase extraction. Therefore, the HPLC solution was neutralized with an aqueous solution of NaHCO₃ and the volume of the fractions were partially reduced at the rotary evaporator. Then the product was extracted with a Phenomenex StrataX cartridge (33 μm Polymeric Reversed Phase, 100 mg, 3 mL; 8B-S100-EB) which was eluted with EtOH (5 mL). The extracted product showed an RCP of >99% and the specific activity (SA) was determined to be 10.7 Ci/mmol (396 GBq/mmol, determined by MS). Two batches 250 μCi (9.25 MBq) in 0.25 mL EtOH (1mCi/mL) and 38.8 mCi in 5 mL EtOH of [3H]-ligand were isolated.

### Preparation of Final Compounds

### E1. Preparation of Final Compounds 1, 2 and 3

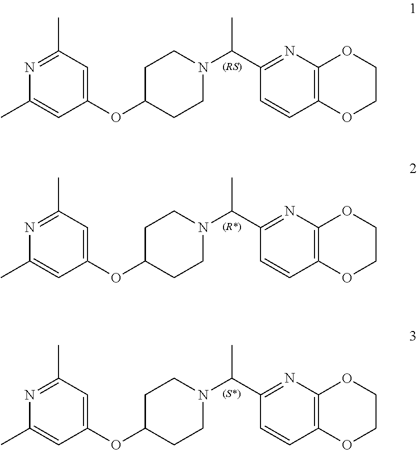 [SMILES: Cc1cc(OC2CCN(C(C)c3ccc4c(n3)OCCO4)CC2)cc(C)n1 ; Cc1cc(OC2CCN(C(C)c3ccc4c(n3)OCCO4)CC2)cc(C)n1 ; Cc1cc(OC2CCN(C(C)c3ccc4c(n3)OCCO4)CC2)cc(C)n1]

Method 1: 2,3-dihydro-[1,4]dioxino[2,3-b]pyridine-6-carboxaldehyde (CAS: 615568-24-6, 184 mg, 1.11 mmol) and titanium (IV) isopropoxide (CAS: 546-68-9, 0.44 mL, 1.52 mmol) were added to a stirred solution of intermediate 6 (209 mg, 1.01 mmol) in DCM (3.4 mL) at rt and under N₂. The mixture was stirred at rt for 3 h. Then it was cooled at 0° C. and methyl magnesium bromide (CAS: 75-16-1, 3.62 mL, 5.07 mmol, 1.4 M in THF/toluene) was added dropwise. The mixture was stirred at this temperature for 5 min and at rt for 16 h. The mixture was treated with aq. sat. sol. NH₄Cl, diluted with DCM. The organic layer was separated, washed with aq. sat. sol. NaCl, dried (Na₂SO₄), filtered and the solvents evaporated in vacuo. The crude product was purified by RP HPLC (stationary phase: XBridge C18 50×100 mm, 5 μm, mobile phase: gradient from 80% NH₄HCO₃ 0.25% solution in water, 20% CH₃CN to 63% NH₄HCO₃ 0.25% solution in water, 37% CH₃CN). The desired fractions were collected and evaporated in vacuo to yield compound 1 as a brown syrup (78 mg, 21%).

Compound 1 (78 mg) was purified via chiral SFC (stationary phase: CHIRACEL OJ-H 5 μm 250*20 mm, mobile phase: 84% CO₂, 16% MeOH (0.3% iPrNH₂)) yielding compound 2 (30 mg, 8%) and compound 3 (31 mg, 8%) both as oils. Compounds 2 and 3 were dissolved in Et₂O and then HCl (2N in Et₂O) was added. The resulting solids were filtered and dried to give compounds 2 (27.3 mg, 7%, HCl salt) and 3 (30 mg, 7%, HCl salt) both as white solids.

Method 2: Potassium carbonate (CAS: 584-08-7, 2.63 g, 19.05 mmol) was added to a stirred solution of intermediate 6 (1.31 g, 6.35 mmol) and intermediate 21 (1.27 g, 6.35 mmol) in acetonitrile (50 mL) at rt. The mixture was stirred at 70° C. for 36 h. The reaction was diluted with water and extracted with EtOAc (3×). The organic layer was separated, dried (Na₂SO₄), filtered and the solvents evaporated in vacuo. The crude product was purified by flash column chromatography (silica; 7M solution of ammonia in MeOH in DCM 0/100 to 3/97). The desired fractions were collected and the solvents evaporated in vacuo to yield compound 1 as a pale-yellow oil (1.77 g, 75%).

### E2. Preparation of Final Compounds 4,148 and 149

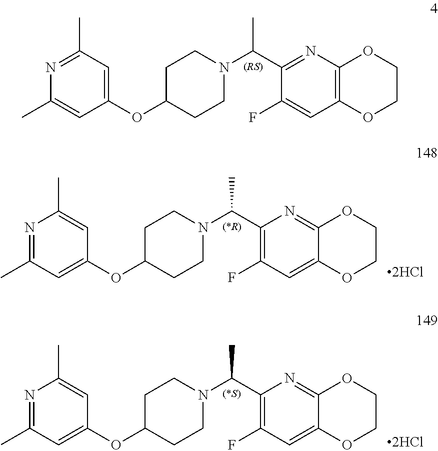 [SMILES: Cc1cc(OC2CCN(C(C)c3nc4c(cc3F)OCCO4)CC2)cc(C)n1 ; Cc1cc(OC2CCN([C@H](C)c3nc4c(cc3F)OCCO4)CC2)cc(C)n1 ; Cc1cc(OC2CCN([C@@H](C)c3nc4c(cc3F)OCCO4)CC2)cc(C)n1]

Compound 4 was prepared following an analogous procedure to the one described as Method 2 for the synthesis of compound 1 using intermediate 6 (159 mg, 0.77 mmol) and intermediate 20 (120 mg, 0.55 mmol) as starting materials. Compound 4 was purified by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and partially concentrated in vacuo. The aqueous phase was extracted with EtOAc (3×), separated, dried (Na₂SO₄), filtered and the solvents evaporated in vacuo to yield compound 4 (34 mg, 16%) as a colorless oil.

Compound 4 (1.20 g) was purified via chiral SFC (stationary phase: CHIRACEL OJ-H 5 μm 250*30 mm, mobile phase: 80% CO₂, 20% EtOH (0.3% i-PrNH₂)) to afford 2 fractions: fraction A (461 mg) and fraction B (468 mg).

Fraction A (460 mg, 1.19 mmol) was dissolved in tert-butyl methyl ether (3 mL) and HCl (2M in Et₂O, 1.79 mL, 3.56 mmol) was added under stirring. The resulting precipitate was filtered off and dried at 50° C. under vacuum to give compound 148 (525 mg, 96%).

Compound 149 (545 mg, 98%) was obtained following an analogous procedure to the one reported for the synthesis of compound 148 (468 mg), using fraction B as starting material.

### E3. Preparation of Final Compound 5

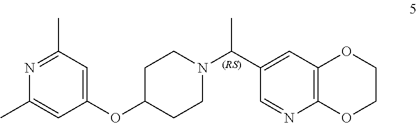 [SMILES: Cc1cc(OC2CCN(C(C)c3cnc4c(c3)OCCO4)CC2)cc(C)n1]

Compound 5 was prepared following an analogous procedure to the one described as Method 2 for the synthesis of compound 1 using intermediate 6 (150 mg, 0.73 mmol) and intermediate 22 (140 mg, 0.71 mmol) as starting materials. Compound 5 was purified by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and partially concentrated in vacuo. The aqueous phase was extracted with EtOAc (3×), separated, dried (Na₂SO₄), filtered and the solvents evaporated in vacuo to yield compound 5 (150 mg, 56%) as a colorless oil.

### E4. Preparation of Final Compound 6

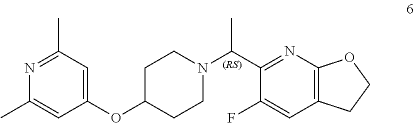 [SMILES: Cc1cc(OC2CCN(C(C)c3nc4c(cc3F)CCO4)CC2)cc(C)n1]

Compound 6 was prepared following an analogous procedure to the one described as Method 2 for the synthesis of compound 1 using intermediate 6 (70 mg, 0.34 mmol) and intermediate 36 (68 mg, 0.34 mmol) as starting materials. Compound 6 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and partially concentrated in vacuo. The aqueous phase was extracted with EtOAc (3×), separated, dried (Na₂SO₄), filtered and the solvents evaporated in vacuo to yield compound 6 (71 mg, 57%) as a pale-orange oil.

### E130. Preparation of Final Compounds 143,144 and 145

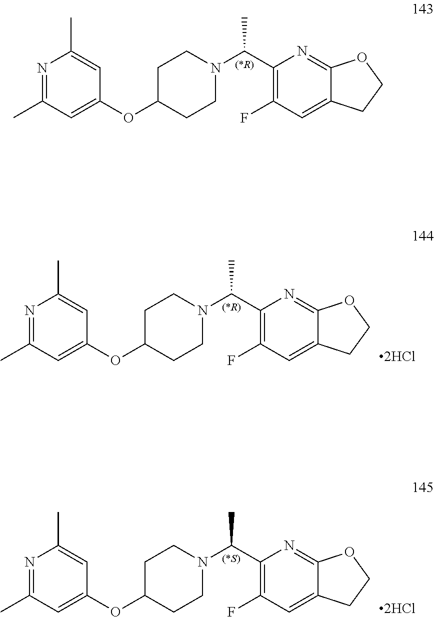 [SMILES: Cc1cc(OC2CCN([C@H](C)c3nc4c(cc3F)CCO4)CC2)cc(C)n1 ; Cc1cc(OC2CCN([C@H](C)c3nc4c(cc3F)CCO4)CC2)cc(C)n1 ; Cc1cc(OC2CCN([C@@H](C)c3nc4c(cc3F)CCO4)CC2)cc(C)n1]

A purification of compound 6 (230 mg) was performed via chiral SFC (stationary phase: CHIRACEL OJ-H 5 μm 250*20 mm, mobile phase: 85% CO₂, 15% MeOH (0.3% i-PrNH₂)) to afford compound 143 (91 mg) and fraction B (92 mg) as yellow oils. HCl (2M in Et₂O, 49.2 μL, 98.5 μmol) was added to a stirred solution of compound 143 (18.3 mg, 49.3 μmol) in Et₂O (0.3 mL). The mixture was stirred at room temperature for 5 min. The suspension was filtered and the solid was dried under vacuum at 50° C. for 3 days to give compound 144 (14 mg, 64%) as a white solid.

Compound 145 (102 mg, 93%) was prepared following an analogous procedure to the one reported for the synthesis of compound 144 using fraction B (92 mg) as starting material.

### E5. Preparation of Final Compound 7

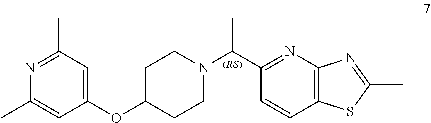

Compound 7 was prepared following an analogous procedure to the one described as Method 1 for the synthesis of compound 1 using intermediate 6 (100 mg, 0.48 mmol) and intermediate 30 (104 mg, 0.58 mmol) as starting materials yielding compound 7 (63 mg, 34%) as a yellow sticky solid.

### E6. Preparation of Final Compound-8

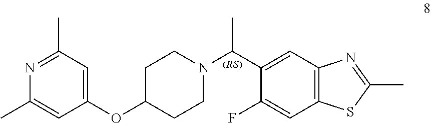

Compound 8 was prepared following an analogous procedure to the one described as Method 2 for the synthesis of compound 1 using intermediate 6 (50 mg, 0.22 mmol) and intermediate 37 (49 mg, 0.24 mmol) as starting materials. Compound 8 was purified by RP HPLC (stationary phase: C18 XBridge 30×100 mm 5 μm, mobile phase: gradient from 80% NH₄HCO₃ 0.25% solution in water, 20% CH₃CN to 0% NH₄HCO₃ 0.25% solution in water, 100% CH₃CN). The desired fractions were collected and partially concentrated in vacuo. The aqueous phase was extracted with EtOAc (3×), separated, dried (Na₂SO₄), filtered and the solvents evaporated in vacuo to yield yielding compound 8 (55 mg, 64%) as a colorless oil.

### E7. Preparation of Final Compounds 9, 10 and 11

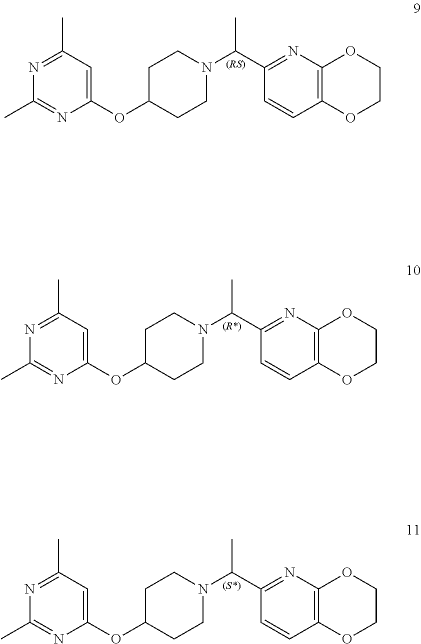 [SMILES: Cc1cc(OC2CCN(C(C)c3ccc4c(n3)OCCO4)CC2)nc(C)n1 ; Cc1cc(OC2CCN(C(C)c3ccc4c(n3)OCCO4)CC2)nc(C)n1 ; Cc1cc(OC2CCN(C(C)c3ccc4c(n3)OCCO4)CC2)nc(C)n1]

Compound 9 was prepared following an analogous procedure to the one described as Method 1 for the synthesis of compound 1 using intermediate 7 (186 mg, 0.9 mmol) and 2,3-dihydro-[1,4]dioxino[2,3-b]pyridine-6-carboxaldehyde (CAS: 615568-24-6, 163 mg, 0.99 mmol) as starting materials yielding compound 9 (163 mg, 49%) as brown syrup.

Compound 9 (160 mg) was purified via chiral SFC (stationary phase: CHIRALPAK AD-H 5 μm 250*30 mm, mobile phase: 92% CO₂, 8% iPrOH (0.3% iPrNH₂)) yielding compound 10 (65 mg, 20%) and compound 11 (56 mg, 17%) both as oils. Compounds 10 and 11 were dissolved in Et₂O and then HCl (2N in Et₂O) was added. The resulting solids were filtered and dried to give compounds 10 (64 mg, 18%, HCl salt) and 11 (54 mg, 15%, HCl salt) both as white solids.

### E8. Preparation of Final Compound 12

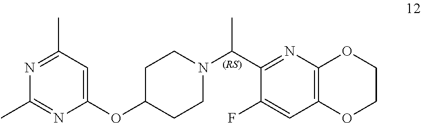 [SMILES: Cc1cc(OC2CCN(C(C)c3nc4c(cc3F)OCCO4)CC2)nc(C)n1]

Compound 12 was prepared following an analogous procedure to the one described as Method 2 for the synthesis of compound 1 using intermediate 7 (211 mg, 0.77 mmol) and intermediate 20 (120 mg, 0.55 mmol) as starting materials. Compound 12 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and partially concentrated in vacuo. The aqueous phase was extracted with EtOAc (3×), separated, dried (Na₂SO₄), filtered and the solvents evaporated in vacuo to yield compound 12 (102 mg, 48%) as a colorless oil.

### E9. Preparation of Final Compound 13

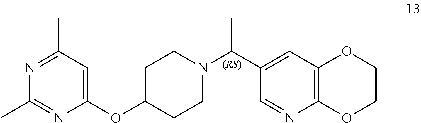 [SMILES: Cc1cc(OC2CCN(C(C)c3cnc4c(c3)OCCO4)CC2)nc(C)n1]

Compound 13 was prepared following an analogous procedure to the one described as Method 2 for the synthesis of compound 1 using intermediate 7 (192 mg, 0.93 mmol) and intermediate 22 (167 mg, 0.83 mmol) as starting materials. Compound 13 was purified by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and partially concentrated in vacuo. The aqueous phase was extracted with EtOAc (3×), separated, dried (Na₂SO₄), filtered and the solvents evaporated in vacuo to yield compound 13 (92 mg, 27%) as a yellow sticky solid.

### E10. Preparation of Final Compound 14

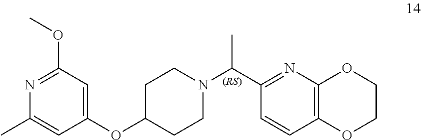 [SMILES: COc1cc(OC2CCN(C(C)c3ccc4c(n3)OCCO4)CC2)cc(C)n1]

Compound 14 was prepared following an analogous procedure to the one described as Method 2 for the synthesis of compound 1 using intermediate 8 (162 mg, 0.73 mmol) and intermediate 21 (135 mg, 0.68 mmol) as starting materials yielding compound 14 (160 mg, 57%) as a colorless oil.

### E10. Preparation of Final Compound 15

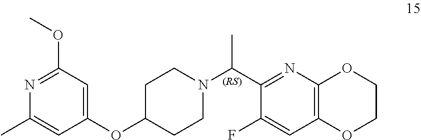 [SMILES: COc1cc(OC2CCN(C(C)c3nc4c(cc3F)OCCO4)CC2)cc(C)n1]

Compound 15 was prepared following an analogous procedure to the one described as Method 2 for the synthesis of compound 1 using intermediate 8 (51 mg, 0.23 mmol) and intermediate 20 (50 mg, 0.23 mmol) as starting materials. Compound 15 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and partially concentrated in vacuo. The aqueous phase was extracted with EtOAc (3×), separated, dried (Na₂SO₄), filtered and the solvents evaporated in vacuo to yield compound 15 (38 mg, 41%) as a yellow sticky solid.

### E12. Preparation of Final Compound 16

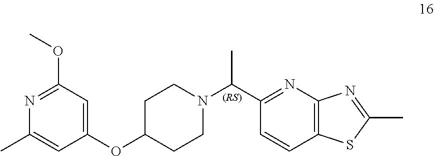

Compound 16 was prepared following an analogous procedure to the one described as Method 2 for the synthesis of compound 1 using intermediate 8 (185 mg, 0.83 mmol) and intermediate 30 (180 mg, 1 mmol) as starting materials yielding compound 16 (205 mg, 83%) as a yellow oil.

### E13. Preparation of Final Compounds 17,146 and 147

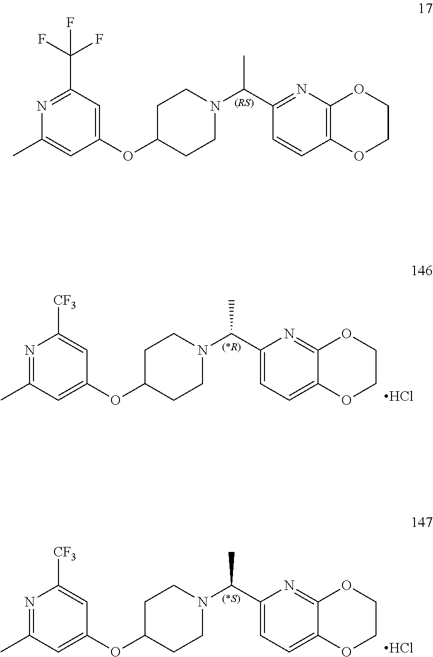 [SMILES: Cc1cc(OC2CCN(C(C)c3ccc4c(n3)OCCO4)CC2)cc(C(F)(F)F)n1 ; Cc1cc(OC2CCN([C@H](C)c3ccc4c(n3)OCCO4)CC2)cc(C(F)(F)F)n1 ; Cl ; Cc1cc(OC2CCN([C@@H](C)c3ccc4c(n3)OCCO4)CC2)cc(C(F)(F)F)n1 ; Cl]

Compound 17 was prepared following an analogous procedure to the one described as Method 2 for the synthesis of compound 1 using intermediate 9 (150 mg, 0.58 mmol) and intermediate 21 (104 mg, 0.52 mmol) as starting materials. Compound 17 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and partially concentrated in vacuo. The aqueous phase was extracted with EtOAc (3×), separated, dried (Na₂SO₄), filtered and the solvents evaporated in vacuo to yield compound 17 (99 mg, 41%) as a yellow sticky solid.

A purification was performed via chiral SFC (stationary phase: CHIRACEL OJ-H 5 μm 250*20 mm, mobile phase: 80% CO₂, 20% MeOH (0.3% i-PrNH₂)) to deliver the two fractions: fraction A (34 mg) and fraction B (36 mg). The fractions were separately purified via Reverse phase (stationary phase: YMC-actus Triart C18 10 μm 30*150 mm, mobile phase: NH₄HCO₃ (0.2%)/CH₃CN, gradient from 50:50 to 25:75) to afford fraction A (23 mg) and fraction B (31 mg).

HCl (2N in Et₂O, 81.5 μL, 0.16 mmol) was added to a solution of fraction A (23 mg, 54.3 μmol) in Et₂O (0.17 mL). The mixture was stirred at room temperature for 1 h. The solid was filtered off, washed with Et₂O and dried to afford compound 146 (21 mg, 84%) as a white solid.

Compound 147 (25 mg, 74%) was obtained following an analogous procedure to the one reported for the synthesis of compound 146 using fraction B as starting material.

### E14. Preparation of Final Compound 18

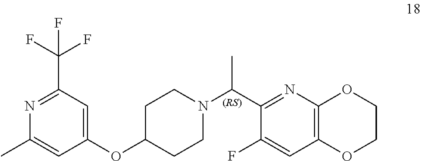 [SMILES: Cc1cc(OC2CCN(C(C)c3nc4c(cc3F)OCCO4)CC2)cc(C(F)(F)F)n1]

Compound 18 was prepared following an analogous procedure to the one described as Method 2 for the synthesis of compound 1 using intermediate 9 (60 mg, 0.23 mmol) and intermediate 20 (50 mg, 0.23 mmol) as starting materials. Compound 18 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and partially concentrated in vacuo. The aqueous phase was extracted with EtOAc (3×), separated, dried (Na₂SO₄), filtered and the solvents evaporated in vacuo to yield compound 18 (40 mg, 39%) as a yellow sticky solid.

### E15. Preparation of Final Compound 19

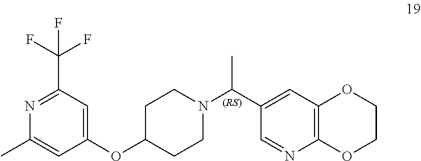 [SMILES: Cc1cc(OC2CCN(C(C)c3cnc4c(c3)OCCO4)CC2)cc(C(F)(F)F)n1]

Compound 19 was prepared following an analogous procedure to the one described as Method 2 for the synthesis of compound 1 using intermediate 9 (150 mg, 0.58 mmol) and intermediate 22 (104 mg, 0.52 mmol) as starting materials. Compound 19 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and partially concentrated in vacuo. The aqueous phase was extracted with EtOAc (3×), separated, dried (Na₂SO₄), filtered and the solvents evaporated in vacuo to yield compound 19 (103 mg, 42%) as a yellow sticky solid.

### E16. Preparation of Final Compound 20

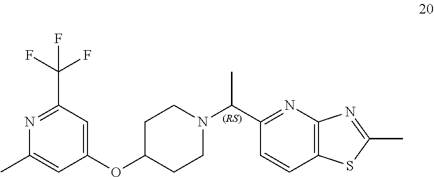

Compound 20 was prepared following an analogous procedure to the one described as Method 2 for the synthesis of compound 1 using intermediate 9 (100 mg, 0.38 mmol) and intermediate 30 (86 mg, 0.46 mmol) as starting materials. Compound 20 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and partially concentrated in vacuo. The aqueous phase was extracted with EtOAc (3×), separated, dried (Na₂SO₄), filtered and the solvents evaporated in vacuo to yield compound 20 (78 mg, 47%) as a yellow sticky solid.

### E17. Preparation of Final Compound 21

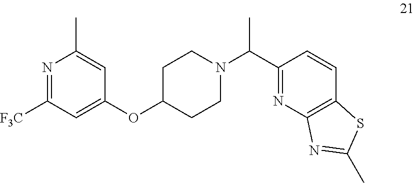 [SMILES: Cc1cc(OC2CCN(C(C)c3ccc4sc(C)nc4n3)CC2)cc(C(F)(F)F)n1]

Ti(Oi-Pr)₄ (0.19 mL, 0.65 mmol) was added to a stirred mixture of intermediate 9 (100 mg, 0.38 mmol) and intermediate 30 (85.7 mg, 0.46 mmol) in DCM (1.70 mL) at room temperature under N₂ atmosphere. The reaction mixture was stirred at room temperature for 16 h, cooled at 0° C. and Methylmagnesium bromide (1.4 M, 1.37 mL, 1.92 mmol) was added dropwise. The reaction mixture was stirred at this temperature for 15 min and at room temperature for 2 h. The mixture was treated with NH₄Cl (sat., aq.) and extracted with DCM. The phases were filtered through Celite®. The organic layer was dried (Na₂SO₄), filtered and the solvents were evaporated in vacuo. The crude product was purified by flash column chromatography (silica, DCM/MeOH, gradient from 100:0 to 99:1). A second purification was performed by RP HPLC (stationary phase: C18 XBridge 30×100 mm 5 μm), mobile phase: NH₄HCO₃ (0.25% solution in water)/CH₃CN, gradient from 60:40 to 43:57%). The desired fractions were collected and the solvents were partially concentrated in vacuo. The aqueous phase was extracted with EtOAc. The combined organic phases were dried (Na₂SO₄), filtered and the solvent was evaporated in vacuo to afford compound 21 (78.2 mg, 47%) as a yellow sticky solid.

### E18. Preparation of Final Compound 22

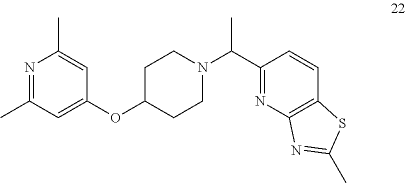

Compound 22 was prepared following an analogous procedure to the one described for the synthesis of compound 21 using intermediates 7 and 30 as starting materials. The crude product was purified by flash column chromatography (silica; NH₃ (7M in MeOH)/DCM, gradient from 100:0 to 98.5:1.5). A second purification was performed by RP HPLC (stationary phase: C18 XBridge 30×100 mm 5 μm), mobile phase: NH₄HCO₃ (0.25% solution in water)/CH₃CN, gradient from 75:25 to 57:43). The desired fractions were collected and the solvents were partially concentrated in vacuo. The aqueous phase was extracted with EtOAc. the combined organic layers were dried (Na₂SO₄), filtered and the solvent was evaporated in vacuo to afford compound 22 (97.7 mg, 53%) as a yellow oil.

### E19. Preparation of Final Compounds 23 and 24

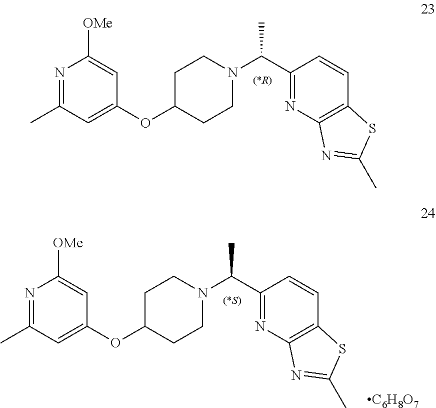

Compounds 23 and 24 were was prepared following an analogous procedure to the one described for the synthesis of compound 21 using intermediates 8 and 30 as starting materials.

The crude product was purified by flash column chromatography (silica, NH₃ (7N in MeOH)/DCM, gradient from 0:100 to 2:98). The desired fractions were collected and the solvents were evaporated in vacuo to afford a mixture of enantiomers (52 mg, 58%) as a yellow oil.

The mixture was combined with another fraction (152 mg) and purified by RP HPLC (stationary phase: C18 XBridge 30×100 mm 5 μm), mobile phase: NH₄HCO₃ (0.25% solution in water)/CH₃CN, gradient from 75:2 to 57:43). The desired fractions were collected and partially concentrated in vacuo. The aqueous phase was extracted with EtOAc (3 times). The combined organic extracts were dried (Na₂SO₄), filtered and the solvent was evaporated in vacuo to afford a mixture of enantiomers (171 mg) as yellow film.

A purification was performed via chiral SFC (stationary phase: CHIRACEL OJ-H 5 μm 250*20 mm, mobile phase: 70% CO₂, 30% EtOH (0.3% i-PrNH₂)) to give compound 23 (72 mg) and another fraction (72 mg) as yellow oils.

A solution of citric acid (30.8 mg, 0.16 mmol) in 1,4-dioxane (1 mL) was added to a stirred solution of the isolated fraction (64 mg, 0.16 mmol) in Et₂O (1 mL). The mixture was stirred at room temperature for 1 h. The mixture was completely dissolved in MeOH (1 mL) and evaporated in vacuo. The residue was triturated with tert-butylmethylether, filtered and the solid was dried under vacuum at 50° C. for 1 day to give compound 24 (85 mg, 90%) as a beige solid.

### E20. Preparation of Final Compound 25

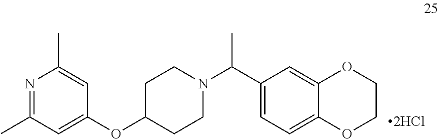 [SMILES: Cc1cc(OC2CCN(C(C)c3ccc4c(c3)OCCO4)CC2)cc(C)n1]

Ti(Oi-Pr)₄ (0.21 mL, 0.73 mmol) was added to a stirred mixture of intermediate 6 (100 mg, 0.49 mmol) and 1,4-benzodioxan-6-carboxaldehyde (CAS: 29668-44-8; 87.5 mg, 0.53 mmol) in DCM (3.1 mL) under N₂ atmosphere. The reaction mixture was stirred at room temperature for 16 h. Methylmagnesium bromide (3.2M solution, 0.45 mL, 1.45 mmol) was added at 0° C. and the reaction mixture was stirred for 30 min and at room temperature. NH₄Cl (3 mL) was added and the mixture was diluted with water (10 mL).

The aqueous phase was extracted with DCM. The combined organic layers were dried (Na₂SO₄), filtered and concentrated in vacuo. The crude mixture was purified by flash column chromatography (silica, MeOH in DCM, gradient from 0:100 to 15:85). The desired fractions were collected and solvents were concentrated in vacuo. The residue was purified by RP HPLC (stationary phase: C18 XBridge 30×100 mm 5 μm), mobile phase: NH₄HCO₃ (0.25% solution in water)/CH₃CN, gradient from 75:25 to 40:60). The residue (54.5 mg) was treated with HCl (2N in Et₂O). The solid was filtered off and dried to afford compound 25 (50.2 mg, 23%) as a white solid.

### E21. Preparation of Final Compound 26

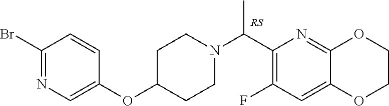 [SMILES: CC(c1nc2c(cc1F)OCCO2)N1CCC(Oc2ccc(Br)nc2)CC1]

Intermediate 20 (74.48 mg, 0.342 mmol) and K₂CO₃ (128.99 mg, 0.933 mmol) were added to a stirred solution of intermediate 202 (80 mg, 0.311 mmol) in CH₃CN (1.606 mL). The mixture was stirred at 80° C. for 18 h. Water was added, and the mixture was extracted with EtOAc. The organic phase was separated, dried (MgSO₄), filtered and evaporated under vacuum. The crude product was purified by flash column chromatography (silica; MeOH in DCM 0/100 to 10/90). The desired fractions were collected and concentrated in vacuo to yield a mixture of stereoisomers. The mixture was purified by RP HPLC (Stationary phase: C18 XBridge 30×100 mm 5 μm, mobile phase: gradient from 67% 0.1% NH₄CO₃H/NH₄OH pH 9 solution in water, 33% CH₃CN to 50% 0.1% NH₄CO₃H/NH₄OH pH 9 solution in water, 50% CH₃CN). The desired fractions were collected and concentrated in vacuo to afford compound 26 (69 mg, 51%) as a light yellow solid (sticky).

### E22. Preparation of Final Compound 27

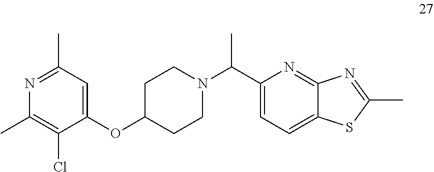

Intermediate 30 (77.7 mg, 0.44 mmol) and Ti(O-iPr)₄ (0.18 mL, 0.62 mmol) were added to a solution of intermediate 79 (100 mg, 0.42 mmol) in DCM (1.33 mL). The reaction mixture was stirred at room temperature for 16 h, cooled to 0° C. and methylmagnesium bromide (1.4M solution, 0.89 mL, 1.25 mmol) was added dropwise. The reaction mixture was stirred at room temperature for 2 h, quenched with NaHCO₃ (sat., aq.) and extracted with DCM. The organic layer was dried (MgSO₄), filtered and the solvents were evaporated in vacuo. The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: NH₄HCO₃ (0.25% solution in water)/CH₃CN, gradient from 90:10 to 60:40) to give compound 27 (85 mg, 49%) as a light yellow solid.

### E23. PREPARATION of Final Compound 28

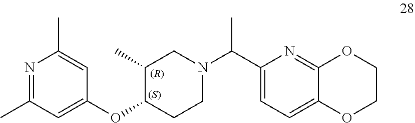

2,3-Dihydro-[1,4]dioxino[2,3-b]pyridine-6-carbaldehyde (CAS:615568-24-6; 216 mg, 1.31 mmol) and Ti(Oi-Pr)₄ (0.96 mL, 3.27 mmol) were added to a stirred solution of intermediate 103 (240 mg, 1.09 mmol) in DCM (5.08 mL) at room temperature and under N₂ atmosphere. The reaction mixture was stirred for 16 h. The mixture was cooled at 0° C. and methylmagnesium bromide (1.4M in THF, 3.89 mL, 5.45 mmol) was added dropwise. The reaction mixture was stirred at this temperature for 25 min and at room temperature for 2 h. The mixture was treated with NH₄Cl (sat., aq.) and filtered through Celite®. The aqueous phase was washed with DCM. The combined organic layers were washed with H₂O, dried (Na₂SO₄), filtered and the solvent was evaporated in vacuo. The crude product was purified by flash column chromatography (silica, MeOH in DCM, gradient from 0:100 to 4:96). The desired fractions were collected and concentrated in vacuo to yield compound 28 (180 mg, 43%) as a sticky oil.

### E24. Preparation of Final Compound 29

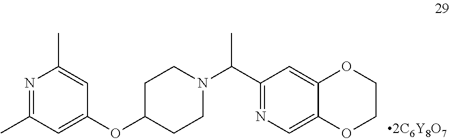 [SMILES: Cc1cc(OC2CCN(C(C)c3cc4c(cn3)OCCO4)CC2)cc(C)n1]

2H,3H-[1,4]Dioxino[2,3-c]pyridine-7-carbaldehyde (CAS: 443955-90-6; 62.1 mg, 0.38 mmol) and Ti(Oi-Pr)₄ (0.16 mL, 0.54 mmol) were added to a solution of intermediate 6 (100 mg, 0.36 mmol) in methylmagnesium bromide (1.4M solution, 1.28 mL, 1.79 mmol). The reaction mixture was stirred at room temperature for 16 h, cooled to 0° C. and DCM (30 μL) was added dropwise. The mixture was stirred at room temperature for 2 h and NH₄Cl (sat., aq.) was added. The mixture was stirred for 10 min, basified with Na₂CO₃ (sat., aq.) and extracted with EtOAc. The organic layer was dried (Na₂SO₄), filtered and the solvents were evaporated in vacuo. The crude product was purified by flash column chromatography (silica, MeOH in DCM, gradient from 0:100 to 5:95) The desired fractions were collected and concentrated in vacuo. The residue (68 mg) was dissolved in EtOAc and a solution of citric acid (35.4 mg, 0.18 mmol) dissolved in EtOAc was added. The mixture was stirred at room temperature and the solid was filtered off to give compound 29 (65 mg, 24%) as a white solid.

### E25. Preparation of Final Compound 30

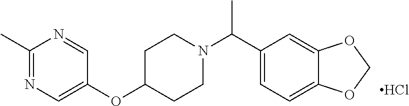 [SMILES: Cc1ncc(OC2CCN(C(C)c3ccc4c(c3)OCO4)CC2)cn1 ; Cl]

Piperonal (CAS: 120-57-0; 127 mg, 0.85 mmol) and Ti(Oi-Pr)₄ (0.63 mL, 2.11 mmol) were added to a solution of intermediate 73 (136 mg, 0.70 mmol) in anhydrous THF (1.8 mL) at room temperature. The reaction mixture was stirred for 18 h. The mixture was distilled and dried under vacuum. Anhydrous THF (1.8 mL) was added and the mixture was cooled to 0° C. methylmagnesium bromide (1.4M in THF, 2.51 mL, 3.52 mmol) was added dropwise. The reaction mixture was stirred at 0° C. for 15 min and at room temperature for 15 h. NH₄Cl (sat., aq.) was added and the mixture was extracted with DCM (3 times). The combined organic layers were dried (MgSO₄), filtered and concentrated in vacuo. The crude product was purified by flash column chromatography (silica, MeOH in DCM, gradient from 0:100 to 4:96). The desired fractions were collected and concentrated in vacuo. The residue (132 mg) was diluted in DCM and treated with HCl (4N in 1,4-dioxane, 1 eq). The solvents were evaporated in vacuo. The product was triturated with DIPE to give compound 30 (122 mg, 45%) as a white solid.

### E26. Preparation of Final Compound 31

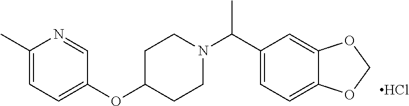 [SMILES: Cc1ccc(OC2CCN(C(C)c3ccc4c(c3)OCO4)CC2)cn1 ; Cl]

Compound 31 was prepared following an analogous procedure to the one described for the synthesis of compound 30 using piperonal (CAS: 120-57-0) and intermediate 89 as starting materials.

The crude product was purified by flash column chromatography (silica, MeOH in DCM, gradient from 0:100 to 4:96). The desired fractions were collected and concentrated in vacuo. The residue (13 mg) was diluted in DCM and treated with HCl (4N in 1,4-dioxane). The solvents were evaporated in vacuo. The product was triturated with DIPE to give compound 31 (7 mg, 3%) as a white solid.

### E27. Preparation of Final Compound 32

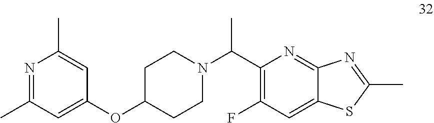

Sodium cyanoborohydride (18.3 mg, 0.29 mmol) was added to a stirred mixture of intermediate 6 (50.0 mg, 0.24 mmol), intermediate 128 (53.5 mg, 0.25 mmol) and Ti(O-iPr)₄ (106 μL, 0.36 mmol) in THF (1.78 mL) at room temperature under N₂ atmosphere.

The reaction mixture was stirred at 70° C. for 16 h. Water was added and the mixture was extracted with EtOAc. The organic layer was dried (MgSO₄), filtered and the solvents were evaporated in vacuo. The residue was purified by RP HPLC (stationary phase: C18 XBridge 30×100 mm 5 μm), mobile phase: NH₄HCO₃ (0.25% solution in water)/CH₃CN, gradient from 75:25 to 57:43) to afford compound 32 (13 mg, 13%) as an off white solid.

### E28. Preparation of Final Compound 33

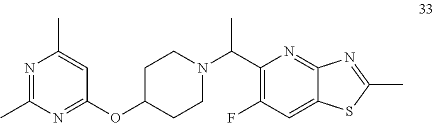

Compound 33 was prepared following an analogous procedure to the one described for the synthesis of compound 32 using intermediate 7 and intermediate 128 as starting materials.

The residue was purified by RP HPLC (stationary phase: C18 XBridge 30×100 mm 5 μm), mobile phase: NH₄HCO₃ (0.25% solution in water)/CH₃CN, gradient from 75:25 to 57:43) to give compound 33 (20 mg, 21%) as an off white solid.

### E29. Preparation of Final Compound 34

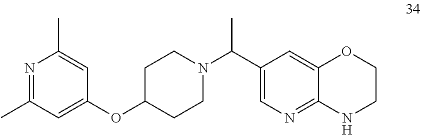 [SMILES: Cc1cc(OC2CCN(C(C)c3cnc4c(c3)OCCN4)CC2)cc(C)n1]

Intermediate 144 (135 mg, 0.49 mmol) followed by Ti(Oi-Pr)₄ (0.21 mL, 0.73 mmol) were added to a stirred solution of intermediate 6 (100 mg, 0.49 mmol) in THF (3.57 mL) at room temperature and under N₂ atmosphere. The reaction mixture was stirred at 80° C. overnight. Then the mixture was cooled down to room temperature and sodium cyanoborohydride (36.6 mg, 0.58 mmol) was added. The reaction mixture was stirred at 80° C. for another 24 h and diluted with water. The mixture was extracted with EtOAc. The organic layer was dried (MgSO₄), filtered and the solvents were evaporated in vacuo. The residue was purified by flash column chromatography (silica, MeOH in DCM, gradient from 0:100 to 5:95). The desired fractions were collected and concentrated in vacuo to afford compound 34 (85 mg, 48%) as a white solid.

### E30. Preparation of Final Compound 35

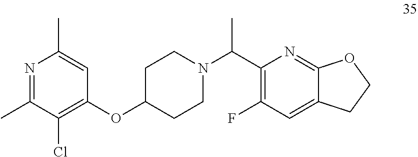 [SMILES: Cc1cc(OC2CCN(C(C)c3nc4c(cc3F)CCO4)CC2)c(Cl)c(C)n1]

Intermediate 25 (79.0 mg, 0.44 mmol) and Ti(Oi-Pr)₄ (0.18 mL, 0.62 mmol) were added to a solution of intermediate 79 (100 mg, 0.42 mmol) in DCM (2 mL). The reaction mixture was stirred at room temperature for 16 h, cooled to 0° C. and sodium cyanoborohydride (78.3 mg, 1.25 mmol) was added dropwise. The reaction mixture was stirred at room temperature for 2 h, quenched with NH₄Cl (sat., aq.) and extracted with DCM. The organic layer was dried (MgSO₄), filtered and the solvents were evaporated in vacuo. The crude product was purified by flash column chromatography (silica, MeOH in DCM, gradient from 0:100 to 10:90). The residue was further purified twice by RP HPLC (stationary phase: C18 XBridge 30×100 mm 5 μm), mobile phase: NH₄HCO₃ (0.25% solution in water)/CH₃CN, gradient from 90:10 to 60:40) to yield compound 35 (35 mg, 21%) as a white solid.

### E31. Preparation of Final Compound 36

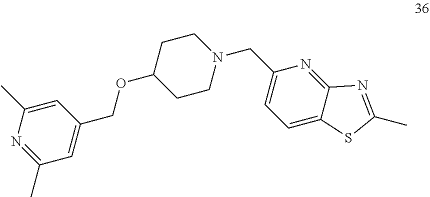

Ti(O-iPr)₄ (73.7 μL, 0.25 mmol) was added to a stirred solution of intermediate 63 (37.0 mg, 0.17 mmol) and intermediate I-30 (33.3 mg, 0.19 mmol) in DCM (1.08 mL). The reaction mixture was stirred at room temperature for 7 h. Sodium triacetoxyborohydride (107 mg, 0.50 mmol) was added and the reaction mixture was stirred for 16 h. The mixture was diluted with NaHCO₃ (sat., aq.) and extracted with DCM. The organic layer was dried (MgSO₄), filtered and the solvents were evaporated in vacuo. The crude mixture was purified by flash column chromatography (silica, MeOH in EtOAc, gradient from 0:100 to 3:97). The desired fractions were collected and the solvents were evaporated in vacuo. The residue was purified by RP HPLC (stationary phase: C18 XBridge 30×100 nm 5 um), mobile phase: (0.1% NH₄CO₃H/NH₄OH pH=9 solution in water)/CH₃CN, gradient from 74:26 to 58:42) to give compound 36 (35 mg, 54%) as a white solid.

### E32. Preparation of Final Compound 37

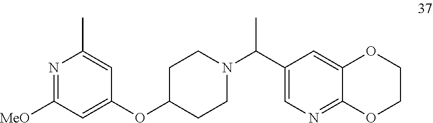 [SMILES: COc1cc(OC2CCN(C(C)c3cnc4c(c3)OCCO4)CC2)cc(C)n1]

K₂CO₃ (187 mg, 1.35 mmol) was added to a stirred mixture of intermediate 8 (100 mg, 0.45 mmol) and intermediate 22 (80.8 mg, 0.41 mmol) in CH₃CN (3.51 mL). The reaction mixture was stirred at 70° C. for 20 h. The reaction mixture was diluted with EtOAc and filtered through Celite®. The solvents were evaporated in vacuo. The crude product was purified by flash column chromatography (silica, NH₃ (7M in MeOH)/DCM, gradient from 0:100 to 2:98). The desired fractions were collected and the solvents were evaporated in vacuo to afford compound 37 (84 mg, 48%) as a yellow oil.

### E33. Preparation of Final Compound 38

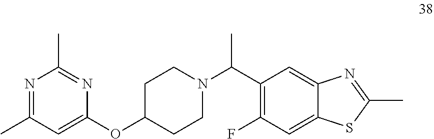

Compound 38 was prepared following an analogous procedure to the one described for the synthesis of compound 37 using intermediate 7 and intermediate 37 as starting materials.

The crude product was purified by flash column chromatography (silica, NH₃ (7M in MeOH)/DCM, gradient from 0:100 to 1:99). The desired fractions were collected and the solvents were evaporated in vacuo. A second purification was performed by RP HPLC (stationary phase: C18 XBridge 30×100 mm 5 μm), mobile phase: NH₄HCO₃ (0.25% solution in water)/CH₃CN, gradient from 60:40 to 43:57). The aqueous phase was extracted with EtOAc. The combined organic extracts were dried (Na₂SO₄), filtered and the solvent was evaporated in vacuo to afford compound 38 (72.8 mg, 38%) as a yellow sticky solid.

### E34. Preparation of Final Compound 39

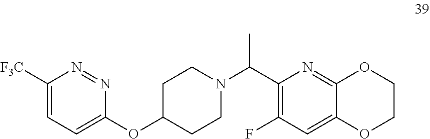 [SMILES: CC(c1nc2c(cc1F)OCCO2)N1CCC(Oc2ccc(C(F)(F)F)nn2)CC1]

Compound 39 was prepared following an analogous procedure to the one described for the synthesis of compound 37 using intermediate 41 and intermediate 20 as starting materials.

The crude product was purified by flash column chromatography (silica, NH₃ (7m in MeOH)/DCM, gradient from 0:100 to 1:99). The desired fractions were collected and the solvents were evaporated in vacuo to afford compound 39 (138 mg, 58%) as a yellow solid.

### E35. Preparation of Final Compound 40

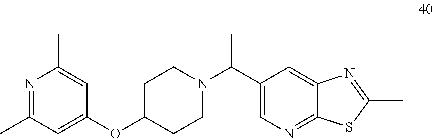

Compound 40 was prepared following an analogous procedure to the one described for the synthesis of compound 37 using intermediate 6 and intermediate 124 as starting materials.

The crude product was purified by flash column chromatography (silica, NH₃ (7N in MeOH)/DCM, gradient from 0:100 to 10:90). A second purification was performed by RP HPLC (stationary phase: C18 XBridge 30×100 mm 5 μm), mobile phase: NH₄HCO₃ (0.25% solution in water)/CH₃CN, gradient from 75:25 to 57:43). The desired fractions were collected and concentrated in vacuo. The residue was dissolved in EtOAc and washed with NaHCO₃ (sat., aq.). The organic phase was dried (Na₂SO₄), filtered and concentrated in vacuo to afford compound 40 (13 mg, 17%) as a colourless oil.

### E36. Preparation of Final Compound 41

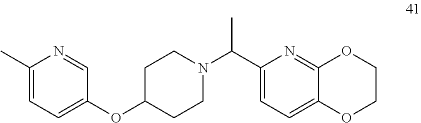 [SMILES: Cc1ccc(OC2CCN(C(C)c3ccc4c(n3)OCCO4)CC2)cn1]

K₂CO₃ (216 mg, 1.56 mmol) was added to a stirred mixture of intermediate 89 (100 mg, 0.52 mmol) and intermediate 21 (104 mg, 0.52 mmol) in CH₃CN (78.8 mL). The reaction mixture was stirred at 70° C. for 12 h and diluted with water. The aqueous phase was extracted with EtOAc. The combined organic layers were dried (Na₂SO₄), filtered and the solvents were evaporated in vacuo. The crude mixture was purified by RP HPLC (stationary phase: C18 XBridge 30×100 mm 5 μm), mobile phase: NH₄HCO₃ (0.25% solution in water)/CH₃CN, gradient from 80:20 to 60:40). The desired fractions were collected and evaporated in vacuo to afford compound 41 (35 mg, 19%) as a colorless oil. This fraction was taken into DCM and treated with 1 eq of HCl 4N in dioxane (0.1 ml). The solvents were evaporated in vacuo and the product was tritured with diethyl ether to afford compound 41 (125 mg, 36%) as a white solid.

### E37. Preparation of Final Compound 42

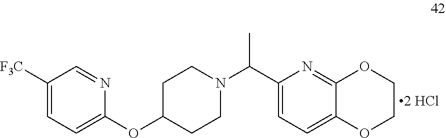 [SMILES: CC(c1ccc2c(n1)OCCO2)N1CCC(Oc2ccc(C(F)(F)F)cn2)CC1]

Compound 42 was prepared following an analogous procedure to the one described for the synthesis of compound 41 using intermediate 95.TFA and intermediate 21 as starting materials.

The crude product was purified by RP HPLC (stationary phase: C18 XBridge 30×100 mm 5 μm), mobile phase: NH₄HCO₃ (0.25% solution in water)/CH₃CN, gradient from 54:46 to 36:64).

The residue (56 mg) was suspended in Et₂O and treated with HCl (2N solution in Et₂O, 4 eq) at room temperature. The white precipitate was filtered off and dried to afford compound 42 (29.6 mg, 17%) as a white solid.

### E38. Preparation of Final Compound 43

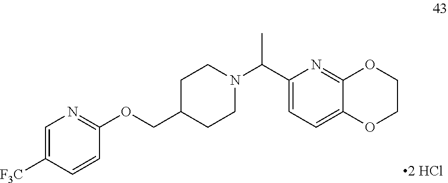 [SMILES: CC(c1ccc2c(n1)OCCO2)N1CCC(COc2ccc(C(F)(F)F)cn2)CC1]

Compound 43 was prepared following an analogous procedure to the one described for the synthesis of compound 41 using intermediate 71 and intermediate 21 as starting materials.

The crude product was purified by RP HPLC (stationary phase: C18 XBridge 30×100 mm 5 μm), mobile phase: NH₄HCO₃ (0.25% solution in water)/CH₃CN, gradient from 80:20 to 0:100). The desired fractions were collected and evaporated in vacuo.

The residue (123.7 mg) was suspended in Et₂O and treated with HCl (2N solution in Et₂O, 4 eq) at room temperature. The white precipitate was filtered off and dried to afford compound 43 (123.1 mg, 50%) as a white solid.

### E39. Preparation of Final Compound 44

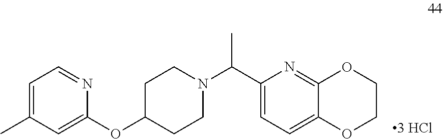 [SMILES: Cc1ccnc(OC2CCN(C(C)c3ccc4c(n3)OCCO4)CC2)c1]

Compound 44 was prepared following an analogous procedure to the one described for the synthesis of compound 41 using intermediate 91 and intermediate 21 as starting materials.

The crude product was purified by RP HPLC (stationary phase: XBridge C18 50×100 mm, 5 μm), mobile phase: NH₄HCO₃ (0.25% solution in water)/CH₃CN, gradient from 80:20 to 0:100). The desired fractions were collected and the volatiles were evaporated in vacuo to afford a brown oil (346 mg).

A fraction of the residue (322 mg) was suspended in Et₂O and treated with HCl (2N solution in Et₂O, 4 eq) at room temperature. The white precipitate was filtered off and dried to give compound 44 (305 mg) as a pale-cream solid.

### E40. Preparation of Final Compound 45

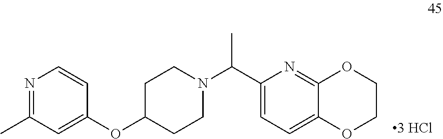 [SMILES: Cc1cc(OC2CCN(C(C)c3ccc4c(n3)OCCO4)CC2)ccn1]

Compound 45 was prepared following an analogous procedure to the one described for the synthesis of compound 41 using intermediate 67 and intermediate 21 as starting materials.

The crude product was purified by RP HPLC (stationary phase: C18 XBridge 30×100 mm 5 μm), mobile phase: NH₄HCO₃ (0.25% solution in water)/CH₃CN, gradient from 80:20 to 0:100). The desired fractions were evaporated in vacuo a pale yellow oil (121 mg).

The residue (113 mg) was suspended in Et₂O and treated with HCl (2N solution in Et₂O, 4 eq) at room temperature. The white precipitate was filtered off and dried to give compound 45 (131.9 mg, 39%) as a pale-cream solid.

### E41. Preparation of Final Compound 46

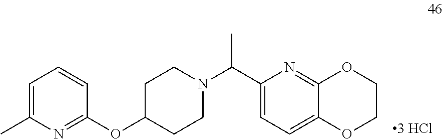 [SMILES: Cc1cccc(OC2CCN(C(C)c3ccc4c(n3)OCCO4)CC2)n1]

Compound 46 was prepared following an analogous procedure to the one described for the synthesis of compound 41 using intermediate 69 and intermediate 21 as starting materials.

The crude product was purified by RP HPLC (stationary phase: C18 XBridge 30×100 mm 5 μm), mobile phase: NH₄HCO₃ (0.25% solution in water)/CH₃CN, gradient from 80:20 to 0:100). The desired fractions were evaporated in vacuo to afford a colorless oil (112.6 mg).

The residue (105 mg) was suspended in Et₂O and treated with HCl (2N solution in Et₂O, 4 eq) at room temperature. The white precipitate was filtered off and dried to give compound 46 (117 mg, 39%) as a pale-cream solid.

### E42. Preparation of Final Compound 47

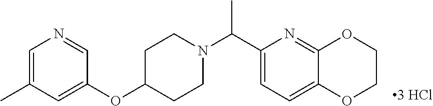 [SMILES: Cc1cncc(OC2CCN(C(C)c3ccc4c(n3)OCCO4)CC2)c1]

Compound 47 was prepared following an analogous procedure to the one described for the synthesis of compound 41 using intermediate 108 and intermediate 21 as starting materials.

The crude product was purified by RP HPLC (stationary phase: C18 XBridge 30×100 mm 5 μm), mobile phase: NH₄HCO₃ (0.25% solution in water)/CH₃CN, gradient from 80:20 to 0:100). The desired fractions were evaporated in vacuo to afford a colorless oil (97 mg).

The residue (76 mg) was suspended in Et₂O and treated with HCl (2N solution in Et₂O, 4 eq) at room temperature. The white precipitate was filtered off and dried to give compound 47 (74 mg, 21%) as a pale-cream solid.

### E43. Preparation of Final Compound 48

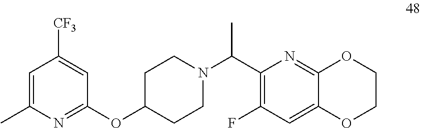 [SMILES: Cc1cc(C(F)(F)F)cc(OC2CCN(C(C)c3nc4c(cc3F)OCCO4)CC2)n1]

Compound 48 was prepared following an analogous procedure to the one described for the synthesis of compound 41 using intermediate 45 and intermediate 20 as starting materials.

The crude product was purified by RP HPLC (stationary phase: C18 XBridge 30×100 mm 5 μm), mobile phase: NH₄HCO₃ (0.25% solution in water)/CH₃CN, gradient from 80:20 to 0:100). The desired fractions were evaporated in vacuo to give compound 48 (148 mg, 71%) as a colorless oil which solidified upon standing.

### E44. Preparation of Final Compound 49

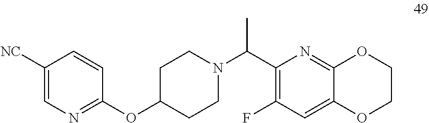 [SMILES: CC(c1nc2c(cc1F)OCCO2)N1CCC(Oc2ccc(C#N)cn2)CC1]

Compound 49 was prepared following an analogous procedure to the one described for the synthesis of compound 41 using intermediate 47 and intermediate 20 as starting materials.

The crude product was purified by RP HPLC (stationary phase: C18 XBridge 30×100 mm 5 μm), mobile phase: NH₄HCO₃ (0.25% solution in water)/CH₃CN, gradient from 80:20 to 0:100). The desired fractions were evaporated in vacuo to give compound 49 (43 mg, 17%) as a colorless oil.

### E45. Preparation of Final Compound 50

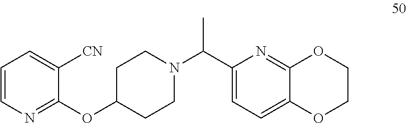 [SMILES: CC(c1ccc2c(n1)OCCO2)N1CCC(Oc2ncccc2C#N)CC1]

Compound 50 was prepared following an analogous procedure to the one described for the synthesis of compound 41 using intermediate 53 and intermediate 21 as starting materials.

The crude product was purified by flash column chromatography (silica, heptane/EtOAc, gradient from 100:0 to 0:100). A second purification was performed by RP HPLC (stationary phase: C18 XBridge 30×100 mm 5 μm), mobile phase: NH₄HCO₃ (0.25% solution in water)/CH₃CN, gradient from 80:20 to 0:100). The residue was washed with EtOAc and NaHCO₃ (sat., aq.). The organic layer was dried (Na₂SO₄), filtered and the solvents were evaporated in vacuo to afford compound 50 (92 mg, 56%) as a pale yellow oil.

### E46. Preparation of Final Compound 51

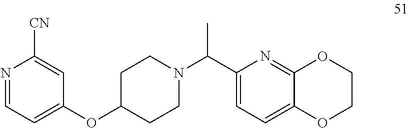 [SMILES: CC(c1ccc2c(n1)OCCO2)N1CCC(Oc2ccnc(C#N)c2)CC1]

Compound 51 was prepared following an analogous procedure to the one described for the synthesis of compound 41 using intermediate 55 and intermediate 21 as starting materials.

The crude product was purified by flash column chromatography (silica, heptane/EtOAc, gradient from 100:0 to 0:100). A second purification was performed by RP HPLC (stationary phase: C18 XBridge 30×100 mm 5 μm), mobile phase: NH₄HCO₃ (0.25% solution in water)/CH₃CN, gradient from 80:20 to 0:100). Another purification by RP HPLC (stationary phase: C18 XBridge 30×100 mm 5 μm), mobile phase: NH₄HCO₃ (0.25% solution in water)/CH₃CN, gradient from 80:20 to 0:100) delivered compound 51 (101 mg, 62%) as a pale yellow oil.

### E47. Preparation of Final Compound 52

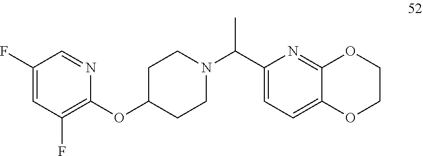 [SMILES: CC(c1ccc2c(n1)OCCO2)N1CCC(Oc2ncc(F)cc2F)CC1]

Compound 52 was prepared following an analogous procedure to the one described for the synthesis of compound 41 using intermediate 57 and intermediate 21 as starting materials.

The crude product was purified by flash column chromatography (silica, heptane/EtOAc, gradient from 100:0 to 0:100). A second purification was performed by RP HPLC (stationary phase: C18 XBridge 30×100 mm 5 μm), mobile phase: NH₄HCO₃ (0.25% solution in water)/CH₃CN, gradient from 80:20 to 0:100). The organic layer was evaporated in vacuo and the aqueous phase was washed with EtOAc and NaHCO₃ (sat., aq.). The organic layer was dried (Na₂SO₄), filtered and the solvents were evaporated in vacuo to afford compound 52 (135 mg, 84%) as a colorless film.

### E48. Preparation of Final Compounds 53 and 54

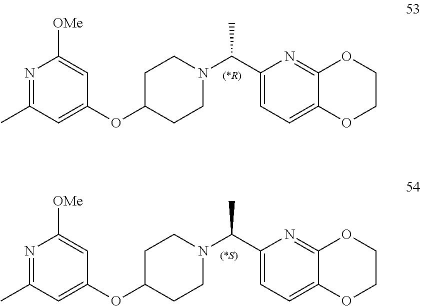 [SMILES: COc1cc(OC2CCN([C@H](C)c3ccc4c(n3)OCCO4)CC2)cc(C)n1 ; COc1cc(OC2CCN([C@@H](C)c3ccc4c(n3)OCCO4)CC2)cc(C)n1]

Compounds 52 and 53 were prepared following an analogous procedure to the one described for the synthesis of compound 41 using intermediate 8 and intermediate 21 as starting materials.

The crude product was purified by flash column chromatography (silica, NH₃ (7N in MeOH)/DCM, gradient from 0:100 to 2:98). The desired fractions were collected and the solvents were evaporated in vacuo to afford a mixture of products (160 mg). A purification was performed via chiral SFC (stationary phase: CHIRACEL OJ-H 5 μm 250*20 mm, mobile phase: 75% C02,25% EtOH (0.3% i-PrNH₂)) to give compound 53 (65 mg, 23%) and compound 54 (66 mg, 23%) as yellow oils.

### E49. Preparation of Final Compound 55

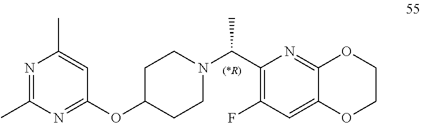 [SMILES: Cc1cc(OC2CCN([C@H](C)c3nc4c(cc3F)OCCO4)CC2)nc(C)n1]

Compound 55 was prepared following an analogous procedure to the one described for the synthesis of compound 41 using intermediate 7 and intermediate 20 as starting materials.

The crude product was purified by flash column chromatography (silica, NH₃ (7M in MeOH)/DCM, gradient from 0:100 to 5:95). A second purification was performed by flash column chromatography (silica, NH₃ (7M in MeOH)/DCM, gradient from 0:100 to 2:98). The residue was further purified by RP HPLC (stationary phase: C18 XBridge 30×100 mm 5 μm), mobile phase: NH₄HCO₃ (0.25% solution in water)/CH₃CN, gradient from 75:25 to 57:43). The desired fractions were collected and partially concentrated in vacuo. The aqueous phase was extracted with EtOAc (3 times), dried (Na₂SO₄), filtered and the solvent was evaporated in vacuo to afford a colorless oil (102 mg). A purification was performed via chiral SFC (stationary phase: CHIRACEL OJ-H 5 μm 250*20 mm, mobile phase: 90% CO₂, 10% EtOH (0.3% i-PrNH₂)) to afford 2 fractions: fraction A (44 mg) and fraction B (44 mg).

Fraction A (44 mg) was dissolved in Et₂O (1 mL) and HCl (2N in Et₂O, 0.8 mL) was added. The mixture was stirred at room temperature for 16 h and the solvent was concentrated in vacuo. tert-Butyl methyl ether was added and the mixture was sonicated for 10 min. The solvent was evaporated in vacuo. The process was repeated until the obtention of a solid (50 mg). The product was further purified by RP HPLC (stationary phase: C18 XBridge 30×100 mm 5 μm), mobile phase: NH₄HCO₃ (0.25% solution in water)/CH₃CN, gradient from 75:25 to 57:43). The desired fractions were collected and partially concentrated in vacuo. The aqueous phase was extracted with EtOAc (3 times), dried (Na₂SO₄), filtered and the solvent was evaporated in vacuo to give compound 55 (17.2 mg) as colorless oil.

### E50. Preparation of Final Compound 56

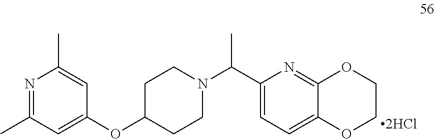 [SMILES: Cc1cc(OC2CCN(C(C)c3ccc4c(n3)OCCO4)CC2)cc(C)n1]

Compound 56 was prepared following an analogous procedure to the one described for the synthesis of compound 41 using intermediate 6 and intermediate 133 as starting materials.

The crude product was purified by RP HPLC (stationary phase: C18 XBridge 30×100 mm 5 μm), mobile phase: NH₄HCO₃ (0.25% solution in water)/CH₃CN, gradient from 85:15 to 55:45).

HCl (2N in Et₂O, 0.12 mL, 0.24 mmol) was added to a solution of the residue (30 mg) in Et₂O (0.26 mL). The mixture was stirred at room temperature for 30 min. The solid was filtered off, washed with Et₂O and dried to afford compound 56 (25 mg, 44%) as a white solid.

### E51. Preparation of Final Compound 57

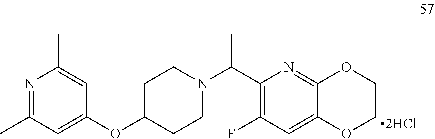 [SMILES: Cc1cc(OC2CCN(C(C)c3nc4c(cc3F)OCCO4)CC2)cc(C)n1]

Compound 57 was prepared following an analogous procedure to the one described for the synthesis of compound 41 using intermediate 6 and intermediate 20 as starting materials.

The crude product was purified by RP HPLC (stationary phase: C18 XBridge 30×100 mm 5 μm), mobile phase: NH₄HCO₃ (0.25% solution in water)/CH₃CN, gradient from 70:30 to 35:65). The residue was purified again by using an Isolute® SCX-2 cartridge which was washed with MeOH and the product was eluted with NH₃ (7N in MeOH) and the fraction was evaporated in vacuo.

HCl (2N in Et₂O, 0.21 mL, 0.42 mmol) was added to a solution of the residue (55 mg) in Et₂O (0.45 mL). The mixture was stirred at room temperature for 30 min. The solid was filtered off, washed with Et₂O, and dried to give compound 57 (55 mg, 56%) as a white solid.

### E52. Preparation of Final Compound 58

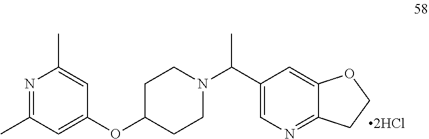 [SMILES: Cc1cc(OC2CCN(C(C)c3cnc4c(c3)OCC4)CC2)cc(C)n1]

Compound 58 was prepared following an analogous procedure to the one described for the synthesis of compound 41 using intermediate 6 and intermediate 136 as starting materials.

The crude product was purified by RP HPLC (stationary phase: C18 XBridge 30×100 mm 5 μm), mobile phase: NH₄HCO₃ (0.25% solution in water)/CH₃CN, gradient from 85:15 to 55:45).

The product was converted into the corresponding HCl salt. HCl (2N in Et₂O, 0.49 mL, 0.98 mmol) was added to a solution of the residue (115 mg) in Et₂O (1 mL). The mixture was stirred at room temperature for 30 min. The solid was filtered off, washed with Et₂O, and dried to give compound 58 (135 mg, 66%) as a white solid.

### E53. Preparation of Final Compound 59

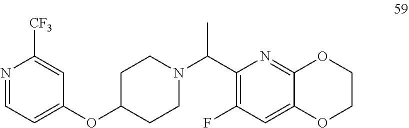 [SMILES: CC(c1nc2c(cc1F)OCCO2)N1CCC(Oc2ccnc(C(F)(F)F)c2)CC1]

Compound 59 was prepared following an analogous procedure to the one described for the synthesis of compound 41 using intermediate 105 and intermediate 20 as starting materials.

The crude product was purified by flash column chromatography (silica, DCM/MeOH, gradient from 100:0 to 95:5). A second purification was performed by flash column chromatography (silica, DCM/EtOAc, gradient from 50:50 to 0:100). The desired fractions were collected and evaporated in vacuo.

The product (123 mg) was dissolved in Et₂O and HCl (˜5M in i-PrOH) was added. The solid was filtered off and dried under vacuum at 50° C. for 3 days to give compound 59 (122 mg, 46%) as a white solid.

### E54. Preparation of Final Compounds 60, 61 and 62

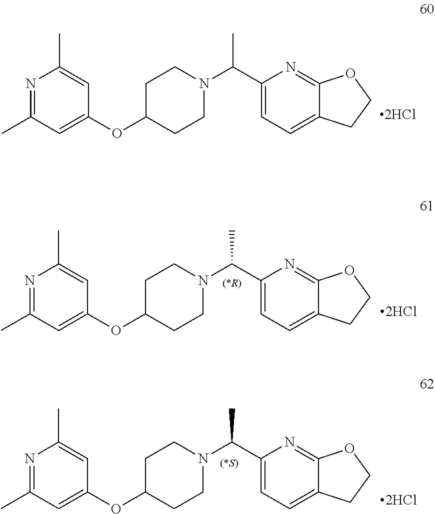 [SMILES: Cc1cc(OC2CCN(C(C)c3ccc4c(n3)OCC4)CC2)cc(C)n1 ; Cc1cc(OC2CCN([C@H](C)c3ccc4c(n3)OCC4)CC2)cc(C)n1 ; Cc1cc(OC2CCN([C@@H](C)c3ccc4c(n3)OCC4)CC2)cc(C)n1]

Compounds 60, 61 and 62 were prepared following an analogous procedure to the one described for the synthesis of compound 41 using intermediate 6 and intermediate 147 as starting materials.

The crude product was purified by flash column chromatography (silica, MeOH in DCM, gradient from 0:100 to 5:95). The desired fractions were collected and the solvents were evaporated in vacuo to afford an oil (167 mg).

A fraction of the residue (35 mg) was diluted in Et₂O (2 mL) and treated with HCl (1M in Et₂O, 0.1 mL, 0.1 mmol). The mixture was stirred for 30 min at room temperature.

The white solid was filtered off to give compound 60 (30 mg) as a white solid.

Another fraction of the residue was purified via chiral SFC (stationary phase: CHIRALPAK AD-H 5 μm 250*30 mm, mobile phase: 80% CO₂, 20% EtOH (0.3% i-PrNH₂) to give 2 fractions: fraction A (52 mg) and fraction B (53 mg).

Fraction A (52 mg, 0.15 mmol) was diluted in Et₂O (15 μL) and treated with HCl (1M in Et₂O, 0.15 mL, 0.15 mmol). The mixture was stirred at room temperature for 30 min. The solid was filtered off to give compound 61 (50.6 mg) as a solid.

Compound 62 (47.8 mg) was prepared following an analogous procedure using fraction B as starting material.

### E55. Preparation of Final Compound 63

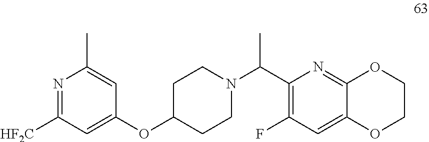

Compound 63 was prepared following an analogous procedure to the one described for the synthesis of compound 41 using intermediate 20 and intermediate 87 as starting materials.

The crude product was purified by flash column chromatography (silica, MeOH in DCM, gradient from 0:100 to 10:90). The desired fractions were collected and concentrated in vacuo. A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 63 (149 mg, 85%).

### E56. Preparation of Final Compound 64

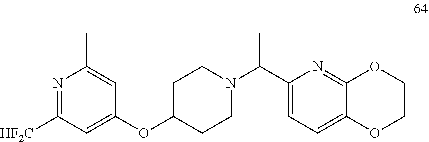

Compound 64 was prepared following an analogous procedure to the one described for the synthesis of compound 41 using intermediate 21 and intermediate 87 as starting materials.

The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 64 (198 mg, 59%) as a light yellow solid.

### E57. Preparation of Final Compound 65

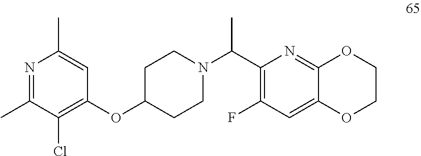 [SMILES: Cc1cc(OC2CCN(C(C)c3nc4c(cc3F)OCCO4)CC2)c(Cl)c(C)n1]

Compound 65 was prepared following an analogous procedure to the one described for the synthesis of compound 41 using intermediate 20 and intermediate 79 as starting materials.

The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 65 (74 mg, 42%) as a light yellow solid.

### E58. Preparation of Final Compound 66

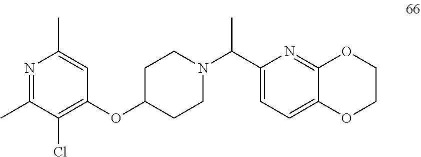 [SMILES: Cc1cc(OC2CCN(C(C)c3ccc4c(n3)OCCO4)CC2)c(Cl)c(C)n1]

Compound 66 was prepared following an analogous procedure to the one described for the synthesis of compound 41 using intermediate 21 and intermediate 79 as starting materials.

The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 66 (98 mg, 58%) as a light yellow solid.

### E59. Preparation of Final Compound 67

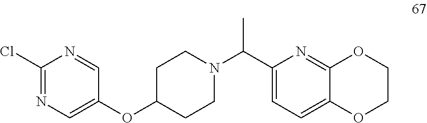 [SMILES: CC(c1ccc2c(n1)OCCO2)N1CCC(Oc2cnc(Cl)nc2)CC1]

Compound 67 was prepared following an analogous procedure to the one described for the synthesis of compound 41 using intermediate 21 and intermediate 81 as starting materials.

The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 67 (214 mg, 61%) as a light yellow solid.

### E60. Preparation of Final Compound 68

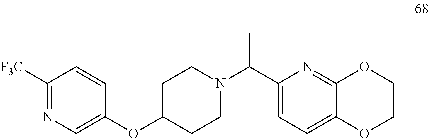 [SMILES: CC(c1ccc2c(n1)OCCO2)N1CCC(Oc2ccc(C(F)(F)F)nc2)CC1]

Compound 68 was prepared following an analogous procedure to the one described for the synthesis of compound 41 using intermediate 21 and intermediate 83 as starting materials.

The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 68 (118 mg, 71%) as a light yellow solid.

### E61. Preparation of Final Compound 69

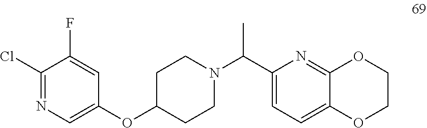 [SMILES: CC(c1ccc2c(n1)OCCO2)N1CCC(Oc2cnc(Cl)c(F)c2)CC1]

Compound 69 was prepared following an analogous procedure to the one described for the synthesis of compound 41 using intermediate 21 and intermediate 85 as starting materials.

The crude product was purified by flash column chromatography (silica, MeOH in DCM, gradient from 0:100 to 10:90). A second purification was performed by RP HPLC (stationary phase: C18 XBridge 30×100 mm 5 μm), mobile phase: (0.1% NH₄CO₃H/NH₄OH pH 9 solution in water)/CH₃CN, gradient from 67:33 to 50:50). The desired fractions were collected and concentrated in vacuo to afford compound 69 (105 mg, 61%) as a light yellow solid.

### E62. Preparation of Final Compound 70

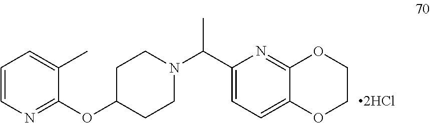 [SMILES: Cc1cccnc1OC1CCN(C(C)c2ccc3c(n2)OCCO3)CC1]

Intermediate 65 (80.9 mg, 0.42 mmol) was dissolved in anhydrous CH₃CN (3.16 mL). intermediate 1-21 (80.0 mg, 0.40 mmol) and K₂CO₃ (166 mg, 1.20 mmol) were added.

The reaction mixture was stirred at 80° C. overnight. The mixture was diluted with water and the mixture was extracted with DCM. The combined organic extracts were dried (Na₂SO₄), filtered and evaporated in vacuo. The crude product was purified twice by flash column chromatography (silica, NH₃ (7N in MeOH)/DCM, gradient from 0:100 to 10:90). The desired fractions were collected and concentrated in vacuo. Another purification was performed by RP HPLC (stationary phase: C18 XBridge 30×100 mm 5 μm), mobile phase: NH₄HCO₃ (0.25% solution in water)/CH₃CN, gradient from 67:33 to 50:50). The desired fractions were collected and concentrated in vacuo. The residue was dissolved in EtOAc and washed with NaHCO₃ (sat., aq.). The organic layer was dried (Na₂SO₄), filtered and concentrated in vacuo to afford a dark oil (44.5 mg).

HCl (6M in i-PrOH, 95.6 μL, 0.57 mmol) was added to a stirred solution of the residue (34 mg) in Et₂O (0.1 mL). The mixture was stirred at room temperature for 1 h and concentrated in vacuo. Tert-Butyl methyl ether was added and the mixture was sonicated.

The solvent was removed in vacuo. The process was repeated until the obtention of a solid which was dried under vacuum at 50° C. for 72 h to give compound 70 (40 mg, 98%) as a white solid.

### E63. Preparation of Final Compound 71

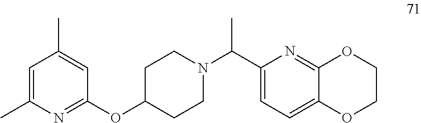 [SMILES: Cc1cc(C)nc(OC2CCN(C(C)c3ccc4c(n3)OCCO4)CC2)c1]

Compound 71 was prepared following an analogous procedure to the one described for the synthesis of compound 70 using intermediate 49 and intermediate 21 as starting materials.

The crude product was purified twice by flash column chromatography (silica, NH₃ (7N in MeOH)/DCM, gradient from 0:100 to 10:90). Another purification was performed by RP HPLC (stationary phase: C18 XBridge 30×100 mm 5 μm), mobile phase: NH₄HCO₃ (0.25% solution in water)/CH₃CN, gradient from 80:20 to 0:100). The residue was dissolved in EtOAc and washed with NaHCO₃ (sat., aq.). The organic phase was dried (Na₂SO₄), filtered and concentrated in vacuo to afford compound 71 (78.9 mg, 43%) as a white solid.

### E64. Preparation of Final Compound 72

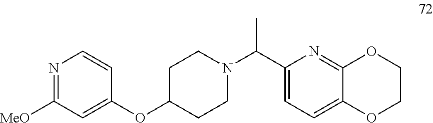 [SMILES: COc1cc(OC2CCN(C(C)c3ccc4c(n3)OCCO4)CC2)ccn1]

Compound 72 was prepared following an analogous procedure to the one described for the synthesis of compound 70 using intermediate 51 and intermediate 21 as starting materials.

The crude product was purified twice by flash column chromatography (silica, NH₃ (7N in MeOH)/DCM, gradient from 0:100 to 10:90). Another purification was performed by RP HPLC (stationary phase: C18 XBridge 30×100 mm 5 μm), mobile phase: NH₄HCO₃ (0.25% solution in water)/CH₃CN, gradient from 80:20 to 0:100), The desired fractions were collected and concentrated in vacuo. The residue was dissolved in EtOAc and washed with NaHCO₃ (sat., aq.). The organic phase was dried (Na₂SO₄), filtered and concentrated in vacuo to give compound 72 (79.9 mg, 61%) as a colorless oil.

### E65. Preparation of Final Compound 73

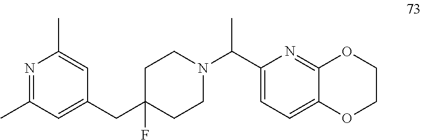 [SMILES: Cc1cc(CC2(F)CCN(C(C)c3ccc4c(n3)OCCO4)CC2)cc(C)n1]

Compound 73 was prepared following an analogous procedure to the one described for the synthesis of compound 70 using intermediate 114 and intermediate 21 as starting materials.

The crude product was purified by flash column chromatography (silica, NH₃ (7N in MeOH)/DCM, gradient from 0:100 to 10:90). Another purification was performed by RP HPLC (stationary phase: C18 XBridge 30×100 mm 5 μm), mobile phase: NH₄HCO₃ (0.25% solution in water)/CH₃CN, gradient from 54:46 to 36:64). The desired fractions were collected and concentrated in vacuo. The residue was dissolved in EtOAc and washed with NaHCO₃ (sat., aq.). The organic layer was dried (Na₂SO₄), filtered and concentrated in vacuo to give compound 73 (27 mg, 17%) as a colorless oil.

### E66 Preparation of Final Compound 74

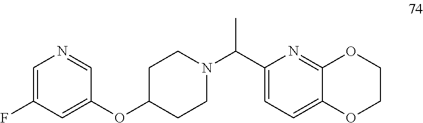 [SMILES: CC(c1ccc2c(n1)OCCO2)N1CCC(Oc2cncc(F)c2)CC1]

Compound 74 was prepared following an analogous procedure to the one described for the synthesis of compound 70 using intermediate 75 and intermediate 21 as starting materials.

The crude product was purified by flash column chromatography (silica, NH₃ (7N in MeOH)/DCM, gradient from 0:100 to 10:90). The desired fractions were collected and concentrated in vacuo to give compound 74 (35 mg, 39%) as a light yellow oil.

### E67. Preparation of Final Compound 75

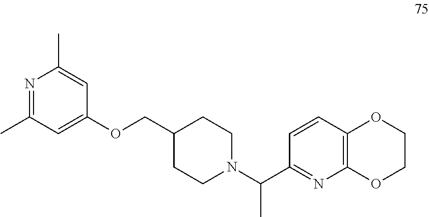 [SMILES: Cc1cc(OCC2CCN(C(C)c3ccc4c(n3)OCCO4)CC2)cc(C)n1]

Compound 75 was prepared following an analogous procedure to the one described for the synthesis of compound 70 using intermediate 93 and intermediate 21 as starting materials.

The crude mixture was combined with another fraction (0.15 mmol) and purified by Prep HPLC (Column Boston Prime C18 150*30 mm 5m, mobile phase: water (0.05% ammonia hydroxide v/v)/CH₃CN). The pure fractions were collected and the solvent was evaporated in vacuo to afford compound 75 (145.4 mg, 59%) as white solid.

### E68. Preparation of Final Compound 76

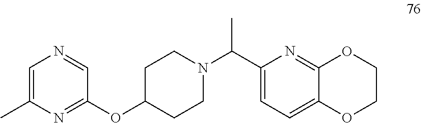 [SMILES: Cc1cncc(OC2CCN(C(C)c3ccc4c(n3)OCCO4)CC2)n1]

Compound 76 was prepared following an analogous procedure to the one described for the synthesis of compound 70 using intermediate 97 and intermediate 21 as starting materials.

The crude product was purified by flash column chromatography (silica, NH₃ (7N in MeOH)/DCM, gradient from 0:100 to 5:95). The residue was purified by RP HPLC (stationary phase: C18 XBridge 30×100 nm 5 um), mobile phase: (0.1% NH₄CO₃H/NH₄OH pH=9 solution in water)/CH₃CN, gradient from 74:26 to 58:42)) to afford compound 76 (60.3 mg, 34%) as a yellow oil which was became solid by adding Et₂O.

### E69. Preparation of Final Compound 77

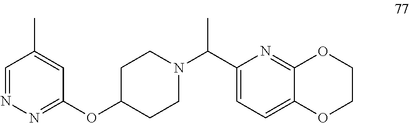 [SMILES: Cc1cnnc(OC2CCN(C(C)c3ccc4c(n3)OCCO4)CC2)c1]

Compound 77 was prepared following an analogous procedure to the one described for the synthesis of compound 70 using intermediate 99 and intermediate 21 as starting materials.

The crude product was purified by flash column chromatography (silica, NH₃ (7N in MeOH)/DCM, gradient from 0:100 to 5:95) to afford compound 77 (49.1 mg, 28%) as a brown oil.

### E70. Preparation of Final Compound 78

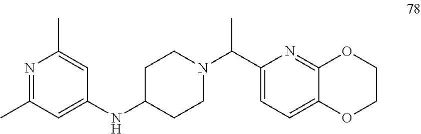 [SMILES: Cc1cc(NC2CCN(C(C)c3ccc4c(n3)OCCO4)CC2)cc(C)n1]

Compound 78 was prepared following an analogous procedure to the one described for the synthesis of compound 70 using intermediate 101 and intermediate 21 as starting materials.

The crude product was purified by flash column chromatography (silica, NH₃ (7N in MeOH)/DCM, gradient from 0:100 to 5:95) to afford compound 78 (75.9 mg, 29%) as a yellow oil.

### 71. Preparation of Final Compound 79

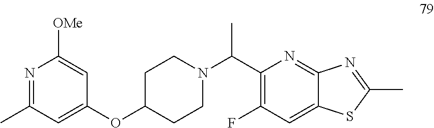

Compound 79 was prepared following an analogous procedure to the one described for the synthesis of compound 70 using intermediate 8 and intermediate 130 as starting materials.

The crude product was purified by flash column chromatography (silica, MeOH/DCM, gradient from 0:100 to 5:95). The desired fractions were collected and concentrated in vacuo to give compound 79 (165 mg, 75%) as a yellow oil.

### E72. PREPARATION OF FINAL COMPOUNDS 80 AND 81

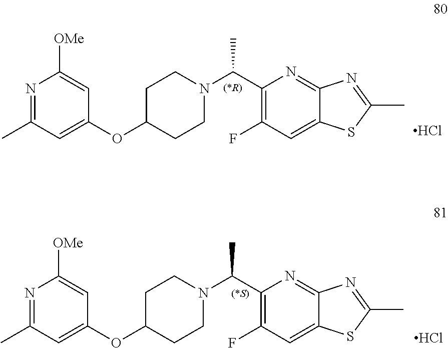

A purification of compound 79 was performed via chiral SFC (stationary phase: Chiralcel OD-H 5 μm 250×21.2 mm, mobile phase: 75% CO₂, 25% i-PrOH (0.3% i-PrNH₂)) to deliver 2 fractions: fraction A (70 mg) and fraction B (72 mg).

Fraction A (35 mg, 84 μmol) was dissolved in Et₂O (1.75 mL) and HCl (2N in Et₂O, 0.13 mL, 0.26 mmol) was added. The mixture was stirred for 5 min and filtered to give compound 80 (25 mg, 66%) as a white solid.

Compound 81 (47.4 mg) was prepared following an analogous procedure to the one described for compound 80 using fraction B (60 mg) as starting material.

### E73. Preparation of Final Compounds 82 and 83

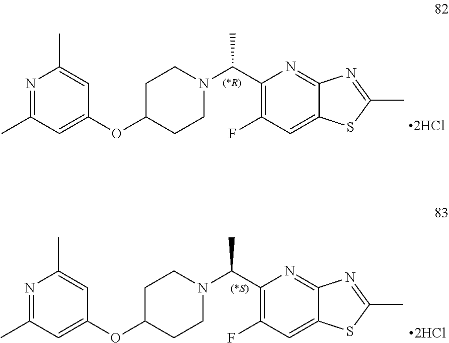

Compounds 82 and 83 were prepared following an analogous procedure to the one described for the synthesis of compound 70 using intermediate 6 and intermediate 130 as starting materials.

The crude product was purified by flash column chromatography (silica, NH₃ (7N in MeOH)/DCM, gradient from 0:100 to 5:95). The desired fractions were collected and concentrated in vacuo. A purification performed via chiral SFC (Stationary phase: CHIRALCEL OD-H 5 μm 250*30 mm, Mobile phase: 70% CO₂, 30% iPrOH (0.3% iPrNH₂) delivered 2 fractions: fraction A (56 mg) and fraction B (60 mg).

Fraction A (56 mg) was dissolved in Et₂O and HCl (2N in Et₂O) was added. The mixture was stirred for 5 min and filtered to give compound 82 (48 mg, 19%) as a white solid.

Fraction B was converted into compound 83 (48 mg) following an analogous procedure.

### E74. Preparation of Final Compound 84

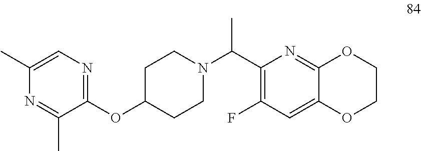 [SMILES: Cc1cnc(OC2CCN(C(C)c3nc4c(cc3F)OCCO4)CC2)c(C)n1]

Compound 84 was prepared following an analogous procedure to the one described for the synthesis of compound 70 using intermediate 59 and intermediate 20 as starting materials.

The crude product was purified by flash column chromatography (silica, NH₃ (7N in MeOH)/DCM, gradient from 0:100 to 5:95). The desired fractions were collected and concentrated in vacuo. A second purification was performed by RP HPLC (stationary phase: C18 XBridge 30×100 nm 5 um), mobile Phase: (0.1% NH₄CO₃H/NH₄OH pH=9 solution in water)/CH₃CN, gradient from 74:26 to 58:42) to afford a colorless oil (135 mg).

To a fraction of the residue (30 mg) in Et₂O was added HCl (2N in Et₂O). The mixture was stirred at room temperature for 1 h and the solid was filtered off to give compound 84 (22 mg).

### E75. Preparation of Final Compounds 85 and 86

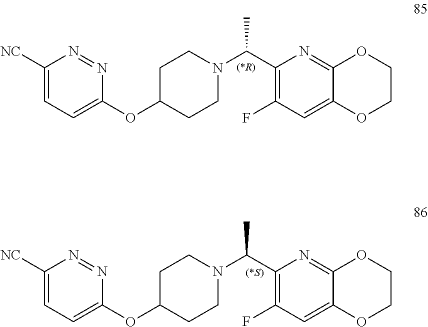 [SMILES: C[C@H](c1nc2c(cc1F)OCCO2)N1CCC(Oc2ccc(C#N)nn2)CC1 ; C[C@@H](c1nc2c(cc1F)OCCO2)N1CCC(Oc2ccc(C#N)nn2)CC1]

Compounds 85 and 86 were prepared following an analogous procedure to the one described for the synthesis of compound 70 using intermediate 43 and intermediate 20 as starting materials.

The crude product was purified by flash column chromatography (silica, MeOH in DCM, gradient from 0:100 to 5:95). The desired fractions were collected and the solvents evaporated in vacuo to afford a yellow solid. The solid was taken up in MeOH and the product was filtered off to give a white solid (124 mg). The filtrate was concentrated in vacuo and the residue was purified by RP HPLC (stationary phase: C18 XBridge 30×100 mm 5 μm), mobile phase: NH₄HCO₃ (0.25% solution in water)/CH₃CN, gradient from 75:25 to 40:60) to afford a white solid (28.3 mg).

The solid (124 mg) was purified via chiral SFC (stationary phase: CHIRACEL OJ-H 5 μm 250*20 mm, mobile phase: 75% CO₂, 25% MeOH (0.3% i-PrNH₂)) to deliver 2 fractions: fraction A (60 mg) and fraction B (56 mg). The fractions were independently purified via preparative LC (stationary phase: irregular bare silica, mobile phase: 0.1% NH₄OH, 98% DCM, 2% MeOH) to give compound 85 (19 mg, 4%) and compound 86 (23 mg, 5%).

### E76. Preparation of Final Compound 87

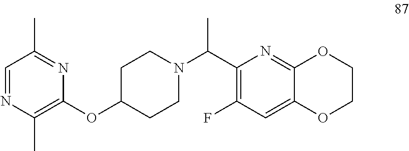 [SMILES: Cc1cnc(C)c(OC2CCN(C(C)c3nc4c(cc3F)OCCO4)CC2)n1]

Compound 87 was prepared following an analogous procedure to the one described for the synthesis of compound 70 using intermediate 59 and intermediate 20 as starting materials.

The crude mixture was purified by flash column chromatography (silica, MeOH in DCM, gradient from 0:100 to 5:95) to give compound 87 (81.7 mg, 51%) as a white solid.

### E77. Preparation of Final Compound 88

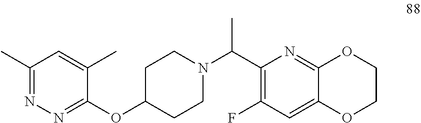 [SMILES: Cc1cc(C)c(OC2CCN(C(C)c3nc4c(cc3F)OCCO4)CC2)nn1]

Compound 88 was prepared following an analogous procedure to the one described for the synthesis of compound 70 using intermediate 61 and intermediate 20 as starting materials.

The crude mixture was purified by flash column chromatography (silica, DCM/MeOH, gradient from 100:0 to 95:5) to afford a colorless oil (44.6 mg).

The residue (44.6 mg, 0.12 mmol) was dissolved in Et₂O (0.3 mL) and HCl (2M in Et₂O, 0.17 mL, 0.34 mmol) was added under stirring. The precipitate was filtered and the product was dried under vacuum for 48 h at room temperature to give compound 88 (45 mg, 92%) as a white solid.

### E78. Preparation of Final Compound 89

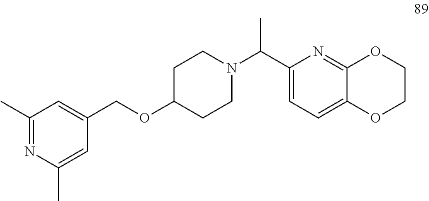 [SMILES: Cc1cc(COC2CCN(C(C)c3ccc4c(n3)OCCO4)CC2)cc(C)n1]

Compound 89 was prepared following an analogous procedure to the one described for the synthesis of compound 70 using intermediate 93.2HCl and intermediate 21 as starting materials.

The crude product was purified by prep. HPLC (column: Boston Prime C18 150*30 mm 5 μm, mobile phase: water (0.05% ammonia hydroxide v/v)-CH₃CN) to afford compound 89 (60.1 mg, 57%) as a white solid.

### E79. Preparation of Final Compound 90

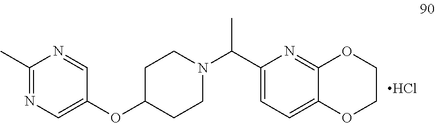 [SMILES: Cc1ncc(OC2CCN(C(C)c3ccc4c(n3)OCCO4)CC2)cn1 ; Cl]

Intermediate 21 (169 mg, 0.85 mmol) was added to a mixture of intermediate 73 (136 mg, 0.70 mmol) and K₂C03 (195 mg, 1.41 mmol) in CH₃CN (5 mL) at room temperature and the reaction mixture was stirred at 75° C. for 48 h. The solvent was removed in vacuo and the crude product was purified by flash column chromatography (silica, MeOH in DCM, gradient from 0:100 to 4:96). The desired fractions were collected and concentrated in vacuo to afford a colorless oil (136 mg).

The residue (136 mg) was diluted with DCM and treated with HCl (4N in 1,4-dioxane, 1 eq). The solvents were evaporated in vacuo and the product was triturated with DIPE to afford compound 90 (131 mg, 47%) as a white solid.

### E80. Preparation of Final Compound 91

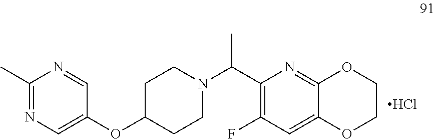 [SMILES: Cc1ncc(OC2CCN(C(C)c3nc4c(cc3F)OCCO4)CC2)cn1 ; Cl]

Compound 91 was prepared following an analogous procedure to the one described for the synthesis of compound 90 using intermediate 73 and intermediate 20 as starting materials.

The crude product purified by flash column chromatography (silica, MeOH in DCM, gradient from 0:100 to 4:96). The desired fractions were collected and concentrated in vacuo. The product was triturated with Et₂O to afford a colorless oil (78 mg).

The residue (78 mg) was diluted with DCM and treated with HCl (4N in 1,4-dioxane, 1 eq). The solvents were evaporated in vacuo and the product was triturated with DIPE to give compound 91 (80 mg, 29%) as a white solid.

### E81. Preparation of Final Compound 92

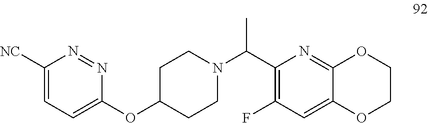 [SMILES: CC(c1nc2c(cc1F)OCCO2)N1CCC(Oc2ccc(C#N)nn2)CC1]

Intermediate 20 (548 mg, 2.52 mmol) and K₂C03 (1.16 g, 8.40 mmol) were added to a stirred solution of intermediate 43 (673 mg, 2.80 mmol) in anhydrous CH₃CN (10 mL) and DMF (5 mL). The reaction mixture was stirred at 70° C. for 20 h. The reaction mixture was diluted with EtOAc and filtered through Celite®. The solvents were evaporated in vacuo. The crude product was purified by flash column chromatography (silica, NH₃ (7M in MeOH)/DCM, gradient from 0:100 to 2:98). The desired fractions were collected and the solvents were evaporated in vacuo to afford compound 92 (227 mg, 21%) as a white solid.

### E82. Preparation of Final Compound 93

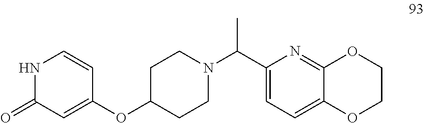 [SMILES: CC(c1ccc2c(n1)OCCO2)N1CCC(Oc2cc[nH]c(=O)c2)CC1]

A solution of citric acid (73.4 mg, 0.38 mmol) in 1,4-dioxane (1.22 mL) was added to a solution of compound 72 (71.0 mg, 0.19 mmol) in Et₂O (3.6 mL). The mixture was stirred at room temperature for 72 h. The precipitated was filtered off and washed with Et₂O. The solid was dissolved in MeOH and Et₂O was added. The mixture was concentrated in vacuo and the residue was dried at 50° C. for 3 days. The residue was treated with NaHCO₃ (sat., aq.) and extracted with EtOAc and THF (8:2). The organic layer was dried (Na₂SO₄), filtered and concentrated in vacuo. The product was dissolved in Et₂O (0.2 ml) and HCl (7N in IPA, 0.2 mL) was added. The mixture was stirred at room temperature for 24 h. tert-Butyl methyl ether was added and the mixture was sonicated. The solvent was concentrated under in vacuo. The process was repeated until the obtention of a solid.

The later was purified by RP HPLC (stationary phase: C18 XBridge 30×100 mm 5 μm), mobile phase: NH₄HCO₃ (0.25% solution in water)/CH₃CN, gradient from 90:10 to 65:35). The fractions were collected and concentrated in vacuo. The product was dissolved in EtOAc and washed with NaHCO₃ (sat., aq.). The organic phase was dried (Na₂SO₄), filtered and concentrated in vacuo to give compound 93 (24 mg, 35%).

### E83. Preparation of Final Compound 94

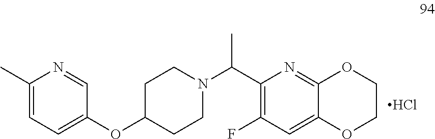 [SMILES: Cc1ccc(OC2CCN(C(C)c3nc4c(cc3F)OCCO4)CC2)cn1 ; Cl]

A mixture of intermediate 89 (300 mg, 1.56 mmol), intermediate 20 (339 mg, 1.56 mmol) and DIPEA (1.08 mL, 6.24 mmol) in anhydrous CH₃CN (6 mL) was stirred at 70° C. for 20 h. The reaction mixture was diluted with water and extracted with EtOAc. The organic layer was dried (Na₂SO₄), filtered and the solvents were evaporated in vacuo. The crude product was purified by flash column chromatography (silica, MeOH/DCM, gradient from 0:100 to 5:95) to afford a yellow oil (174 mg, 30%).

The yellow oil was combined with another batch and the residue (298 mg) was dissolved in Et₂O (2.02 mL) and HCl (2M in Et₂O, 1.20 mL, 2.40 mmol, 3 eq) was added under stirring. The resulting precipitate was filtered off and dried under vacuum for 48 h at room temperature to give compound 94 (315 mg, 96%) as a white solid.

### E84. Preparation of Final Compound 95

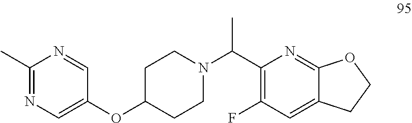 [SMILES: Cc1ncc(OC2CCN(C(C)c3nc4c(cc3F)CCO4)CC2)cn1]

Compound 95 was prepared following an analogous procedure to the one described for the synthesis of compound 94 using intermediate 73 and intermediate 36 as starting materials.

The crude product was purified by flash column chromatography (silica, MeOH in DCM, gradient from 0:100 to 5:95). The desired fractions were collected and evaporated in vacuo to give compound 95 (193 mg, 42%) as a yellow oil which became a light yellow solid after treatment with Et₂O.

### E85. Preparation of Final Compound 96

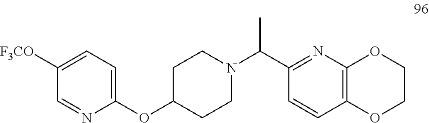 [SMILES: CC(c1ccc2c(n1)OCCO2)N1CCC(Oc2ccc(OC(F)(F)F)cn2)CC1]

To a mixture of NaH (60% dispersion in mineral oil, 22.7 mg, 0.57 mmol) in DMF (0.84 mL) at 0° C. were added intermediate 116 (50.0 mg, 0.19 mmol) and 15-crown-5 (37.8 L, 0.23 mmol). Then 2-bromo-5-(trifluoromethoxy)pyridine (CAS: 888327-36-4; 64.1 mg, 0.27 mmol) was added. The reaction mixture was stirred at 80° C. for 16 h. The mixture was cooled down and diluted with water. The solvents were evaporated in vacuo. The crude product was purified by RP HPLC (stationary phase: C18 XBridge 30×100 mm 5 μm), mobile phase: NH₄HCO₃ (0.25% solution in water)/CH₃CN, gradient from 90:10 to 0:100). A second purification was performed by RP HPLC (stationary phase: C18 XBridge 30×100 mm 5 μm), mobile phase: NH₄HCO₃ (0.25% solution in water)/CH₃CN, gradient from 60:40 to 25:75) to afford compound 96 (15 mg, 19%) as a yellow oil.

### E86. Preparation of Final Compound 97

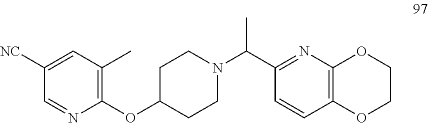 [SMILES: Cc1cc(C#N)cnc1OC1CCN(C(C)c2ccc3c(n2)OCCO3)CC1]

Compound 97 was prepared following an analogous procedure to the one described for the synthesis of compound 96 using intermediate 116 and 6-chloro-5-methylnicotinonitrile (CAS: 66909-33-9) as starting materials.

The crude mixture was purified by flash column chromatography (silica, MeOH in DCM, gradient from 0:100 to 3:97). A second purification was performed by RP HPLC (stationary phase: C18 XBridge 30×100 mm 5 μm), mobile phase: NH₄HCO₃ (0.25% solution in water)/CH₃CN, gradient from 60:40 to 25:75) to afford compound 97 (8 mg, 11%) as a colorless oil.

### E87. Preparation of Final Compound 98

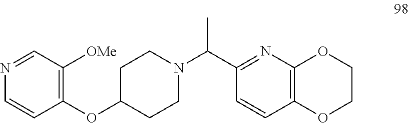 [SMILES: COc1cnccc1OC1CCN(C(C)c2ccc3c(n2)OCCO3)CC1]

Compound 98 was prepared following an analogous procedure to the one described for compound 96 using intermediate 116 and 4-bromo-3-methoxypyridine (CAS: 109911-38-8) as starting materials.

The crude product was purified by RP HPLC (stationary phase: C18 XBridge 30×100 mm 5 μm), mobile phase: NH₄HCO₃ (0.25% solution in water)/CH₃CN, gradient from 85:15 to 60:40) to afford compound 98 (8 mg, 11%) as a colorless oil.

### E88. Preparation of Final Compound 99

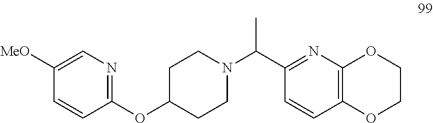 [SMILES: COc1ccc(OC2CCN(C(C)c3ccc4c(n3)OCCO4)CC2)nc1]

Compound 99 was prepared following an analogous procedure to the one described for compound 96 using intermediate 116 and 2-bromo-5-methoxypyridine (CAS: 105170-27-2) as starting materials.

The crude mixture was purified by flash column chromatography (silica, NH₃ (7N in MeOH)/DCM, gradient from 0:100 to 97:3). The residue was purified by RP HPLC (stationary phase: C18 XBridge 30×100 mm 5 μm), mobile phase: NH₄HCO₃ (0.25% solution in water)/CH₃CN, gradient from 75:25 to 40:60) to give compound 99 (8 mg, 19%) as a colorless oil.

### E89. Preparation of Final Compound 100

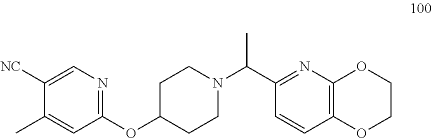 [SMILES: Cc1cc(OC2CCN(C(C)c3ccc4c(n3)OCCO4)CC2)ncc1C#N]

NaOtBu (54.5 mg, 0.57 mmol) was added to a solution of intermediate 116 (50.0 mg, 0.19 mmol) in CH₃CN (1.33 mL) in a sealed tube and N₂ atmosphere. 6-Chloro-2-methylnicotinonitrile (CAS: 66909-36-2; 40.4 mg, 0.27 mmol) was slowly added. The reaction mixture was stirred at 60° C. for 16 h. The mixture was diluted with water and stirred for 15 min. Solvents were concentrated in vacuo. The crude product was purified by flash column chromatography (silica, NH₃ (7N in MeOH)/DCM, gradient from 0:100 to 3:97). The residue was purified by RP HPLC (stationary phase: C18 XBridge 30×100 mm 5 μm), mobile phase: NH₄HCO₃ (0.25% solution in water)/CH₃CN, gradient from 75:25 to 40:60) to afford compound 100 (38.2 mg, 53%) as a light yellow solid.

### E90. Preparation of Final Compound 101

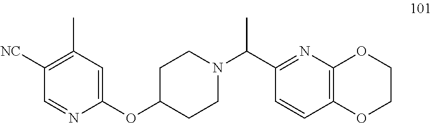 [SMILES: Cc1cc(OC2CCN(C(C)c3ccc4c(n3)OCCO4)CC2)ncc1C#N]

Compound 101 was prepared following an analogous procedure to the one described for the synthesis of compound 100 using intermediate 116 and 6-chloro-4-methylnicotinonitrile (CAS: 66909-35-1) as starting materials.

The crude product was purified by flash column chromatography (silica, NH₃ (7N in MeOH)/DCM, gradient from 0:100 to 3:97). The desired fractions were collected and concentrated in vacuo. The residue was purified by RP HPLC (stationary phase: C18 XBridge 30×100 mm 5 μm), mobile phase: NH₄HCO₃ (0.25% solution in water)/CH₃CN, gradient from 75:25 to 40:60) to give compound 101 (37.4 mg, 52%) as a light yellow solid.

### E91. Preparation of Final Compound 102

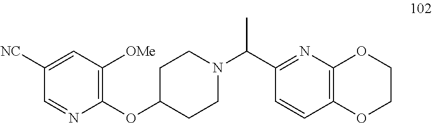 [SMILES: COc1cc(C#N)cnc1OC1CCN(C(C)c2ccc3c(n2)OCCO3)CC1]

Compound 102 was prepared following an analogous procedure to the one described for the synthesis of compound 100 using intermediate 116 and 6-chloro-5-methoxynicotinonitrile (CAS: 125683-79-6) as starting materials.

The crude product was purified by RP HPLC (stationary phase: C18 XBridge 0 50×100 mm 5 μm), mobile phase: NH₄HCO₃ (0.25% solution in water)/CH₃CN, gradient from 64:36 to 47:53) to give compound 102 (8.2 mg, 11%) as a solid.

### E92. Preparation of Final Compound 103

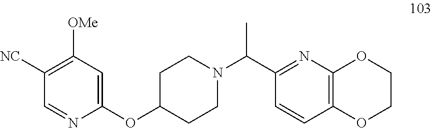 [SMILES: COc1cc(OC2CCN(C(C)c3ccc4c(n3)OCCO4)CC2)ncc1C#N]

Compound 103 was prepare following an analogous procedure to the one described for the synthesis of compound 100 using intermediate 116 and 6-chloro-4-methoxynicotinonitrile (CAS: 1187190-69-7) as starting materials.

The crude product was purified by RP HPLC (stationary phase: C18 XBridge 50×100 mm 5 μm), mobile phase: NH₄HCO₃ (0.25% solution in water)/CH₃CN, gradient from 64:36 to 47:53) to give compound 103 (9.8 mg, 13%) as a solid.

### E93. Preparation of Final Compound 104

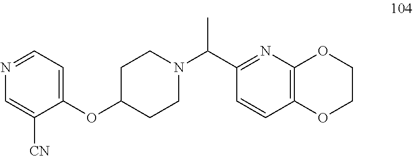 [SMILES: CC(c1ccc2c(n1)OCCO2)N1CCC(Oc2ccncc2C#N)CC1]

Compound 104 was prepared following an analogous procedure to the one described for the synthesis of compound 100 using intermediate 116 and 4-bromopyridine-3-carbonitrile (CAS: 154237-70-4) as starting materials.

The crude product was purified by flash column chromatography (silica, DCM/MeOH, gradient from 100:0 to 97:3). The residue was purified by using an Isolute® SCX-2 cartridge which was washed with MeOH and the product was eluted with NH₃ (7N in MeOH). The fraction was concentrated in vacuo to afford compound 104 (30 mg, 43%) as a yellow solid.

### E94. Preparation of Final Compound 105

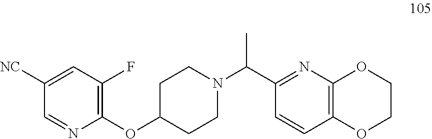 [SMILES: CC(c1ccc2c(n1)OCCO2)N1CCC(Oc2ncc(C#N)cc2F)CC1]

Compound 105 was prepared following an analogous procedure to the one described for the synthesis of compound 100 using intermediate 116 and 6-chloro-5-fluoronicotinonitrile (CAS: 102025-31-0) as starting materials.

The crude product was purified by RP HPLC (stationary phase: C18 XBridge 30×100 mm 5 μm), mobile phase: NH₄HCO₃ (0.25% solution in water)/CH₃CN, gradient from 67:33 to 50:50) to give compound 105 (15.8 mg, 36%) as a yellow oil.

### E95. Preparation of Final Compound 106

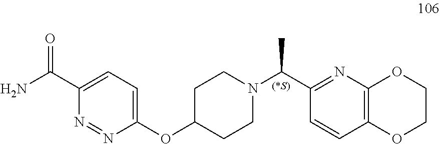 [SMILES: C[C@@H](c1ccc2c(n1)OCCO2)N1CCC(Oc2ccc(C(N)=O)nn2)CC1]

Compound 106 was prepared following an analogous procedure to the one described for the synthesis of compound 100 using intermediate 118 and 6-chloro-5-pyridiazinecarbonitrile (CAS: 35857-89-7) as starting materials.

The crude product was purified by flash column chromatography (silica, DCM/MeOH, gradient from 100:0 to 95:5) to afford compound 106 (44.2 mg, 60%) as a yellow solid.

### E96. Preparation of Final Compound 107

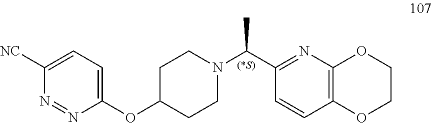 [SMILES: C[C@@H](c1ccc2c(n1)OCCO2)N1CCC(Oc2ccc(C#N)nn2)CC1]

NaOtBu (30.5 mg, 0.32 mmol) was added to a solution of intermediate 118 (70.0 mg, 0.27 mmol) in CH₃CN (1.87 mL) under N₂ atmosphere. 6-Chloro-5-pyridiazinecarbonitrile (CAS: 35857-89-7; 51.7 mg, 0.37 mmol) was slowly added. The reaction mixture was stirred at room temperature for 16 h. Water was added and the mixture was extracted with EtOAc (2×10 mL). The combined organic layers were dried (Na₂SO₄), filtered and concentrated in vacuo. The crude mixture was purified by flash column chromatography (silica, DCM/MeOH, gradient from 100:0 to 95:5; NH₃ (7N in MeOH)/DCM, gradient from 0:100 to 5:95). The residue was purified by RP HPLC (stationary phase: C18 XBridge 30×100 mm 5 μm), mobile phase: NH₄HCO₃ (0.25% solution in water)/CH₃CN, gradient from 90:10 to 60:40) to afford compound 107 (10 mg, 10%) as a yellow oil.

### E97. Preparation of Final Compound 108

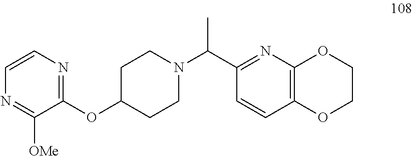 [SMILES: COc1nccnc1OC1CCN(C(C)c2ccc3c(n2)OCCO3)CC1]

To a mixture of intermediate 116 (50.0 mg, 0.19 mmol) in CH₃CN (2 mL) under N₂ atmosphere was added NaOtBu (36.4 mg, 0.38 mmol). 2-Chloro-3-methoxypyrazine (CAS: 40155-28-0; 38.3 mg, 0.27 mmol) was added and the reaction mixture was stirred at 80° C. for 16 h. The mixture was diluted with water at 0° C. and extracted with DCM. The combined organic layers were dried, filtered and concentrated in vacuo. The crude mixture was purified by RP HPLC (stationary phase: C18 XBridge 30×100 mm 5 μm), mobile phase: NH₄HCO₃ (0.25% solution in water)/CH₃CN, gradient from 75:25 to 40:60) to give compound 108 (36.3 mg, 52%) as a white solid.

### E98. Preparation of Final Compound 109

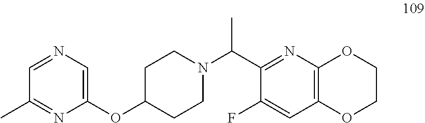 [SMILES: Cc1cncc(OC2CCN(C(C)c3nc4c(cc3F)OCCO4)CC2)n1]

Compound 109 was prepared following an analogous procedure to the one described for the synthesis of compound 108 using intermediate 119 and 2-chloro-6-methylpyrazine (CAS: 38557-71-0) as starting materials.

The crude product was purified by RP HPLC (stationary phase: C18 XBridge 30×100 mm 5 μm), mobile phase: NH₄HCO₃ (0.25% solution in water)/CH₃CN, gradient from 75:25 to 40:60) to give compound 109 (48 mg, 61%) as a white solid.

### E99. Preparation of Final Compound 110

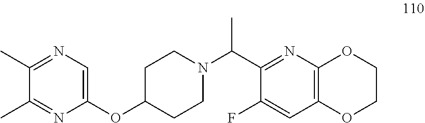 [SMILES: Cc1ncc(OC2CCN(C(C)c3nc4c(cc3F)OCCO4)CC2)nc1C]

Compound 110 was prepared following an analogous procedure to the one described for the synthesis of compound 108 using intermediate 119 and 5-chloro-2,3-dimethylpyrazine (CAS: 182500-28-3) as starting materials.

The crude product was purified by RP HPLC (stationary phase: C18 XBridge 30×100 mm 5 μm), mobile phase: NH₄HCO₃ (0.25% solution in water)/CH₃CN, gradient from 75:25 to 40:60) to give compound 110 (12 mg, 15%) as a yellow oil.

### E100. Preparation of Final Compound 111

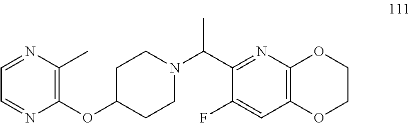

Compound 111 was prepared following an analogous procedure to the one described for the synthesis of compound 108 using intermediate 119 and 2-chloro-3-methylpyrazine (CAS: 95-58-9) as starting materials.

The crude product was purified by RP HPLC (stationary phase: C18 XBridge 30×100 mm 5 μm), mobile phase: NH₄HCO₃ (0.25% solution in water)/CH₃CN, gradient from 75:25 to 40:60) to afford a colorless oil (45.1 mg).

The residue (45.1 mg, 0.12 mmol) was dissolved in Et₂O (0.3 mL) and HCl (2M in Et₂O, 0.18 mL, 0.36 mmol) was added under stirring. The resulting precipitate was filtered and the product was dried under vacuum for 48 h at room temperature to deliver compound 111 (41.4 mg, 84%) as a white solid.

### E101. Preparation of Final Compound 112

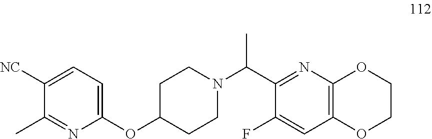 [SMILES: Cc1nc(OC2CCN(C(C)c3nc4c(cc3F)OCCO4)CC2)ccc1C#N]

To a mixture of intermediate 119 (50.0 mg, 0.18 mmol) in CH₃CN (1.25 mL) was added NaOtBu (51.1 mg, 0.53 mmol). The reaction mixture was stirred at room temperature for 15 min and 6-chloro-3-methylnicotinonitrile (CAS: 66909-36-2; 40.5 mg, 0.27 mmol) was added. The reaction mixture was stirred at 60° C. for 72 h. The mixture was filtered and the filtrate was evaporated in vacuo. The residue was purified by flash column chromatography (silica, MeOH in DCM, gradient from 0:100 to 5:95). The desired fractions were collected and evaporated in vacuo. A second purification was performed by RP HPLC (stationary phase: C18 XBridge 30×100 mm 5 μm), mobile phase: NH₄HCO₃ (0.25% solution in water)/CH₃CN, gradient from 80:20 to 0:100) to give compound 112 (37 mg, 52%) as a yellow solid.

### E102. Preparation of Final Compound 113

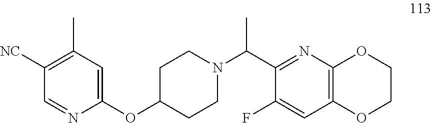 [SMILES: Cc1cc(OC2CCN(C(C)c3nc4c(cc3F)OCCO4)CC2)ncc1C#N]

Compound 113 was prepared following an analogous procedure to the one described for the synthesis of compound 112 using intermediate 119 and 6-chloro-4-methylnicotinonitrile (CAS: 66909-35-1) as starting materials.

The crude mixture was purified by flash column chromatography (silica, MeOH in DCM, gradient from 0:100 to 5:95). A second purification was performed by RP HPLC (stationary phase: C18 XBridge 30×100 mm 5 μm), mobile phase: NH₄HCO₃ (0.25% solution in water)/CH₃CN, gradient from 80:20 to 0:100) to give compound 113 (17 mg, 24%) as a yellow solid.

### E103. Preparation of Final Compound 114

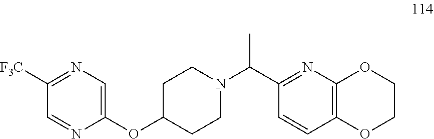 [SMILES: CC(c1ccc2c(n1)OCCO2)N1CCC(Oc2cnc(C(F)(F)F)cn2)CC1]

NaOtBu (32.7 mg, 0.34 mmol) was added to a solution of intermediate 116 (30.0 mg, 0.11 mmol) in anhydrous CH₃CN (0.8 mL) in a sealed tube and under N₂ atmosphere. 2-Chloro-5-(trifluoromethyl)pyrazine (CAS: 799557-87-2; 29.0 mg, 0.16 mmol) was slowly added. The reaction mixture was stirred at 80° C. for 16 h and concentrated in vacuo. The residue was diluted with water and extracted with EtOAc. The organic layer was dried (Na₂SO₄), filtered and evaporated in vacuo. The crude product was purified by flash column chromatography (silica, MeOH in DCM, gradient from 0:100 to 5:95). The desired fractions were collected and concentrated in vacuo to afford compound 114 (15.3 mg, 33%).

### E104. Preparation of Final Compound 115

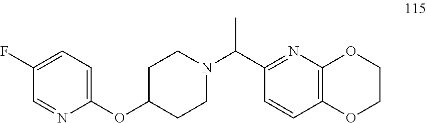 [SMILES: CC(c1ccc2c(n1)OCCO2)N1CCC(Oc2ccc(F)cn2)CC1]

DBAD (CAS: 870-50-8; 43.6 mg, 0.19 mmol) was added to a stirred mixture of intermediate 116 (20.0 mg, 75.7 μmol), 5-fluoro-2-hydroxypyridine (CAS: 51173-05-8; 21.4 mg, 0.19 mmol) and triphenylphosphine (49.6 mg, 0.19 mmol) in THF (0.36 mL) at room temperature under N₂ atmosphere. The reaction mixture was stirred for 16 h and the solvent was evaporated in vacuo. The crude mixture was purified by RP HPLC (stationary phase: C18 XBridge 30×100 mm 5 μm), mobile phase: NH₄HCO₃ (0.25% solution in water)/CH₃CN, gradient from 67:33 to 50:50) to afford compound 115 (10 mg, 37%) as a colorless oil.

### E105. Preparation of Final Compound 116

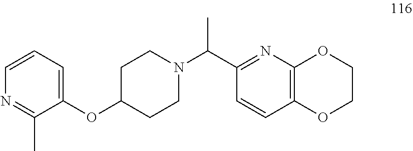 [SMILES: Cc1ncccc1OC1CCN(C(C)c2ccc3c(n2)OCCO3)CC1]

Compound 116 was prepared following an analogous procedure to the one described for the synthesis of compound 115 using intermediate 116 and 3-hydroxy-2-methylpyridine (CAS: 1121-25-1) as starting materials.

The crude mixture was purified by RP HPLC (stationary phase: C18 XBridge 50×100 mm 5 μm), mobile phase: NH₄HCO₃ (0.25% solution in water)/CH₃CN, gradient from 90:10 to 60:40) to give compound 116 (16 mg, 24%) as a colorless oil.

### E106. Preparation of Final Compound 117

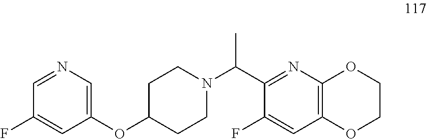 [SMILES: CC(c1nc2c(cc1F)OCCO2)N1CCC(Oc2cncc(F)c2)CC1]

10 Compound 117 was prepared following an analogous procedure to the one described for the synthesis of compound 115 using intermediate 119 and 5-fluoropyridin-3-ol (CAS: 209328-55-2) as starting materials.

The crude product was purified by flash column chromatography (silica, MeOH in DCM, gradient from 0:100 to 5:95). The desired fractions were collected and evaporated in vacuo. A second purification was performed by RP HPLC (stationary phase: C18 XBridge 30×100 mm 5 μm), mobile phase: NH₄HCO₃ (0.25% solution in water)/CH₃CN, gradient from 80:20 to 0:100) to afford compound 117 (33 mg, 49%) as a white sticky solid.

### E107. Preparation of Final Compound 118

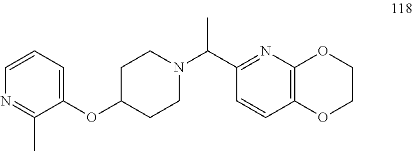 [SMILES: Cc1ncccc1OC1CCN(C(C)c2ccc3c(n2)OCCO3)CC1]

Compound 118 was prepared following an analogous procedure to the one described for the synthesis of compound 115 using intermediate 116 and 3-hydroxy-2-methylpyridine (CAS: 1121-25-1) as starting materials.

The crude mixture was purified by RP HPLC (stationary phase: C18 XBridge 50×100 mm 5 μm), mobile phase: NH₄HCO₃ (0.25% solution in water)/CH₃CN, gradient from 90:10 to 60:40) to afford compound 118 (16 mg, 24%) as a colorless oil. E108. Preparation of Final Compound 119

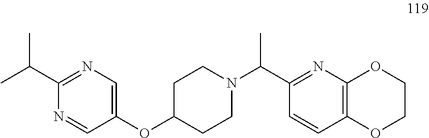 [SMILES: CC(C)c1ncc(OC2CCN(C(C)c3ccc4c(n3)OCCO4)CC2)cn1]

2-Propylzine bromide solution (0.5M, 2.12 mL, 1.06 mmol) was added to a mixture of compound 67 (100 mg, 0.27 mmol) and Pd(t-Bu₃P)₂ (13.6 mg, 26.5 μmol) in THF (1 mL) under N₂ atmosphere. The reaction mixture was stirred at 65° C. for 18 h, treated with a mixture of NH₄Cl (sat., aq.) and NH₄OH (1:1) and extracted with EtOAc. The organic layer was separated, dried (MgSO₄), filtered and the solvents were evaporated in vacuo. The crude product was purified by flash column chromatography (silica, MeOH/DCM, gradient from 0:100 to 5:95). A second purification was performed by RP HPLC (stationary phase: C18 XBridge 30×100 mm 5 μm), mobile phase: NH₄HCO₃ (0.25% solution in water)/CH₃CN, gradient from 85:15 to 55:45) to yield compound 119 (62 mg, 61%) as a colorless film.

### E109. Preparation of Final Compound 120

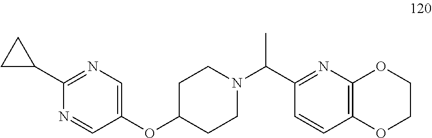 [SMILES: CC(c1ccc2c(n1)OCCO2)N1CCC(Oc2cnc(C3CC3)nc2)CC1]

Compound 120 was prepared following an analogous procedure to the one described for the synthesis of compound 119 using compound 67 and a cyclopropylzinc bromide solution.

The crude product was purified by flash column chromatography (silica, MeOH/DCM, gradient from 0:100 to 5:95). A second purification was performed by RP HPLC (stationary phase: C18 XBridge 30×100 mm 5 μm), mobile phase: NH₄HCO₃ (0.25% solution in water)/CH₃CN, gradient from 85:15 to 55:45) to afford compound 120 (20 mg, 33%) as a colorless oil.

### E110. Preparation of Final Compound 121

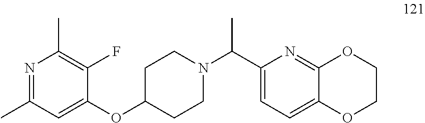 [SMILES: Cc1cc(OC2CCN(C(C)c3ccc4c(n3)OCCO4)CC2)c(F)c(C)n1]

PdCl₂(dppf) (16.2 mg, 22.1 μmol) and Na₂CO₃ (sat., aq.) were added to a stirred mixture of intermediate 188 (100 mg, 0.22 mmol) and methylboronic acid (66.2 mg, 1.11 mmol) in 1,4-dioxane (1.72 mL). The reaction mixture was purged with N₂ for 5 min and stirred at 150° C. for 30 min under microwave irradiation. The mixture was cooled down, washed with H₂O and extracted with DCM. The organic layer was dried (MgSO₄), filtered and the solvents were evaporated in vacuo. The crude product was purified by flash column chromatography (silica, EtOAc in heptane, gradient from 0:100 to 15:85). The desired fractions were collected and concentrated in vacuo to yield compound 121 (45 mg, 52%) as a as a white solid.

### E111. Preparation of Final Compound 122

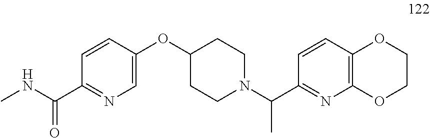 [SMILES: CNC(=O)c1ccc(OC2CCN(C(C)c3ccc4c(n3)OCCO4)CC2)cn1]

HATU (CAS: 148893-10-1; 60.1 mg, 0.16 mmol) was added to a stirred mixture of intermediate 193 and DIPEA (82.6 μL, 0.47 mmol) in DMF (4.89 mL). The reaction mixture was stirred at room temperature for 30 min and methylamine hydrochloride (10.7 mg, 0.16 mmol) was added. The reaction mixture was stirred at room temperature for 18 h. The mixture was diluted with NaHCO₃ (sat., aq.) and extracted with EtOAc. The organic layer was dried (MgSO₄), filtered and the solvents were evaporated in vacuo. The crude product was purified by flash chromatography (silica, heptane/EtOAc, gradient from 100:0 to 50:50). A second purification was performed by reverse phase chromatography ([25 mM NH₄HCO₃]/[MeCN: MeOH 1:1], gradient from 75:25 to 38:62). The desired fractions were collected and concentrated in vacuo to give compound 122 (29 mg, 46%) as a white solid.

### E112. Preparation of Final Compound 123

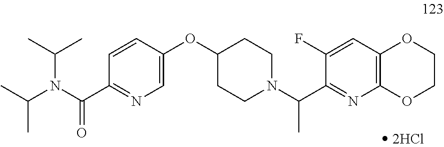 [SMILES: CC(c1nc2c(cc1F)OCCO2)N1CCC(Oc2ccc(C(=O)N(C(C)C)C(C)C)nc2)CC1]

Compound 123 was prepared following an analogous procedure to the one reported for the synthesis of compound 122 using intermediate 194 and diisopropylamine as starting materials.

The crude product was purified by reverse phase chromatography ([25 mM NH₄HCO₃]/[CH₃CN/MeOH, 1:1], gradient from 59:41 to 17:83). A second purification was performed by reverse phase chromatography ([25 mM NH₄HCO₃]/[MeCN/MeOH, 1:1]), gradient from 59:41 to 17:83). The desired fractions were collected and concentrated in vacuo to give a colorless oil (21 mg).

The residue (21 mg) was diluted with DCM and treated with HCl (4N in 1,4-dioxane, 2 eq). The solvents were evaporated in vacuo and the product was triturated with DIPE to yield compound 123 (11 mg, 8%) and as a white solid.

### E113. Preparation of Final Compound 124

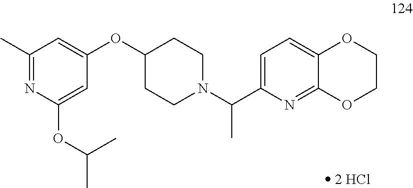 [SMILES: Cc1cc(OC2CCN(C(C)c3ccc4c(n3)OCCO4)CC2)cc(OC(C)C)n1]

Intermediate 21 (100 mg, 0.51 mmol) was added to a mixture of intermediate 198 (104 mg, 0.42 mmol) and K₂C03 (115 mg, 0.84 mmol) in CH₃CN (5 mL) at room temperature. The reaction mixture was stirred at 75° C. for 48 h. The solvent was removed in vacuo and the crude product was purified by reverse phase flash column chromatography ([65 mM NH₄OAc/CH₃CN, 90:10]/[CH₃CN/MeOH, 1:1], gradient from 70:30 to 27:73). A second purification was performed by flash column chromatography (silica, EtOAc in heptane, gradient from 0/100 to 100/0) to afford a colorless oil (30 mg).

The residue (30 mg) was taken into DCM and treated with HCl (4N in 1,4-dioxane, 1 eq). The solvents were evaporated in vacuo and the product was triturated with Et₂O to give compound 124 (20 mg, 10%) as a white solid.

### E114. Preparation of Final Compound 125

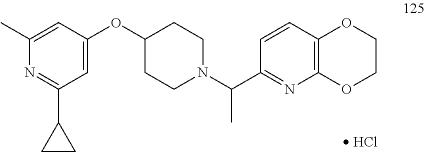 [SMILES: Cc1cc(OC2CCN(C(C)c3ccc4c(n3)OCCO4)CC2)cc(C2CC2)n1 ; "· HCl"]

Intermediate 21 (150 mg, 0.75 mmol) was added to a stirred mixture of intermediate 156 (149 mg, 0.58 mmol) and K₂C03 (160 mg, 1.16 mmol) in CH₃CN (7.3 mL) at room temperature. The reaction mixture was stirred at 75° C. for 16 h. Additional quantity of intermediate 21 (34.6 mg, 0.17 mmol) was added and the reaction mixture was stirred at 75° C. for another 16 h The reaction was quenched with water and extracted with EtOAc. The organic layer was dried (MgSO₄), filtered and the solvents were evaporated in vacuo. The crude mixture was purified by reverse phase ([25 mM NH₄HCO₃]/[CH₃CN/MeOH, 1:1], gradient from 59:41 to 17:83). The desired fractions were collected and concentrated in vacuo to afford a colorless oil (120 mg).

The residue (120 mg) was dissolved in DCM and treated with HCl (4N in 1,4-dioxane, 1 eq). The solvents were evaporated in vacuo. The product was triturated with Et₂O to afford compound 125 (79.5 mg, 32%) as a white solid.

### E115. Preparation of Final Compound 126

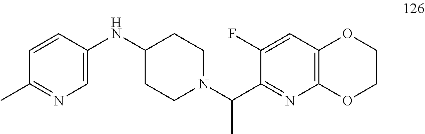 [SMILES: Cc1ccc(NC2CCN(C(C)c3nc4c(cc3F)OCCO4)CC2)cn1]

Compound 126 was prepared following an analogous procedure to the one described for the synthesis of compound 125 using intermediate 20 and intermediate 162 as starting materials.

The crude mixture was purified by flash column chromatography (silica, DCM in MeOH, gradient from 0:100 to 10:90). The desired fractions were collected and concentrated in vacuo. The residue was dissolved in Et₂O and concentrated in vacuo. The product was triturated in heptane, filtered and dried to give compound 126 (107 mg, 49%) as a white solid.

### E116. Preparation of Final Compound 127

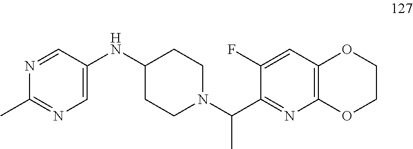 [SMILES: Cc1ncc(NC2CCN(C(C)c3nc4c(cc3F)OCCO4)CC2)cn1]

Compound 127 was prepared following an analogous procedure to the one described for the synthesis of compound 125 using intermediate 20 and intermediate 164 as starting materials.

The crude mixture was purified by flash column chromatography (silica, DCM in MeOH, gradient from 0:100 to 10:90). The desired fractions were collected and concentrated in vacuo. The residue was dissolved in Et₂O and concentrated in vacuo. The product was triturated with DIPE, filtered and dried to give compound 127 (106.7 mg, 48%) as a white solid.

### E117. Preparation of Final Compound 128

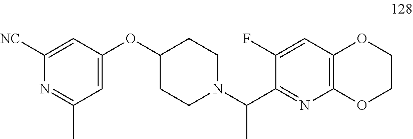 [SMILES: Cc1cc(OC2CCN(C(C)c3nc4c(cc3F)OCCO4)CC2)cc(C#N)n1]

Compound 128 was prepared following an analogous procedure to the one described for the synthesis of compound 125 using intermediate 20 and intermediate 160 as starting materials.

The crude mixture was purified by flash column chromatography (silica, heptane/EtOAc, gradient from 100:0 to 0:100, DCM/MeOH, gradient from 80:20 to 60:40). The desired fractions were collected and concentrated in vacuo. A second purification was performed by reverse phase (Phenomenex Gemini C18 100×30 mm 5 μm; [25 mM NH₄HCO₃]/[CH₃CN/MeOH, 1:1), gradient from 59:41 to 17:83). The desired fractions were collected and concentrated in vacuo to give compound 128 (98.4 mg, 54%) as a white foam.

### E118. Preparation of Final Compound 129

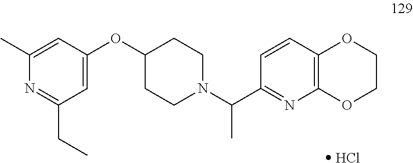 [SMILES: CCc1cc(OC2CCN(C(C)c3ccc4c(n3)OCCO4)CC2)cc(C)n1 ; "· HCl"]

Compound 129 was prepared following an analogous procedure to the one described for the synthesis of compound 125 using intermediate 21 and intermediate 201 as starting materials.

The crude product was purified by reverse flash column chromatography (silica, [25 mM NH₄HCO₃]/[CH₃CN/MeOH 1:1], gradient from 70:30 to 27:73).

The residue (60 mg) was combined with another fraction and dissolved in DCM. The mixture was treated with HCl (4N in 1,4-dioxane, 1 eq.). The solvents were evaporated in vacuo and the product was triturated with DIPE and filtered to deliver compound 129 as a white solid.

### E119. Preparation of Final Compound 130

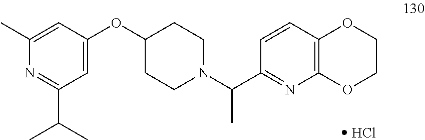 [SMILES: Cc1cc(OC2CCN(C(C)c3ccc4c(n3)OCCO4)CC2)cc(C(C)C)n1 ; "· HCl"]

Compound 130 was prepared following an analogous procedure to the one described for the synthesis of compound 125 using intermediate 21 and intermediate 167 as starting materials.

The crude product was purified by flash column chromatography (silica, [DCM/MeOH 9:1]/DCM, gradient from 0:100 to 100:0). The desired fractions were collected and concentrated in vacuo to give a colorless oil (25.8 mg).

The residue (25.8 mg) was taken into DCM and treated with HCl (4N in 1,4-dioxane, 1 eq). The solvents were evaporated in vacuo to afford compound 130 (19 mg, 8%) as a white solid.

### E120. Preparation of Final Compound 131

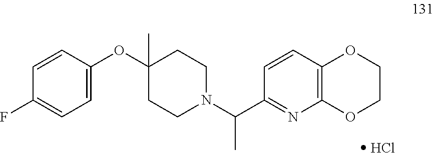 [SMILES: CC(c1ccc2c(n1)OCCO2)N1CCC(C)(Oc2ccc(F)cc2)CC1 ; "· HCl"]

Compound 131 was prepared following an analogous procedure to the one described for the synthesis of compound 125 using intermediate 21 and intermediate 178 as starting materials.

The crude product was purified by reverse flash column chromatography ([65 mM NH₄OAc/CH₃CN, 90:10]/[CH₃CN/MeOH, 1:1], gradient from 72:28 to 36:64). The desired fractions were collected and concentrated in vacuo to give a colorless oil (47 mg).

The residue (47 mg) was taken into DCM and treated with HCl (4N in 1,4-dioxane, 1 eq). The solvents were evaporated in vacuo to afford compound 131 (20 mg, 15%) as a white solid.

### E121. Preparation of Final Compound 132

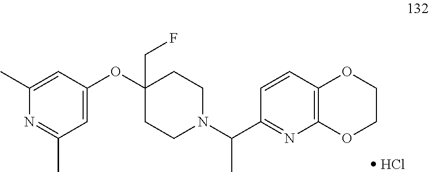 [SMILES: Cc1cc(OC2(CF)CCN(C(C)c3ccc4c(n3)OCCO4)CC2)cc(C)n1 ; "· HCl"]

Compound 132 was prepared following an analogous procedure to the one described for the synthesis of compound 125 using intermediate 21 and intermediate 182 as starting materials.

The crude product was purified by reverse flash column chromatography ([65 mM NH₄OAc/CH₃CN, 90:10]/[CH₃CN/MeOH 1:1], gradient from 81:19 to 45:55). A second purification was performed by reverse flash column chromatography ([25 mM NH₄HCO₃]/[CH₃CN/MeOH 1:1], gradient from 81:19 to 45:55). The desired fractions were collected and concentrated in vacuo. The product was triturated with Et₂O to afford a colorless oil (32.9 mg).

The residue (32.9 mg) was taken into DCM and treated with HCl (4N in 1,4-dioxane, 1 eq). The solvents were evaporated in vacuo to afford compound 132 (20 mg, 19%) as a white powder.

### E122. Preparation of Final Compound 133

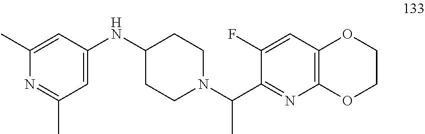 [SMILES: Cc1cc(NC2CCN(C(C)c3nc4c(cc3F)OCCO4)CC2)cc(C)n1]

Compound 133 was prepared following an analogous procedure to the one described for the synthesis of compound 125 using intermediate 20 and intermediate 184 as starting materials.

The crude product was purified by reverse phase flash column chromatography (silica, NH₃ in MeOH (5%) in DCM, gradient from 0:100 to 10:90). The desired fractions were collected and concentrated in vacuo to give compound 133 (75 mg, 49%) as a pale white solid.

### E123. Preparation of Final Compound 134

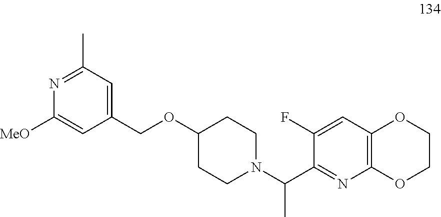 [SMILES: COc1cc(COC2CCN(C(C)c3nc4c(cc3F)OCCO4)CC2)cc(C)n1]

Compound 134 was prepared following an analogous procedure to the one described for the synthesis of compound 125 using intermediate 20 and intermediate 169 as starting materials.

The crude product was purified by reverse phase flash column chromatography (silica, heptane/EtOAc, gradient from 100:0 to 60:40). The desired fractions were collected and concentrated in vacuo to afford compound 134 (55 mg, 52%) as a yellowish oil.

### E124. Preparation of Final Compounds 135,136 and 137

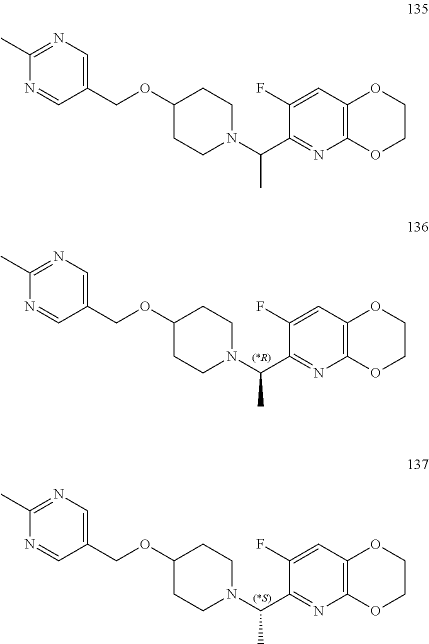 [SMILES: Cc1ncc(COC2CCN(C(C)c3nc4c(cc3F)OCCO4)CC2)cn1 ; Cc1ncc(COC2CCN([C@H](C)c3nc4c(cc3F)OCCO4)CC2)cn1 ; Cc1ncc(COC2CCN([C@@H](C)c3nc4c(cc3F)OCCO4)CC2)cn1]

Compounds 135, 136 and 137 were prepared following an analogous procedure to the one described for the synthesis of compound 125 using intermediate 20 and intermediate 173 as starting materials.

The crude product was purified by reverse phase flash column chromatography (silica, MeOH in DCM, gradient from 0:100 to 5:95). The desired fractions were collected and concentrated in vacuo to afford compound 135 (197 mg, 72%) as a pale white solid.

The enantiomers were separated by semi preparative HPLC chromatography (Amylose-2 column, Heptane/EtOH, gradient from 75:25 to 0:100). The desired fractions were collected and concentrated in vacuo to afford compound 136 (35 mg, 21%) and compound 137 (39.1 mg, 24%) as white solids.

### E125. Preparation of Final Compound 138

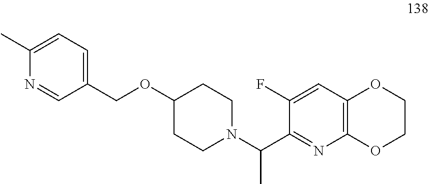 [SMILES: Cc1ccc(COC2CCN(C(C)c3nc4c(cc3F)OCCO4)CC2)cn1]

Compound 138 was prepared following an analogous procedure to the one described for the synthesis of compound 125 using intermediate 20 and intermediate 171 as starting materials.

The crude product was purified by flash column chromatography (silica, [DCM/MeOH, 9:1]/DCM, gradient from 0:100 to 100:0). The desired fractions were collected and concentrated in vacuo to afford compound 138 (46.9 mg, 33%) as a brown oil.

### E126. Preparation of Final Compound 139

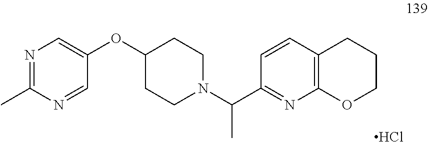 [SMILES: Cc1ncc(OC2CCN(C(C)c3ccc4c(n3)OCCC4)CC2)cn1 ; Cl]

Compound 139 was prepared following an analogous procedure to the one described for the synthesis of compound 125 using intermediate 73 and intermediate 133 as starting materials.

The crude product was purified by flash column chromatography (silica, MeOH in DCM, gradient from 0/100 to 7/93). The desired fractions were collected and concentrated in vacuo to afford a yellow sticky solid (105 mg).

The residue compound 139 (105 mg) was taken into DCM and treated with HCl (4N in 1,4-dioxane, 1 eq). The solvents were evaporated in vacuo. The product was triturated in Et₂O. filtered and dried to afford compound 139 (96 mg, 39%) as a pale orange solid.

### E127. Preparation of Final Compound 140

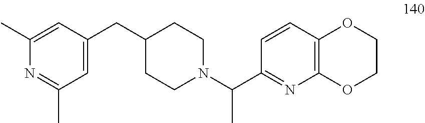 [SMILES: Cc1cc(CC2CCN(C(C)c3ccc4c(n3)OCCO4)CC2)cc(C)n1]

Intermediate 21 (174 mg, 0.8/mmol) was added to a stirred mixture of intermediate 150 (148 mg, 0.72 mmol) and K₂CO₃ (200 mg, 1.45 mmol) in CH₃CN (7 mL) at room temperature. The reaction mixture was stirred at 75° C. for 16 h. The solvent was removed in vacuo. The residue was dissolved in MeOH (47.5 mL) and Amberlyst®A26 hydroxide form (CAS: 39339-85-0; 453 mg, 1.45 mmol) was added. The mixture was stirred at room temperature for 15 min. The reaction was filtered and washed with MeOH several times. The filtrate was evaporated in vacuo and the crude product was purified by reverse phase (InterChim Uptisphere Strategy C-18-HQ 100×30 mm PREP-LC Column (P/N USC18HQ-100/30); from 72% [25 mM NH₄CO₃]-28% [ACN:MeOH (1:1)] to 36% [25 nM NH₄CO₃]-64% [ACN:MeOH (1:1)]. The desired fractions were collected and concentrated in vacuo to give compound 140 (176 mg, 65%) as a white solid.

### E128. Preparation of Final Compound 141

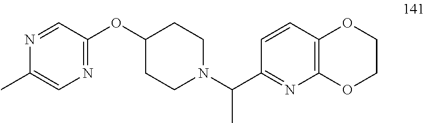 [SMILES: Cc1cnc(OC2CCN(C(C)c3ccc4c(n3)OCCO4)CC2)cn1]

Compound 141 was prepared following an analogous procedure to the one described for the synthesis of compound 140 using intermediate 152 and intermediate 21 as starting materials.

The crude product was purified by flash column chromatography (silica, heptane/EtOAc, gradient from 100:0 to 80:20). The desired fractions were collected and concentrated in vacuo to give compound 141 (110 mg, 73%) as a colorless solid.

### E129. Preparation of Final Compound 142

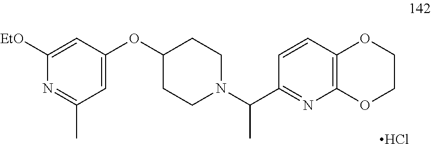 [SMILES: CCOc1cc(OC2CCN(C(C)c3ccc4c(n3)OCCO4)CC2)cc(C)n1 ; Cl]

Intermediate 21 (105 mg, 0.53 mmol) was added to a mixture of intermediate 158 (104 mg, 0.44 mmol) and K₂CO₃ (122 mg, 0.88 mmol) in DMF (5 mL). The reaction mixture was stirred at 75° C. for 48 h. Additional amount of K₂CO₃ (61 mg, 0.44 mmol) was added at room temperature and the reaction mixture was stirred at 75° C. for another 12 h. The solvent was removed in vacuo and the crude product was purified by flash column chromatography (silica, heptane/EtOAC, gradient from 100:0 to 20:80). The desired fractions were collected and concentrated in vacuo. A second purification was performed by reverse phase ([25 mM NH₄HCO₃]/[CH₃CN/MeOH, 1:1], gradient from 59:41 to 17:83). The desired fractions were collected and concentrated in vacuo to afford a colorless oil (41 mg).

The residue (41 mg) was dissolved in DCM and treated with HCl (4N in 1,4-dioxane, 1 eq). The solvents were evaporated in vacuo and the product was triturated with DIPE to give compound 142 (33 mg, 17%) as a white solid.

### E131. Preparation of Final Compound 150

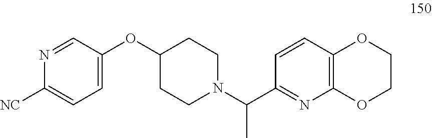 [SMILES: CC(c1ccc2c(n1)OCCO2)N1CCC(Oc2ccc(C#N)nc2)CC1]

Intermediate 21 (118 mg, 0.59 mmol) was added to a stirred solution of intermediate 203 (100 mg, 0.49 mmol) and K₂CO₃ (136 mg, 0.98 mmol) in CH₃CN (3 mL). The reaction mixture was stirred at 75° C. for 6 h. The solvent was evaporated in vacuo. The crude product was purified by reverse phase ([25 mM NH₄HCO₃]/[MeCN:MeOH, 1:1], gradient from 72:28 to 36:64). The desired fractions were collected and concentrated in vacuo to give compound 150 (155 mg, 85%) as a white solid.

### E132. Preparation of Final Compound 151

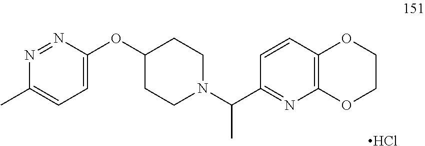 [SMILES: Cc1ccc(OC2CCN(C(C)c3ccc4c(n3)OCCO4)CC2)nn1 ; Cl]

Intermediate 21.HCl (302 mg, 1.28 mmol) was added to a mixture of intermediate 205 (206 mg, 1.07 mmol) and K₂CO₃ (442 mg, 3.20 mmol) in CH₃CN (8 mL). The reaction mixture was stirred at 65° C. for 26 h. The solvent was removed and the crude product purified by reverse phase ([25 mM NH₄HCO₃]/[ACN:MeOH, 1:1], gradient from 81:19 to 45:55). The desired fractions were collected and concentrated in vacuo to afford a yellow oil (192 mg).

The residue (192 mg) was taken into DCM and treated with HCl (4N in dioxane, 1 eq). The solvents were evaporated in vacuo and the product was tritured with Et₂O to afford compound 151 (170 mg, 40%) as a white solid.

### E133. Preparation of Final Compound 152

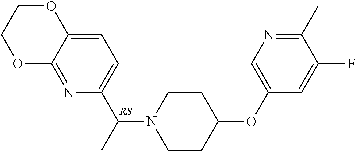 [SMILES: Cc1ncc(OC2CCN(C(C)c3ccc4c(n3)OCCO4)CC2)cc1F]

Compound 152 was prepared following an analogous procedure to the one described for the synthesis of compound 121 using compound 69 as starting material. The crude product was purified by reverse phase HPLC (stationary phase: C18 XBridge 30×100 mm 5 m, mobile phase: gradient from 85% NH₄HCO₃ 0.25% solution in water, 15% CH₃CN to 55% NH₄HCO₃ 0.25% solution in water, 45% CH₃CN), to yield compound 152 (43 mg, 91%) as a colourless oil.

### E134. Preparation of Final Compound 153

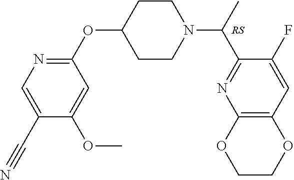 [SMILES: COc1cc(OC2CCN(C(C)c3nc4c(cc3F)OCCO4)CC2)ncc1C#N]

Compound 153 was prepared following an analogous procedure to the one described for the synthesis of compound 100 using intermediate 145 and 6-chloro-4-methoxynicotinonitrile (CAS: 1187190-69-7) as starting materials. The crude product was purified by flash column chromatography (silica, MeOH in DCM 0/100 to 10/90). The desired fractions were collected and evaporated in vacuo to give compound 153 (41.2 mg, 53%) as a colourless oil.

### E135. Preparation of Final Compound 154

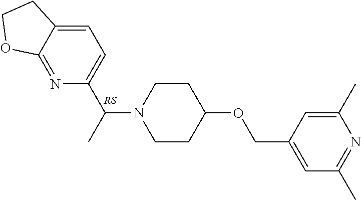 [SMILES: Cc1cc(COC2CCN(C(C)c3ccc4c(n3)OCC4)CC2)cc(C)n1]

Compound 154 was prepared following an analogous procedure to the one described for the synthesis of compound 21 using intermediate 63 and 2,3-dihydro-furo[2,3-b]pyridine carboxaldehyde (CAS: 1557979-76-6) as starting materials. The crude product was purified by flash column chromatography (silica, MeOH in DCM 0/100 to 10/90). The desired fractions were collected and evaporated in vacuo to give compound 154 (78.9 mg, 79%) as a colourless oil.

### E136. Preparation of Final Compound 155

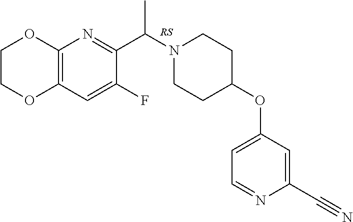 [SMILES: CC(c1nc2c(cc1F)OCCO2)N1CCC(Oc2ccnc(C#N)c2)CC1]

Compound 155 was prepared following an analogous procedure to the one described for the synthesis of compound 94 using intermediate 55 and intermediate 20 as starting materials. The crude product was purified by flash column chromatography (silica, MeOH in DCM 0/100 to 5/95). The desired fractions were collected and evaporated in vacuo to give 220 mg of compound 155, which was further purified by reverse phase HPLC (stationary phase: C18 XBridge 50×100 mm 5 μm, mobile phase: gradient from 75% NH₄HCO₃ 0.25% solution in water, 25% CH₃CN to 40% NH₄HCO₃ 0.25% solution in water, 60% CH₃CN) yielding compound 155 (91 mg, 26%) as a light yellow solid.

### E137. Preparation of Final Compound 156

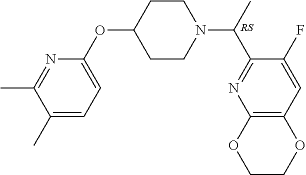 [SMILES: Cc1ccc(OC2CCN(C(C)c3nc4c(cc3F)OCCO4)CC2)nc1C]

Compound 156 was prepared following an analogous procedure to the one described for the synthesis of compound 94 using intermediate 121 and intermediate 20 as starting materials. The crude product was purified by flash column chromatography (silica, MeOH in DCM 0/100 to 5/95). The desired fractions were collected and evaporated in vacuo to give compound 156 (74.8 mg, 36%) as a pale yellow oil.

### E138. Preparation of Final Compound 157

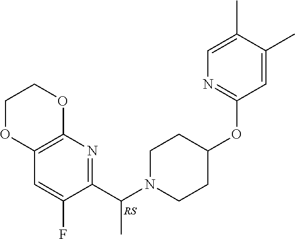 [SMILES: Cc1cnc(OC2CCN(C(C)c3nc4c(cc3F)OCCO4)CC2)cc1C]

Compound 157 was prepared following an analogous procedure to the one described for the synthesis of compound 94 using intermediate 200 and intermediate 20 as starting materials. The crude product was purified by flash column chromatography (silica, MeOH in DCM 0/100 to 5/95). The desired fractions were collected and evaporated in vacuo to give compound 157 (50 mg, 35%) as a pale yellow oil, which was treated with HCl (2 N in Et₂O) to yield compound 157. HCl (78 mg, 50%) as a white solid.

### E139. Preparation of Final Compound 158

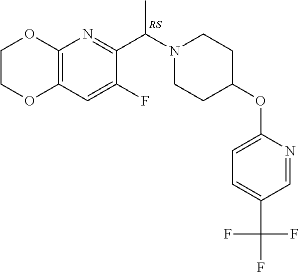 [SMILES: CC(c1nc2c(cc1F)OCCO2)N1CCC(Oc2ccc(C(F)(F)F)cn2)CC1]

Method 1: Compound 158 was prepared following an analogous procedure to the one described for the synthesis of compound 94 using intermediate 95. TFA (100 mg, 0.41 mmol) and intermediate 20 (88.38 mg, 0.41 mmol) as starting materials. The crude was combined with the batch obtained from method 2 and purified together.

Method 2: Compound 158 was also prepared following an analogous procedure to the one described for the synthesis of compound 100 using intermediate 145 (50 mg, 0.177 mmol) and 2-chloro-5-(trifluoromethyl)pyridine (CAS: 52334-81-3, 45.01 mg, 0.248 mmol) as starting materials.

The combined crude batches were purified by flash column chromatography (silica, MeOH in DCM/0100 to 10/90). The desired fractions were collected and concentrated in vacuo to yield compound 158 (117.2 mg, 43%) as a colourless oil.

### E140. Preparation of Final Compound 159

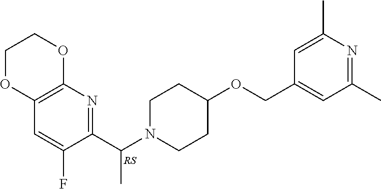 [SMILES: Cc1cc(COC2CCN(C(C)c3nc4c(cc3F)OCCO4)CC2)cc(C)n1]

DIPEA (0.424 mL, 2.46 mmol) was added dropwise to a suspension of intermediate 63 2HCl (150 mg, 0.41 mmol) in CH₃CN (2 mL). Then a solution of intermediate 20 (93.61 mg, 0.43 mmol) in CH₃CN (1 mL) was added dropwise. The mixture was stirred at 80° C. for 24h. Then, the solvent was evaporated in vacuo. The residue was taken into EtOAc and sat Na₂CO₃ was added. The organic layer separated, dried (Na₂SO₄), filtered and evaporated in vacuo. The crude product was purified by flash column chromatography (silica; MeOH in DCM 0/100 to 5/95). The desired fractions were collected and solvents evaporated in vacuo to yield a light yellow oil which was purified by reverse phase HPLC (Stationary phase: C18 XBridge 50×100 mm 5 μm, mobile phase: gradient from 70% NH₄HCO₃ 0.25% solution in water, 30% CH₃CN to 35% NH₄HCO₃ 0.25% solution in water, 65% CH₃CN), yielding compound 159 (110 mg, 67%) as an oil.

Compound 159 was dissolved in Et₂O (1.067 mL) and HCl (2N in Et₂O, 0.478 mL) was added and the mixture was stirred at RT for 1 h. Then, the solid was filtered off and washed with Et₂O. The solid was dried in a dessicator without heating for 2 days to yield compound 159.2 HCl (106 mg, 93%) as a white solid.

### E141. Preparation of Final Compound 160

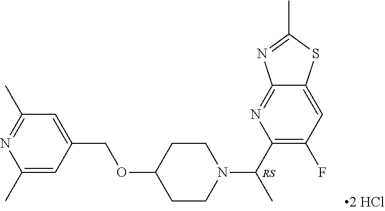 [SMILES: Cc1cc(COC2CCN(C(C)c3nc4nc(C)sc4cc3F)CC2)cc(C)n1]

K₂CO₃ (143.78 mg, 1.04 mmol) was added to a solution of intermediate 130 (60 mg, 0.26 mmol) and intermediate 63 (57.30 mg, 0.26 mmol) in CH₃CN (1.90 mL) in a sealed tube and under nitrogen. The mixture was stirred for 18h at 60° C. Then, the reaction was diluted with water and extracted with EtOAc. The organic layer was separated, dried (MgSO₄), filtered and the solvents evaporated in vacuo. The crude product was purified by flash column chromatography (silica, MeOH in DCM 0/100 to 10/90). The desired fractions were collected and evaporated in vacuo to give compound 160 (77 mg, 71%) as a colourless oil.

Compound 160 (77 mg, 0.186 mmol) was dissolved in Et₂O (0.541 mL) and HCl (2N in Et₂O, 0.279 mL) was added under stirring. The resulting precipitate was filtered and the compound was immediately dried under vacuum for 24 h at rt to yield compound 160.2HCl (47.8 mg, 53%) as a white solid.

### E142. Preparation of Final Compound-161

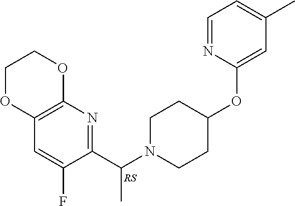 [SMILES: Cc1ccnc(OC2CCN(C(C)c3nc4c(cc3F)OCCO4)CC2)c1]

Compound 161 was prepared following an analogous procedure to the one described for the synthesis of compound 160 using intermediate 20 and intermediate 91 as starting materials. The crude was purified by flash column chromatography (silica, MeOH in DCM 0/100 to 10/90). The desired fractions were collected and concentrated in vacuo to yield compound 161 (39.8 mg, 41%) as a colourless oil.

### E143. Preparation of Final Compound 162

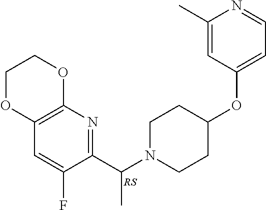 [SMILES: Cc1cc(OC2CCN(C(C)c3nc4c(cc3F)OCCO4)CC2)ccn1]

Compound 162 was prepared following an analogous procedure to the one described for the synthesis of compound 160 using intermediate 20 and intermediate 67 as starting materials. The crude was purified by flash column chromatography (silica, MeOH in DCM 0/100 to 5/95) and then by reverse phase HPLC (Stationary phase: C18 XBridge 30×100 mm 5 μm, mobile phase: gradient from 90% NH₄HCO₃ 0.25% solution in water, 10% CH₃CN to 60% NH₄HCO₃ 0.25% solution in water, 40% CH₃CN), yielding compound 162 (29.2 mg, 15%) as a colourless oil.

### E144. Preparation of Final Compound 163

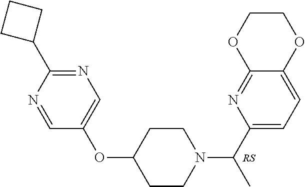 [SMILES: CC(c1ccc2c(n1)OCCO2)N1CCC(Oc2cnc(C3CCC3)nc2)CC1]

Compound 163 was prepared following an analogous procedure to the one described for the synthesis of compound 119 using compound 67 as starting material. The crude was purified by flash column chromatography (silica, MeOH in DCM 0/100 to 5/95). The desired fractions were collected and concentrated in vacuo. The product was further purified by reverse phase HPLC (Stationary phase: C18 XBridge 30×100 mm 5 μm, mobile phase: gradient from 85% NH₄HCO₃ 0.25% solution in water, 15% CH₃CN to 55% NH₄HCO₃ 0.25% solution in water, 45% CH₃CN), yielding compound 163 (48 mg, 57%) as a colourless film.

### E145. Preparation of Final Compound 164

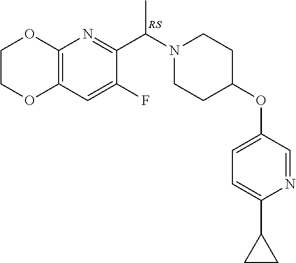 [SMILES: CC(c1nc2c(cc1F)OCCO2)N1CCC(Oc2ccc(C3CC3)nc2)CC1]

Cyclopropylzine bromide solution (0.5 M in THF, 0.457 mL, 0.228 mmol) was added to a solution of compound 26 (50 mg, 0.114 mmol) and Pd(t-Bu₃P)₂ (2.9 mg, 0.006 mmol)) in THF (0.43 mL) at room temperature and under a N₂ atmosphere. The mixture was stirred at room temperature for 18 h. Then additional more cyclopropylzinc bromide solution (0.5 M in THF, 0.457 mL, 0.228 mmol) and Pd(t-Bu₃P)₂ (0.05 eq) were added and the mixture was stirred at 60° C. for 18 h. Then, the mixture was treated with a mixture of sat. NH₄Cl and NH₄OH (1:1) and extracted with EtOAc. The organic layer was separated, dried (MgSO₄), filtered and the solvents evaporated in vacuo. The crude product was purified by flash column chromatography (silica; methanol in DCM 0/100 to 5/95). The desired fractions were collected and concentrated in vacuo. The product was further purified by RP HPLC (Stationary phase: C18 XBridge 30×100 mm 5 μm, mobile phase: gradient from 85% NH₄HCO₃ 0.25% solution in water, 15% CH₃CN to 55% NH₄HCO₃ 0.25% solution in water, 45% CH₃CN), to yield compound 164 (25 mg, 55%) as a colourless film.

### E146. Preparation of Final Compound 165

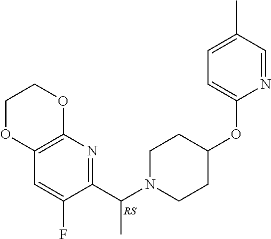 [SMILES: Cc1ccc(OC2CCN(C(C)c3nc4c(cc3F)OCCO4)CC2)nc1]

DBAD (548.61 mg, 2.38 mmol) was added to a solution of 2-hydroxy-5-methylpyridine (CAS: 1003-68-5, 200 mg, 1.83 mmol), intermediate 145 (532.77 mg, 1.83 mmol) and PPh₃ (624.92 mg, 2.38 mmol) in toluene (7.99 mL) under N₂ at 0° C. and the reaction mixture was stirred at 0° C. for 2 h. Then, the mixture was concentrated in vacuo and the crude product was purified by flash column chromatography (silica, MeOH in DCM 0/100 to 5/95). The desired fractions were collected and evaporated in vacuo to yield 240 mg of compound 165 as a yellow oil. The compound was purified by reverse phase HPLC (Stationary phase: C18 XBridge 50×100 mm 5 μm, mobile phase: gradient from 75% NH₄HCO₃ 0.25% solution in water, 25% CH₃CN to 40% NH₄HCO₃ 0.25% solution in water, 60% CH₃CN), yielding compound 165 (76.9 mg, 11%) as a colourless oil.

The compound was treated with 2N HCl in Et₂O to yield compound 165. HCl (80 mg, 11%) as a white solid. NMR revealed it contained NH₄.

Therefore, the sample was suspended in Na₂CO₃ saturated aq. solution and extracted with EtOAc. The organic layer was separated, dried, and solvent concentrated in vacuo to give an oil which was dissolved in Et₂O and treated with 2N HCl solution in Et₂O to give compound 165. HCl (55.8 mg, 7%) as a white solid.

### E147. Preparation of Final Compound 166

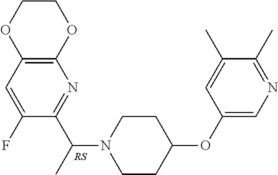 [SMILES: Cc1cc(OC2CCN(C(C)c3nc4c(cc3F)OCCO4)CC2)cnc1C]

Compound 166 was prepared following an analogous procedure to the one described for the synthesis of compound 165 using intermediate 145 and 5,6-dimethylpyridin-3-ol (CAS: 61893-00-3) as starting materials. The crude was purified by flash column chromatography (silica, MeOH in DCM 0/100 to 3/97). The desired fractions were collected and concentrated in vacuo to yield a white solid, which was purified again by flash column chromatography (silica, MeOH in DCM 0/100 to 3/97). The desired fractions were collected and concentrated in vacuo to yield compound 166 (35.1 mg, 18%) as a white solid.

### E148. Preparation of Final Compound 168

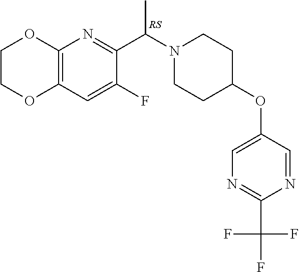 [SMILES: CC(c1nc2c(cc1F)OCCO2)N1CCC(Oc2cnc(C(F)(F)F)nc2)CC1]

Compound 168 was prepared following an analogous procedure to the one described for the synthesis of compound 70 using intermediate 207 and intermediate 20 as starting materials. The crude was purified by flash column chromatography (silica, MeOH in DCM 0/100 to 10/90). The desired fractions were collected and concentrated in vacuo to yield a mixture of stereoisomers. The mixture was purified by reverse phase HPLC (Stationary phase: C18 XBridge 30×100 mm 5 μm, mobile phase: gradient from 67% 0.1% NH₄HCO₃/NH₄OH pH 9 solution in water, 33% CH₃CN to 50% 0.1% NH₄HCO₃/NH₄OH pH 9 solution in water, 50% CH₃CN), yielding compound 168 (108 mg, 62%) as a white solid (sticky).

### E149. Preparation of Final Compound 169

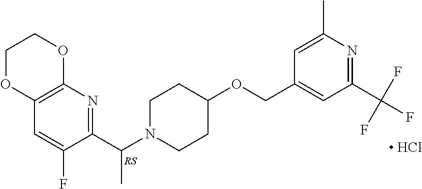 [SMILES: Cc1cc(COC2CCN(C(C)c3nc4c(cc3F)OCCO4)CC2)cc(C(F)(F)F)n1 ; "· HCl"]

Intermediate 205 (103.98 mg, 0.379 mmol) was added to a stirred solution of intermediate 20 (75 mg, 0.345 mmol) and K₂C03 (142.89 mg, 0.379 mmol) in CH₃CN (3 mL) at rt. The mixture was stirred at 75° C. for 40 h. The mixture was diluted with NaHCO₃ sat. and extracted with EtOAc. The organic layer was dried (MgSO₄), filtered and the solvents evaporated in vacuo. The crude was purified by flash column chromatography (silica; EtOAc in heptane, from 0/100 to 0/100). The desired fractions were collected and concentrated to yield a colourless foamy solid, which was purified by reverse phase (Phenomenex Gemini C18 100×30 mm 5 μm Column; from 59% [25 mM NH₄HCO₃]-41% [CH₃CN:MeOH (1:1)] to 17% [25 mM NH₄HCO₃]-83% [CH₃CN:MeOH (1:1)]). The desired fractions were collected and concentrated to yield compound 169 (110 mg, 69%) as a colourless foamy solid. The product was dissolved in DCM and treated with 1.05 eq of HCl 4 M in dioxane (0.063 mL) The solvents were evaporated in vacuo and the product was triturated with diethyl ether, filtered and dried to yield compound 169. HCl (101.9 mg, 60%) as a white foamy solid.

The following compounds were prepared following the methods exemplified in the Experimental Part. In case no salt form is indicated, the compound was obtained as a free base. ‘Ex. No.’ refers to the Example number according to which protocol the compound was synthesized. ‘Co. No.’ means compound number.

TABLE 1

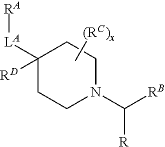

| Co. | Ex. | | |
|---|---|---|---|
| No. | No. | Structure | Salt Form |
| 1 | E1 | 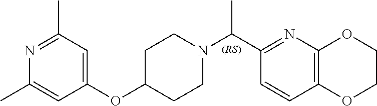 [SMILES: Cc1cc(OC2CCN(C(C)c3ccc4c(n3)OCCO4)CC2)cc(C)n1] | •HCl |
| 2 | E1 | 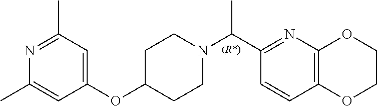 [SMILES: Cc1cc(OC2CCN(C(C)c3ccc4c(n3)OCCO4)CC2)cc(C)n1] | •HCl |
| 3 | E1 | 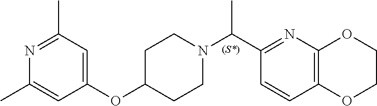 [SMILES: Cc1cc(OC2CCN(C(C)c3ccc4c(n3)OCCO4)CC2)cc(C)n1] | |
| 4 | E2 | 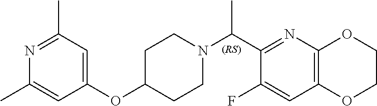 [SMILES: Cc1cc(OC2CCN(C(C)c3nc4c(cc3F)OCCO4)CC2)cc(C)n1] | |
| 5 | E3 | 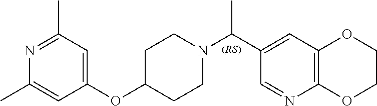 [SMILES: Cc1cc(OC2CCN(C(C)c3cnc4c(c3)OCCO4)CC2)cc(C)n1] | |
| 6 | E4 | 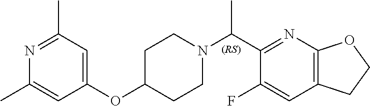 [SMILES: Cc1cc(OC2CCN(C(C)c3nc4c(cc3F)CCO4)CC2)cc(C)n1] | |
| 7 | E5 | 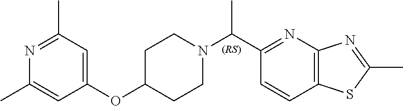 | |
| 8 | E6 | 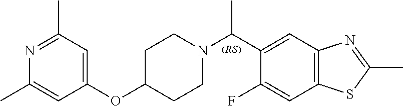 | |
| 9 | E7 | 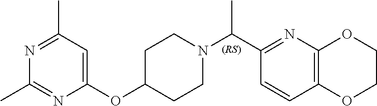 [SMILES: Cc1cc(OC2CCN(C(C)c3ccc4c(n3)OCCO4)CC2)nc(C)n1] | |
| 10 | E7 | 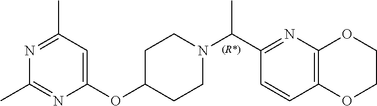 [SMILES: Cc1cc(OC2CCN(C(C)c3ccc4c(n3)OCCO4)CC2)nc(C)n1] | •HCl |
| 11 | E7 | 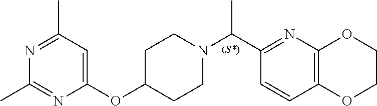 [SMILES: Cc1cc(OC2CCN(C(C)c3ccc4c(n3)OCCO4)CC2)nc(C)n1] | •HCl |
| 12 | E8 | 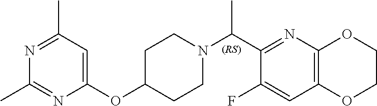 [SMILES: Cc1cc(OC2CCN(C(C)c3nc4c(cc3F)OCCO4)CC2)nc(C)n1] | |
| 13 | E9 | 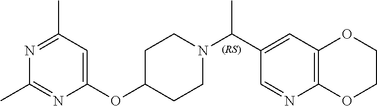 [SMILES: Cc1cc(OC2CCN(C(C)c3cnc4c(c3)OCCO4)CC2)nc(C)n1] | |
| 14 | E10 | 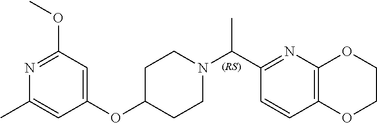 [SMILES: COc1cc(OC2CCN(C(C)c3ccc4c(n3)OCCO4)CC2)cc(C)n1] | |
| 15 | E11 | 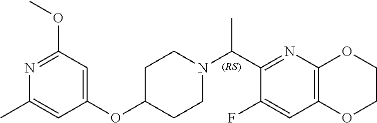 [SMILES: COc1cc(OC2CCN(C(C)c3nc4c(cc3F)OCCO4)CC2)cc(C)n1] | |
| 16 | E12 | 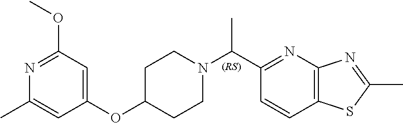 | |
| 17 | E13 | 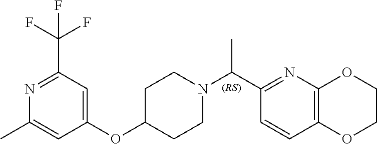 [SMILES: Cc1cc(OC2CCN(C(C)c3ccc4c(n3)OCCO4)CC2)cc(C(F)(F)F)n1] | |
| 18 | E14 | 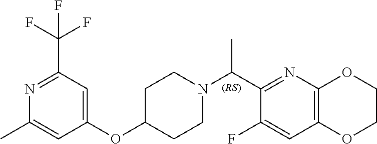 [SMILES: Cc1cc(OC2CCN(C(C)c3nc4c(cc3F)OCCO4)CC2)cc(C(F)(F)F)n1] | |
| 19 | E15 | 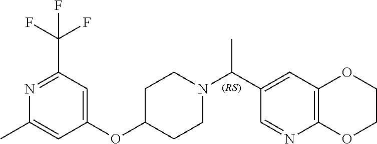 [SMILES: Cc1cc(OC2CCN(C(C)c3cnc4c(c3)OCCO4)CC2)cc(C(F)(F)F)n1] | |
| 20 | E15 | 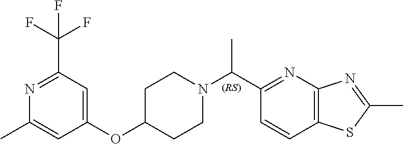 | |
| 21 | E17 | 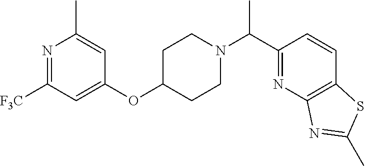 [SMILES: Cc1cc(OC2CCN(C(C)c3ccc4sc(C)nc4n3)CC2)cc(C(F)(F)F)n1] | |
| 22 | E18 | 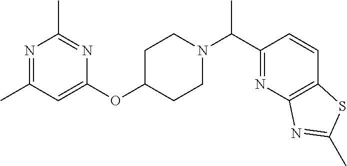 | |
| 23 | E19 | 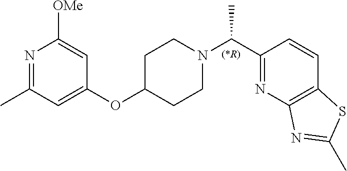 | |
| 24 | E19 | 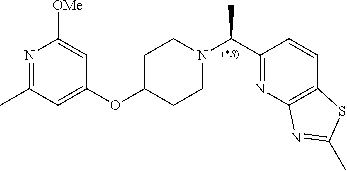 | 2C₆H₈O₇ |
| 25 | E20 | 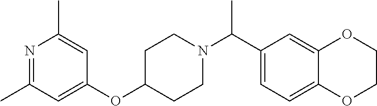 [SMILES: Cc1cc(OC2CCN(C(C)c3ccc4c(c3)OCCO4)CC2)cc(C)n1] | 2HCl |
| 26 | E21 | 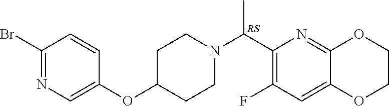 [SMILES: CC(c1nc2c(cc1F)OCCO2)N1CCC(Oc2ccc(Br)nc2)CC1] | |
| 27 | E22 | 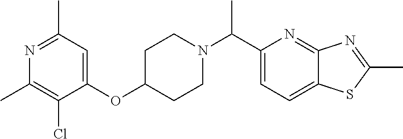 | |
| 28 | E23 | 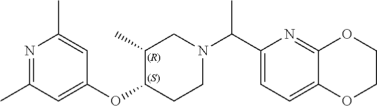 | |
| 29 | E24 | 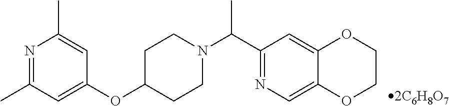 [SMILES: Cc1cc(OC2CCN(C(C)c3cc4c(cn3)OCCO4)CC2)cc(C)n1] | 2C₆H₈O₇ |
| 30 | E25 | 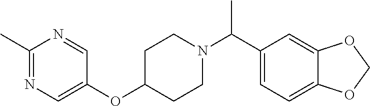 [SMILES: Cc1ncc(OC2CCN(C(C)c3ccc4c(c3)OCO4)CC2)cn1] | HCl |
| 31 | E26 | 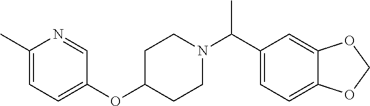 [SMILES: Cc1ccc(OC2CCN(C(C)c3ccc4c(c3)OCO4)CC2)cn1] | HCl |
| 32 | E27 | 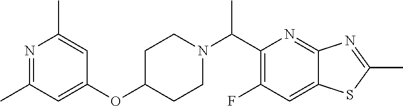 | |
| 33 | E28 | 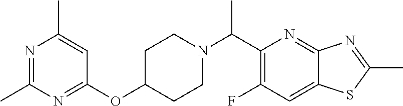 | |
| 34 | E29 | 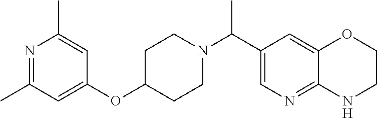 [SMILES: Cc1cc(OC2CCN(C(C)c3cnc4c(c3)OCCN4)CC2)cc(C)n1] | |
| 35 | E30 | 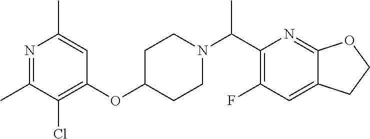 [SMILES: Cc1cc(OC2CCN(C(C)c3nc4c(cc3F)CCO4)CC2)c(Cl)c(C)n1] | |
| 36 | E31 | 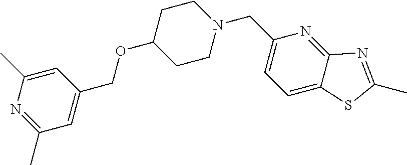 | |
| 37 | E32 | 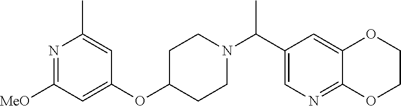 [SMILES: COc1cc(OC2CCN(C(C)c3cnc4c(c3)OCCO4)CC2)cc(C)n1] | |
| 38 | E33 | 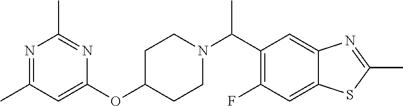 | |
| 39 | E34 | 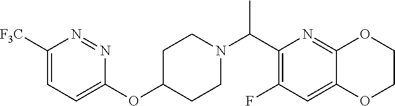 [SMILES: CC(c1nc2c(cc1F)OCCO2)N1CCC(Oc2ccc(C(F)(F)F)nn2)CC1] | |
| 40 | E35 | 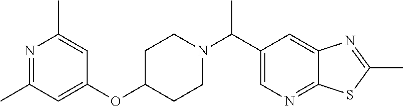 | |
| 41 | E36 | 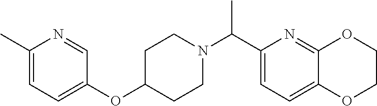 [SMILES: Cc1ccc(OC2CCN(C(C)c3ccc4c(n3)OCCO4)CC2)cn1] | |
| 42 | E37 | 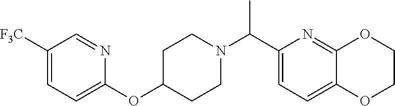 [SMILES: CC(c1ccc2c(n1)OCCO2)N1CCC(Oc2ccc(C(F)(F)F)cn2)CC1] | 2HCl |
| 43 | E38 | 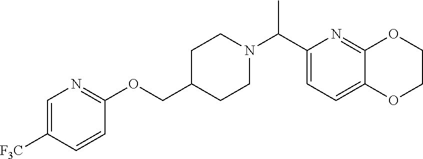 [SMILES: CC(c1ccc2c(n1)OCCO2)N1CCC(COc2ccc(C(F)(F)F)cn2)CC1] | 2HCl |
| 44 | E39 | 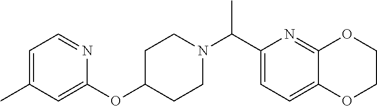 [SMILES: Cc1ccnc(OC2CCN(C(C)c3ccc4c(n3)OCCO4)CC2)c1] | 3HCl |
| 45 | E40 | 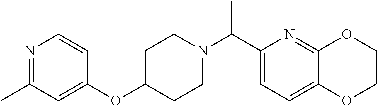 [SMILES: Cc1cc(OC2CCN(C(C)c3ccc4c(n3)OCCO4)CC2)ccn1] | 3HCl |
| 46 | E41 | 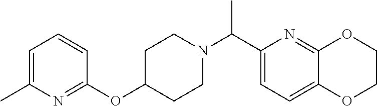 [SMILES: Cc1cccc(OC2CCN(C(C)c3ccc4c(n3)OCCO4)CC2)n1] | 3HCl |
| 47 | E42 | 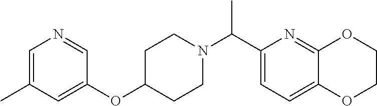 [SMILES: Cc1cncc(OC2CCN(C(C)c3ccc4c(n3)OCCO4)CC2)c1] | 3HCl |
| 48 | E43 | 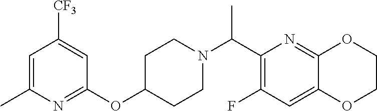 [SMILES: Cc1cc(C(F)(F)F)cc(OC2CCN(C(C)c3nc4c(cc3F)OCCO4)CC2)n1] | |
| 49 | E44 | 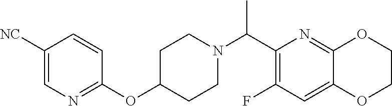 [SMILES: CC(c1nc2c(cc1F)OCCO2)N1CCC(Oc2ccc(C#N)cn2)CC1] | |
| 50 | E45 | 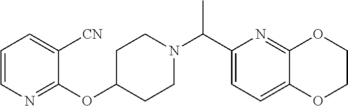 [SMILES: CC(c1ccc2c(n1)OCCO2)N1CCC(Oc2ncccc2C#N)CC1] | |
| 51 | E46 | 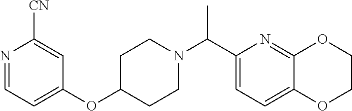 [SMILES: CC(c1ccc2c(n1)OCCO2)N1CCC(Oc2ccnc(C#N)c2)CC1] | |
| 52 | E47 | 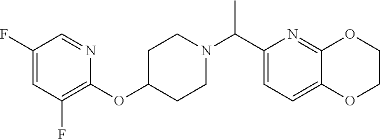 [SMILES: CC(c1ccc2c(n1)OCCO2)N1CCC(Oc2ncc(F)cc2F)CC1] | |
| 53 | E48 | 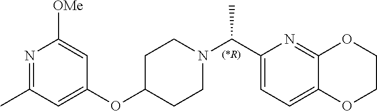 [SMILES: COc1cc(OC2CCN([C@H](C)c3ccc4c(n3)OCCO4)CC2)cc(C)n1] | |
| 54 | E48 | 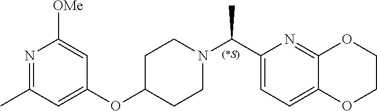 [SMILES: COc1cc(OC2CCN([C@@H](C)c3ccc4c(n3)OCCO4)CC2)cc(C)n1] | |
| 55 | E49 | 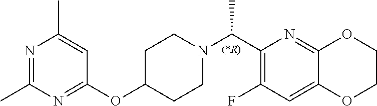 [SMILES: Cc1cc(OC2CCN([C@H](C)c3nc4c(cc3F)OCCO4)CC2)nc(C)n1] | |
| 56 | E50 | 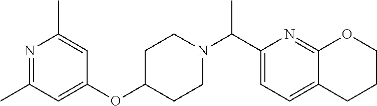 [SMILES: Cc1cc(OC2CCN(C(C)c3ccc4c(n3)OCCC4)CC2)cc(C)n1] | 2HCl |
| 57 | E51 | 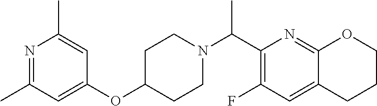 [SMILES: Cc1cc(OC2CCN(C(C)c3nc4c(cc3F)CCCO4)CC2)cc(C)n1] | 2HCl |
| 58 | E52 | 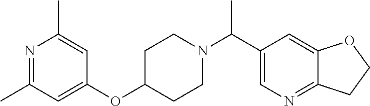 [SMILES: Cc1cc(OC2CCN(C(C)c3cnc4c(c3)OCC4)CC2)cc(C)n1] | 2HCl |
| 59 | E53 | 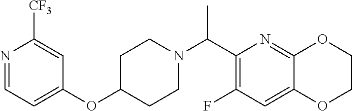 [SMILES: CC(c1nc2c(cc1F)OCCO2)N1CCC(Oc2ccnc(C(F)(F)F)c2)CC1] | |
| 60 | E54 | 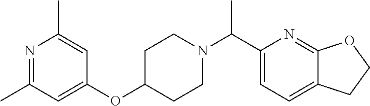 [SMILES: Cc1cc(OC2CCN(C(C)c3ccc4c(n3)OCC4)CC2)cc(C)n1] | 2HCl |
| 61 | E54 | 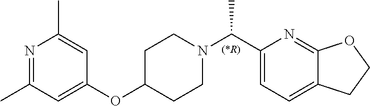 [SMILES: Cc1cc(OC2CCN([C@H](C)c3ccc4c(n3)OCC4)CC2)cc(C)n1] | 2HCl |
| 62 | E54 | 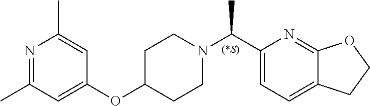 [SMILES: Cc1cc(OC2CCN([C@@H](C)c3ccc4c(n3)OCC4)CC2)cc(C)n1] | 2HCl |
| 63 | E55 | 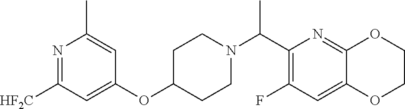 | |
| 64 | E56 | 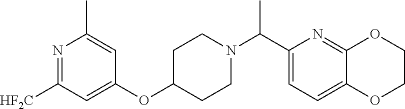 | |
| 65 | E57 | 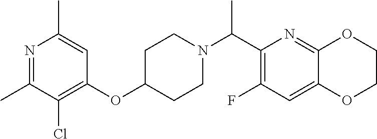 [SMILES: Cc1cc(OC2CCN(C(C)c3nc4c(cc3F)OCCO4)CC2)c(Cl)c(C)n1] | |
| 66 | E58 | 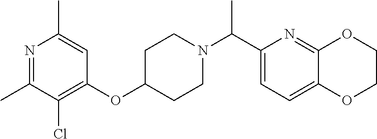 [SMILES: Cc1cc(OC2CCN(C(C)c3ccc4c(n3)OCCO4)CC2)c(Cl)c(C)n1] | |
| 67 | E59 | 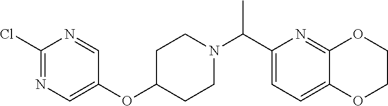 [SMILES: CC(c1ccc2c(n1)OCCO2)N1CCC(Oc2cnc(Cl)nc2)CC1] | |
| 68 | E60 | 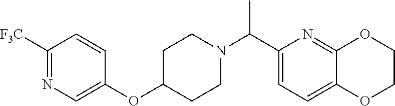 [SMILES: CC(c1ccc2c(n1)OCCO2)N1CCC(Oc2ccc(C(F)(F)F)nc2)CC1] | |
| 69 | E61 | 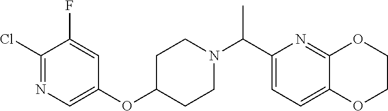 [SMILES: CC(c1ccc2c(n1)OCCO2)N1CCC(Oc2cnc(Cl)c(F)c2)CC1] | |
| 70 | E62 | 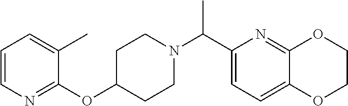 [SMILES: Cc1cccnc1OC1CCN(C(C)c2ccc3c(n2)OCCO3)CC1] | 2HCl |
| 71 | E63 | 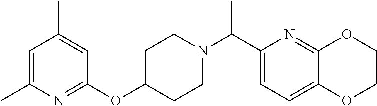 [SMILES: Cc1cc(C)nc(OC2CCN(C(C)c3ccc4c(n3)OCCO4)CC2)c1] | |
| 72 | E64 | 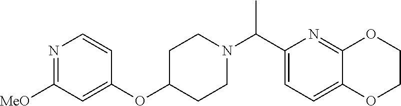 [SMILES: COc1cc(OC2CCN(C(C)c3ccc4c(n3)OCCO4)CC2)ccn1] | |
| 73 | E65 | 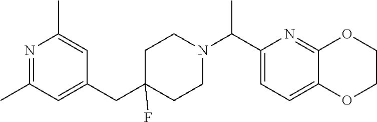 [SMILES: Cc1cc(CC2(F)CCN(C(C)c3ccc4c(n3)OCCO4)CC2)cc(C)n1] | |
| 74 | E66 | 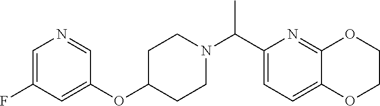 [SMILES: CC(c1ccc2c(n1)OCCO2)N1CCC(Oc2cncc(F)c2)CC1] | |
| 75 | E67 | 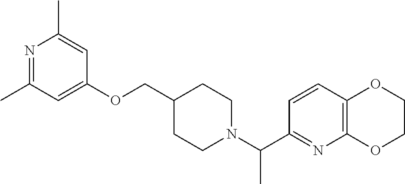 [SMILES: Cc1cc(OCC2CCN(C(C)c3ccc4c(n3)OCCO4)CC2)cc(C)n1] | |
| 76 | E68 | 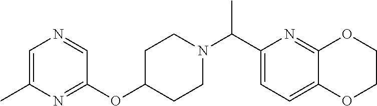 [SMILES: Cc1cncc(OC2CCN(C(C)c3ccc4c(n3)OCCO4)CC2)n1] | |
| 77 | E69 | 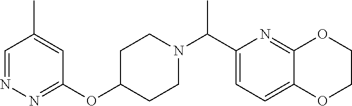 [SMILES: Cc1cnnc(OC2CCN(C(C)c3ccc4c(n3)OCCO4)CC2)c1] | |
| 78 | E70 | 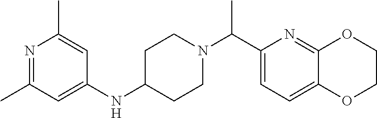 [SMILES: Cc1cc(NC2CCN(C(C)c3ccc4c(n3)OCCO4)CC2)cc(C)n1] | |
| 79 | E71 | 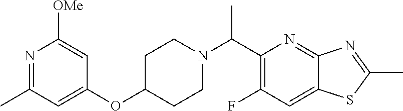 | |
| 80 | E72 | 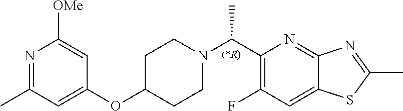 | HCl |
| 81 | E72 | 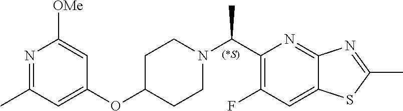 | HCl |
| 82 | E73 | 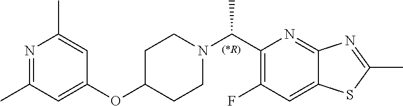 | 2HCl |
| 83 | E73 | 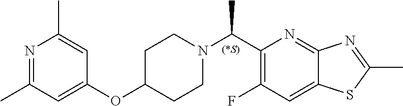 | 2HCl |
| 84 | E74 | 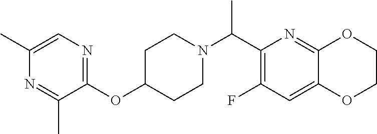 [SMILES: Cc1cnc(OC2CCN(C(C)c3nc4c(cc3F)OCCO4)CC2)c(C)n1] | |
| 85 | E75 | 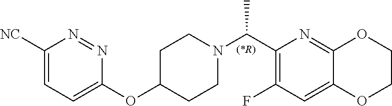 [SMILES: C[C@H](c1nc2c(cc1F)OCCO2)N1CCC(Oc2ccc(C#N)nn2)CC1] | |
| 86 | E75 | 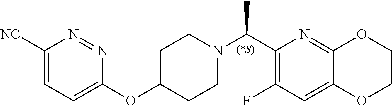 [SMILES: C[C@@H](c1nc2c(cc1F)OCCO2)N1CCC(Oc2ccc(C#N)nn2)CC1] | |
| 87 | E76 | 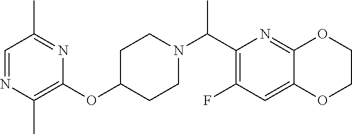 [SMILES: Cc1cnc(C)c(OC2CCN(C(C)c3nc4c(cc3F)OCCO4)CC2)n1] | |
| 88 | E77 | 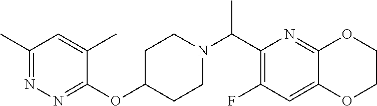 [SMILES: Cc1cc(C)c(OC2CCN(C(C)c3nc4c(cc3F)OCCO4)CC2)nn1] | |
| 89 | E78 | 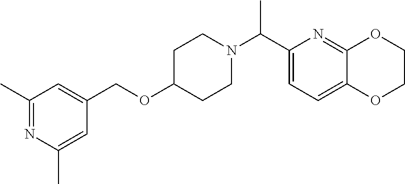 [SMILES: Cc1cc(COC2CCN(C(C)c3ccc4c(n3)OCCO4)CC2)cc(C)n1] | |
| 90 | E79 | 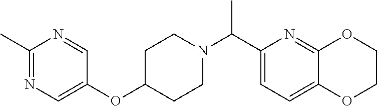 [SMILES: Cc1ncc(OC2CCN(C(C)c3ccc4c(n3)OCCO4)CC2)cn1] | HCl |
| 91 | E80 | 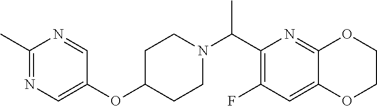 [SMILES: Cc1ncc(OC2CCN(C(C)c3nc4c(cc3F)OCCO4)CC2)cn1] | HCl |
| 92 | E81 | 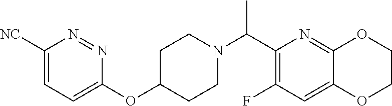 [SMILES: CC(c1nc2c(cc1F)OCCO2)N1CCC(Oc2ccc(C#N)nn2)CC1] | |
| 93 | E82 | 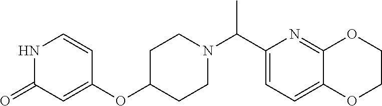 [SMILES: CC(c1ccc2c(n1)OCCO2)N1CCC(Oc2cc[nH]c(=O)c2)CC1] | |
| 94 | E83 | 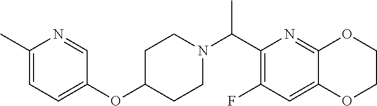 [SMILES: Cc1ccc(OC2CCN(C(C)c3nc4c(cc3F)OCCO4)CC2)cn1] | HCl |
| 95 | E84 | 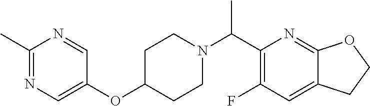 [SMILES: Cc1ncc(OC2CCN(C(C)c3nc4c(cc3F)CCO4)CC2)cn1] | |
| 96 | E85 | 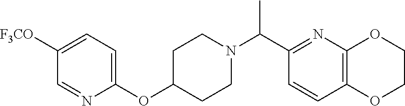 [SMILES: CC(c1ccc2c(n1)OCCO2)N1CCC(Oc2ccc(OC(F)(F)F)cn2)CC1] | |
| 97 | E86 | 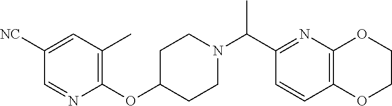 [SMILES: Cc1cc(C#N)cnc1OC1CCN(C(C)c2ccc3c(n2)OCCO3)CC1] | |
| 98 | E87 | 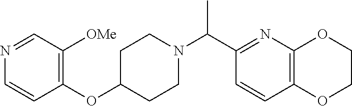 [SMILES: COc1cnccc1OC1CCN(C(C)c2ccc3c(n2)OCCO3)CC1] | |
| 99 | E88 | 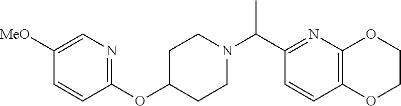 [SMILES: COc1ccc(OC2CCN(C(C)c3ccc4c(n3)OCCO4)CC2)nc1] | |
| 100 | E89 | 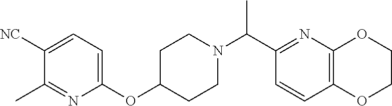 [SMILES: Cc1nc(OC2CCN(C(C)c3ccc4c(n3)OCCO4)CC2)ccc1C#N] | |
| 101 | E90 | 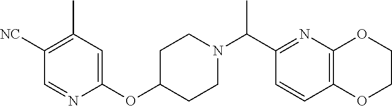 [SMILES: Cc1cc(OC2CCN(C(C)c3ccc4c(n3)OCCO4)CC2)ncc1C#N] | |
| 102 | E91 | 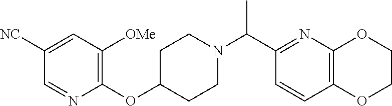 [SMILES: COc1cc(C#N)cnc1OC1CCN(C(C)c2ccc3c(n2)OCCO3)CC1] | |
| 103 | E92 | 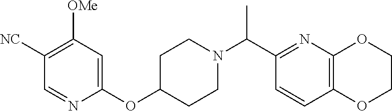 [SMILES: COc1cc(OC2CCN(C(C)c3ccc4c(n3)OCCO4)CC2)ncc1C#N] | |
| 104 | E93 | 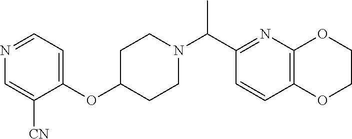 [SMILES: CC(c1ccc2c(n1)OCCO2)N1CCC(Oc2ccncc2C#N)CC1] | |
| 105 | E94 | 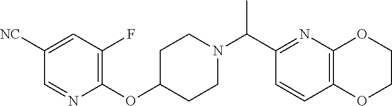 [SMILES: CC(c1ccc2c(n1)OCCO2)N1CCC(Oc2ncc(C#N)cc2F)CC1] | |
| 106 | E95 | 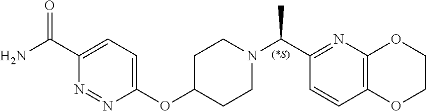 [SMILES: C[C@@H](c1ccc2c(n1)OCCO2)N1CCC(Oc2ccc(C(N)=O)nn2)CC1] | |
| 107 | E96 | 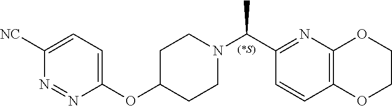 [SMILES: C[C@@H](c1ccc2c(n1)OCCO2)N1CCC(Oc2ccc(C#N)nn2)CC1] | |
| 108 | E97 | 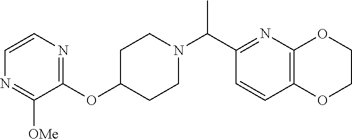 [SMILES: COc1nccnc1OC1CCN(C(C)c2ccc3c(n2)OCCO3)CC1] | |
| 109 | E98 | 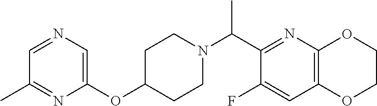 [SMILES: Cc1cncc(OC2CCN(C(C)c3nc4c(cc3F)OCCO4)CC2)n1] | |
| 110 | E99 | 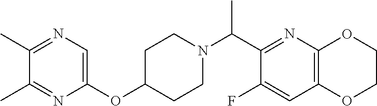 [SMILES: Cc1ncc(OC2CCN(C(C)c3nc4c(cc3F)OCCO4)CC2)nc1C] | |
| 111 | E100 | 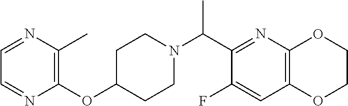 [SMILES: Cc1nccnc1OC1CCN(C(C)c2nc3c(cc2F)OCCO3)CC1] | |
| 112 | E101 | 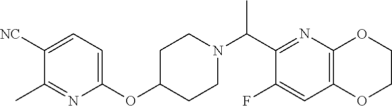 [SMILES: Cc1nc(OC2CCN(C(C)c3nc4c(cc3F)OCCO4)CC2)ccc1C#N] | |
| 113 | E102 | 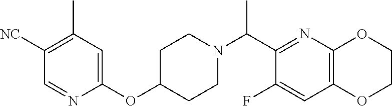 [SMILES: Cc1cc(OC2CCN(C(C)c3nc4c(cc3F)OCCO4)CC2)ncc1C#N] | |
| 114 | E103 | 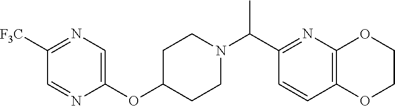 [SMILES: CC(c1ccc2c(n1)OCCO2)N1CCC(Oc2cnc(C(F)(F)F)cn2)CC1] | |
| 115 | E104 | 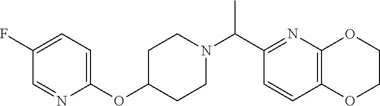 [SMILES: CC(c1ccc2c(n1)OCCO2)N1CCC(Oc2ccc(F)cn2)CC1] | |
| 116 | E105 | 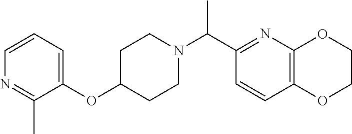 [SMILES: Cc1ncccc1OC1CCN(C(C)c2ccc3c(n2)OCCO3)CC1] | |
| 117 | E106 | 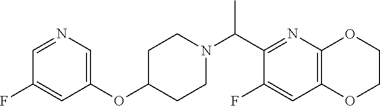 [SMILES: CC(c1nc2c(cc1F)OCCO2)N1CCC(Oc2cncc(F)c2)CC1] | |
| 118 | E107 | 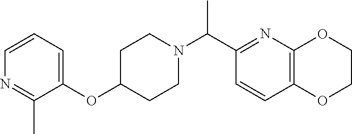 [SMILES: Cc1ncccc1OC1CCN(C(C)c2ccc3c(n2)OCCO3)CC1] | |
| 119 | E108 | 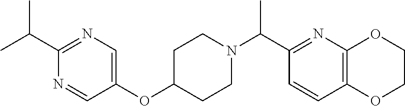 [SMILES: CC(C)c1ncc(OC2CCN(C(C)c3ccc4c(n3)OCCO4)CC2)cn1] | |
| 120 | E109 | 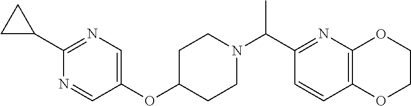 [SMILES: CC(c1ccc2c(n1)OCCO2)N1CCC(Oc2cnc(C3CC3)nc2)CC1] | |
| 121 | E110 | 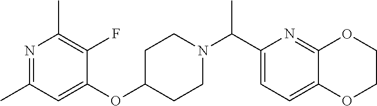 [SMILES: Cc1cc(OC2CCN(C(C)c3ccc4c(n3)OCCO4)CC2)c(F)c(C)n1] | |
| 122 | E111 | 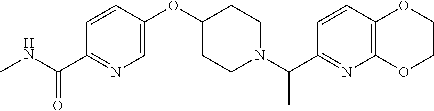 [SMILES: CNC(=O)c1ccc(OC2CCN(C(C)c3ccc4c(n3)OCCO4)CC2)cn1] | |
| 123 | E112 | 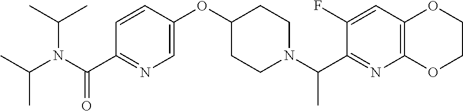 [SMILES: CC(c1nc2c(cc1F)OCCO2)N1CCC(Oc2ccc(C(=O)N(C(C)C)C(C)C)nc2)CC1] | 2HCl |
| 124 | E113 | 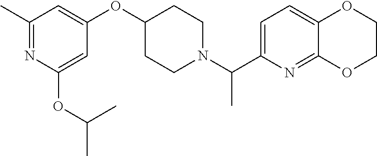 [SMILES: Cc1cc(OC2CCN(C(C)c3ccc4c(n3)OCCO4)CC2)cc(OC(C)C)n1] | 2HCl |
| 125 | E114 | 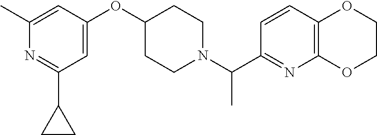 [SMILES: Cc1cc(OC2CCN(C(C)c3ccc4c(n3)OCCO4)CC2)cc(C2CC2)n1] | HCl |
| 126 | E115 | 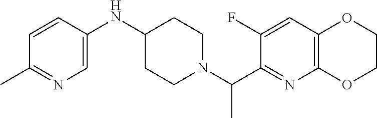 [SMILES: Cc1ccc(NC2CCN(C(C)c3nc4c(cc3F)OCCO4)CC2)cn1] | |
| 127 | E116 | 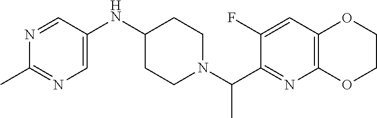 [SMILES: Cc1ncc(NC2CCN(C(C)c3nc4c(cc3F)OCCO4)CC2)cn1] | |
| 128 | E117 | 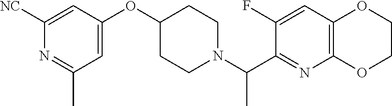 [SMILES: Cc1cc(OC2CCN(C(C)c3nc4c(cc3F)OCCO4)CC2)cc(C#N)n1] | |
| 129 | E118 | 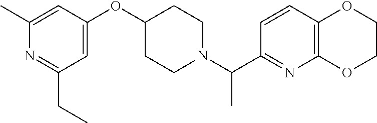 [SMILES: CCc1cc(OC2CCN(C(C)c3ccc4c(n3)OCCO4)CC2)cc(C)n1] | HCl |
| 130 | E119 | 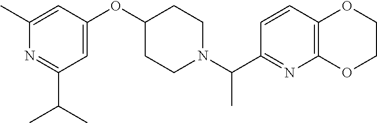 [SMILES: Cc1cc(OC2CCN(C(C)c3ccc4c(n3)OCCO4)CC2)cc(C(C)C)n1] | HCl |
| 131 | E120 | 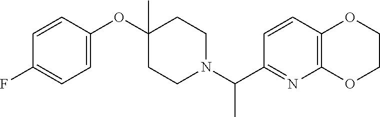 [SMILES: CC(c1ccc2c(n1)OCCO2)N1CCC(C)(Oc2ccc(F)cc2)CC1] | HCl |
| 132 | E121 | 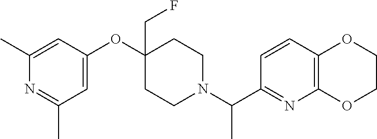 [SMILES: Cc1cc(OC2(CF)CCN(C(C)c3ccc4c(n3)OCCO4)CC2)cc(C)n1] | HCl |
| 133 | E122 | 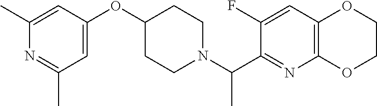 [SMILES: Cc1cc(OC2CCN(C(C)c3nc4c(cc3F)OCCO4)CC2)cc(C)n1] | |
| 134 | E123 | 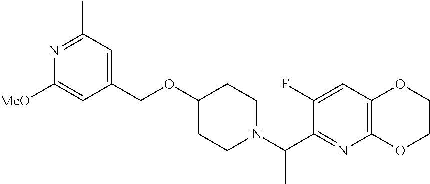 [SMILES: COc1cc(COC2CCN(C(C)c3nc4c(cc3F)OCCO4)CC2)cc(C)n1] | |
| 135 | E124 | 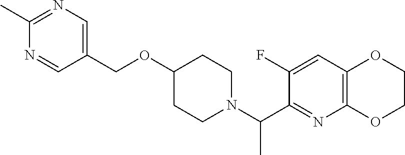 [SMILES: Cc1ncc(COC2CCN(C(C)c3nc4c(cc3F)OCCO4)CC2)cn1] | |
| 136 | E124 | 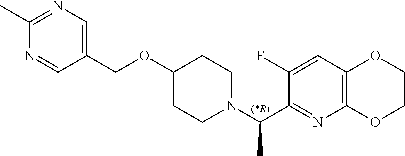 [SMILES: Cc1ncc(COC2CCN([C@H](C)c3nc4c(cc3F)OCCO4)CC2)cn1] | |
| 137 | E124 | 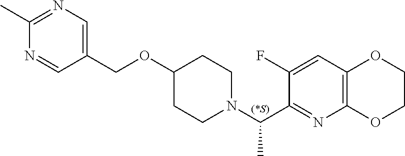 [SMILES: Cc1ncc(COC2CCN([C@@H](C)c3nc4c(cc3F)OCCO4)CC2)cn1] | |
| 138 | E125 | 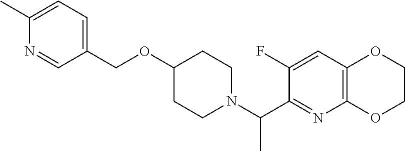 [SMILES: Cc1ccc(COC2CCN(C(C)c3nc4c(cc3F)OCCO4)CC2)cn1] | |
| 139 | E126 | 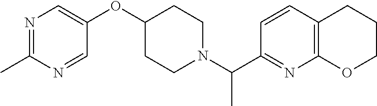 [SMILES: Cc1ncc(OC2CCN(C(C)c3ccc4c(n3)OCCC4)CC2)cn1] | HCl |
| 140 | E127 | 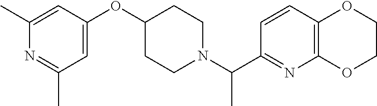 [SMILES: Cc1cc(OC2CCN(C(C)c3ccc4c(n3)OCCO4)CC2)cc(C)n1] | |
| 141 | E128 | 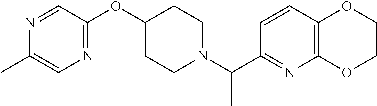 [SMILES: Cc1cnc(OC2CCN(C(C)c3ccc4c(n3)OCCO4)CC2)cn1] | |
| 142 | E129 | 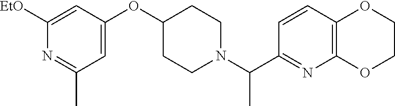 [SMILES: CCOc1cc(OC2CCN(C(C)c3ccc4c(n3)OCCO4)CC2)cc(C)n1] | HCl |
| 143 | E130 | 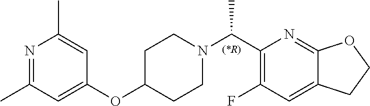 [SMILES: Cc1cc(OC2CCN([C@H](C)c3nc4c(cc3F)CCO4)CC2)cc(C)n1] | |
| 144 | E130 | 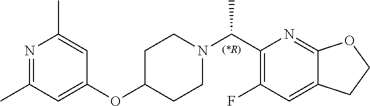 [SMILES: Cc1cc(OC2CCN([C@H](C)c3nc4c(cc3F)CCO4)CC2)cc(C)n1] | 2HCl |
| 145 | E130 | 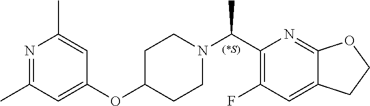 [SMILES: Cc1cc(OC2CCN([C@@H](C)c3nc4c(cc3F)CCO4)CC2)cc(C)n1] | 2HCl |
| 146 | E13 | 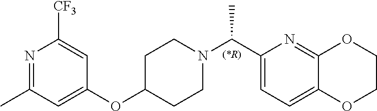 [SMILES: Cc1cc(OC2CCN([C@H](C)c3ccc4c(n3)OCCO4)CC2)cc(C(F)(F)F)n1] | HCl |
| 147 | E13 | 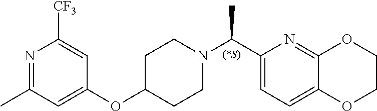 [SMILES: Cc1cc(OC2CCN([C@@H](C)c3ccc4c(n3)OCCO4)CC2)cc(C(F)(F)F)n1] | HCl |
| 148 | E2 | 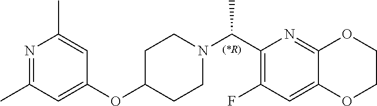 [SMILES: Cc1cc(OC2CCN([C@H](C)c3nc4c(cc3F)OCCO4)CC2)cc(C)n1] | 2HCl |
| 149 | E2 | 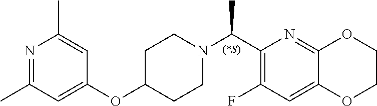 [SMILES: Cc1cc(OC2CCN([C@@H](C)c3nc4c(cc3F)OCCO4)CC2)cc(C)n1] | 2HCl |
| 150 | E131 | 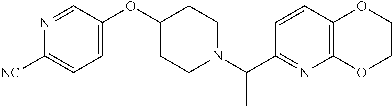 [SMILES: CC(c1ccc2c(n1)OCCO2)N1CCC(Oc2ccc(C#N)nc2)CC1] | |
| 151 | E132 | 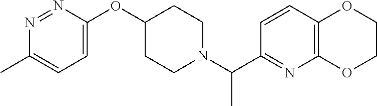 [SMILES: Cc1ccc(OC2CCN(C(C)c3ccc4c(n3)OCCO4)CC2)nn1] | HCl |
| 152 | E133 | 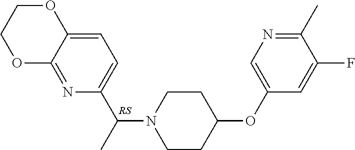 [SMILES: Cc1ncc(OC2CCN(C(C)c3ccc4c(n3)OCCO4)CC2)cc1F] | |
| 153 | E134 | 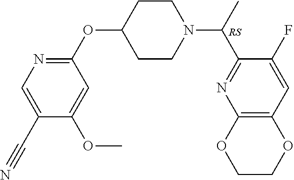 [SMILES: COc1cc(OC2CCN(C(C)c3nc4c(cc3F)OCCO4)CC2)ncc1C#N] | |
| 154 | E135 | 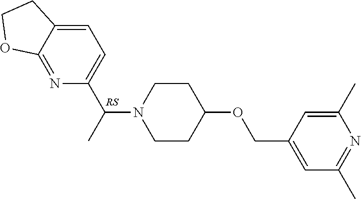 [SMILES: Cc1cc(COC2CCN(C(C)c3ccc4c(n3)OCC4)CC2)cc(C)n1] | |
| 155 | E136 | 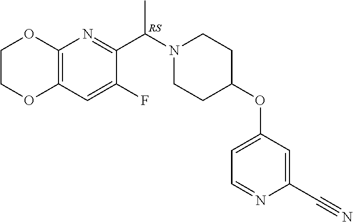 [SMILES: CC(c1nc2c(cc1F)OCCO2)N1CCC(Oc2ccnc(C#N)c2)CC1] | |
| 156 | E137 | 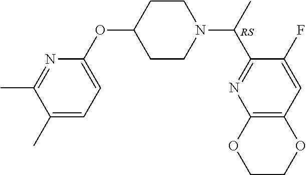 [SMILES: Cc1ccc(OC2CCN(C(C)c3nc4c(cc3F)OCCO4)CC2)nc1C] | |
| 157 | E138 | 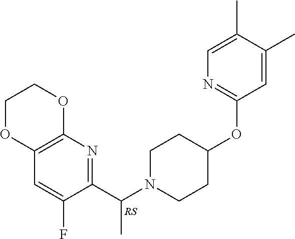 [SMILES: Cc1cnc(OC2CCN(C(C)c3nc4c(cc3F)OCCO4)CC2)cc1C] | •HCl |
| 158 | E139 | 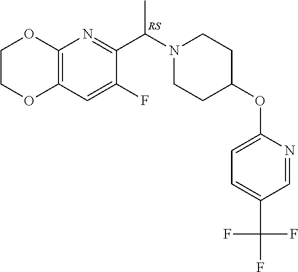 [SMILES: CC(c1nc2c(cc1F)OCCO2)N1CCC(Oc2ccc(C(F)(F)F)cn2)CC1] | |
| 159 | E140 | 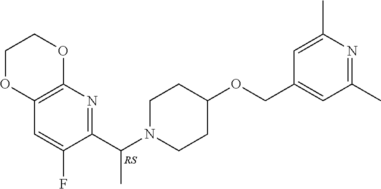 [SMILES: Cc1cc(COC2CCN(C(C)c3nc4c(cc3F)OCCO4)CC2)cc(C)n1] | •2HCl |
| 160 | E141 | 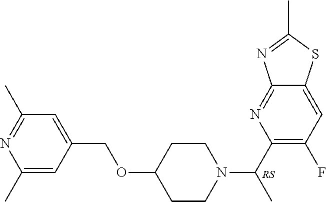 [SMILES: Cc1cc(COC2CCN(C(C)c3nc4nc(C)sc4cc3F)CC2)cc(C)n1] | •2HCl |
| 161 | E142 | 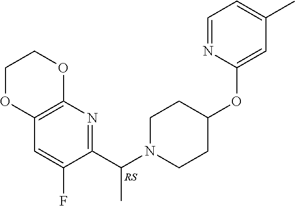 [SMILES: Cc1ccnc(OC2CCN(C(C)c3nc4c(cc3F)OCCO4)CC2)c1] | |
| 162 | E143 | 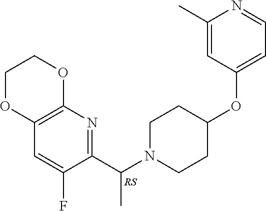 [SMILES: Cc1cc(OC2CCN(C(C)c3nc4c(cc3F)OCCO4)CC2)ccn1] | |
| 163 | E144 | 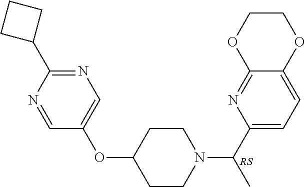 [SMILES: CC(c1ccc2c(n1)OCCO2)N1CCC(Oc2cnc(C3CCC3)nc2)CC1] | |
| 164 | E145 | 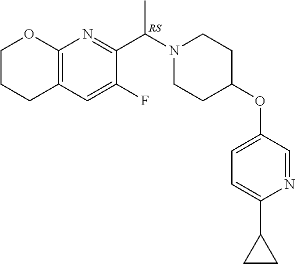 [SMILES: CC(c1nc2c(cc1F)CCCO2)N1CCC(Oc2ccc(C3CC3)nc2)CC1] | |
| 165 | E146 | 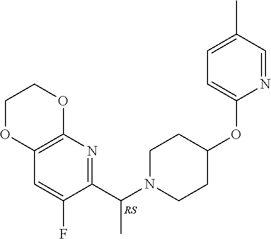 [SMILES: Cc1ccc(OC2CCN(C(C)c3nc4c(cc3F)OCCO4)CC2)nc1] | •HCl |
| 166 | E147 | 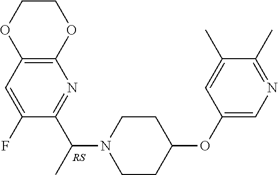 [SMILES: Cc1cc(OC2CCN(C(C)c3nc4c(cc3F)OCCO4)CC2)cnc1C] | |
| 167 | I-188 | 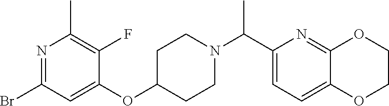 [SMILES: Cc1nc(Br)cc(OC2CCN(C(C)c3ccc4c(n3)OCCO4)CC2)c1F] | |
| 168 | E148 | 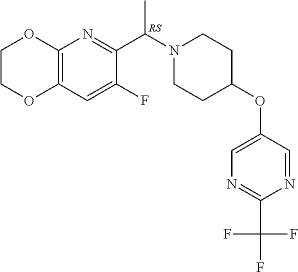 [SMILES: CC(c1nc2c(cc1F)OCCO2)N1CCC(Oc2cnc(C(F)(F)F)nc2)CC1] | |
| 169 | E149 | 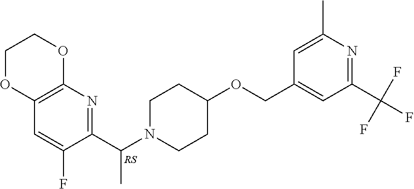 [SMILES: Cc1cc(COC2CCN(C(C)c3nc4c(cc3F)OCCO4)CC2)cc(C(F)(F)F)n1] | •HCl |

The values of salt stoichiometry or acid content in the compounds as provided herein, are those obtained experimentally. The content of hydrochloric acid reported herein was determined by ¹HNMR integration and/or elemental analysis.

Analytical Part

Melting Points

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values (A).

Mettler Toledo MP50: Melting points were measured with a temperature gradient of 1, 3, 5 or 10° C./minute. Maximum temperature was 300° C. The melting point was read from a digital display.

LCMS

General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rₜ) and ions. If not specified differently in the table of data, the reported molecular ion corresponds to the [M+H]⁺(protonated molecule) and/or [M−H]⁻ (deprotonated molecule). For molecules with multiple isotopic patterns (Br, Cl), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 2
LC-MS Methods (Flow expressed in mL/min; column
temperature (T) in ° C.; Run time in min).

| | | | | | Flow | |
|---|---|---|---|---|---|---|
| | | | | | ------- | Run |
| Method | Instrument | Column | Mobile Phase | Gradient | Col T | Time |
| 1 | Waters: | Waters: | A: 95% | From 95% A | 1 | 5 |
| | Acquity ® | BEH C18 | CH3COONH4 | to 5% A in | ------- | |
| | IClass | (1.7 μm, | 6.5 mM + 5% | 4.6 min, held | 50 | |
| | UPLC ® - | 2.1 × 50 mm) | CH3CN, | for 0.4 min | | |
| | DAD and | | B: CH3CN | | | |
| | Xevo G2-S | | | | | |
| | QTOF | | | | | |
| 2 | Agilent | YMC-pack | A: 0.1% | From 95% A | 2.6 | 6.2 |
| | 1100 | ODS-AQ C18 | HCOOH in | to 5% A in | ------- | |
| | HPLC | (50 × 4.6 mm, | H2O | 4.8 min, held | 35 | |
| | DAD | 3 μm) | B: CH3CN | for 1.0 min, | | |
| | LC/MS | | | to 95% A in | | |
| | G1956A | | | 0.2 min. | | |
| 3 | Waters: | Agilent: | A: 95% | From 95% A | 0.8 | 2.5 |
| | Acquity ® | RRHD | CH3COONH4 | to 5% A in | ------- | |
| | IClass | (1.8 μm, | 6.5 mM + 5% | 2.0 min, held | 50 | |
| | UPLC ® - | 2.1 × 50 mm) | CH3CN, | for 0.5 min | | |
| | DAD and | | B: CH3CN | | | |
| | SQD | | | | | |
| 4 | Waters: | Waters: | A: 95% | 84.2% A for | 0.343 | 6.2 |
| | Acquity | BEH C18 | CH3COONH4 | 0.49 min, to | ------- | |
| | UPLC ® - | (1.7 μm, | 7 mM/5% | 10.5% Ain | 40 | |
| | DAD and | 2.1 × 100 mm) | CH3CN, | 2.18 min, held | | |
| | Quattro | | B: CH3CN | for 1.94 min, | | |
| | MicroTM | | | back to | | |
| | | | | 84.2% A in | | |
| | | | | 0.73 min, held | | |
| | | | | for 0.73 min. | | |
| 5 | Waters: | Waters: | A: 95% | From 95% A | 0.8 | 2.5 |
| | Acquity ® | BEH C18 | CH3COONH4 | to 5% A in | ------- | |
| | UPLC ® - | (1.7 μm, | 6.5 mM + 5% | 2.0 min, held | 50 | |
| | DAD and | 2.1 × 50 mm) | CH3CN, | for 0.5 min | | |
| | SQD | | B: CH3CN | | | |
| 6 | Waters: | Waters: | A: 95% | From 84.2% | 0.343 | 6.1 |
| | Acquity | BEH C18 | CH3COONH4 | A to 10.5% A | ------- | |
| | UPLC ® H- | (1.7 μm, | 7 mM/5% | in 2.18 min, | 40 | |
| | Class - | 2.1 × 100 mm) | CH3CN, | held for | | |
| | DAD and | | B: CH3CN | 1.94 min, | | |
| | SQD 2 | | | back to | | |
| | | | | 84.2% A in | | |
| | | | | 0.73 min, held | | |
| | | | | for 0.73 min. | | |
| 7 | Waters: | Waters: | A: 95% | From 95% A | 0.8 | 5.0 |
| | Acquity ® | BEH C18 | CH3COONH4 | to 5% A in | ------- | |
| | UPLC ® - | (1.7 μm, | 6.5 mM + 5% | 4.5 min, held | 50 | |
| | DAD and | 2.1 × 50 mm) | CH3CN, | for 0.5 min | | |
| | SQD | | B: CH3CN | | | |
| 8 | Waters: | Waters: | A: 95% | From 95% A | 1 | 2.0 |
| | Acquity ® | BEH C18 | CH₃COONH₄ | to 40% A in | ------- | |
| | IClass | (1.7 μm, | 6.5 mM + 5% | 1.2 min, to | 50 | |
| | UPLC ® - | 2.1 × 50 mm) | CH₃CN, | 5% A in | | |
| | DAD and | | B: CH₃CN | 0.6 min, held | | |
| | Xevo G2-S | | | for 0.2 min | | |
| | QTOF | | | | | |
| 9 | Agilent | Waters: | A: TFA | 100% A kept | 0.8 | 10 |
| | 1200 | Xbridge-C18, | 0.04%, | 1 minute, to | ------- | |
| | HPLC | 50 × 2 mm × | B: CH₃CN + | 40% A in | 50 | |
| | DAD | 5 μm | 0.02% TFA | 4 min, to | | |
| | MSD | | | 15% A in 2.5 | | |
| | 6110 | | | min, back to | | |
| | | | | 100% A in | | |
| | | | | 2.0 min, held | | |
| | | | | for 0.5 min | | |
| 10 | Agilent | Waters: | A: TFA | 100% A kept | 0.6 | 10 |
| | 1200 | Xbridge-C18, | 0.04%, | 1 minute, to | ------- | |
| | HPLC | 50 × 2 mm × | B: CH₃CN + | 70% A in | 40 | |
| | DAD | 5 μm | 0.02% TFA | 4 min, to | | |
| | MSD | | | 45% A in 2.5 | | |
| | 6110 | | | min, back to | | |
| | | | | 100% A in | | |
| | | | | 2.0 min, held | | |
| | | | | for 0.5 min | | |

TABLE 3
Analytical data - LCMS: [M + H]⁺ means the
protonated mass of the free base of the compound, [M −
H]⁻ means the deprotonated mass of the free base of the compound or
the type of adduct specified [M + CH₃COO]⁻).
Rₜ means retention time (in min). For some compounds, exact
mass was determined.

| Co. | | | | LCMS |
|---|---|---|---|---|
| No. | m.p. (° C.) | [M + H]⁺ | Rₜ | Method |
| 1 | n.d. | 370 | 1.31 | 1 |
| 2 | n.d. | 370 | 1.27 | 1 |
| 3 | n.d. | 370 | 1.26 | 1 |
| 4 | n.d. | 388 | 1.60 | 1 |
| 5 | n.d. | 370 | 1.30 | 1 |
| 6 | n.d. | 372 | 1.54 | 1 |
| 7 | n.d. | 383 | 1.47 | 1 |
| 8 | n.d. | 399 | 2.22 | 1 |
| 9 | n.d. | 371 | 1.29 | 1 |
| 10 | n.d. | 371 | 1.31 | 1 |
| 11 | n.d. | 371 | 1.30 | 1 |
| 12 | n.d. | 389 | 1.65 | 1 |
| 13 | n.d. | 371 | 1.39 | 1 |
| 14 | n.d. | 386 | 1.72 | 1 |
| 15 | n.d. | 404 | 2.06 | 1 |
| 16 | n.d. | 399 | 1.93 | 1 |
| 17 | n.d. | 424 | 2.03 | 1 |
| 18 | n.d. | 442 | 2.34 | 1 |
| 19 | n.d. | 424 | 2.09 | 1 |
| 20 | n.d. | 437 | 2.23 | 1 |
| 21 | n.d. | 437.16 | 2.2283 | 1 |
| 22 | n.d. | 384.2 | 1.55 | 1 |
| 23 | n.d. | 399.3 | 2.77 | 4 |
| | | 457.2 | | |
| | | [M + CH3COO]⁻ | | |
| 23 | n.d. | 399.4 | 2.78 | 4 |
| | | 457.4 | | |
| | | [M + CH3COO]⁻ | | |
| 24 | n.d. | 399.2 | 2.76 | 4 |
| free | | 457.4 | | |
| base | | [M + CH3COO]⁻ | | |
| 24 | n.d. | 399.3 | 2.77 | 4 |
| free | | 457.3 | | |
| base | | [M + CH3COO]⁻ | | |
| 24 | n.d. | 399.18 | 1.93 | 1 |
| 25 | n.d. | 369.2 | 2.02 | 1 |
| free | | | | |
| base | | | | |
| 25 | n.d. | 369.2 | 1.84 | 1 |
| 26 | n.d. | 438.1 | 1.3 | 5 |
| | | 436 | | |
| 26 | n.d. | 438 | 1.23 | 8 |
| 27 | n.d. | 417.2 | 2.02 | 1 |
| 28 | n.d. | 384 | 1.05 | 3 |
| 29 | n.d. | 370.2 | 0.96 | 3 |
| 2C6H8O7 | | | | |
| 30 | 213.2° C. | 342.2 | 1.634 | 2 |
| | (Mettler Toledo | | | |
| | MP50) | | | |
| 31 | 278.4° C. | 341.2 | 1.24 | 2 |
| | (Mettler Toledo | | | |
| | MP50) | | | |
| 32 | n.d. | 401.2 | 1.73 | 1 |
| 32 | n.d. | 399.17 | 1.72 | 1 |
| 33 | n.d. | 402.2 | 1.8 | 1 |
| 34 | n.d. | 369.23 | 1.16 | 1 |
| 35 | n.d. | 406.2 | 2.17 | 1 |
| 36 | n.d. | 383.2 | 1.5 | 1 |
| 37 | n.d. | 386.21 | 1.79 | 1 |
| 38 | n.d. | 401.18 | 2.32 | 1 |
| 39 | n.d. | 429.2 | 2.14 | 1 |
| 40 | n.d. | 383.2 | 1.67 | 1 |
| 41 | n.d. | 356.2 | 1.28 | 1 |
| free | | | | |
| base | | | | |
| 41 | 226.7° C. | 356 | 0.948 | 2 |
| | (Mettler Toledo | | | |
| | (MP50)) | | | |
| 42 | n.d. | 410.2 | 2.21 | 1 |
| free | | | | |
| base | | | | |
| 42 | n.d. | 410.2 | 2.21 | 1 |
| 43 | n.d. | 424.2 | 2.01 | 1 |
| free | | | | |
| base | | | | |
| 43 | n.d. | 424.2 | 2.03 | 1 |
| 44 | n.d. | 356.2 | 1.61 | 1 |
| free | | | | |
| base | | | | |
| 44 | n.d. | 356.2 | 1.59 | 1 |
| 45 | n.d. | 356.2 | 1.21 | 1 |
| free | | | | |
| base | | | | |
| 45 | n.d. | 356.2 | 1.24 | 1 |
| 46 | n.d. | 356.2 | 1.66 | 1 |
| free | | | | |
| base | | | | |
| 46 | n.d. | 356.2 | 1.73 | 1 |
| 47 | n.d. | 356.2 | 1.43 | 1 |
| free | | | | |
| base | | | | |
| 47 | n.d. | 356.2 | 1.38 | 1 |
| 48 | n.d. | 442.2 | 2.88 | 1 |
| 49 | n.d. | 385.2 | 1.88 | 1 |
| 50 | n.d. | 367.18 | 1.45 | 1 |
| 51 | n.d. | 367.18 | 1.42 | 1 |
| 52 | n.d. | 378.16 | 1.82 | 1 |
| 53 | n.d. | 386.4 | 2.65 | 4 |
| | | 444.2 | | |
| | | [M + CH3COO]⁻ | | |
| 54 | n.d. | 386.2 | 2.62 | 4 |
| | | 444.2 | | |
| | | [M + CH3COO]⁻ | | |
| 55 | n.d. | 389.2 | 1.67 | 1 |
| 56 | n.d. | 368.2 | 1.67 | 1 |
| 57 | n.d. | 386.2 | 1.74 | 1 |
| free | | | | |
| base | | | | |
| 57 | n.d. | 386.2 | 1.72 | 1 |
| 58 | n.d. | 354.2 | 1.4 | 1 |
| 58 | n.d. | 354.2 | 1.41 | 1 |
| 59 | n.d. | 428.2 | 1.37 | 5 |
| 60 | n.d. | 354.3 | 0.89 | 5 |
| free | | | | |
| base | | | | |
| 60 | n.d. | 354.2 | 0.89 | 8 |
| 61 | n.d. | 354.2 | 2.2 | 4 |
| free | | 414.1 | | |
| base | | M + (CH3COO)⁻ | | |
| 61 | n.d. | 354.3 | 0.89 | 5 |
| 62 | n.d. | 354.2 | 2.2 | 4 |
| | | 414.5 | | |
| | | M + (CH3COO)⁻ | | |
| 62 | n.d. | 354.3 | 0.89 | 5 |
| 63 | n.d. | 424.2 | 2.16 | 1 |
| 64 | n.d. | 406.2 | 1.78 | 1 |
| 65 | n.d. | 422.2 | 2.16 | 1 |
| 66 | n.d. | 404.2 | 1.81 | 1 |
| 67 | n.d. | 377.1 | 1.53 | 1 |
| 67 | n.d. | 377.1 | 0.99 | 5 |
| 68 | n.d. | 410.2 | 1.92 | 1 |
| 69 | n.d. | 394.1 | 1.93 | 1 |
| 70 | n.d. | 356.2 | 1.64 | 1 |
| free | | | | |
| base | | | | |
| 70 | n.d. | 356.2 | 1.62 | 1 |
| 71 | n.d. | 370.2 | 1.89 | 1 |
| 72 | n.d. | 372.2 | 1.51 | 1 |
| 73 | n.d. | 386.2 | 1.63 | 1 |
| 74 | n.d. | 360.17 | 1.37 | 1 |
| 75 | n.d. | 384.2 | 2.652 | 10 |
| 76 | 91.71° C./ | | | |
| | −65.80 J/g | | | |
| 77 | n.d. | 357.27 | 0.8 | 5 |
| 79 | n.d. | 417.8 | 2.89 | 6 |
| | | 475.6[M + | | |
| | | CH3COO]⁻ | | |
| 79 | n.d. | 417.2 | 2.32 | 1 |
| 79 | n.d. | 417.3 | 1.37 | 5 |
| 80 | n.d. | 417.2 | 2.35 | 1 |
| 81 | n.d. | 417.2 | 2.36 | 1 |
| 82 | n.d. | | 1.8 | 1 |
| 83 | n.d. | 401.18 | 1.82 | 1 |
| 84 | n.d. | 389.2 | 1.92 | 1 |
| 85 | n.d. | 386.1 | 2.55 | 4 |
| | | 444.2 | | |
| | | [M + CH3CCO]⁻ | | |
| 86 | n.d. | 386.1 | 2.58 | 4 |
| | | 444.3 | | |
| | | M + (CH3COO)⁻ | | |
| 87 | n.d. | 389.2 | 1.97 | 1 |
| 88 | n.d. | 389.1 | 1.56 | 1 |
| free | | | | |
| base | | | | |
| 88 | n.d. | 389.2 | 1.55 | 1 |
| 89 | n.d. | 384.3 | 2.601 | 9 |
| 90 | 72.8° C. | 357.2 | 1.455 | 2 |
| | (Mettler Toledo | | | |
| | MP50) | | | |
| 91 | 134.6° C. | 375.2 | 1.545 | 2 |
| | (Mettler Toledo | | | |
| | MP50) | | | |
| 92 | n.d. | 386.2 | 1.7 | 1 |
| 93 | n.d. | 358.2 | 0.78 | 1 |
| 94 | n.d. | 374.2 | 1.1 | 3 |
| free | | | | |
| base | | | | |
| 94 | n.d. | 374.1 | 1.06 | 5 |
| free | | | | |
| base | | | | |
| 94 | n.d. | 374.2 | 1.61 | 1 |
| free | | | | |
| base | | | | |
| 94 | n.d. | 374.2 | 1.161 | 2 |
| 94 | n.d. | 374.2 | 1.61 | 1 |
| 95 | n.d. | 359.2 | 1.27 | 1 |
| 95 | n.d. | 359.2 | 1.37 | 1 |
| 95 | 127.82° C./ | 359.2 | 1.3 | 1 |
| | −228.46 J/g (A) | | | |
| 96 | n.d. | 426.2 | 2.45 | 1 |
| 97 | n.d. | 381.2 | 1.9 | 1 |
| 98 | n.d. | 372.2 | 1.12 | 1 |
| 100 | n.d. | 381.2 | 1.92 | 1 |
| 101 | n.d. | 381.2 | 1.88 | 1 |
| 102 | n.d. | 397.2 | 1.68 | 1 |
| 103 | n.d. | 397.2 | 1.75 | 1 |
| 104 | n.d. | 367.2 | 1.29 | 1 |
| 105 | n.d. | 385.2 | 1.87 | 1 |
| 106 | n.d. | 386.2 | 0.94 | 1 |
| 107 | n.d. | 368.3 | 1.36 | 7 |
| 108 | n.d. | 373.2 | 1.56 | 1 |
| 109 | n.d. | 375.2 | 1.78 | 1 |
| 110 | n.d. | 389.2 | 1.94 | 1 |
| 111 | n.d. | 375.2 | 1.66 | 1 |
| free | | | | |
| base | | | | |
| 111 | n.d. | 375.2 | 1.64 | 1 |
| 112 | 153.41° C. (two | 399.2 | 2.19 | 1 |
| | crystaline forms | | | |
| | detected. The | | | |
| | highest MP is | | | |
| | reported) (A) | | | |
| 112 | n.d. | 399.2 | 2.19 | 1 |
| 113 | 161.44° C./ | 399.2 | 2.12 | 1 |
| | −66.75 J/g (A) | | | |
| 114 | n.d. | 411.2 | 2.22 | 1 |
| 115 | n.d. | 360.2 | 1.7 | 1 |
| 116 | n.d. | 356.2 | 1.32 | 1 |
| 117 | n.d. | 378.2 | 1.77 | 1 |
| 117. | n.d. | 378.2 | 1.74 | 1 |
| HCl | | | | |
| 118 | n.d. | 368.2 | 1.56 | 1 |
| 119 | n.d. | 385.2 | 1.57 | 1 |
| 120 | n.d. | 383.2 | 1.68 | 1 |
| 121 | n.d. | 388.2 | 1.66 | 1 |
| 122 | 94.5° C. | 399.2 | 1.622 | 2 |
| | (Mettler Toledo | | | |
| | MP50) | | | |
| 123 | 139.7° C. | 487.3 | 2.105 | 2 |
| | (Mettler Toledo | | | |
| | MP50) | | | |
| 124 | 196.6° C. | 414.2 | 1.503 | 2 |
| | (Mettler Toledo | | | |
| | MP50) | | | |
| 125 | 171.4° C. | 396.1 | 1.292 | 2 |
| | (Mettler Toledo | | | |
| | MP50) | | | |
| 126 | 133.54° C. (A) | | | |
| 126 | 138° C. | 373.2 | 1.003 | 2 |
| | (Mettler Toledo | | | |
| | MP50) | | | |
| 127 | 209.19° C. (A) | | | |
| 127 | 209.9° C. | 374.2 | 1.355 | 2 |
| | (Mettler Toledo | | | |
| | MP50) | | | |
| 128 | n.d. | 399.2 | 1.889 | 2 |
| 129 | 66° C. | 384.2 | 1.22 | 2 |
| | (Mettler Toledo | | | |
| | (MP50) | | | |
| 130 | 186.4° C. | 398 | 1.33 | 2 |
| | (Mettler Toledo | | | |
| | (MP50) | | | |
| 131 | n.d. | 373 | 2.19 | 2 |
| 131 | n.d. | 372.2 | 1.59 | 1 |
| 132 | 189.8° C. | 402.2 | 1.206 | 2 |
| | (Mettler Toledo | | | |
| | (MP50) | | | |
| 133 | 250° C. | 387 | 1.19 | 2 |
| | (Mettler Toledo | | | |
| | (MP50) | | | |
| 134 | n.d. | 418 | 1.77 | 2 |
| 135 | 132.9° C. | 389 | 1.54 | 2 |
| | (Mettler Toledo | | | |
| | (MP50) | | | |
| 136 | 64.4° C. | 389 | 1.57 | 2 |
| | (Mettler Toledo | | | |
| | (MP50) | | | |
| 137 | 70.1° C. | 389 | 1.57 | 2 |
| | (Mettler Toledo | | | |
| | (MP50) | | | |
| 138 | n.d. | 388.2 | 1.085 | 2 |
| 139 | 208.1° C. | 355.2 | 1.637 | 2 |
| | (Mettler Toledo | | | |
| | MP50) | | | |
| 140 | 144.6° C. | 368 | 1.08 | 2 |
| | (Mettler Toledo | | | |
| | MP50) | | | |
| 141 | 119.6° C. | 357.2 | 1.694 | 2 |
| | (Mettler Toledo | | | |
| | MP50) | | | |
| 141 | n.d. | 357.19 | 1.44 | 1 |
| 141 | n.d. | 357.2 | 1.41 | 1 |
| 142 | 119.6° C. | 400.2 | 1.399 | 2 |
| | (Mettler Toledo | | | |
| | MP50) | | | |
| 143 | n.d. | 372.1 | 2.39 | 4 |
| 143 | n.d. | 372.2 | 2.42 | 4 |
| | | 432.1 | | |
| | | [M + CH3COO]⁻ | | |
| 143 | 105.30° C./ | 372.2 | 2.49 | 4 |
| | −75.40 J/g (A) | 430.2 | | |
| | | M + (CH3COO)⁻ | | |
| 144 | n.d. | 372.2 | 1.53 | 1 |
| 144 | n.d. | 372.3 | 1.02 | 5 |
| 145 | n.d. | 372.1 | 2.4 | 4 |
| free | | | | |
| base | | | | |
| 145 | n.d. | 372.2 | 2.43 | 4 |
| free | | 432.9 | | |
| base | | [M + CH3COO]⁻ | | |
| 145 | 103.27° C./ | 372.2 | 2.47 | 4 |
| free | −69.76 J/g (A) | 430.1 | | |
| base | | M + (CH3COO)⁻ | | |
| 145 | n.d. | 372.3 | 1.02 | 5 |
| 146 | n.d. | 424.2 | 2.14 | 1 |
| 147 | n.d. | 424.2 | 2.14 | 1 |
| 148 | 255.75° C. (A) | 388.2 | 1.62 | 1 |
| 149 | n.d. | 388.2 | 1.61 | 1 |
| 150 | 133.0° C. | 367 | 1.67 | 2 |
| | (Mettler Toledo | | | |
| | MP50) | | | |
| 151 | 168.0° C. | 357.2 | 1.346 | 2 |
| | (Mettler Toledo | | | |
| | MP50) | | | |
| 152 | n.d. | 374.2 | 1.62 | 1 |
| 152 | n.d. | 374.2 | 1.59 | 1 |
| 153 | n.d. | 415.2 | 1.97/ | 1 |
| | | | 2.03 | |
| 154 | n.d. | 368.2 | 1.27 | 1 |
| 155 | n.d. | 385.2 | 1.77 | 1 |
| 156 | 140.60 (A) | 388.2 | 2.31 | 1 |
| 157 | n.d. | 388.2 | 2.2 | 1 |
| 158 | n.d. | 428.2 | 2.62 | 1 |
| 159 | n.d. | 402.2 | 1.59 | 1 |
| 160 | n.d. | 415.2 | 1.73 | 1 |
| 161 | n.d. | 374.2 | 2.02 | 1 |
| 162 | n.d. | 374.19 | 1.59 | 1 |
| 163 | n.d. | 397.2 | 1.74 | 1 |
| 164 | n.d. | 400.2 | 2.04 | 1 |
| 165 | n.d. | 374.2 | 2.02 | 1 |
| 166 | n.d. | 388.2 | 1.83 | 1 |
| 168 | n.d. | 429.2 | 2.18 | 1 |
| 169 | n.d. | 456.2 | 2.2 | 2 |

SFCMS-Methods

General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 4
Analytical SFC-MS Methods (Flow expressed in mL/min; column temperature
(T) in ° C.; Run time in minutes, Backpressure (BPR) in bars).

| | | | | Flow | Run time |
|---|---|---|---|---|---|
| Method | | | | --------- | ------------ |
| code | column | mobile phase | gradient | Col T | BPR |
| 1 | Daicel | A: CO₂ | 20% B hold | 3.5 | 3 |
| | Chiralcel ® OJ-3 | B: MeOH | 3 min | ------- | ------- |
| | column (3 μm, | (+0.3% | | 35 | 103 |
| | 100 × 4.6 mm) | iPrNH₂) | | | |
| 2 | Daicel | A: CO₂ | 10% B hold | 3.5 | 6 |
| | Chiralpak ® AD-3 | B: iPrOH | 6 min | ------- | ------- |
| | column (3 μm, | (+0.3% | | 35 | 103 |
| | 100 × 4.6 mm) | iPrNH₂) | | | |
| 3 | Daicel | A: CO2 | 15% B hold | 3.5 | 3 |
| | Chiralcel ® OJ-3 | B: | 3 min, | ------- | ------- |
| | column (3 μm, | MeOH(+0.3% | | 35 | 103 |
| | 100 × 4.6 mm) | iPrNH2) | | | |
| 4 | Daicel | A: CO2 | 25% B hold | 3.5 | 3 |
| | Chiralcel ® OJ-3 | B: | 3 min, | ------- | ------- |
| | column (3 μm, | MeOH(+0.3% | | 35 | 103 |
| | 100 × 4.6 mm) | iPrNH2) | | | |
| 5 | Daicel | A: CO₂ | 30% B hold | 3.5 | 3 |
| | Chiralcel ® OJ-3 | B: EtOH | 3 min, | ------- | ------- |
| | column (3 μm, | (+0.3% | | 35 | 103 |
| | 100 × 4.6 mm) | iPrNH₂) | | | |
| 6 | Daicel | A: CO₂ | 25% B hold | 3.5 | 3 |
| | Chiralcel ® OJ-3 | B: EtOH | 3 min, | ------- | ------- |
| | column (3 μm, | (+0.3% | | 35 | 103 |
| | 100 × 4.6 mm) | iPrNH₂) | | | |
| 7 | Daicel | A: CO₂ | 20% B hold | 3.5 | 3 |
| | Chiralpak ® AD-3 | B: EtOH | 3 min | ------- | ------- |
| | column (3 μm, | (+0.3% | | 35 | 103 |
| | 100 × 4.6 mm) | iPrNH₂) | | | |
| 8 | Daicel | A: CO₂ | 25% B hold | 3.5 | 3 |
| | Chiralcel ® OD-3 | B: IPOH | 3 min | ------- | -------- |
| | column (3 μm, | (+0.3% | | 35 | 103 |
| | 100 × 4.6 mm) | iPrNH₂) | | | |
| 9 | Daicel | A: CO₂ | 15% B hold | 3.5 | 3 |
| | Chiralcel ® OJ-3 | B: | 3 min, | ------- | ------- |
| | column (3μm, | EtOH(+0.3% | | 35 | 103 |
| | 100 × 4.6 mm) | iPrNH₂) | | | |

TABLE 5
Analytical SFC data - Rₜ means retention time (in
minutes), [M + H]⁺ means the protonated
mass of the compound, method refers to the method
used for (SFC)MS analysis of enantiomerically pure compounds.

| | | | | | Isomer |
|---|---|---|---|---|---|
| | | | | | Elution |
| Co. No. | Rₜ | [M + H]⁺ | UV Area % | Method | Order |
| 2 | 1.05 | 370 | 100 | 1 | A |
| 3 | 1.34 | 370 | 100 | 1 | B |
| 10 | 3.06 | 371 | 100 | 2 | A |
| 11 | 3.38 | 371 | 100 | 2 | B |
| 23 | 1.20, | 399 | 100 | 5 | A |
| | 1.73 | | | | |
| 23 | 1.29, | 399 | 100 | 5 | A |
| | 1.87 | | | | |
| 24 free | 1.20, | 399 | 100 | 5 | B |
| base | 1.73 | | | | |
| 24 free | 1.29, | 399 | 100 | 5 | B |
| base | 1.88 | | | | |
| 53 | 1.15, | 386 | 100 | 6 | A |
| | 1.58 | | | | |
| 54 | 1.14, | 386 | 99.24 | 6 | B |
| | 1.58 | | | | |
| 60 | 1.05, | 354 | 49.77, | 7 | |
| | 1.34 | | 50.23 | | |
| 61 free | 1.05, | 354 | 100 | 7 | A |
| base | 1.34 | | | | |
| 62 | 1.06, | 354 | 99.02 | 7 | B |
| | 1.34 | | | | |
| 79 | 1.23, | 417 | 50.00, | 8 | |
| | 1.65 | | 50.00 | | |
| 85 | 1.05, | 386 | 100 | 4 | A |
| | 1.46 | | | | |
| 86 | 1.05, | 386 | 100 | 4 | B |
| | 1.46 | | | | |
| 143 | 0.84, | 372 | 100 | 3 | A |
| | 1.08 | | | | |
| 143 | 1.10, | 372 | 100 | 9 | A |
| | 1.56 | | | | |
| 143 | 1.14, | 372 | 100 | 9 | A |
| | 1.64 | | | | |
| 145 free | 0.84, | 372 | 100 | 3 | B |
| base | 1.08 | | | | |
| 145 free | 1.12, | 372 | 100 | 9 | B |
| base | 1.55 | | | | |
| 145 free | 1.16, | 372 | 99.81 | 9 | B |
| base | 1.62 | | | | |

NMR

For a number of compounds, ¹H NMR spectra were recorded on a Bruker DPX-400 spectrometer operating at 400 MHz, on a Bruker Avance I operating at 500 MHz, using CHLOROFORM-d (deuterated chloroform, CDCl₃) or DMSO-d₆ (deuterated DMSO, dimethyl-d6 sulfoxide) as solvent. Chemical shifts (6) are reported in parts per million (ppm) relative to tetramethylsilane (TMS), which was used as internal standard.

TABLE 6
¹H NMR results

| Co. | |
|---|---|
| No. | ¹H NMR result |
| 4 | 1H NMR (500 MHz, CHLOROFORM-d) δ ppm 1.45 (d, J = 6.94 Hz. 3H), |
| | 1.68-1.86 (m, 2H), 1.92-2.04 (m, 2H), 2.43 (s, 6H), 2.80-2.96 |
| | (m, 2H), 4.09 (qd, J = 6.94, 1.45 Hz, 1H), 4.19-4.32 (m, 3H) 4.37- |
| | 4.52 (m, 2H) 6.43 (s, 2H) 6.97 (d, J = 9.25 Hz, 1H). |
| 144 | 1H NMR (400 MHz, DMSO-d6) δ ppm 1.49-1.84 (m, 3H) 1.97-2.36 |
| | (m, 4H) 2.56-2.69 (m, 7H) 2.96-3.19 (m, 2H) 3.22-3.74 (m, 5H) |
| | 4.50-5.20 (m, 4H) 7.21-7.44 (m, 2H) 7.68-7.92 (m, 1H) 10.76- |
| | 11.59 (m, 1H) 14.97-15.39 (m, 1H) |
| 148 | 1H NMR (500 MHz, DMSO-d6) δ ppm 1.57-1.63 (m, 3H) 1.66 (d, |
| | J = 6.65 Hz, 3H) 1.95-2.33 (m, 4H) 2.58-2.64 (m, 6H) 2.99 (bd d, |
| | J = 8.38 Hz, 1H) 3.05-3.17 (m, 1H) 3.26 (br d J = 12.72 Hz, 1H) 3.51- |
| | 3.70 (m, 2H) 4.27-4.41 (m, 2H) 4.42-4.54 (m, 2H) 4.76 (br d, |
| | J = 6.36 HZ, 1H) 4.81-4.89 (m, 1H) 5.09 (br s, 1H) 7.30 (s, 1H) 7.33 |
| | (s, 1H) 7.54-7.64 (m 1H) 7.68-7.92 (m, 1H) 10.53-10.86 (m, 1H) |
| | 11.14 (br d, J = 8.38 Hz, 1H) 14.75-15.20 (m, 1H) |
| 83 | 1H NMR (500 MHz, DMSO-d6) δ ppm 1.67-1.86 (m, 3H) 2.08 (br d, |
| | J = 11.56 Hz, 1H) 2.26 (br s, 2H) 2.57-2.67 (m, 7H) 2.90 (s, 3H) 3.03 |
| | (br s, 1H) 3.16 (br d, J = 10.69 Hz, 1H) 3.25 (br s, 1H) 3.60-3.83 (m, |
| | 2H) 4.72-4.93 (m, 1H) 4.98-5.19 (m, 1H) 7.31 (s, 2H) 8.55-8.87 |
| | (m 1H) 11.09 (br s, 1H) 11.59 (br s, 1H) 14.84-15.38 (m, 1H) |
| 121 | 1H NMR (400 MHz, CHLOROFORM-d) δ ppm 1.38 (d, J = 6.94 Hz, 3H) |
| | 1.76-1.89 (m, 2H) 1.99 (br s, 2H) 2.41-2.45 (m, 6H) 2.76-2.92 |
| | (m, 2H) 3.58 (q, J = 6.70 Hz, 1H) 4.23-4.27 (m, 2H) 4.32 (tt, J = 8.12, |
| | 3.90 Hz, 1H) 4.44 (tt, J = 3.76, 2.17 Hz, 2H) 6.54 (d, J = 5.78 Hz 1H) |
| | 6.96 (d, J = 8.09 Hz 1H) 7.16 (d, J = 8.09 Hz 1H) |
| 133 | 1H NMR (300 MHz, CHLOROFORM-d) δ ppm 1.41 (d, J = 6.9 Hz, 3H) |
| | 1.73-1.49 (m, 2H) 1.91 (s, 2H) 2.17 (dd, J = 13.4, 8.7 Hz, 2H) 2.46 (s, |
| | 6H) 3.00 (dd, J = 16.3, 12.7 Hz, 2H) 3.20 (dt, J = 10.3, 8.8 Hz, 1H) 4.13- |
| | 3.99 (m, 1H) 4.29-4.18 (m, 2H) 4.41 (d, J = 3.1 Hz, 2H) 6.33 (s, 2H) |
| | 6.69 (s, 1H) 6.95 (d, J = 9.1 Hz, 1H) |
| 95 | 1H NMR (400 MHz, CHLOROFORM-d) δ ppm 1.34-1.43 (m, 3H) |
| | 1.62-1.80 (m, 2H) 1.85-2.02 (m, 2H) 2.23-2.40 (m, 2H) 2.52- |
| | 2.64 (m, 3H) 2.74-2.89 (m, 2H) 3.14-3.25 (m, 2H) 3.98-4.08 (m, |
| | 1H) 4.10-4.19 (m, 1H) 4.51-4.70 (m, 3H) 7.12-7.17 (m, 1H) 8.07- |
| | 8.28 (m, 2H) 11.87 (br d, J = 5.09 Hz, 1H) 14.92-15.29 (m, 1H) |
| 58 | 1H NMR (400 MHz, DMSO-d6) δ ppm 1.60-1.83 (m, 3H) 1.97-2.41 |
| | (m, 4H) 2.56-2.66 (m, 6H) 2.70-3.13 (m, 2H) 3.25-3.83 (m, 4H) |
| | 4.51-5.23 (m, 4H) 7.19-7.81 (m, 1H) 8.10-8.28 (m, 1H) |
| 135 | 1H NMR (400 MHz, CHLOROFORM-d) δ ppm 1.43 (d, J = 6.5 Hz, 3H) |
| | 1.71-1.55 (m, 2H) 1.88 (d, J = 12.8 Hz, 2H) 2.21 (s, 2H) 2.71 (s, 3H) |
| | 2.87 (s, 2H) 3.30 (s, 1H) 4.07 (d, J = 5.5 Hz, 1H) 4.31-4.20 (m, 2H) |
| | 4.41 (dd, J = 8.3, 4.0 Hz, 2H) 4.45 (s, 2H) 6.95 (d, J = 9.1 Hz, 1H) |
| | 8.56 (s, 2H) |
| 165 | 1H NMR (400 MHz, DMSO-d6) δ ppm 1.52-1.71 (m, 3H) 2.20 (m, 6H) |
| | 2.96-3.15 (m, 2H) 3.23 (br d, J = 11.79 Hz, 1H) 3.41-3.64 (m, 2H) |
| | 4.26-4.40 (m, 2H) 4.40-4.52 (m, 2H) 4.62-4.81 (m, 1H) 5.00- |
| | 5.09 (m, 2H) 7.42-7.69 (m, 2H) 7.83-8.09 (m, 1H) 10.54-11.18 |
| | (m, 1H) |
| 159 | 1H NMR (400 MHz, DMSO-d6) δ ppm 1.62 (dd, J = 6.70, 3.47 Hz, 3H) |
| | 1.76-2.26 (m, 4H) 2.72 (s, 6H) 2.81-2.95 (m, 1H) 2.99-3.15 (m, 1H) |
| | 3.21 (br d, J = 10.63 Hz, 1H) 3.41-3.49 (m, 1H) 3.82 (br s, 1H) |
| | 4.33 (dd, J = 4.05, 2.66 Hz, 1H) 4.45 (br d, J = 3.24 Hz 2H) 4.60-4.82 |
| | (m, 3H) 7.56 (dd, J = 9.71, 8.55 Hz, 1H) 7.59 (s, 2H) 10.30-11.33 (m, |
| | 1H) 15.51-16.58 (m, 1H) |
| 154 | 1H NMR (400 MHz, CHLOROFORM-d) δ ppm 1.38 (d, J = 6.70 Hz, 3H) |
| | 1.62-1.74 (m, 2H) 1.92 (br d, J = 3.47 Hz, 2H) 2.13-2.26 (m, 2H) 2.51 |
| | (s, 6H) 2.76-2.85 (m, 1H) 2.87-2.95 (m, 1H) 3.19-3.26 |
| | (m, 2H) 3.30-3.38 (m, 1H) 3.54 (q, J = 7.09 Hz, 1H) 4.45 (s, 2H) |
| | 4.62 (t, J = 8.67 Hz, 2H) 6.87 (d, J = 7.40 Hz, 1H) 6.92 (s, 2H) 7.43 (d, |
| | J = 7.40 Hz, 1H) |

### Pharmacological Examples

1) OGA-Biochemical Assay

The assay is based on the inhibition of the hydrolysis of fluorescein mono-B-D-N-Acetyl-Glucosamine (FM-GcNAc) (Mariappa et al. 2015, Biochem J470:255) by the recombinant human Meningioma Expressed Antigen 5 (MGEA5), also referred to as O-GlcNAcase (OGA). The hydrolysis FM-GlcNAc (Marker Gene technologies, cat #M1485) results in the formation of B-D-N-glucosamineacetate and fluorescein. The fluorescence of the latter can be measured at excitation wavelength 485 nm and emission wavelength 538 nm. An increase in enzyme activity results in anincrease in fluorescence signal. Full length OGA enzyme was purchased at OriGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%.

Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting 0-GlcNAcylated residues as previously described (Dorfmueller et al. 2010 Chemistry & biology, 17:1250). Inhibition of OGA will result in an increase of 0-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100p of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50p and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 7
Results in the biochemical and cellular assays,

| | Enzymatic | | Cellular | |
|---|---|---|---|---|
| | hOGA; | Enzymatic | hOGA; | Cellular |
| Co. No. | pIC₅₀ | Eₘₐₓ (%) | pEC₅₀ | Eₘₐₓ (%) |
| 1 | 7.2 | 101 | | |
| 2 | <5 | 29 | <6 | −14 |
| 3 | 7.3 | 103 | 6.6 | 83 |
| 4 | 7.9 | 99 | 7.4 | 76 |
| 5 | 5.9 | 93 | <6 | 22 |
| 6 | 8.4 | 101 | 7.76 | 87 |
| 7 | 7.2 | 100 | 6.4 | 71 |
| 8 | 7.8 | 100 | 7.2 | 89 |
| 9 | 7.0 | 102 | | |
| 10 | 7.3 | 100 | 6.2 | 73 |
| 11 | 5.6 | 84 | | |
| 12 | 7.8 | 99 | 7.1 | 74 |
| 13 | 6.3 | 96 | <6 | 8 |
| 14 | 7.7 | 100 | 6.9 | 82 |
| 15 | 8.3 | 102 | 7.3 | 72 |
| 16 | 7.6 | 99 | 6.9 | 91 |
| 17 | 7.4 | 100 | 6.5 | 61 |
| 18 | 8.2 | 101 | 7.2 | 79 |
| 19 | 6.0 | 92 | <6 | 17 |
| 21 | 7.3 | 101 | 6.09 | 55 |
| 22 | 7.0 | 100 | <6 | 37 |
| 23 | 5.4 | 59 | <6 | −8 |
| 24 | 7.7 | 98 | 7.0 | 72 |
| 25 | 6.3 | 96 | 6.2 | 56 |
| 27 | 7.0 | 99 | 6.3 | 76 |
| 28 | 8.0 | 101 | 6.9 | 68 |
| 29 | 5.3 | 60 | <6 | 7 |
| 30 | 6.1 | 100 | <6 | 15 |
| 31 | 6.1 | 96 | <6 | 21 |
| 32 | 7.8 | 100 | 7.1 | 72 |
| 33 | 7.8 | 100 | 6.6 | 65 |
| 34 | 6.8 | 102 | 6.8 | 77 |
| 35 | 8.1 | 101 | 7.6 | 98 |
| 36 | 5.9 | 90 | <6 | 7 |
| 37 | 6.4 | 98 | <6 | 23 |
| 38 | 7.8 | 101 | 6.6 | 64 |
| 39 | 7.7 | 101 | 6.5 | 73 |
| 40 | 6.9 | 101 | 6.2 | 59 |
| 41 | 7.3 | 99 | 6.3 | 74 |
| 42 | 7.0 | 101 | <6 | 21 |
| 43 | 6.3 | 96 | <6 | 25 |
| 44 | 6.7 | 98 | 6.3 | 57 |
| 45 | 7.0 | 96 | 6.2 | 59 |
| 46 | 6.8 | 98 | <6 | 33 |
| 47 | 7.2 | 99 | 6.5 | 67 |
| 48 | 7.7 | 98 | 6.6 | 84 |
| 49 | 7.7 | 99 | 6.54 | 81 |
| 50 | 6.0 | 95 | <6 | 9 |
| 51 | 7.2 | 100 | 6.2 | 60 |
| 52 | 6.3 | 96 | <6 | 16 |
| 53 | 5.1 | 55 | <6 | −8 |
| 54 | 7.9 | 100 | 6.9 | 83 |
| 55 | 6.7 | 97 | <6 | 14 |
| 56 | 6.5 | 98 | 6.4 | 74 |
| 57 | 7.4 | 97 | 6.9 | 89 |
| 58 | 5.9 | 89 | 6.0 | 38 |
| 60 | 7.4 | 95 | 6.93 | 84 |
| 61 | 7.8 | 95 | 7.1 | 87 |
| 62 | 6.0 | 90 | <6 | 6 |
| 63 | 8.0 | 96 | 7.1 | 80 |
| 64 | 7.5 | 94 | 6.5 | 61 |
| 65 | 7.9 | 99 | 7.2 | 101 |
| 66 | 7.1 | 96 | 6.6 | 73 |
| 67 | 6.9 | 102 | 6.2 | 60 |
| 68 | 7.1 | 94 | 6.1 | 57 |
| 69 | 7.1 | 95 | 6.3 | 68 |
| 70 | 6.4 | 98 | <6 | 46 |
| 71 | 6.9 | 100 | <6 | 35 |
| 72 | 7.5 | 101 | 6.6 | 60 |
| 73 | 5.7 | 90 | <6 | 18 |
| 74 | 7.0 | 99 | <6 | 44 |
| 75 | 6.4 | 99 | <6 | 39 |
| 76 | 7.1 | 99 | <6 | 36 |
| 77 | 6.7 | 97 | <6 | 35 |
| 78 | 7.0 | 99 | 6.09 | 55 |
| 79 | 8.5 | 101 | 7.4 | 72 |
| 80 | 6.2 | 96 | <6 | 7 |
| 81 | 8.8 | 93 | 7.5 | 87 |
| 82 | 6.2 | 97 | <6 | 18 |
| 83 | 8.3 | 101 | 7.5 | 86 |
| 84 | 7.8 | 101 | 6.5 | 73 |
| 85 | <5 | −11 | <6 | −3 |
| 86 | 7.8 | 94 | 7.0 | 100 |
| 87 | 7.8 | 97 | 6.45 | 62 |
| 88 | 7.0 | 98 | 6.3 | 59 |
| 89 | 6.9 | 97 | ~6.3 | 63 |
| 90 | 7.0 | 100 | <6 | 48 |
| 91 | 7.8 | 105 | 6.7 | 86 |
| 92 | 7.6 | 97 | 6.9 | 66 |
| 93 | 6.6 | 98 | <6 | 34 |
| 94 | 8.0 | 96 | 7.02 | 82 |
| 95 | 8.3 | 96 | 7.4 | 92 |
| 96 | 7.2 | 99 | <6 | 41 |
| 97 | 6.4 | 94 | <6 | 20 |
| 98 | 6.7 | 94 | 6.3 | 47 |
| 99 | 6.8 | 98 | <6 | 34 |
| 100 | 7.4 | 102 | <6 | 47 |
| 101 | 7.0 | 94 | 6.3 | 53 |
| 102 | 6.3 | 95 | <6 | 10 |
| 103 | 7.1 | 97 | 6.0 | 52 |
| 104 | 6.2 | 95 | <6 | 12 |
| 105 | 6.4 | 97 | <6 | 16 |
| 106 | 6.9 | 95 | 6.2 | 48 |
| 107 | 6.8 | 96 | 6.2 | 51 |
| 108 | 6.2 | 101 | <6 | 15 |
| 109 | 7.9 | 99 | 6.7 | 77 |
| 110 | 8.1 | 101 | 7.0 | 79 |
| 111 | 7.3 | 98 | 6.7 | 77 |
| 112 | 8.0 | 96 | 6.8 | 87 |
| 113 | 7.8 | 96 | 6.9 | 83 |
| 114 | 6.5 | 97 | <6 | 31 |
| 115 | 6.7 | 96 | <6 | 25 |
| 116 | 6.9 | 96 | <6 | 42 |
| 118 | 6.9 | 99 | 6.0 | 42 |
| 119 | 7.2 | 100 | 6.48 | 68 |
| 120 | 7.3 | 98 | 6.6 | 87 |
| 121 | 7.0 | 96 | 6.4 | 59 |
| 122 | 7.1 | 100 | 6.1 | 48 |
| 123 | 7.9 | 93 | 7.0 | 59 |
| 124 | 7.5 | 98 | 7.0 | 64 |
| 125 | 7.5 | 96 | 6.9 | 91 |
| 126 | 7.6 | 99 | 7.2 | 73 |
| 127 | 7.7 | 98 | 6.87 | 81 |
| 128 | 8.1 | 102 | 7.3 | 95 |
| 129 | 7.2 | 97 | 6.9 | 83 |
| 130 | 7.4 | 100 | 6.61 | 59 |
| 131 | 5.4 | 70 | <6 | −4 |
| 132 | 5.2 | 63 | <6 | 24 |
| 133 | 7.6 | 97 | 7.3 | 87 |
| 134 | 8.0 | 94 | 7.2 | 81 |
| 135 | 7.5 | 97 | 7.1 | 97 |
| 136 | 7.9 | 95 | 7.3 | 81 |
| 137 | 6.4 | 96 | <6 | 25 |
| 138 | 7.7 | 100 | 6.8 | 79 |
| 139 | 6.7 | 95 | <6 | 31 |
| 140 | 6.4 | 98 | 6.6 | 64 |
| 141 | 6.7 | 97 | 6.1 | 51 |
| 142 | 7.6 | 104 | 6.8 | 73 |
| 143 | 8.3 | 99 | 7.76 | 100 |
| 144 | 8.5 | 97 | 8.0 | 81 |
| 145 | 5.8 | 86 | <6 | 8 |
| 146 | 5.2 | 58 | <6 | 2 |
| 147 | 7.6 | 96 | 6.7 | 81 |
| 148 | 8.2 | 98 | 7.3 | 82 |
| 149 | 5.2 | 58 | <6 | −2 |
| 150 | 7.1 | 95 | 6.4 | 57 |
| 151 | 6.7 | 98 | <6 | 30 |
| 152 | 7.0 | 93 | 6.0 | 42 |
| 153 | 7.5 | 95 | 6.7 | 67 |
| 154 | 7.4 | 94 | 6.8 | 81 |
| 155 | 7.8 | 94 | 7.1 | 67 |
| 156 | 7.7 | 102 | 6.7 | 67 |
| 157 | 7.7 | 94 | 6.8 | 80 |
| 158 | 7.8 | 99 | 6.6 | 77 |
| 159 | 7.9 | 94 | 7.2 | 75 |
| 160 | 7.9 | 96 | 7.4 | 87 |
| 161 | 7.7 | 97 | 6.8 | 83 |
| 162 | 7.9 | 98 | 7.2 | 83 |
| 163 | 6.9 | 92 | <6 | 34 |
| 164 | 8.1 | 96 | 7.17 | 72 |
| 165 | 7.8 | 94 | 6.5 | 72 |
| 166 | 8.0 | 98 | 7.2 | 85 |
| 168 | 7.9 | 96 | 6.7 | 75 |
| 169 | 7.9 | 96 | 7.29 | 87 |

n.d. means not determined.

3) Ex Vivo OGA Occupancy Assay Using [3H]-Ligand Drug Treatment and Tissue Preparation

Male NMRI or C57Bl6j mice were treated by oral (p.o.) administration of vehicle or compound. Animals were sacrificed 24 hours after administration. Brains were immediately removed from the skull, hemispheres were separated and the right hemisphere, for ex vivo OGA occupancy assay, was rapidly frozen in dry-ice cooled 2-methylbutane (−40° C.). Twenty m-thick sagittal sections were cut using a Leica CM 3050 cryostat-microtome (Leica, Belgium), thaw-mounted on microscope slides (SuperFrost Plus Slides, Thermo Fisher Scientific) and stored at −20° C. until use. After thawing, sections were dried under a cold stream of air. The sections were not washed prior to incubation. The 10 minutes incubation with 3 nM [³H]-ligand was rigorously controlled. All brain sections (from compound-treated and vehicle-treated animals) were incubated in parallel. After incubation, the excess of [³H]-ligand was washed off in ice-cold buffer (PBS 1× and 1% BSA) 2 times 10 minutes, followed by a quick dip in distilled water. The sections were then dried under a stream of cold air.

QUANTITATIVE AUTORADIOGRAPHY AND DATA ANALYSIS Radioactivity in the forebrain area of brain slices was measured using a P-imager with M3 vision analysis software (Biospace Lab, Paris). Specific binding was calculated as the difference between total binding and non-specific binding measured in Thiamet-G (10 μM) treated sections. Specific binding in sections from drug treated animals was normalised to binding in sections from vehicle treated mice to calculate percentage of OGA occupancy by the drug.

| Co. | Time | Dose | Occupancy | |
|---|---|---|---|---|
| No. | (h) | (mg/kg) | (% +/− sd) | Experiment |
| 144 | 24 | 25 | 94.67 +/− 4.04 | n/a |
| 148 | 24 | 25 | 84.33 +/− 3.06 | 1 |
| 148 | 24 | 25 | 77.67 +/− 10.5 | 2 |
| 83 | 24 h | 25 mg/kg | 97 +/− 1.53 | n/a |
| 95 | 24 h | 25 mg/kg | 20 +/− 17.93 | n/a |

1. A compound of Formula (I) 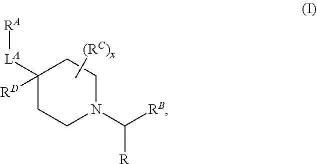
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl;
or is phenyl; each of which may be optionally substituted with 1, 2 or 3 substituents, in particular 2 substituents, each independently selected from the group consisting of halo;
cyano; OH; C₁₋₄alkyl optionally substituted with 1, 2, or 3 independently selected halo substituents; C₃₋₆cycloalkyl; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —CH₂—, —O—, —OCH₂—, —CH₂O—, —NH—, —N(CH₃)—, —NHCH₂— and —CH₂NH—;
R is H or CH₃; and
R^{B} is an aromatic heterobicyclic radical selected from the group consisting of (b-1) to (b-6) 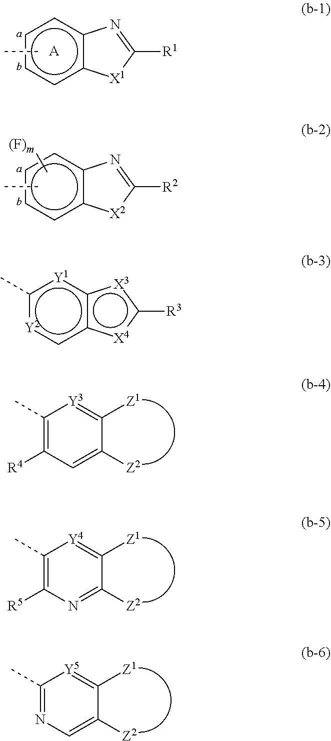
wherein
a and b represent the position of attachment to CHR;
ring A represents a 6-membered aromatic ring optionally having one Nitrogen atom;
X¹ and X² each represent S or O;
m represents 1 or 2;
Y¹ and Y² are each independently selected from N and CF; with the proviso that when Y¹ is N, Y² is CF, and when Y¹ is CF, Y² is N;
X³ and X⁴ are each independently selected from N, S and O; with the proviso that when X³ is N then X⁴ is S or O, and when X⁴ is N then X³ is S or O;
Y³, Y⁴ and Y⁵ each represent CH, CF or N;
—Z¹-Z²— forms a bivalent radical selected from the group consisting of
—O(CH₂)ₙO— (c-1);
—O(CH₂)ₚ— (c-2);
—(CH₂)ₚO— (c-3);
wherein
n represents 1 or 2;
p represents 2 or 3;
R¹, R², and R³ are each selected from C₁₋₄alkyl;
R⁴ and R⁵ are each selected from the group consisting of hydrogen, fluoro and methyl;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
x represents 0, 1 or 2;
with the provisos that
a) R^{C} is not hydroxy or methoxy when present at the carbon atom adjacent to the nitrogen atom of the piperidinediyl ring;
b) R^{C} and R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;
or a pharmaceutically acceptable addition salt or a solvate thereof.

2. The compound according to claim 1, wherein.
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents.

3. The compound according to claim 1, wherein
L^{A} is selected from the group consisting of —CH₂—, —O—, —OCH₂—, —CH₂O—, —NH—, —N(CH₃)—, —NHCH₂— and —CH₂NH—.

4. The compound of claim 1, wherein
R^{B} is an aromatic heterobicyclic radical selected from the group consisting of (b-1), (b-2), (b-3), (b-4) and (b-5).

5. The compound according to any one of claim 1, wherein
R^{B} is an aromatic heterobicyclic radical selected from the group consisting of (b-3) and (b-4); wherein —Z¹-Z²— forms a bivalent radical selected from the group consisting of (c-1) and (c-2), wherein n and p each represent 2; and wherein Y¹ is N, Y² is CF, and R³ is C₁₋₄alkyl.

6. The compound of claim 1, wherein
R^{B} is an aromatic heterobicyclic radical selected from the group consisting of

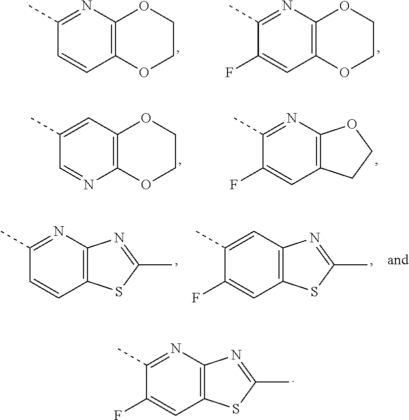

7. The compound of claim 1, wherein x is 0 or 1; and R^{C} when present, is fluoro or methyl.

8. The compound of claim 1, wherein x is 0.

9. The compound of claim 1, wherein R^{D} is hydrogen.

10. A pharmaceutical composition comprising a prophylactically or a therapeutically effective amount of a compound of claim 1 and a pharmaceutically acceptable carrier.

11. (canceled)

12. (canceled)

13. (canceled)

14. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound of claim 1.

15. (canceled)
